(19) United States
(12) Patent Application Publication
(10) Pub. No.: US 2026/0365666 A1
(43) Pub. Date: Nov. 19, 2026

(54) ORGANIC ELECTROLUMINESCENT COMPOUND AND ORGANIC ELECTROLUMINESCENT DEVICE COMPRISING THE SAME

(71) Applicant: DuPont Specialty Materials Korea Ltd., Chungcheongnam-do (KR)

(72) Inventors: Ji-Song JUN, Gyeonggi-do (KR); Su-Hyun LEE, Gyeonggi-do (KR); Sang-Hee CHO, Gyeonggi-do (KR); Dong-Hyung LEE, Gyeonggi-do (KR)

(21) Appl. No.: 19/725,643

(22) Filed: Jun. 30, 2026

Related U.S. Application Data
(63) Continuation of application No. 17/406,741, filed on Aug. 19, 2021, now abandoned.

(30) Foreign Application Priority Data
Aug. 24, 2020 (KR) 10-2020-0106142

(51) Int. Cl. C07D 493/00; C07D 209/82; C07D 495/00; C09K 11/06; H10K 50/11; H10K 85/60; H10K 101/40

(52) U.S. Cl. CPC C07D 493/00; C07D 209/82; C07D 495/00; C09K 11/06; H10K 85/622; H10K 85/626; H10K 85/633; H10K 85/636; H10K 85/6572; H10K 85/6574; H10K 85/6576; C09K 2211/1018; H10K 50/11; H10K 2101/40

(57) ABSTRACT

The present disclosure relates to an organic electroluminescent compound and an organic electroluminescent device comprising the same. By comprising the organic electroluminescent compound according to the present disclosure in a hole transport zone, it is possible to produce an organic electroluminescent device having improved luminous efficiency and/or lifespan characteristics.

### CLAIM OF BENEFIT OF PRIOR APPLICATION

This application is a continuation of U.S. patent application Ser. No. 17/406,741, filed Aug. 19, 2021, which is incorporated by reference herein in its entirety.

### TECHNICAL FIELD

The present disclosure relates to an organic electroluminescent compound and an organic electroluminescent device comprising the same.

### BACKGROUND ART

A small molecular green organic electroluminescent device (OLED) was first developed by Tang, et al., of Eastman Kodak in 1987 by using TPD/ALq3 bi-layer consisting of a light-emitting layer and a charge transport layer. Thereafter, the development of OLEDs was rapidly effected and OLEDs have been commercialized. At present, OLEDs primarily use phosphorescent materials having excellent luminous efficiency in panel implementation. In many applications such as TV and lighting, the problem that lifespan of OLEDs is insufficient is encountered, and high efficiency of OLEDs is still required. Generally, the higher the luminance of OLEDs is, the shorter the lifespan of the OLEDs is. Therefore, there is a need for OLEDs having high luminous efficiency and/or long lifespan for long-term use and high resolution of displays.

In order to enhance luminous efficiency, driving voltage and/or lifespan, various materials or concepts for an organic layer of an OLED have been proposed. However, they were not satisfactory in practical use.

Meanwhile, US Patent Application Publication No. 2018/0287062 discloses the following compound as a host.

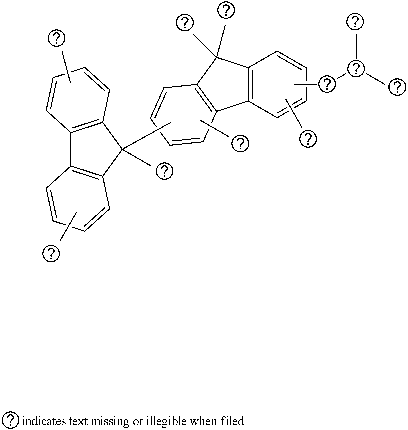

In addition, CN Patent Application Publication No. 108864013 discloses the following compound as a host.

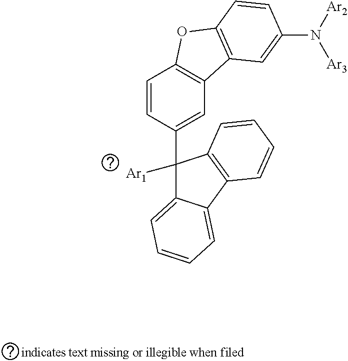

However, there is a need to develop a compound having more improved performance, such as improved driving voltage, luminous efficiency, and/or lifespan characteristics compared to the specific compounds disclosed in the aforementioned references.

### DISCLOSURE OF INVENTION

### Technical Problem

An objective of the present disclosure is to provide an organic electroluminescent compound having a novel structure suitable for applying it to organic electroluminescent devices. Another objective of the present disclosure is to provide an improved organic electroluminescent material which can provide an organic electroluminescent device having improved luminous efficiency and/or lifespan properties. Still another objective of the present disclosure is to provide an organic electroluminescent device having improved luminous efficiency and/or lifespan characteristics by including the compound according to the present disclosure in a hole transport zone.

### Solution to Problem

The present inventors found that the above objective can be achieved by a compound represented by the following formula 1.

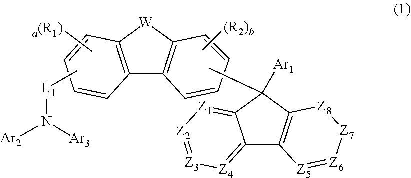

In formula 1,
W represents O, S or —N-L₂-R₃;
Z₁ to Z₃ each independently represent CR₄ or N;
L₁ and L₂ each independently represent a single bond, a substituted or unsubstituted (C6-C30)arylene, or a substituted or unsubstituted (3- to 30-membered)heteroarylene;
Ar₁ to Ar₃ each independently represent a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted fused ring group of a (C3-C30) aliphatic ring(s) and a (C6-C30) aromatic ring(s), a substituted or unsubstituted mono- or di-(C1-C30)alkylamino, a substituted or unsubstituted mono- or di-(C2-C30)alkenylamino, a substituted or unsubstituted mono- or di-(C6-C30)arylamino, a substituted or unsubstituted mono- or di-(3- to 30-membered)heteroarylamino, a substituted or unsubstituted (C1-C30)alkyl(C2-C30)alkenylamino, a substituted or unsubstituted (C1-C30)alkyl(C6-C30)arylamino, a substituted or unsubstituted (C1-C30)alkyl(3- to 30-membered)heteroarylamino, a substituted or unsubstituted (C2-C30)alkenyl(C6-C30)arylamino, a substituted or unsubstituted (C2-C30)alkenyl(3- to 30-membered)heteroarylamino, or a substituted or unsubstituted (C6-C30)aryl(3- to 30-membered)heteroarylamino;
R₁ to R₄ each independently represent hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted fused ring group of a (C3-C30) aliphatic ring(s) and a (C6-C30) aromatic ring(s), a substituted or unsubstituted mono- or di-(C1-C30)alkylamino, a substituted or unsubstituted mono- or di-(C2-C30)alkenylamino, a substituted or unsubstituted mono- or di-(C6-C30)arylamino, a substituted or unsubstituted mono- or di-(3- to 30-membered)heteroarylamino, a substituted or unsubstituted (C1-C30)alkyl(C2-C30)alkenylamino, a substituted or unsubstituted (C1-C30)alkyl(C6-C30)arylamino, a substituted or unsubstituted (C1-C30)alkyl(3- to 30-membered)heteroarylamino, a substituted or unsubstituted (C2-C30)alkenyl(C6-C30)arylamino, a substituted or unsubstituted (C2-C30)alkenyl(3- to 30-membered)heteroarylamino, or a substituted or unsubstituted (C6-C30)aryl(3- to 30-membered)heteroarylamino; or two or more of the adjacent R₁ to R₄ may be linked to each other to form a ring(s);
a and b are each independently an integer of 1 to 3; and
if a plurality of each of R₁, R₂ and R₄ are present, each of R₁, each of R₂, and each of R₄ may be the same or different;
provided that when W is O, the following structure is excluded.

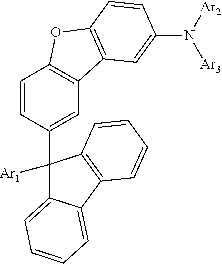

### Advantageous Effects of Invention

An organic electroluminescent compound according to the present disclosure exhibits performances suitable for using it in an organic electroluminescent device. In addition, by comprising the organic electroluminescent compound according to the present disclosure in a hole transport zone, an organic electroluminescent device having improved luminous efficiency and/or lifespan characteristics compared to conventional organic electroluminescent devices is provided.

### MODE FOR THE INVENTION

Hereinafter, the present disclosure will be described in detail. However, the following description is intended to explain the present disclosure, and is not meant to restrict the scope of the present disclosure.

The term “organic electroluminescent compound” in the present disclosure means a compound that may be used in an organic electroluminescent device, and may be comprised in any layer constituting an organic electroluminescent device, as necessary.

The term “organic electroluminescent material” in the present disclosure means a material that may be used in an organic electroluminescent device, and may comprise at least one compound. The organic electroluminescent material may be comprised in any layer constituting an organic electroluminescent device, as necessary. For example, the organic electroluminescent material may be a hole injection material, a hole transport zone material, a light-emitting material (including a host material and a dopant material), an electron buffer material, a hole blocking material, an electron transport material, an electron injection material, etc. The hole transport zone material may be at least one selected from the group consisting of a hole transport material, a hole injection material, an electron blocking material, a hole auxiliary material and a light-emitting auxiliary material.

The term “hole transport zone” in the present disclosure may be composed of one or more layers selected from the group consisting of a hole transport layer, a hole injection layer, an electron blocking layer, a light-emitting auxiliary layer, and a hole auxiliary layer, and each layer may consist of one or more layers. The hole transport zone in the present disclosure may be a second hole transport layer, which may include at least one of a hole auxiliary layer and a light-emitting auxiliary layer. Herein, the hole auxiliary layer or the light-emitting auxiliary layer is placed between the hole transport layer and the light-emitting layer, and regulates the transport rate of holes. The hole auxiliary layer or the light-emitting auxiliary layer provides an effect of improving the efficiency and lifespan of the organic electroluminescent device.

The organic electroluminescent material of the present disclosure may comprise at least one compound represented by formula 1. The compounds of formula 1 may be included in the hole transport zone, but is not limited thereto.

According to one embodiment of the present disclosure, the hole transport zone may comprise a hole transport layer, and the hole transport layer can be multi-layered of which two or more layers are stacked, wherein at least one of the multi-layer may comprise at least one compound represented by formula 1 of the present disclosure. The layer comprising the compound represented by formula 1 or other layers may comprise a compound commonly used as a conventional hole transport zone material.

Herein, the term “(C1-C30)alkyl” in the present disclosure is meant to be a linear or branched alkyl having 1 to 30 carbon atoms constituting the chain, in which the number of carbon atoms is preferably 1 to 10, and more preferably 1 to 6. The above alkyl may include methyl, ethyl, n-propyl, isopropyl, n-butyl, isobutyl, tert-butyl, sec-butyl, etc. The term “(C3-C30)cycloalkyl” is meant to be a mono- or polycyclic hydrocarbon having 3 to 30 ring backbone carbon atoms, in which the number of carbon atoms is preferably 3 to 20, and more preferably 3 to 7. The above cycloalkyl may include cyclopropyl, cyclobutyl, cyclopentyl, cyclohexyl, cyclopentylmethyl, cyclohexylmethyl, etc. The term “(3- to 7-membered)heterocycloalkyl” in the present disclosure is meant to be a cycloalkyl having 3 to 7 ring backbone atoms, and including at least one heteroatom selected from the group consisting of B, N, O, S, Si, and P, preferably at least one heteroatom selected from the group consisting of O, S, and N. The above heterocycloalkyl may include tetrahydrofuran, pyrrolidine, thiolane, tetrahydropyran, etc. The term “(C6-C30)aryl(ene)” in the present disclosure is meant to be a monocyclic or fused ring radical derived from an aromatic hydrocarbon having 6 to 30 ring backbone carbon atoms. The above aryl(ene) may be partially saturated, and may comprise a spiro structure. The above aryl may include phenyl, biphenyl, terphenyl, naphthyl, binaphthyl, phenylnaphthyl, naphthylphenyl, fluorenyl, phenylfluorenyl, diphenylfluorenyl, benzofluorenyl, dibenzofluorenyl, phenanthrenyl, phenylphenanthrenyl, anthracenyl, indenyl, triphenylenyl, pyrenyl, tetracenyl, perylenyl, chrysenyl, naphthacenyl, fluoranthenyl, spirobifluorenyl, spiro[fluorene-benzofluoren]yl, azulenyl, tetramethyldihydrophenanthrenyl, etc. Specifically, the aryl may include phenyl, 1-naphthyl, 2-naphthyl, 1-anthryl, 2-anthryl, 9-anthryl, benzanthryl, 1-phenanthryl, 2-phenanthryl, 3-phenanthryl, 4-phenanthryl, 9-phenanthryl, naphthacenyl, pyrenyl, 1-chrysenyl, 2-chrysenyl, 3-chrysenyl, 4-chrysenyl, 5-chrysenyl, 6-chrysenyl, benzo[c]phenanthryl, benzo[g]chrysenyl, 1-triphenylenyl, 2-triphenylenyl, 3-triphenylenyl, 4-triphenylenyl, 1-fluorenyl, 2-fluorenyl, 3-fluorenyl, 4-fluorenyl, 9-fluorenyl, benzo[a]fluorenyl, benzo[b]fluorenyl, benzo[c]fluorenyl, dibenzofluorenyl, 2-biphenyl, 3-biphenyl, 4-biphenyl, o-terphenyl, m-terphenyl-4-yl, m-terphenyl-3-yl, m-terphenyl-2-yl, p-terphenyl-4-yl, p-terphenyl-3-yl, p-terphenyl-2-yl, m-quaterphenyl, 3-fluoranthenyl, 4-fluoranthenyl, 8-fluoranthenyl, 9-fluoranthenyl, benzofluoranthenyl, o-tolyl, m-tolyl, p-tolyl, 2,3-xylyl, 3,4-xylyl, 2,5-xylyl, mesityl, o-cumenyl, m-cumenyl, p-cumenyl, p-tert-butylphenyl, p-(2-phenylpropyl)phenyl, 4′-methylbiphenyl, 4″-tert-butyl-p-terphenyl-4-yl, 9,9-dimethyl-1-fluorenyl, 9,9-dimethyl-2-fluorenyl, 9,9-dimethyl-3-fluorenyl, 9,9-dimethyl-4-fluorenyl, 9,9-diphenyl-1-fluorenyl, 9,9-diphenyl-2-fluorenyl, 9,9-diphenyl-3-fluorenyl, 9,9-diphenyl-4-fluorenyl, 11,11-dimethyl-1-benzo[a]fluorenyl, 11,11-dimethyl-2-benzo[a]fluorenyl, 11,11-dimethyl-3-benzo[a]fluorenyl, 11,11-dimethyl-4-benzo[a]fluorenyl, 11,11-dimethyl-5-benzo[a]fluorenyl, 11,11-dimethyl-6-benzo[a]fluorenyl, 11,11-dimethyl-7-benzo[a]fluorenyl, 11,11-dimethyl-8-benzo[a]fluorenyl, 11,11-dimethyl-9-benzo[a]fluorenyl, 11,11-dimethyl-10-benzo[a]fluorenyl, 11,11-dimethyl-1-benzo[b]fluorenyl, 11,11-dimethyl-2-benzo[b]fluorenyl, 11,11-dimethyl-3-benzo[b]fluorenyl, 11,11-dimethyl-4-benzo[b]fluorenyl, 11,11-dimethyl-5-benzo[b]fluorenyl, 11,11-dimethyl-6-benzo[b]fluorenyl, 11,11-dimethyl-7-benzo[b]fluorenyl, 11,11-dimethyl-8-benzo[b]fluorenyl, 11,11-dimethyl-9-benzo[b]fluorenyl, 11,11-dimethyl-10-benzo[b]fluorenyl, 11,11-dimethyl-1-benzo[c]fluorenyl, 11,11-dimethyl-2-benzo[c]fluorenyl, 11,11-dimethyl-3-benzo[c]fluorenyl, 11,11-dimethyl-4-benzo[c]fluorenyl, 11,11-dimethyl-5-benzo[c]fluorenyl, 11,11-dimethyl-6-benzo[c]fluorenyl, 11,11-dimethyl-7-benzo[c]fluorenyl, 11,11-dimethyl-8-benzo[c]fluorenyl, 11,11-dimethyl-9-benzo[c]fluorenyl, 11,11-dimethyl-10-benzo[c]fluorenyl, 11,11-diphenyl-1-benzo[a]fluorenyl, 11,11-diphenyl-2-benzo[a]fluorenyl, 11,11-diphenyl-3-benzo[a]fluorenyl, 11,11-diphenyl-4-benzo[a]fluorenyl, 11,11-diphenyl-5-benzo[a]fluorenyl, 11,11-diphenyl-6-benzo[a]fluorenyl, 11,11-diphenyl-7-benzo[a]fluorenyl, 11,11-diphenyl-8-benzo[a]fluorenyl, 11,11-diphenyl-9-benzo[a]fluorenyl, 11,11-diphenyl-10-benzo[a]fluorenyl, 11,11-diphenyl-1-benzo[b]fluorenyl, 11,11-diphenyl-2-benzo[b]fluorenyl, 11,11-diphenyl-3-benzo[b]fluorenyl, 11,11-diphenyl-4-benzo[b]fluorenyl, 11,11-diphenyl-5-benzo[b]fluorenyl, 11,11-diphenyl-6-benzo[b]fluorenyl, 11,11-diphenyl-7-benzo[b]fluorenyl, 11,11-diphenyl-8-benzo[b]fluorenyl, 11,11-diphenyl-9-benzo[b]fluorenyl, 11,11-diphenyl-10-benzo[b]fluorenyl, 11,11-diphenyl-1-benzo[c]fluorenyl, 11,11-diphenyl-2-benzo[c]fluorenyl, 11,11-diphenyl-3-benzo[c]fluorenyl, 11,11-diphenyl-4-benzo[c]fluorenyl, 11,11-diphenyl-5-benzo[c]fluorenyl, 11,11-diphenyl-6-benzo[c]fluorenyl, 11,11-diphenyl-7-benzo[c]fluorenyl, 11,11-diphenyl-8-benzo[c]fluorenyl, 11,11-diphenyl-9-benzo[c]fluorenyl, 11,11-diphenyl-10-benzo[c]fluorenyl, 9,9,10,10-tetramethyl-9,10-dihydro-1-phenanthrenyl, 9,9,10,10-tetramethyl-9,10-dihydro-2-phenanthrenyl, 9,9,10,10-tetramethyl-9,10-dihydro-3-phenanthrenyl, 9,9,10,10-tetramethyl-9,10-dihydro-4-phenanthrenyl, etc.

The term “(3- to 30-membered)heteroaryl(ene)” in the present disclosure is meant to be an aryl or arylene having 3 to 30 ring backbone atoms and including at least one, preferably 1 to 4 heteroatom(s) selected from the group consisting of B, N, O, S, Si, and P. The above heteroaryl(ene) may be a monocyclic ring or a fused ring condensed with at least one benzene ring; may be partially saturated; may be one formed by linking at least one heteroaryl or aryl group to a heteroaryl group via a single bond(s); and may comprise a spiro structure. The above heteroaryl may include a monocyclic ring-type heteroaryl such as furyl, thiophenyl, pyrrolyl, imidazolyl, pyrazolyl, thiazolyl, thiadiazolyl, isothiazolyl, isoxazolyl, oxazolyl, oxadiazolyl, triazinyl, tetrazinyl, triazolyl, tetrazolyl, furazanyl, pyridyl, pyrazinyl, pyrimidinyl, pyridazinyl, etc., and a fused ring-type heteroaryl such as benzofuranyl, benzothiophenyl, isobenzofuranyl, dibenzofuranyl, dibenzothiophenyl, dibenzoselenophenyl, naphthobenzofuranyl, naphthobenzothiophenyl, benzofuroquinolinyl, benzofuroquinazolinyl, benzofuronaphthyridinyl, benzofuropyrimidinyl, naphthofuropyrimidinyl, benzothienoquinolinyl, benzothienoquinazolinyl, benzothienonaphthyridinyl, benzothienopyrimidinyl, naphthothienopyrimidinyl, pyrimidoindolyl, benzopyrimidoindolyl, benzofuropyrazinyl, naphthofuropyrazinyl, benzothienopyrazinyl, naphthothienopyrazinyl, pyrazinoindolyl, benzopyrazinoindolyl, benzimidazolyl, benzothiazolyl, benzoisothiazolyl, benzoisoxazolyl, benzoxazolyl, isoindolyl, indolyl, indazolyl, benzothiadiazolyl, quinolyl, isoquinolyl, cinnolinyl, quinazolinyl, quinoxalinyl, carbazolyl, benzocarbazolyl, dibenzocarbazolyl, phenoxazinyl, phenanthridinyl, benzodioxolyl, dihydroacridinyl, benzotriazolephenazinyl, imidazopyridinyl, chromenoquinazolinyl, thiochromenoquinazolinyl, dimethylbenzoperimidinyl, indolocarbazolyl, indenocarbazolyl, etc. More specifically, the heteroaryl may include 1-pyrrolyl, 2-pyrrolyl, 3-pyrrolyl, pyrazinyl, 2-pyridyl, 2-pyrimidinyl, 4-pyrimidinyl, 5-pyrimidinyl, 6-pyrimidinyl, 1,2,3-triazin-4-yl, 1,2,4-triazin-3-yl, 1,3,5-triazin-2-yl, 1-imidazolyl, 2-imidazolyl, 1-pyrazolyl, 1-indolidinyl, 2-indolidinyl, 3-indolidinyl, 5-indolidinyl, 6-indolidinyl, 7-indolidinyl, 8-indolidinyl, 2-imidazopyridyl, 3-imidazopyridyl, 5-imidazopyridyl, 6-imidazopyridyl, 7-imidazopyridyl, 8-imidazopyridyl, 3-pyridyl, 4-pyridyl, 1-indolyl, 2-indolyl, 3-indolyl, 4-indolyl, 5-indolyl, 6-indolyl, 7-indolyl, 1-isoindolyl, 2-isoindolyl, 3-isoindolyl, 4-isoindolyl, 5-isoindolyl, 6-isoindolyl, 7-isoindolyl, 2-furyl, 3-furyl, 2-benzofuranyl, 3-benzofuranyl, 4-benzofuranyl, 5-benzofuranyl, 6-benzofuranyl, 7-benzofuranyl, 1-isobenzofuranyl, 3-isobenzofuranyl, 4-isobenzofuranyl, 5-isobenzofuranyl, 6-isobenzofuranyl, 7-isobenzofuranyl, 2-quinolyl, 3-quinolyl, 4-quinolyl, 5-quinolyl, 6-quinolyl, 7-quinolyl, 8-quinolyl, 1-isoquinolyl, 3-isoquinolyl, 4-isoquinolyl, 5-isoquinolyl, 6-isoquinolyl, 7-isoquinolyl, 8-isoquinolyl, 2-quinoxalinyl, 5-quinoxalinyl, 6-quinoxalinyl, 1-carbazolyl, 2-carbazolyl, 3-carbazolyl, 4-carbazolyl, 9-carbazolyl, azacarbazolyl-1-yl, azacarbazolyl-2-yl, azacarbazolyl-3-yl, azacarbazolyl-4-yl, azacarbazolyl-5-yl, azacarbazolyl-6-yl, azacarbazolyl-7-yl, azacarbazolyl-8-yl, azacarbazolyl-9-yl, 1-phenanthridinyl, 2-phenanthridinyl, 3-phenanthridinyl, 4-phenanthridinyl, 6-phenanthridinyl, 7-phenanthridinyl, 8-phenanthridinyl, 9-phenanthridinyl, 10-phenanthridinyl, 1-acridinyl, 2-acridinyl, 3-acridinyl, 4-acridinyl, 9-acridinyl, 2-oxazolyl, 4-oxazolyl, 5-oxazolyl, 2-oxadiazolyl, 5-oxadiazolyl, 3-furazanyl, 2-thienyl, 3-thienyl, 2-methylpyrrol-1-yl, 2-methylpyrrol-3-yl, 2-methylpyrrol-4-yl, 2-methylpyrrol-5-yl, 3-methylpyrrol-1-yl, 3-methylpyrrol-2-yl, 3-methylpyrrol-4-yl, 3-methylpyrrol-5-yl, 2-tert-butylpyrrol-4-yl, 3-(2-phenylpropyl)pyrrol-1-yl, 2-methyl-1-indolyl, 4-methyl-1-indolyl, 2-methyl-3-indolyl, 4-methyl-3-indolyl, 2-tert-butyl-1-indolyl, 4-tert-butyl-1-indolyl, 2-tert-butyl-3-indolyl, 4-tert-butyl-3-indolyl, 1-dibenzofuranyl, 2-dibenzofuranyl, 3-dibenzofuranyl, 4-dibenzofuranyl, 1-dibenzothiophenyl, 2-dibenzothiophenyl, 3-dibenzothiophenyl, 4-dibenzothiophenyl, 1-naphtho-[1,2-b]-benzofuranyl, 2-naphtho-[1,2-b]-benzofuranyl, 3-naphtho-[1,2-b]-benzofuranyl, 4-naphtho-[1,2-b]-benzofuranyl, 5-naphtho-[1,2-b]-benzofuranyl, 6-naphtho-[1,2-b]-benzofuranyl, 7-naphtho-[1,2-b]-benzofuranyl, 8-naphtho-[1,2-b]-benzofuranyl, 9-naphtho-[1,2-b]-benzofuranyl, 10-naphtho-[1,2-b]-benzofuranyl, 1-naphtho-[2,3-b]-benzofuranyl, 2-naphtho-[2,3-b]-benzofuranyl, 3-naphtho-[2,3-b]-benzofuranyl, 4-naphtho-[2,3-b]-benzofuranyl, 5-naphtho-[2,3-b]-benzofuranyl, 6-naphtho-[2,3-b]-benzofuranyl, 7-naphtho-[2,3-b]-benzofuranyl, 8-naphtho-[2,3-b]-benzofuranyl, 9-naphtho-[2,3-b]-benzofuranyl, 10-naphtho-[2,3-b]-benzofuranyl, 1-naphtho-[2,1-b]-benzofuranyl, 2-naphtho-[2,1-b]-benzofuranyl, 3-naphtho-[2,1-b]-benzofuranyl, 4-naphtho-[2,1-b]-benzofuranyl, 5-naphtho-[2,1-b]-benzofuranyl, 6-naphtho-[2,1-b]-benzofuranyl, 7-naphtho-[2,1-b]-benzofuranyl, 8-naphtho-[2,1-b]-benzofuranyl, 9-naphtho-[2,1-b]-benzofuranyl, 10-naphtho-[2,1-b]-benzofuranyl, 1-naphtho-[1,2-b]-benzothiophenyl, 2-naphtho-[1,2-b]-benzothiophenyl, 3-naphtho-[1,2-b]-benzothiophenyl, 4-naphtho-[1,2-b]-benzothiophenyl, 5-naphtho-[1,2-b]-benzothiophenyl, 6-naphtho-[1,2-b]-benzothiophenyl, 7-naphtho-[1,2-b]-benzothiophenyl, 8-naphtho-[1,2-b]-benzothiophenyl, 9-naphtho-[1,2-b]-benzothiophenyl, 10-naphtho-[1,2-b]-benzothiophenyl, 1-naphtho-[2,3-b]-benzothiophenyl, 2-naphtho-[2,3-b]-benzothiophenyl, 3-naphtho-[2,3-b]-benzothiophenyl, 4-naphtho-[2,3-b]-benzothiophenyl, 5-naphtho-[2,3-b]-benzothiophenyl, 1-naphtho-[2,1-b]-benzothiophenyl, 2-naphtho-[2,1-b]-benzothiophenyl, 3-naphtho-[2,1-b]-benzothiophenyl, 4-naphtho-[2,1-b]-benzothiophenyl, 5-naphtho-[2,1-b]-benzothiophenyl, 6-naphtho-[2,1-b]-benzothiophenyl, 7-naphtho-[2,1-b]-benzothiophenyl, 8-naphtho-[2,1-b]-benzothiophenyl, 9-naphtho-[2,1-b]-benzothiophenyl, 10-naphtho-[2,1-b]-benzothiophenyl, 2-benzofuro[3,2-d]pyrimidinyl, 6-benzofuro[3,2-d]pyrimidinyl, 7-benzofuro[3,2-d]pyrimidinyl, 8-benzofuro[3,2-d]pyrimidinyl, 9-benzofuro[3,2-d]pyrimidinyl, 2-benzothio[3,2-d]pyrimidinyl, 6-benzothio[3,2-d]pyrimidinyl, 7-benzothio[3,2-d]pyrimidinyl, 8-benzothio[3,2-d]pyrimidinyl, 9-benzothio[3,2-d]pyrimidinyl, 2-benzofuro[3,2-d]pyrazinyl, 6-benzofuro[3,2-d]pyrazinyl, 7-benzofuro[3,2-d]pyrazinyl, 8-benzofuro[3,2-d]pyrazinyl, 9-benzofuro[3,2-d]pyrazinyl, 2-benzothio[3,2-d]pyrazinyl, 6-benzothio[3,2-d]pyrazinyl, 7-benzothio[3,2-d]pyrazinyl, 8-benzothio[3,2-d]pyrazinyl, 9-benzothio[3,2-d]pyrazinyl, 1-silafluorenyl, 2-silafluorenyl, 3-silafluorenyl, 4-silafluorenyl, 1-germafluorenyl, 2-germafluorenyl, 3-germafluorenyl, 4-germafluorenyl, 1-dibenzoselenophenyl, 2-dibenzoselenophenyl, 3-dibenzoselenophenyl, 4-dibenzoselenophenyl, etc. In the present disclosure, the term “halogen” includes F, Cl, Br, and I atoms.

In addition, “ortho (o-)”, “meta (m-)”, and “para (p-)” are prefixes, which represent the relative positions of substituents, respectively. Ortho indicates that two substituents are adjacent to each other, and for example, when two substituents in a benzene derivative occupy positions 1 and 2, it is called an ortho position. Meta indicates that two substituents are at positions 1 and 3, and for example, when two substituents in a benzene derivative occupy positions 1 and 3, it is called a meta position. Para indicates that two substituents are at positions 1 and 4, and for example, when two substituents in a benzene derivative occupy positions 1 and 4, it is called a para position.

In addition, “substituted” in the expression “substituted or unsubstituted” in the present disclosure means that a hydrogen atom in a certain functional group is replaced with another atom or another functional group (i.e., a substituent), and also includes that the hydrogen atom is replaced with a group in which two or more of the substituents are linked. For example, the “substituent in which two or more substituents are linked” may be pyridine-triazine. That is, pyridine-triazine may be interpreted as one heteroaryl substituent, or as substituents in which two heteroaryl substituents are linked. Herein, the substituents of the substituted alkyl, the substituted aryl(ene), the substituted heteroaryl(ene), the substituted cycloalkyl, the substituted alkoxy, the substituted trialkylsilyl, the substituted dialkylarylsilyl, the substituted alkyldiarylsilyl, the substituted triarylsilyl, the substituted fused ring of an aliphatic ring(s) and an aromatic ring(s), the substituted mono- or di-alkylamino, the substituted mono- or di-alkenylamino, the substituted mono- or di-arylamino, the substituted mono- or di-heteroarylamino, the substituted alkylalkenylamino, the substituted alkylarylamino, the substituted alkylheteroarylamino, the substituted alkenylarylamino, the substituted alkenylheteroarylamino, and the substituted arylheteroarylamino, each independently, are at least one selected from the group consisting of deuterium; a halogen; a cyano; a carboxyl; a nitro; a hydroxyl; a phosphine oxide; a (C1-C30)alkyl; a halo(C1-C30)alkyl; a (C2-C30)alkenyl; a (C2-C30)alkynyl; a (C1-C30)alkoxy; a (C1-C30)alkylthio; a (C3-C30)cycloalkyl; a (C3-C30)cycloalkenyl; a (3- to 7-membered)heterocycloalkyl; a (C6-C30)aryloxy; a (C6-C30)arylthio; a (3- to 30-membered)heteroaryl unsubstituted or substituted with a (C6-C30)aryl(s); a (C6-C30)aryl unsubstituted or substituted with a (3- to 30-membered)heteroaryl(s); a tri(C1-C30)alkylsilyl; a tri(C6-C30)arylsilyl; a di(C1-C30)alkyl(C6-C30)arylsilyl; a (C1-C30)alkyldi(C6-C30)arylsilyl; a fused ring group of a (C3-C30) aliphatic ring(s) and a (C6-C30) aromatic ring(s); an amino; a mono- or di-(C1-C30)alkylamino; a mono- or di-(C2-C30)alkenylamino; a mono- or di-(C6-C30)arylamino unsubstituted or substituted with a (C1-C30)alkyl(s); a mono- or di-(3- to 30-membered)heteroarylamino; a (C1-C30)alkyl(C2-C30)alkenylamino; a (C1-C30)alkyl(C6-C30)arylamino; a (C1-C30)alkyl(3- to 30-membered)heteroarylamino; a (C2-C30)alkenyl(C6-C30)arylamino; a (C2-C30)alkenyl(3- to 30-membered)heteroarylamino; a (C6-C30)aryl(3- to 30-membered)heteroarylamino; a (C1-C30)alkylcarbonyl; a (C1-C30)alkoxycarbonyl; a (C6-C30)arylcarbonyl; a (C6-C30)arylphosphine; a di(C6-C30)arylboronyl; a di(C1-C30)alkylboronyl; a (C1-C30)alkyl(C6-C30)arylboronyl; a (C6-C30)aryl(C1-C30)alkyl; and a (C1-C30)alkyl(C6-C30)aryl. According to one embodiment of the present disclosure, the substituents, each independently, are at least one selected from the group consisting of a (C1-C20)alkyl; a (C5-C25)cycloalkyl; a (5- to 25-membered)heteroaryl unsubstituted or substituted with a (C6-C25)aryl(s); a (C6-C25)aryl unsubstituted or substituted with deuterium, (C1-C20)alkyl(s) and/or (C6-C18)aryl(s); and an unsubstituted mono- or di-(C6-C25)arylamino. According to another embodiment of the present disclosure, the substituents, each independently, are at least one selected from the group consisting of an unsubstituted (C1-C10)alkyl, an unsubstituted (5- to 20-membered)heteroaryl, and an unsubstituted (C6-C20)aryl. For example, the substituents, each independently, may be at least one of a methyl and a phenyl.

In the formulas of the present disclosure, a ring formed by a linkage of adjacent substituents means that at least two adjacent substituents are linked to or fused with each other to form a substituted or unsubstituted, mono or polycyclic, (3- to 30-membered) alicyclic ring or aromatic ring, or the combination thereof. The ring may be preferably a substituted or unsubstituted, monocyclic or polycyclic, (3- to 26-membered) alicyclic ring, aromatic ring, or a combination thereof, and even more preferably an unsubstituted, monocyclic or polycyclic, (5- to 20-membered) aromatic ring. In addition, the formed ring may comprise at least one heteroatom selected from B, N, O, S, Si, and P, preferably at least one heteroatom selected from N, O, and S. For example, the ring may be a benzene ring, a cyclopentane ring, an indane ring, a fluorene ring, a phenanthrene ring, an indole ring, a xanthene ring, etc.

In the present disclosure, heteroaryl, heteroarylene, and heterocycloalkyl may each independently comprise at least one heteroatom selected from B, N, O, S, Si, and P. In addition, the heteroatom may be bonded to at least one selected from the group consisting of hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted mono- or di-(C1-C30)alkylamino, a substituted or unsubstituted mono- or di-(C2-C30)alkenylamino, a substituted or unsubstituted mono- or di-(C6-C30)arylamino, a substituted or unsubstituted mono- or di-(3- to 30-membered)heteroarylamino, a substituted or unsubstituted (C1-C30)alkyl(C2-C30)alkenylamino, a substituted or unsubstituted (C1-C30)alkyl(C6-C30)arylamino, a substituted or unsubstituted (C1-C30)alkyl(3- to 30-membered)heteroarylamino, a substituted or unsubstituted (C2-C30)alkenyl(C6-C30)arylamino, a substituted or unsubstituted (C2-C30)alkenyl(3- to 30-membered)heteroarylamino, and a substituted or unsubstituted (C6-C30)aryl(3- to 30-membered)heteroarylamino.

In formula 1, W represents O, S or —N-L₂-R₃. According to one embodiment of the present disclosure, W represents O or S. However, when W is O, the following structure is excluded.

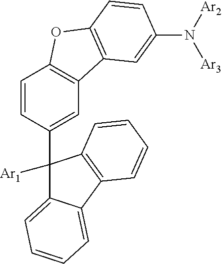

In formula 1, Z₁ to Z₈ each independently represent CR₄ or N. According to one embodiment of the present disclosure, Z₁ to Z₃ each independently represent CR₄.

In formula 1, L₁ and L₂ each independently represent a single bond, a substituted or unsubstituted (C6-C30)arylene, or a substituted or unsubstituted (3- to 30-membered)heteroarylene. According to one embodiment of the present disclosure, L₁ and L₂ each independently represent a single bond, or a substituted or unsubstituted (C6-C25)arylene. According to another embodiment of the present disclosure, L₁ and L₂ each independently represent a single bond or an unsubstituted (C6-C18)arylene. For example, L₁ may represent a single bond, phenylene, biphenylene, etc.

In formula 1, Ar₁ to Ar₃ each independently represent a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted fused ring group of a (C3-C30) aliphatic ring(s) and a (C6-C30) aromatic ring(s), a substituted or unsubstituted mono- or di-(C1-C30)alkylamino, a substituted or unsubstituted mono- or di-(C2-C30)alkenylamino, a substituted or unsubstituted mono- or di-(C6-C30)arylamino, a substituted or unsubstituted mono- or di-(3- to 30-membered)heteroarylamino, a substituted or unsubstituted (C1-C30)alkyl(C2-C30)alkenylamino, a substituted or unsubstituted (C1-C30)alkyl(C6-C30)arylamino, a substituted or unsubstituted (C1-C30)alkyl(3- to 30-membered)heteroarylamino, a substituted or unsubstituted (C2-C30)alkenyl(C6-C30)arylamino, a substituted or unsubstituted (C2-C30)alkenyl(3- to 30-membered)heteroarylamino, or a substituted or unsubstituted (C6-C30)aryl(3- to 30-membered)heteroarylamino. According to one embodiment of the present disclosure, Ar₁ to Ar₃ each independently represent a substituted or unsubstituted (C6-C25)aryl, or a substituted or unsubstituted (5- to 25-membered)heteroaryl. According to another embodiment of the present disclosure, Ar₁ represents a (C6-C18)aryl unsubstituted or substituted with a (C6-C18)aryl(s); and Ar₂ and Ar₃ each independently represent a (C6-C18)aryl unsubstituted or substituted with a (C1-C10)alkyl(s), or an unsubstituted (5- to 20-membered)heteroaryl. For example, Ar₁ may represent a phenyl, a biphenyl, a terphenyl, etc.; and Ar₂ and Ar₃ may each independently represent a phenyl, a biphenyl, a terphenyl, a dimethylfluorenyl, a dibenzofuranyl, a dibenzothiophenyl, etc.

In formula 1, R₁ to R₄ each independently represent hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted fused ring group of a (C3-C30) aliphatic ring(s) and a (C6-C30) aromatic ring(s), a substituted or unsubstituted mono- or di-(C1-C30)alkylamino, a substituted or unsubstituted mono- or di-(C2-C30)alkenylamino, a substituted or unsubstituted mono- or di-(C6-C30)arylamino, a substituted or unsubstituted mono- or di-(3- to 30-membered)heteroarylamino, a substituted or unsubstituted (C1-C30)alkyl(C2-C30)alkenylamino, a substituted or unsubstituted (C1-C30)alkyl(C6-C30)arylamino, a substituted or unsubstituted (C1-C30)alkyl(3- to 30-membered)heteroarylamino, a substituted or unsubstituted (C2-C30)alkenyl(C6-C30)arylamino, a substituted or unsubstituted (C2-C30)alkenyl(3- to 30-membered)heteroarylamino, or a substituted or unsubstituted (C6-C30)aryl(3- to 30-membered)heteroarylamino; or two or more of the adjacent R₁ to R₄ may be linked to each other to form a ring(s). If a plurality of each of R₁, R₂ and R₄ are present, each of R₁, each of R₂, and each of R₄ may be the same or different from each other. According to one embodiment of the present disclosure, R₁ to R₄ each independently represent hydrogen, a substituted or unsubstituted (C1-C20)alkyl, a substituted or unsubstituted (C6-C25)aryl, or a substituted or unsubstituted (5- to 25-membered)heteroaryl; or two or more of the adjacent R₁, R₂, and R₄ may be linked to each other to form a ring(s). According to another embodiment of the present disclosure, two or more of the adjacent R₁ may be linked to each other to form a ring(s), two or more of the adjacent R₂ may be linked to each other to form a ring(s), and two or more of the adjacent R₄ may be linked to each other to form a ring(s). According to still another embodiment of the present disclosure, R₁ and R₂ represent hydrogen; R₄ represents hydrogen, or two or more of the adjacent R₄ may be linked to each other to form a substituted or unsubstituted, mono- or polycyclic, (3- to 30-membered) alicyclic or aromatic ring, or the combination thereof. For example, R₁ and R₂ represent hydrogen; R₄ represents hydrogen, or two or more of the adjacent R₄ may be linked to each other to form a benzene ring(s), etc.

In formula 1, a and b are each independently an integer of 1 to 3; and if a and b are each an integer of 2 or more, each of R₁ and each of R₂ may be the same as or different from each other.

According to one embodiment of the present disclosure, the formula 1 may be represented by any one of the following formulas 1-1 to 1-16.

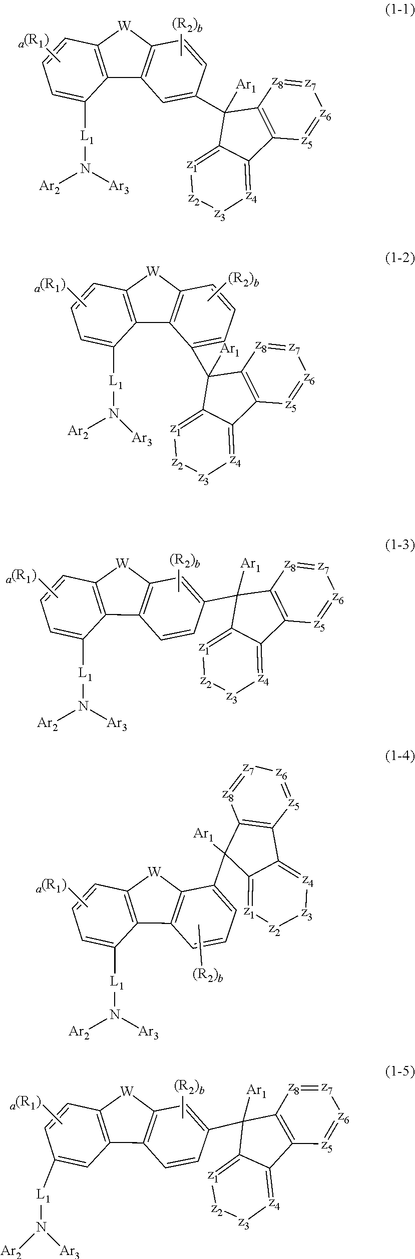

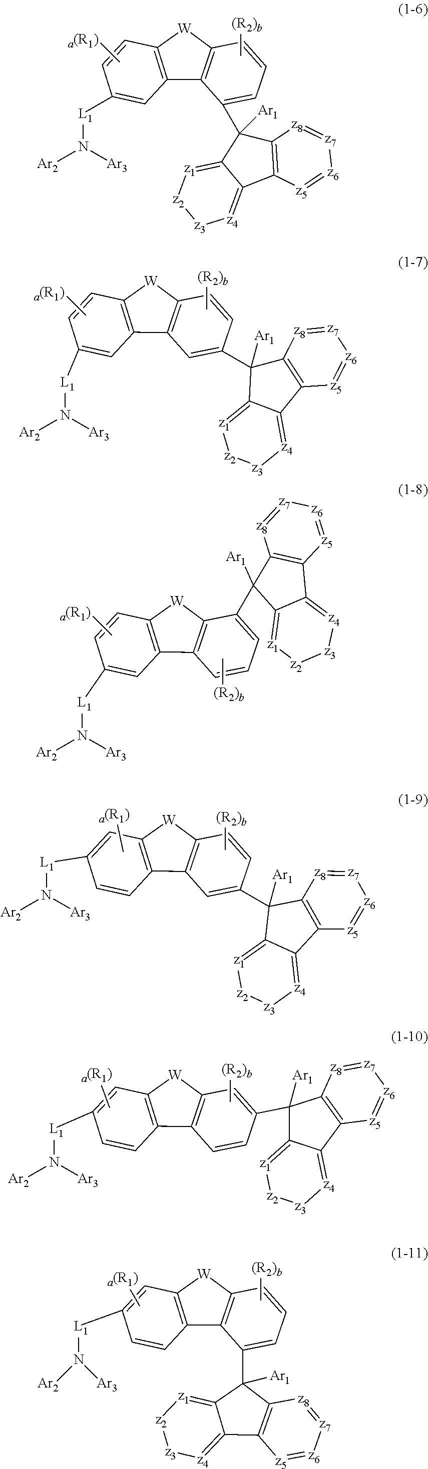

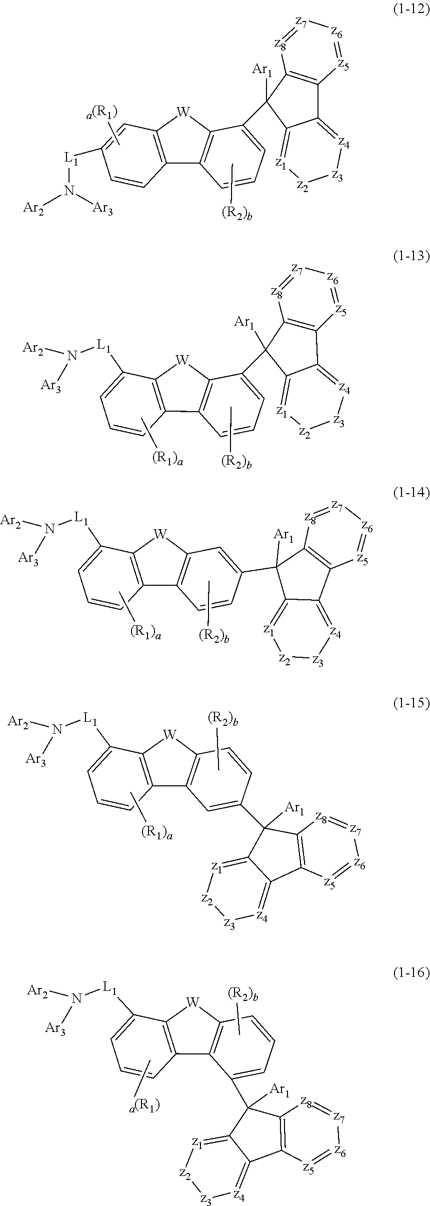

In formulas 1-1 to 1-16, W, Z₁ to Z₃, Ar₁ to Ar₃, L₁, R₁, R₂, a, and b are as defined in formula 1.

According to one embodiment of the present disclosure, formula 1 may be represented by the following formula 2.

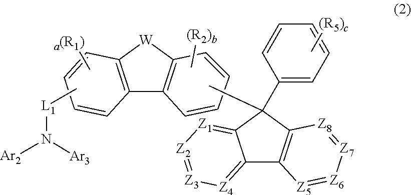

In formula 2, W, Z₁ to Z₈, Ar₂, Ar₃, L₁, R₁, R₂, a, and b are as defined in formula 1.

In formula 2, R₅ represents hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted fused ring group of a (C3-C30) aliphatic ring(s) and a (C6-C30) aromatic ring(s), a substituted or unsubstituted mono- or di-(C1-C30)alkylamino, a substituted or unsubstituted mono- or di-(C2-C30)alkenylamino, a substituted or unsubstituted mono- or di-(C6-C30)arylamino, a substituted or unsubstituted mono- or di-(3- to 30-membered)heteroarylamino, a substituted or unsubstituted (C1-C30)alkyl(C2-C30)alkenylamino, a substituted or unsubstituted (C1-C30)alkyl(C6-C30)arylamino, a substituted or unsubstituted (C1-C30)alkyl(3- to 30-membered)heteroarylamino, a substituted or unsubstituted (C2-C30)alkenyl(C6-C30)arylamino, a substituted or unsubstituted (C2-C30)alkenyl(3- to 30-membered)heteroarylamino, or a substituted or unsubstituted (C6-C30)aryl(3- to 30-membered)heteroarylamino. When a plurality of R₅ are present, each R₅ may be the same as or different from each other. According to one embodiment of the present disclosure, R₅ represents hydrogen, a substituted or unsubstituted (C6-C25)aryl, or a substituted or unsubstituted (5- to 25-membered)heteroaryl. According to another embodiment of the present disclosure, R₅ represents hydrogen or a unsubstituted (C6-C18)aryl. For example, R₅ represents hydrogen, phenyl, etc.

In formula 2, c represents an integer of 1 to 5. When c is 2 or more, each of R₅ may be the same as or different from each other. For example, c is an integer of 1 or 2.

The compound represented by formula 1 may be at least one selected from the following compounds, but is not limited thereto.

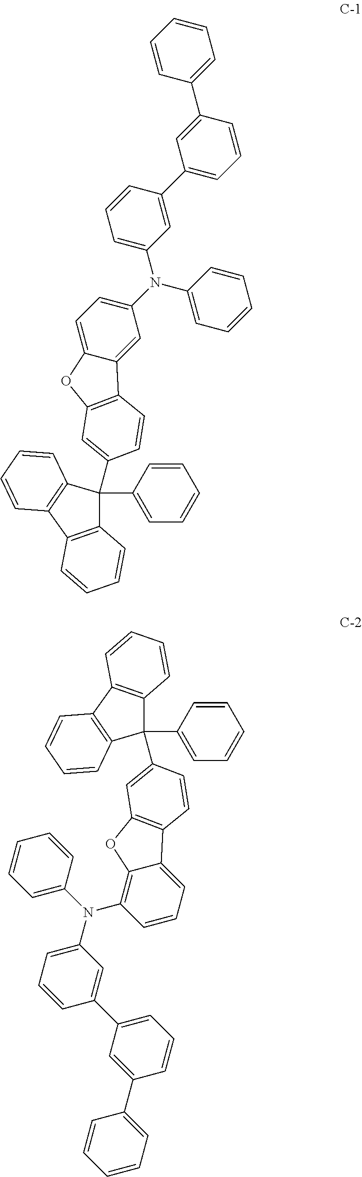 [SMILES: c1ccc(-c2cccc(-c3cccc(N(c4ccccc4)c4ccc5oc6cc(C7(c8ccccc8)c8ccccc8-c8ccccc87)ccc6c5c4)c3)c2)cc1 ; c1ccc(-c2cccc(-c3cccc(N(c4ccccc4)c4cccc5c4oc4cc(C6(c7ccccc7)c7ccccc7-c7ccccc76)ccc45)c3)c2)cc1]

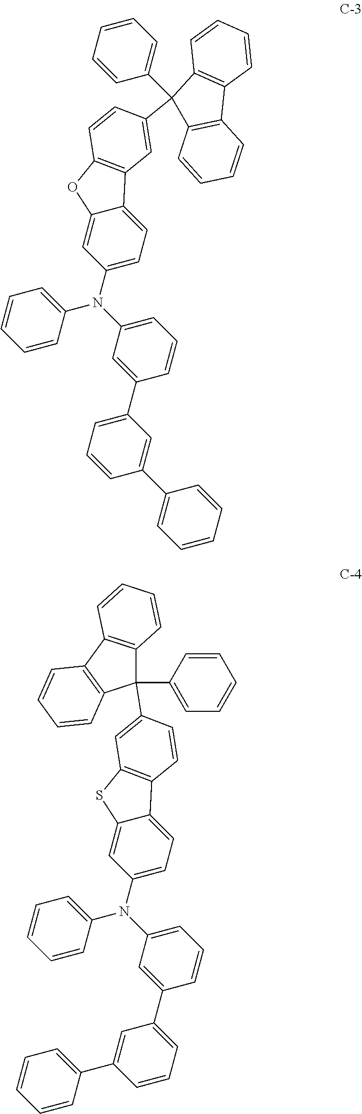 [SMILES: c1ccc(-c2cccc(-c3cccc(N(c4ccccc4)c4ccc5c(c4)oc4ccc(C6(c7ccccc7)c7ccccc7-c7ccccc76)cc45)c3)c2)cc1 ; c1ccc(-c2cccc(-c3cccc(N(c4ccccc4)c4ccc5c(c4)sc4cc(C6(c7ccccc7)c7ccccc7-c7ccccc76)ccc45)c3)c2)cc1]

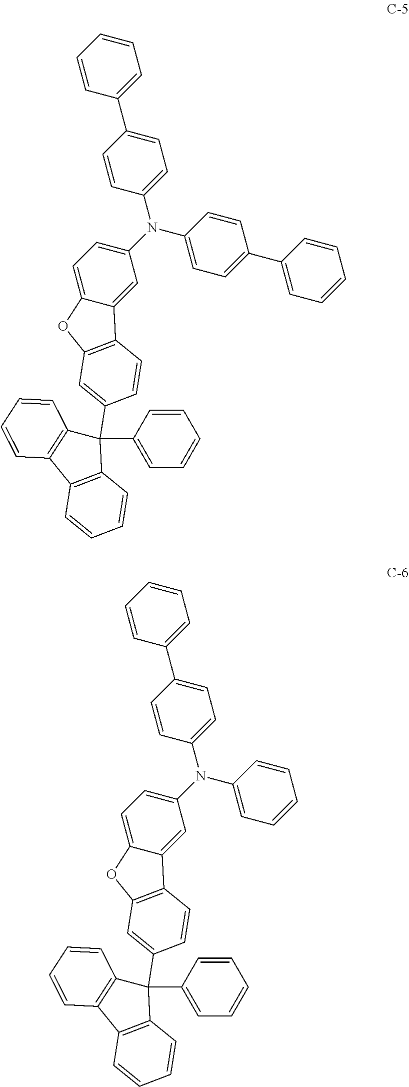 [SMILES: c1ccc(-c2ccc(N(c3ccc(-c4ccccc4)cc3)c3ccc4oc5cc(C6(c7ccccc7)c7ccccc7-c7ccccc76)ccc5c4c3)cc2)cc1 ; c1ccc(-c2ccc(N(c3ccccc3)c3ccc4oc5cc(C6(c7ccccc7)c7ccccc7-c7ccccc76)ccc5c4c3)cc2)cc1]

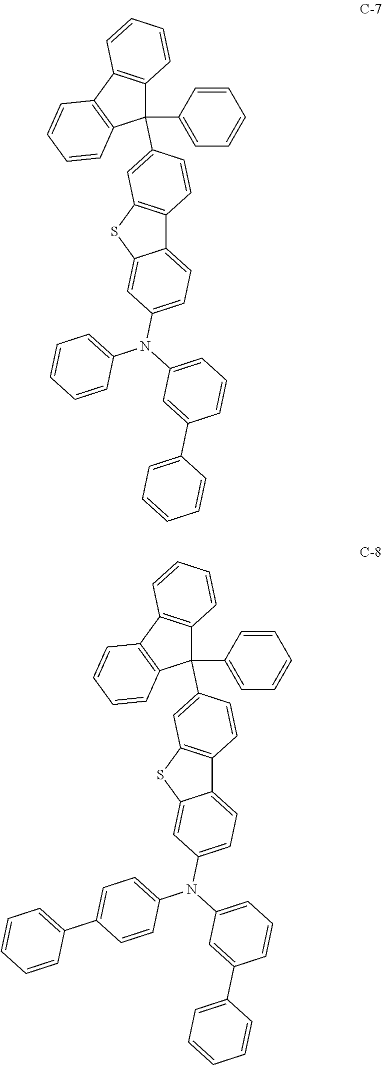

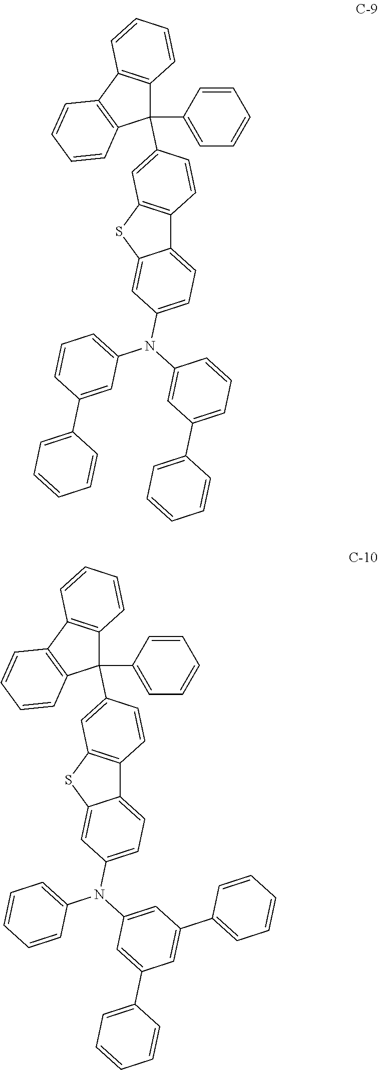 [SMILES: c1ccc(-c2cccc(N(c3cccc(-c4ccccc4)c3)c3ccc4c(c3)sc3cc(C5(c6ccccc6)c6ccccc6-c6ccccc65)ccc34)c2)cc1 ; c1ccc(-c2cc(-c3ccccc3)cc(N(c3ccccc3)c3ccc4c(c3)sc3cc(C5(c6ccccc6)c6ccccc6-c6ccccc65)ccc34)c2)cc1]

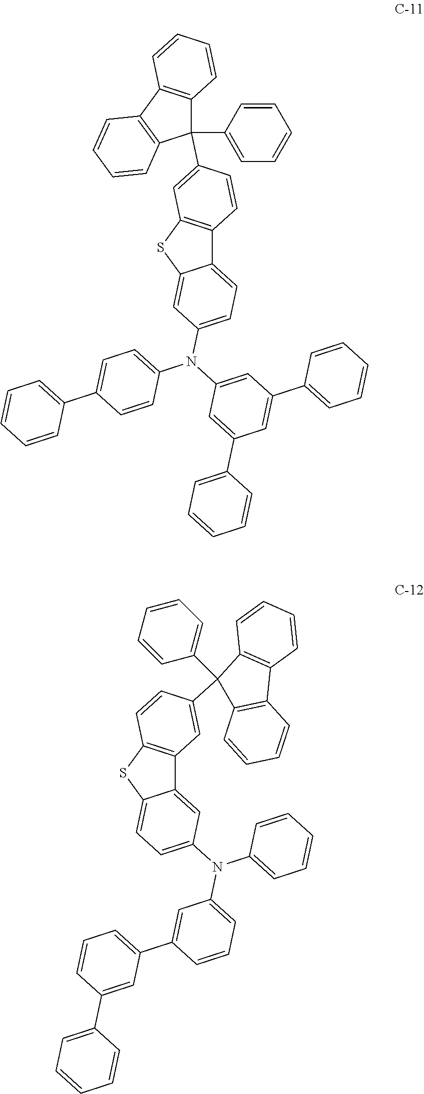 [SMILES: c1ccc(-c2ccc(N(c3cc(-c4ccccc4)cc(-c4ccccc4)c3)c3ccc4c(c3)sc3cc(C5(c6ccccc6)c6ccccc6-c6ccccc65)ccc34)cc2)cc1 ; c1ccc(-c2cccc(-c3cccc(N(c4ccccc4)c4ccc5sc6ccc(C7(c8ccccc8)c8ccccc8-c8ccccc87)cc6c5c4)c3)c2)cc1]

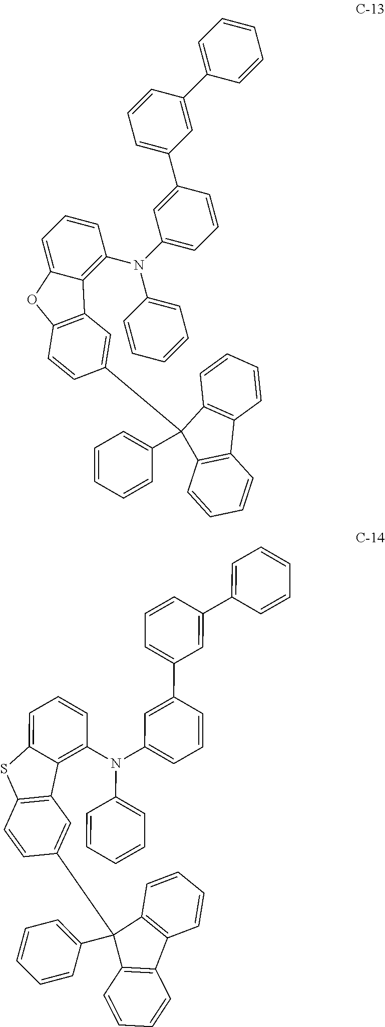

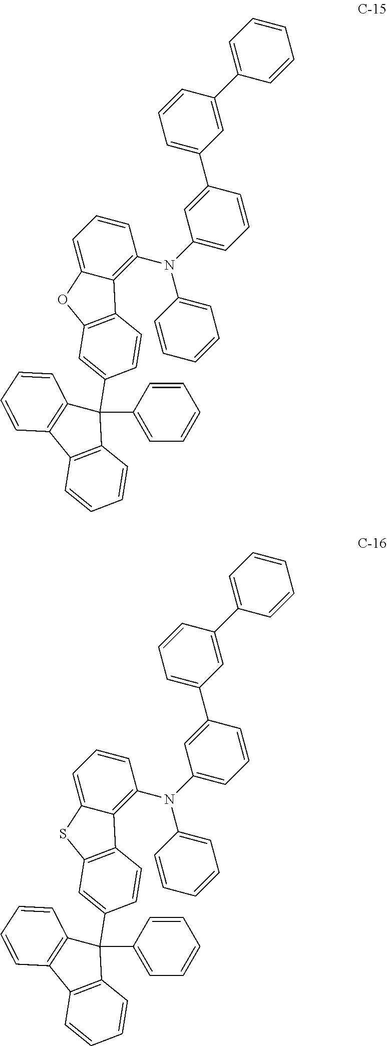 [SMILES: c1ccc(-c2cccc(-c3cccc(N(c4ccccc4)c4cccc5oc6cc(C7(c8ccccc8)c8ccccc8-c8ccccc87)ccc6c45)c3)c2)cc1 ; c1ccc(-c2cccc(-c3cccc(N(c4ccccc4)c4cccc5sc6cc(C7(c8ccccc8)c8ccccc8-c8ccccc87)ccc6c45)c3)c2)cc1]

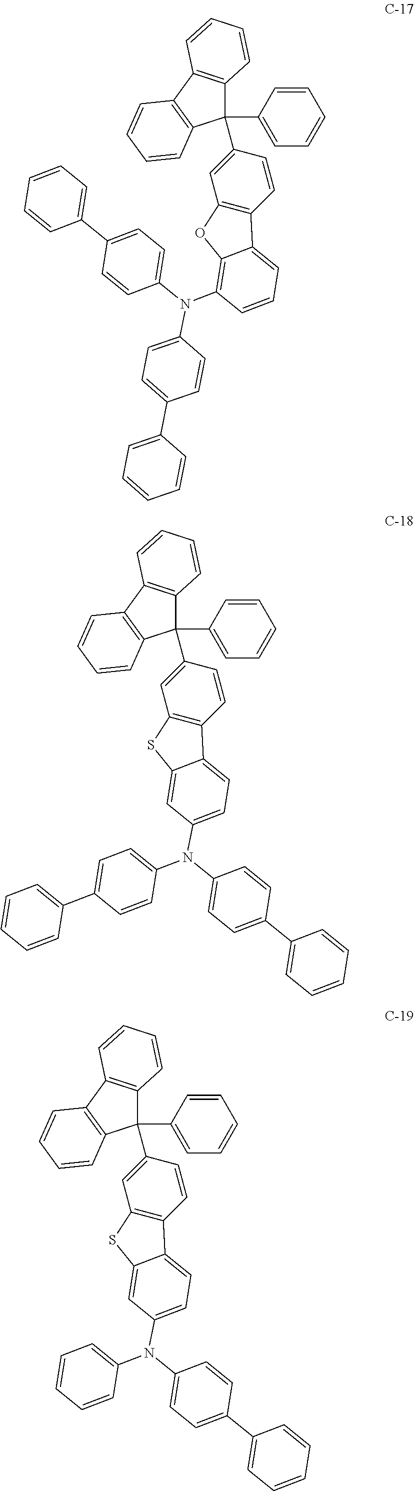

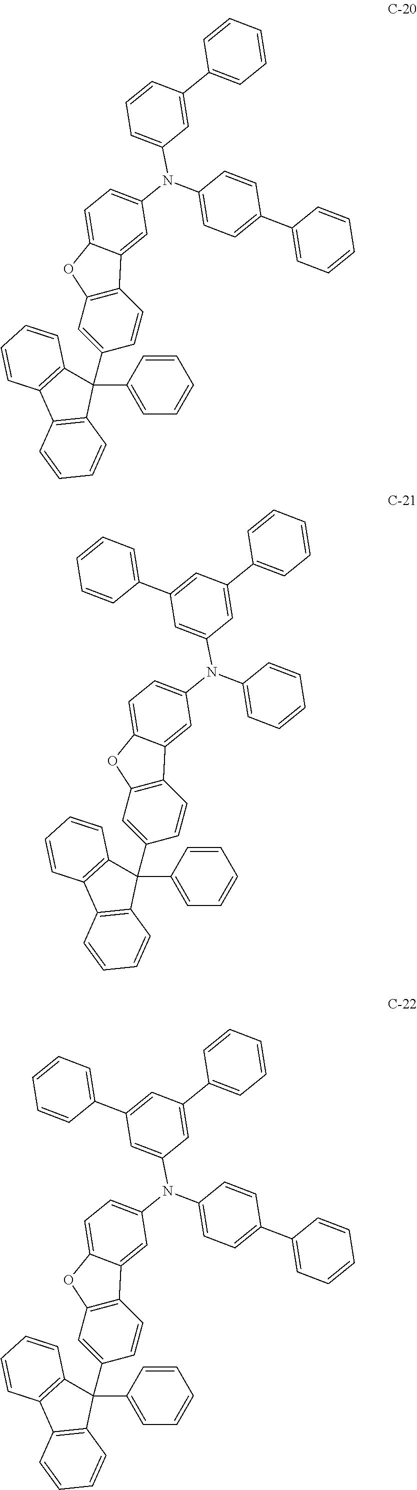 [SMILES: c1ccc(-c2ccc(N(c3cccc(-c4ccccc4)c3)c3ccc4oc5cc(C6(c7ccccc7)c7ccccc7-c7ccccc76)ccc5c4c3)cc2)cc1 ; c1ccc(-c2cc(-c3ccccc3)cc(N(c3ccccc3)c3ccc4oc5cc(C6(c7ccccc7)c7ccccc7-c7ccccc76)ccc5c4c3)c2)cc1 ; c1ccc(-c2ccc(N(c3cc(-c4ccccc4)cc(-c4ccccc4)c3)c3ccc4oc5cc(C6(c7ccccc7)c7ccccc7-c7ccccc76)ccc5c4c3)cc2)cc1]

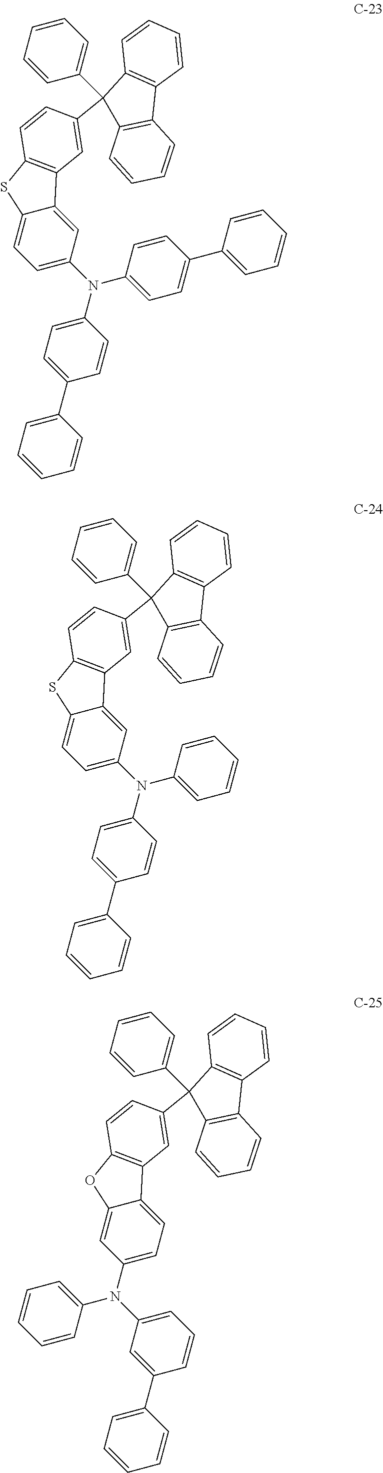 [SMILES: c1ccc(-c2ccc(N(c3ccc(-c4ccccc4)cc3)c3ccc4sc5ccc(C6(c7ccccc7)c7ccccc7-c7ccccc76)cc5c4c3)cc2)cc1 ; c1ccc(-c2ccc(N(c3ccccc3)c3ccc4sc5ccc(C6(c7ccccc7)c7ccccc7-c7ccccc76)cc5c4c3)cc2)cc1 ; c1ccc(-c2cccc(N(c3ccccc3)c3ccc4c(c3)oc3ccc(C5(c6ccccc6)c6ccccc6-c6ccccc65)cc34)c2)cc1]

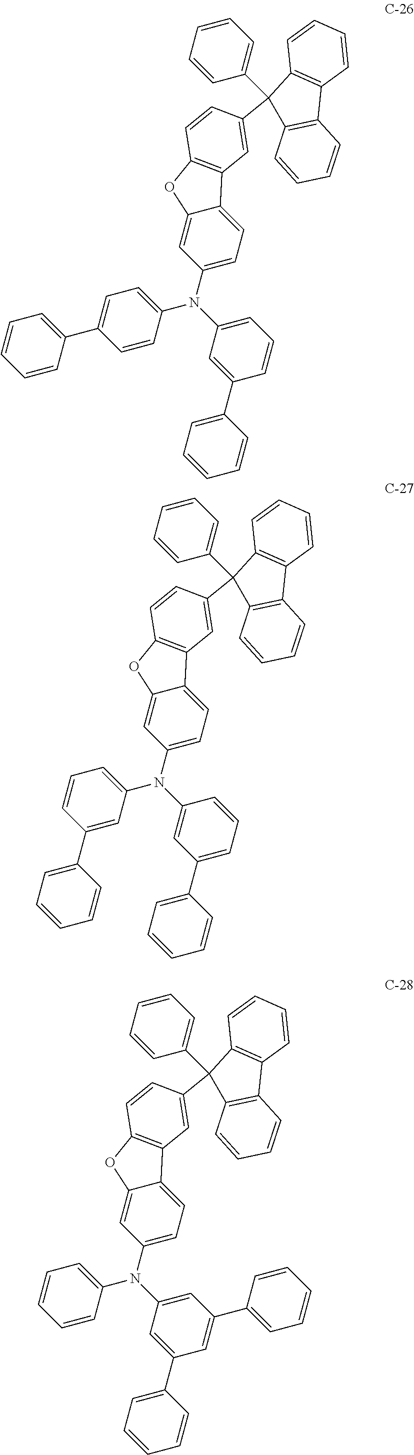

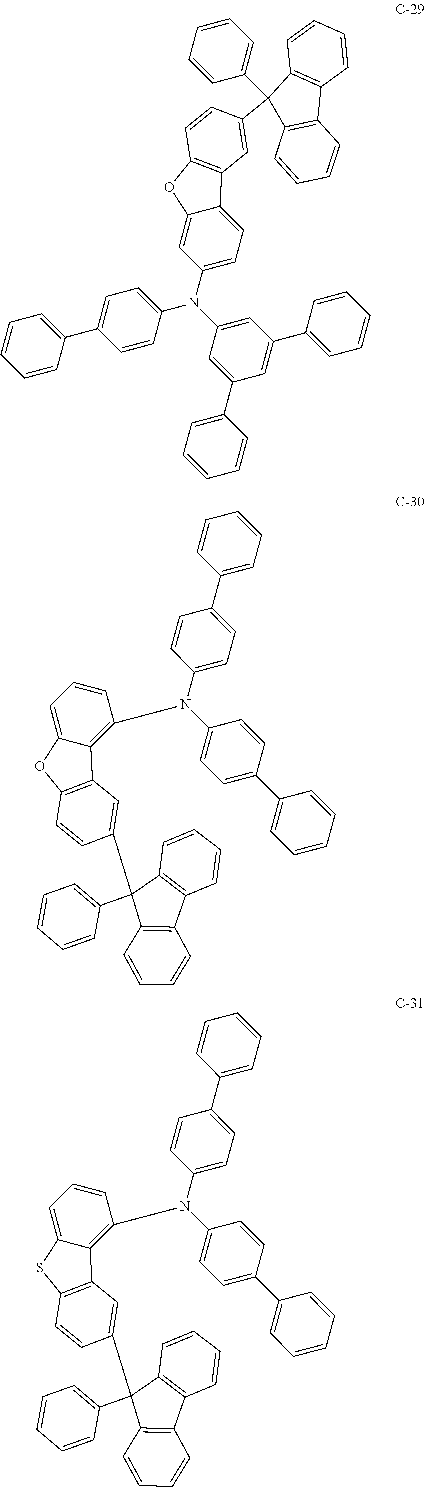

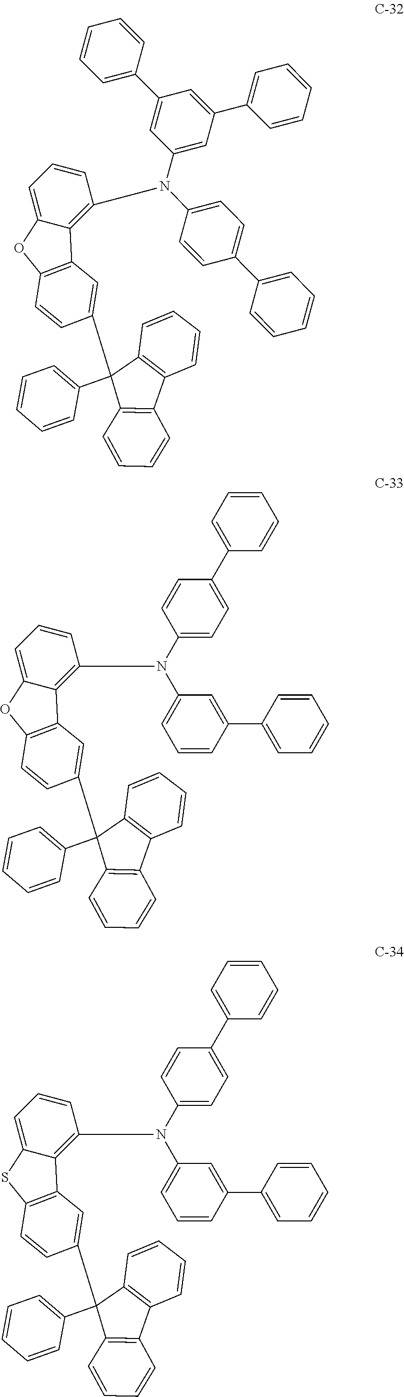 [SMILES: c1ccc(-c2ccc(N(c3cc(-c4ccccc4)cc(-c4ccccc4)c3)c3cccc4oc5ccc(C6(c7ccccc7)c7ccccc7-c7ccccc76)cc5c34)cc2)cc1 ; c1ccc(-c2ccc(N(c3cccc(-c4ccccc4)c3)c3cccc4oc5ccc(C6(c7ccccc7)c7ccccc7-c7ccccc76)cc5c34)cc2)cc1 ; c1ccc(-c2ccc(N(c3cccc(-c4ccccc4)c3)c3cccc4sc5ccc(C6(c7ccccc7)c7ccccc7-c7ccccc76)cc5c34)cc2)cc1]

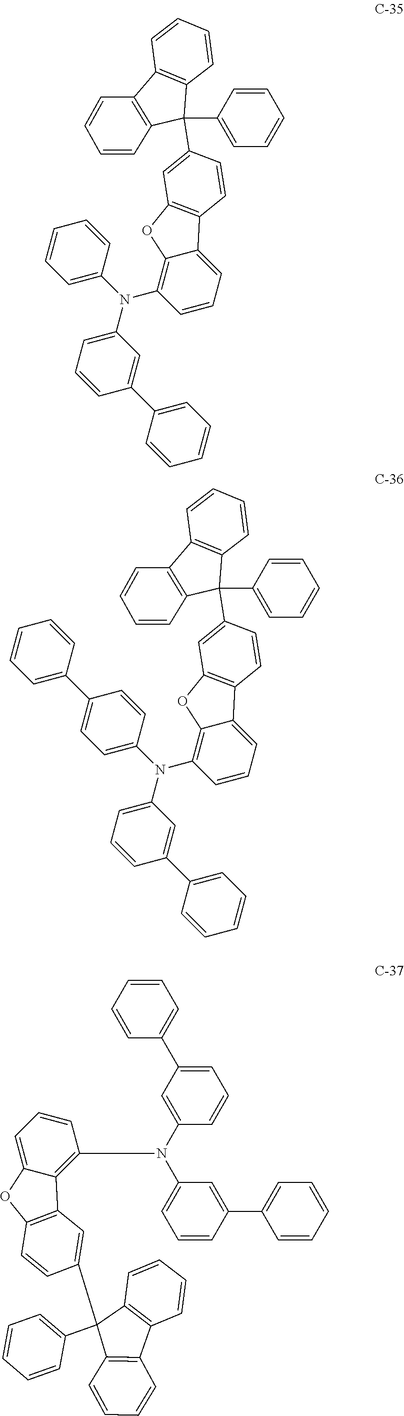

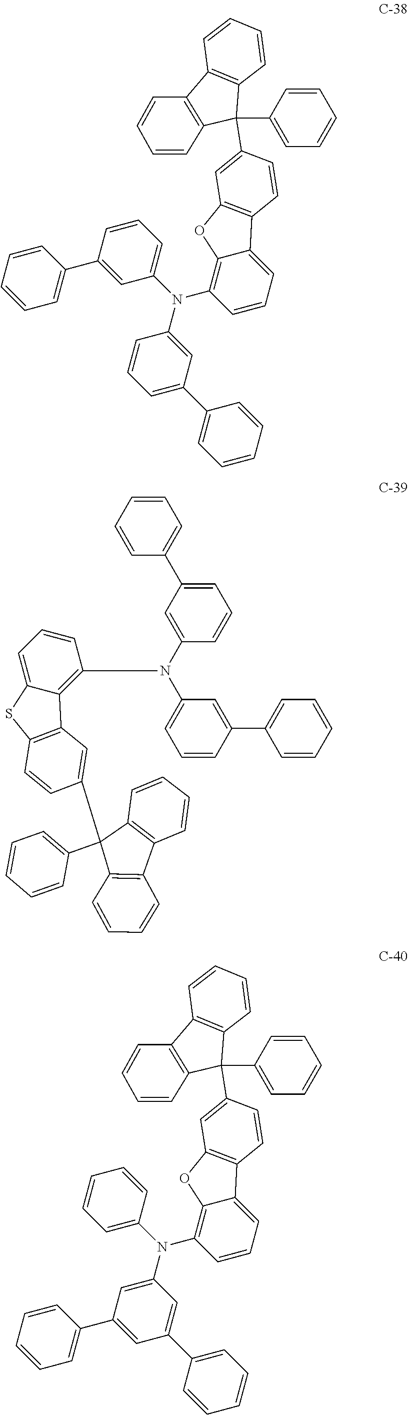 [SMILES: c1ccc(-c2cccc(N(c3cccc(-c4ccccc4)c3)c3cccc4c3oc3cc(C5(c6ccccc6)c6ccccc6-c6ccccc65)ccc34)c2)cc1 ; c1ccc(-c2cccc(N(c3cccc(-c4ccccc4)c3)c3cccc4sc5ccc(C6(c7ccccc7)c7ccccc7-c7ccccc76)cc5c34)c2)cc1 ; c1ccc(-c2cc(-c3ccccc3)cc(N(c3ccccc3)c3cccc4c3oc3cc(C5(c6ccccc6)c6ccccc6-c6ccccc65)ccc34)c2)cc1]

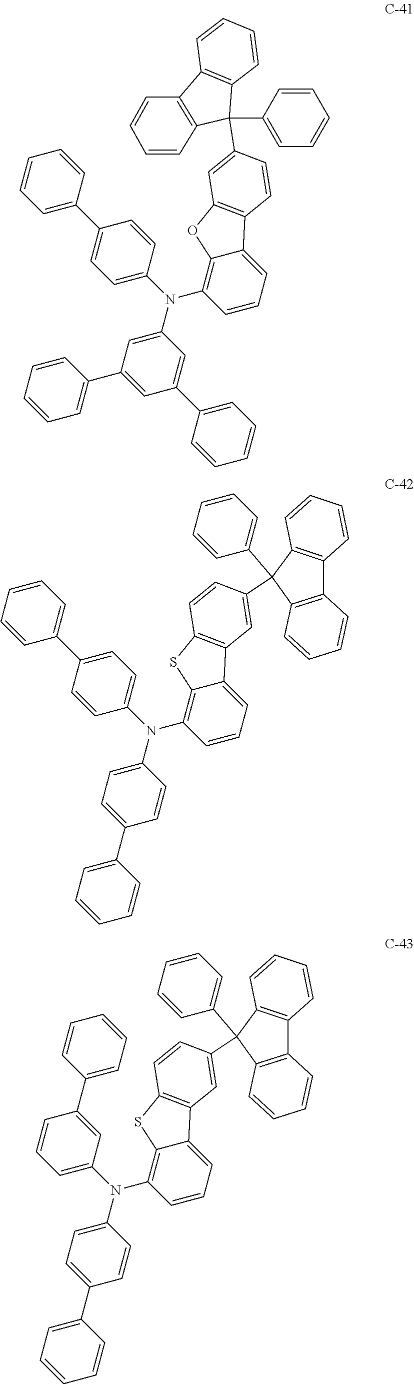

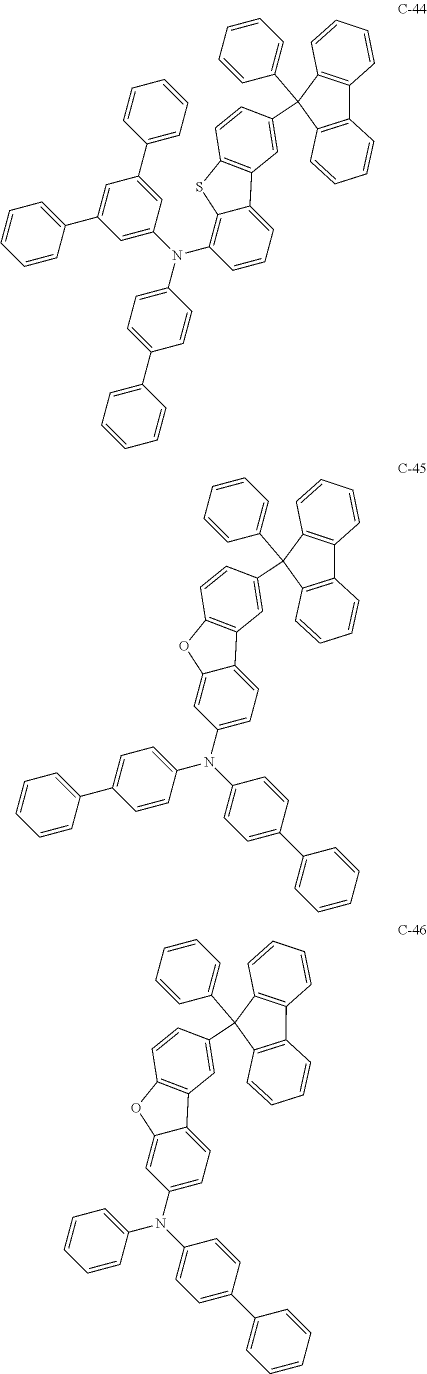

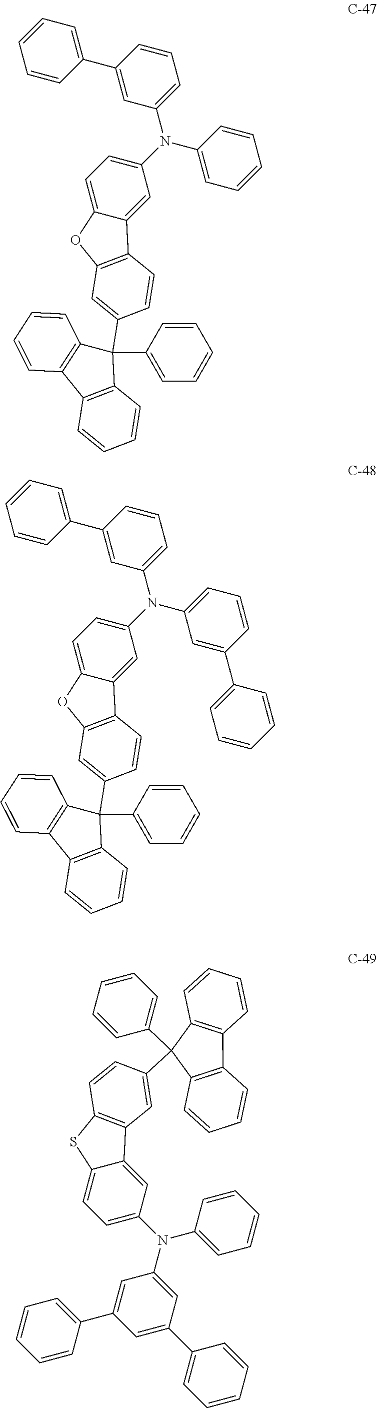 [SMILES: c1ccc(-c2cccc(N(c3ccccc3)c3ccc4oc5cc(C6(c7ccccc7)c7ccccc7-c7ccccc76)ccc5c4c3)c2)cc1 ; c1ccc(-c2cccc(N(c3cccc(-c4ccccc4)c3)c3ccc4oc5cc(C6(c7ccccc7)c7ccccc7-c7ccccc76)ccc5c4c3)c2)cc1 ; c1ccc(-c2cc(-c3ccccc3)cc(N(c3ccccc3)c3ccc4sc5ccc(C6(c7ccccc7)c7ccccc7-c7ccccc76)cc5c4c3)c2)cc1]

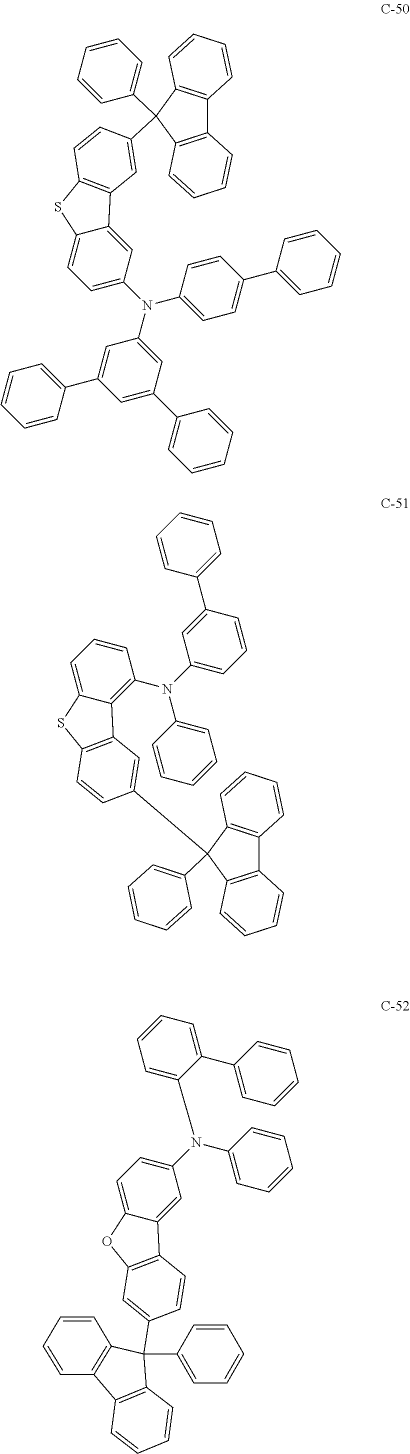

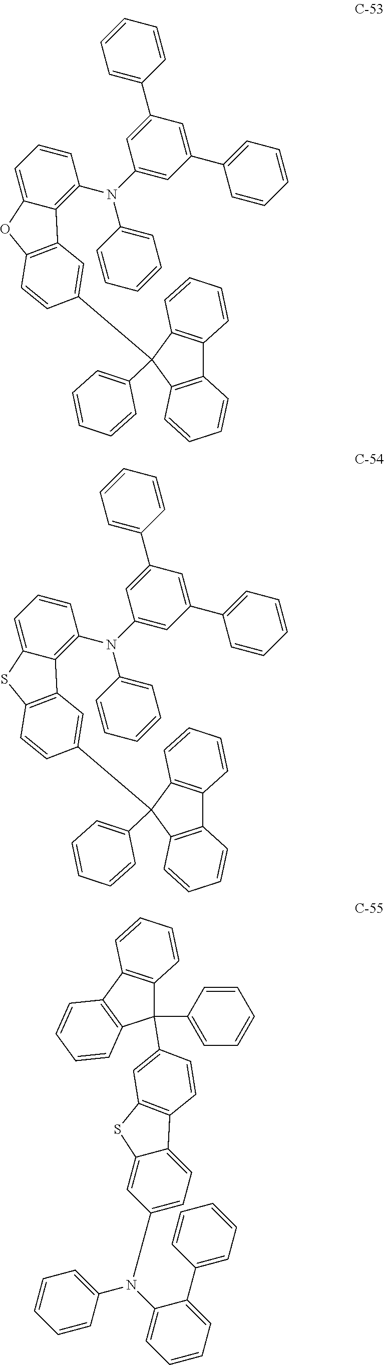

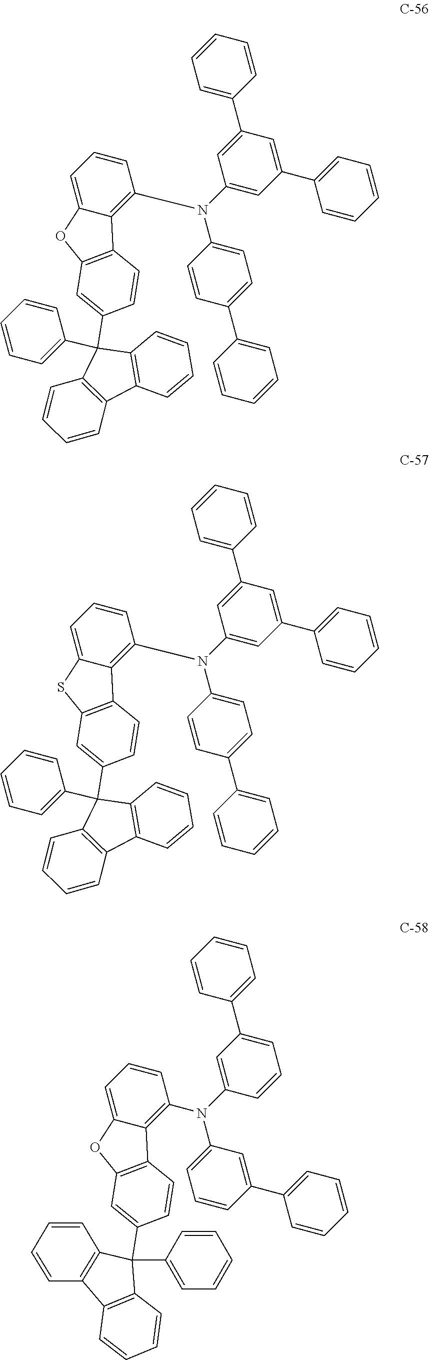 [SMILES: c1ccc(-c2ccc(N(c3cc(-c4ccccc4)cc(-c4ccccc4)c3)c3cccc4oc5cc(C6(c7ccccc7)c7ccccc7-c7ccccc76)ccc5c34)cc2)cc1 ; c1ccc(-c2ccc(N(c3cc(-c4ccccc4)cc(-c4ccccc4)c3)c3cccc4sc5cc(C6(c7ccccc7)c7ccccc7-c7ccccc76)ccc5c34)cc2)cc1 ; c1ccc(-c2cccc(N(c3cccc(-c4ccccc4)c3)c3cccc4oc5cc(C6(c7ccccc7)c7ccccc7-c7ccccc76)ccc5c34)c2)cc1]

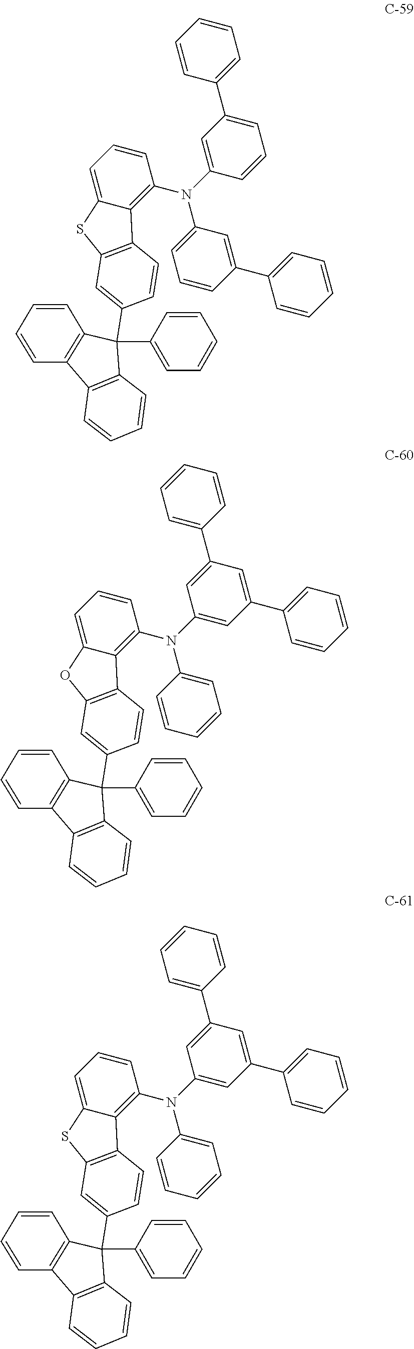 [SMILES: c1ccc(-c2cccc(N(c3cccc(-c4ccccc4)c3)c3cccc4sc5cc(C6(c7ccccc7)c7ccccc7-c7ccccc76)ccc5c34)c2)cc1 ; c1ccc(-c2cc(-c3ccccc3)cc(N(c3ccccc3)c3cccc4oc5cc(C6(c7ccccc7)c7ccccc7-c7ccccc76)ccc5c34)c2)cc1 ; c1ccc(-c2cc(-c3ccccc3)cc(N(c3ccccc3)c3cccc4sc5cc(C6(c7ccccc7)c7ccccc7-c7ccccc76)ccc5c34)c2)cc1]

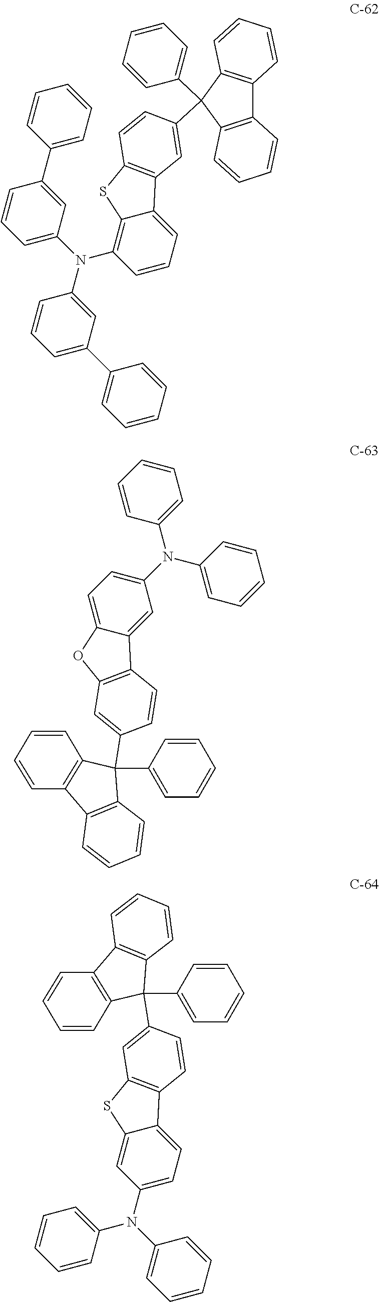

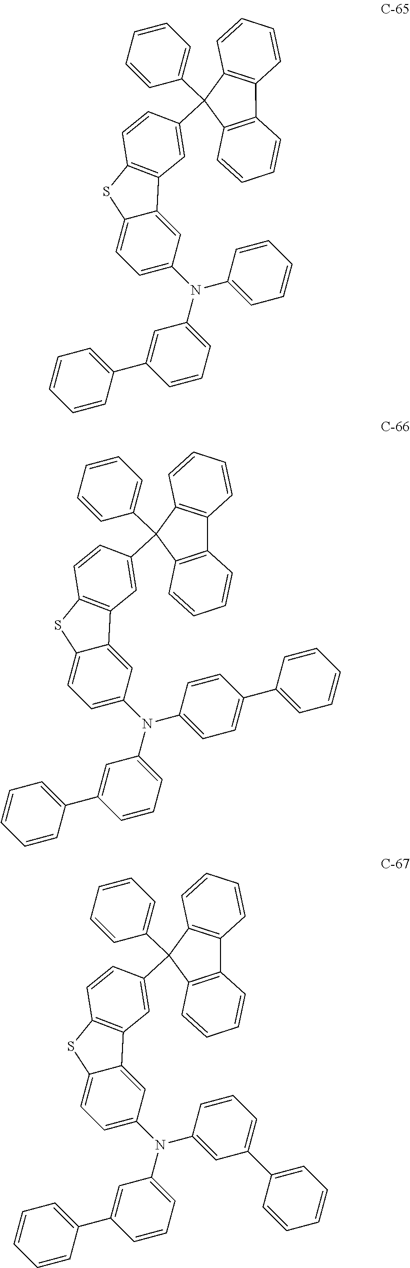 [SMILES: c1ccc(-c2cccc(N(c3ccccc3)c3ccc4sc5ccc(C6(c7ccccc7)c7ccccc7-c7ccccc76)cc5c4c3)c2)cc1 ; c1ccc(-c2ccc(N(c3cccc(-c4ccccc4)c3)c3ccc4sc5ccc(C6(c7ccccc7)c7ccccc7-c7ccccc76)cc5c4c3)cc2)cc1 ; c1ccc(-c2cccc(N(c3cccc(-c4ccccc4)c3)c3ccc4sc5ccc(C6(c7ccccc7)c7ccccc7-c7ccccc76)cc5c4c3)c2)cc1]

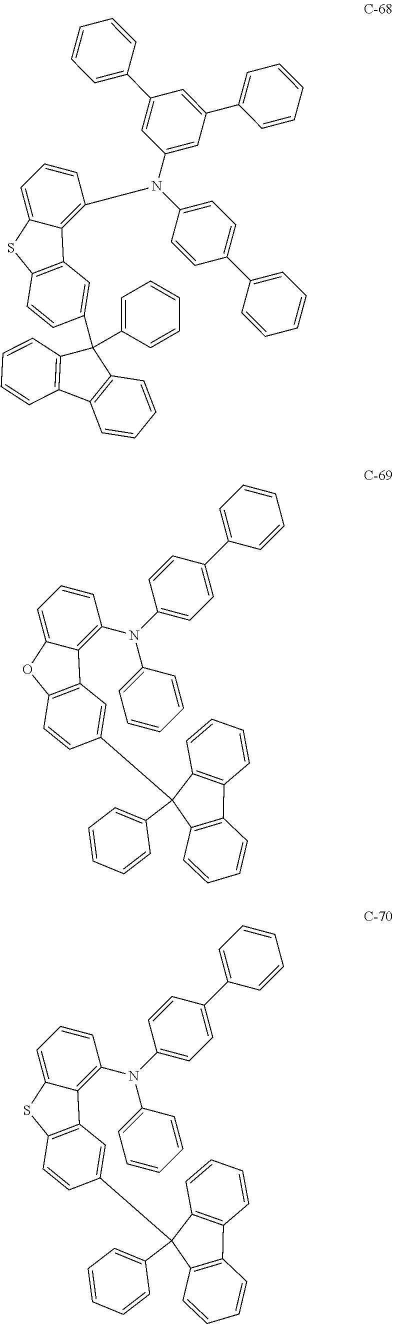 [SMILES: c1ccc(-c2ccc(N(c3cc(-c4ccccc4)cc(-c4ccccc4)c3)c3cccc4sc5ccc(C6(c7ccccc7)c7ccccc7-c7ccccc76)cc5c34)cc2)cc1 ; c1ccc(-c2ccc(N(c3ccccc3)c3cccc4oc5ccc(C6(c7ccccc7)c7ccccc7-c7ccccc76)cc5c34)cc2)cc1 ; c1ccc(-c2ccc(N(c3ccccc3)c3cccc4sc5ccc(C6(c7ccccc7)c7ccccc7-c7ccccc76)cc5c34)cc2)cc1]

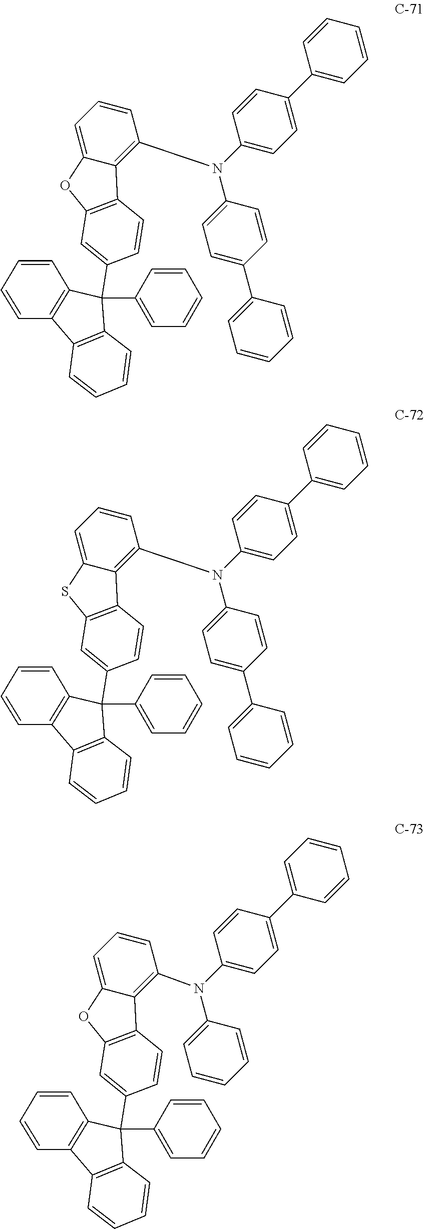 [SMILES: c1ccc(-c2ccc(N(c3ccc(-c4ccccc4)cc3)c3cccc4oc5cc(C6(c7ccccc7)c7ccccc7-c7ccccc76)ccc5c34)cc2)cc1 ; c1ccc(-c2ccc(N(c3ccc(-c4ccccc4)cc3)c3cccc4sc5cc(C6(c7ccccc7)c7ccccc7-c7ccccc76)ccc5c34)cc2)cc1 ; c1ccc(-c2ccc(N(c3ccccc3)c3cccc4oc5cc(C6(c7ccccc7)c7ccccc7-c7ccccc76)ccc5c34)cc2)cc1]

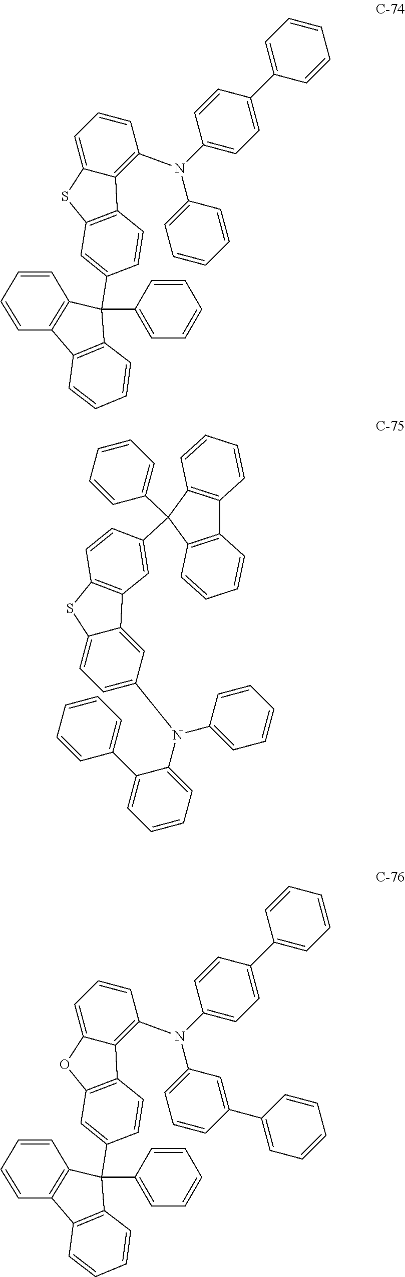

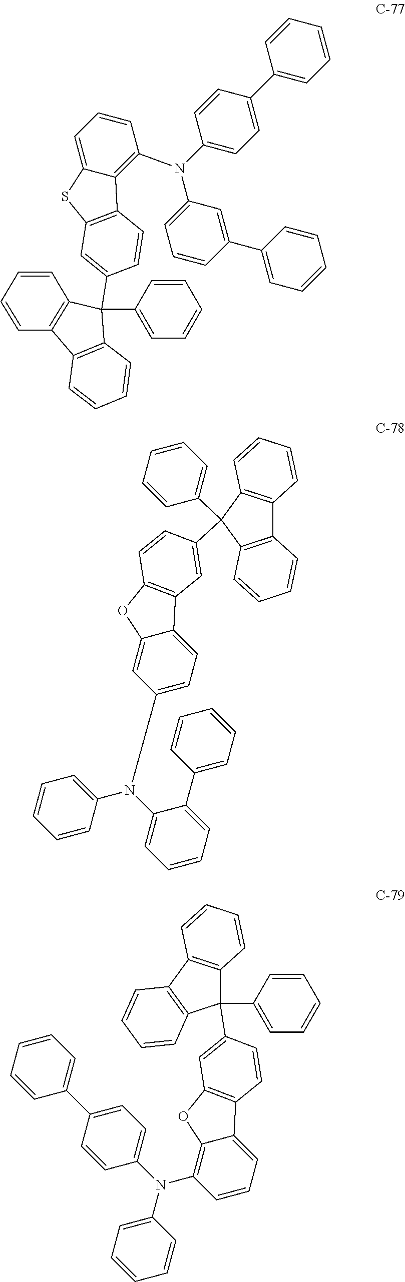

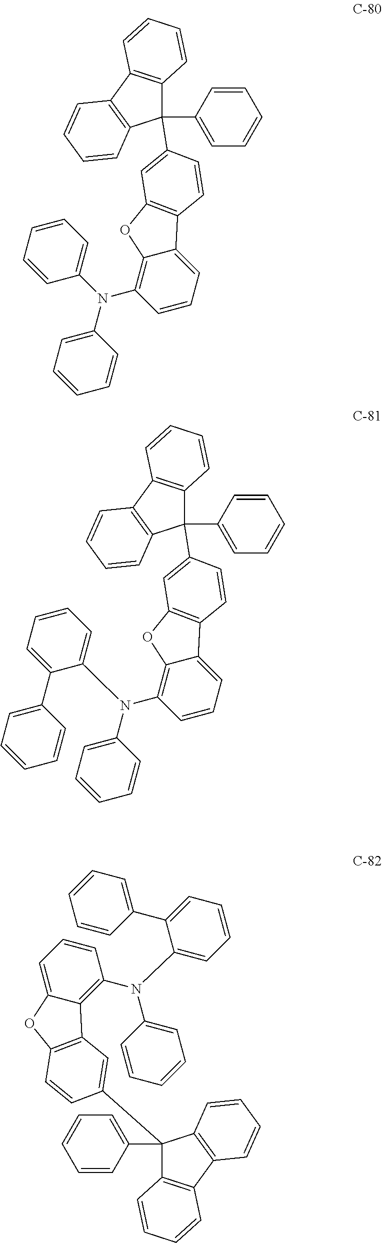 [SMILES: c1ccc(N(c2ccccc2)c2cccc3c2oc2cc(C4(c5ccccc5)c5ccccc5-c5ccccc54)ccc23)cc1 ; c1ccc(-c2ccccc2N(c2ccccc2)c2cccc3c2oc2cc(C4(c5ccccc5)c5ccccc5-c5ccccc54)ccc23)cc1 ; c1ccc(-c2ccccc2N(c2ccccc2)c2cccc3oc4ccc(C5(c6ccccc6)c6ccccc6-c6ccccc65)cc4c23)cc1]

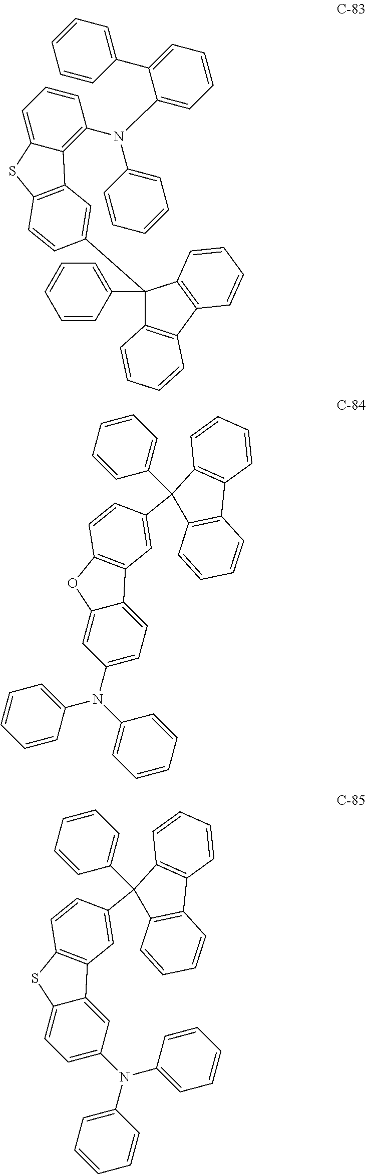

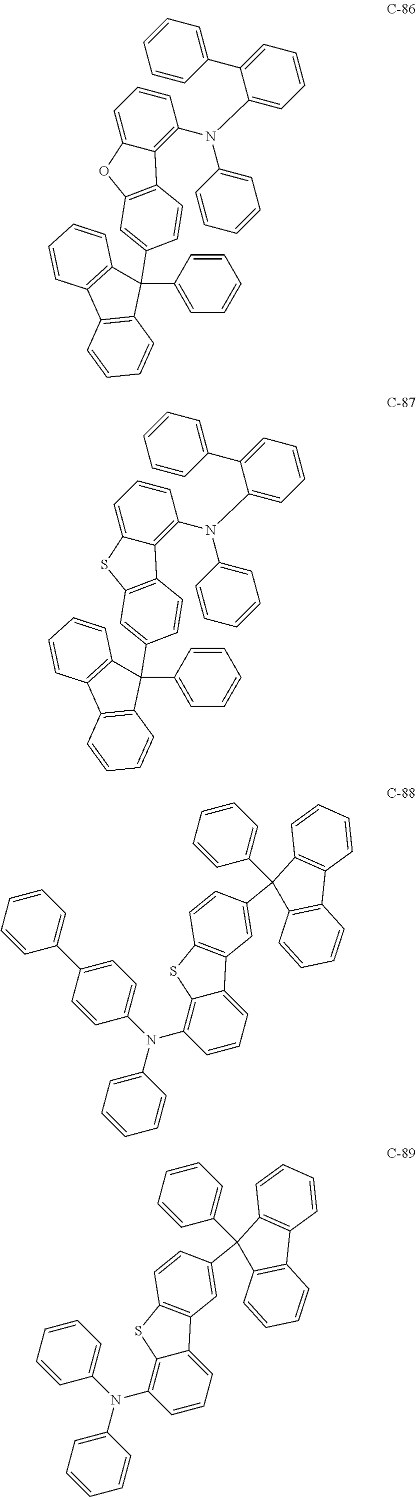

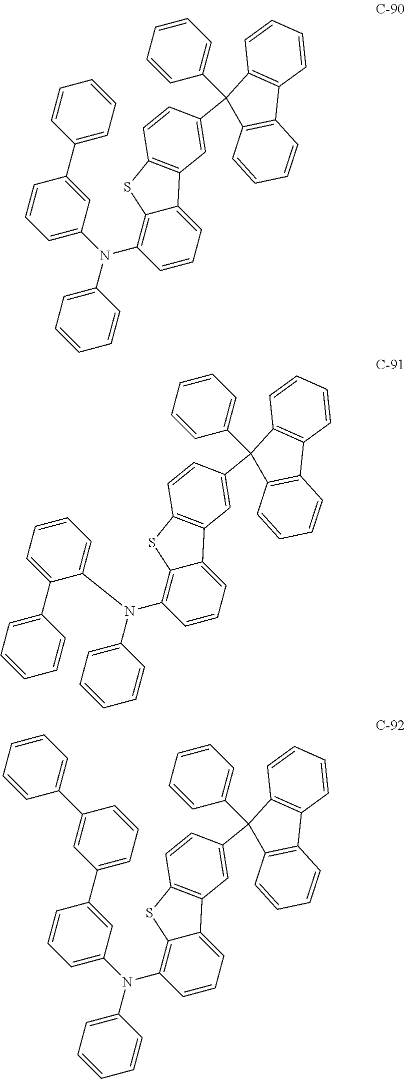 [SMILES: c1ccc(-c2cccc(N(c3ccccc3)c3cccc4c3sc3ccc(C5(c6ccccc6)c6ccccc6-c6ccccc65)cc34)c2)cc1 ; c1ccc(-c2ccccc2N(c2ccccc2)c2cccc3c2sc2ccc(C4(c5ccccc5)c5ccccc5-c5ccccc54)cc23)cc1 ; c1ccc(-c2cccc(-c3cccc(N(c4ccccc4)c4cccc5c4sc4ccc(C6(c7ccccc7)c7ccccc7-c7ccccc76)cc45)c3)c2)cc1]

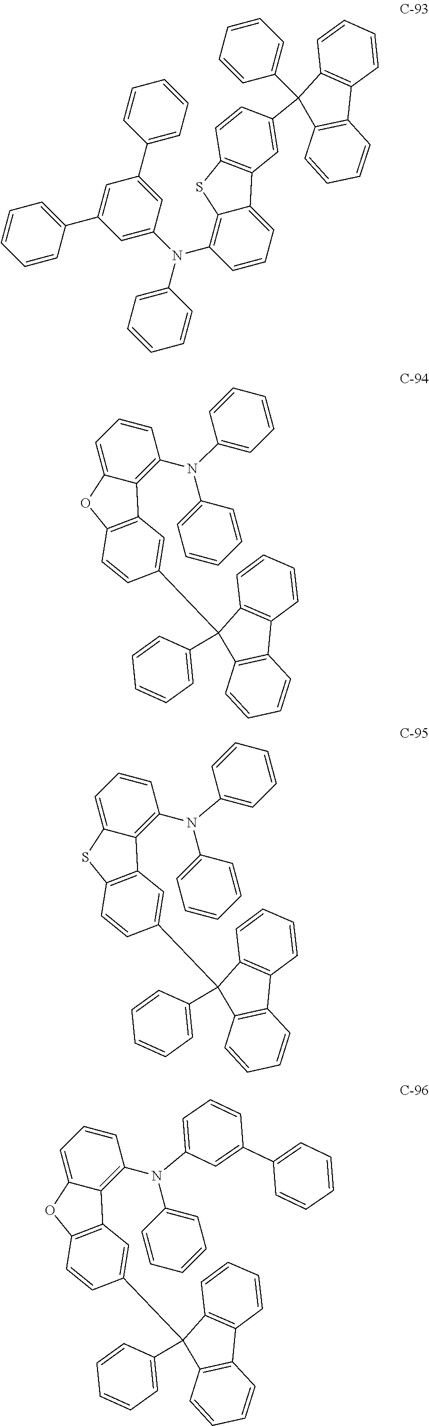

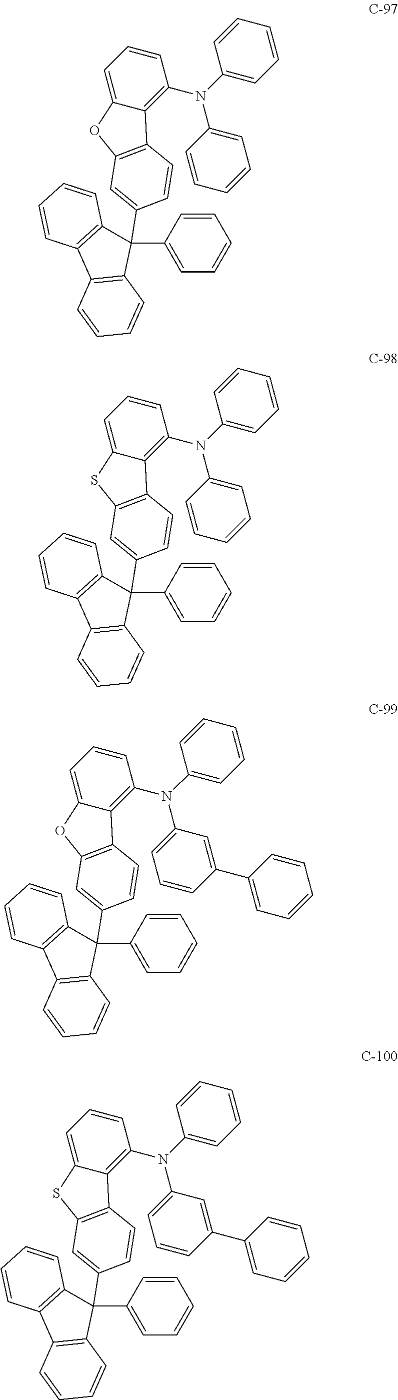 [SMILES: c1ccc(N(c2ccccc2)c2cccc3oc4cc(C5(c6ccccc6)c6ccccc6-c6ccccc65)ccc4c23)cc1 ; c1ccc(N(c2ccccc2)c2cccc3sc4cc(C5(c6ccccc6)c6ccccc6-c6ccccc65)ccc4c23)cc1 ; c1ccc(-c2cccc(N(c3ccccc3)c3cccc4oc5cc(C6(c7ccccc7)c7ccccc7-c7ccccc76)ccc5c34)c2)cc1 ; c1ccc(-c2cccc(N(c3ccccc3)c3cccc4sc5cc(C6(c7ccccc7)c7ccccc7-c7ccccc76)ccc5c34)c2)cc1]

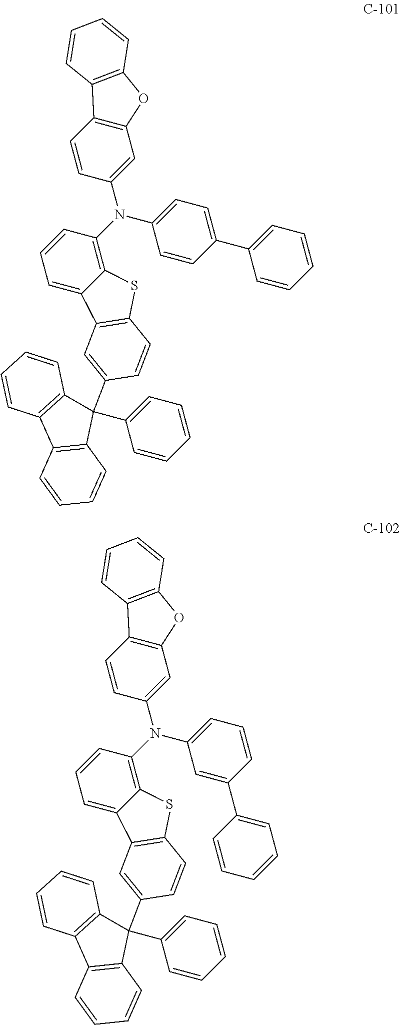 [SMILES: c1ccc(-c2ccc(N(c3ccc4c(c3)oc3ccccc34)c3cccc4c3sc3ccc(C5(c6ccccc6)c6ccccc6-c6ccccc65)cc34)cc2)cc1 ; c1ccc(-c2cccc(N(c3ccc4c(c3)oc3ccccc34)c3cccc4c3sc3ccc(C5(c6ccccc6)c6ccccc6-c6ccccc65)cc34)c2)cc1]

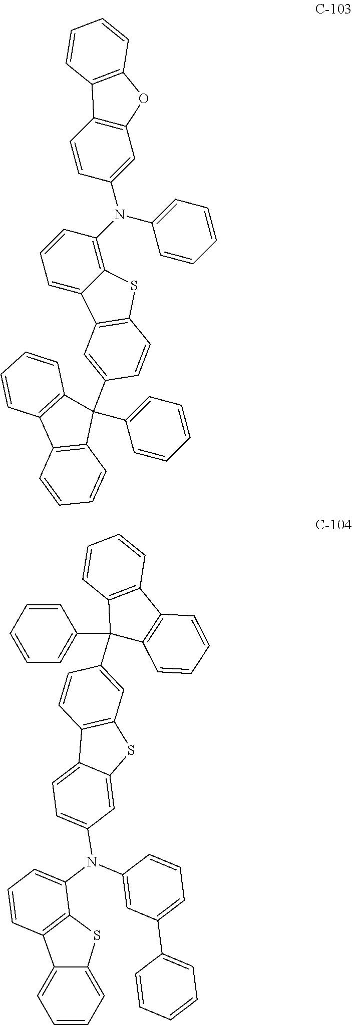

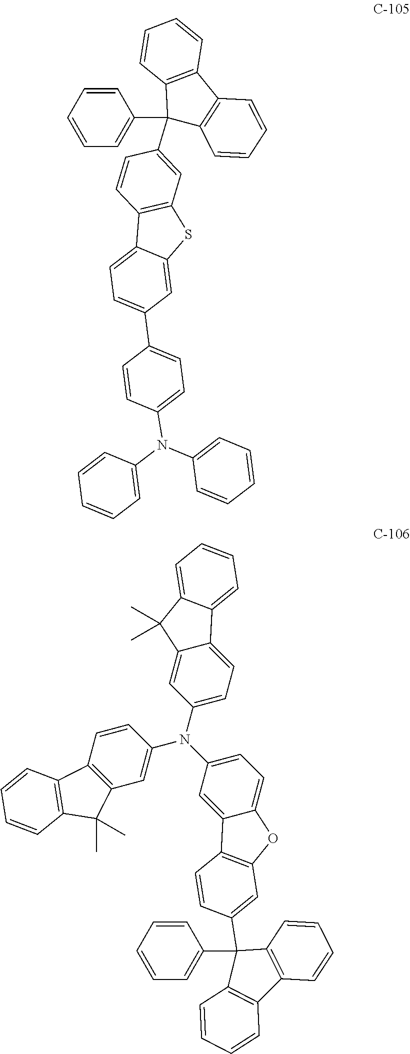 [SMILES: c1ccc(N(c2ccccc2)c2ccc(-c3ccc4c(c3)sc3cc(C5(c6ccccc6)c6ccccc6-c6ccccc65)ccc34)cc2)cc1 ; CC1(C)c2ccccc2-c2ccc(N(c3ccc4c(c3)C(C)(C)c3ccccc3-4)c3ccc4oc5cc(C6(c7ccccc7)c7ccccc7-c7ccccc76)ccc5c4c3)cc21]

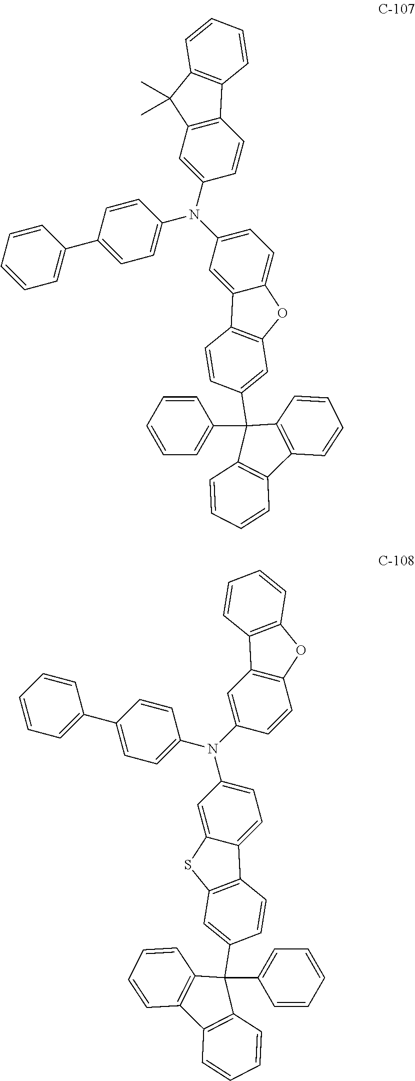

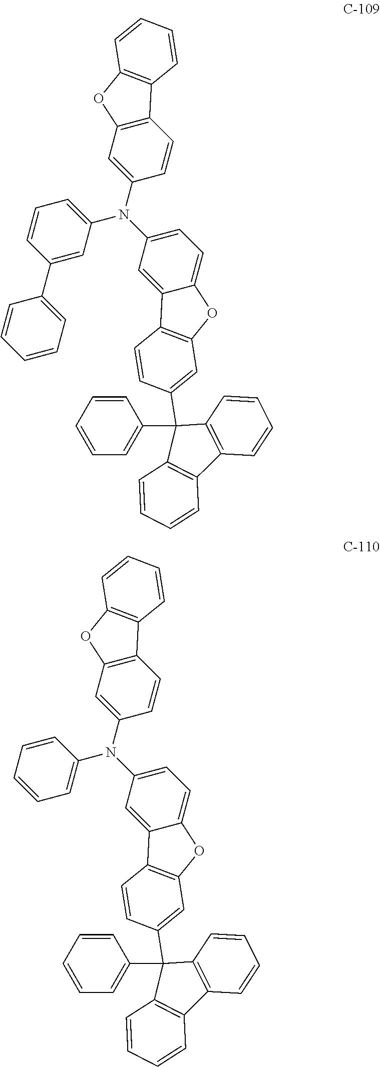

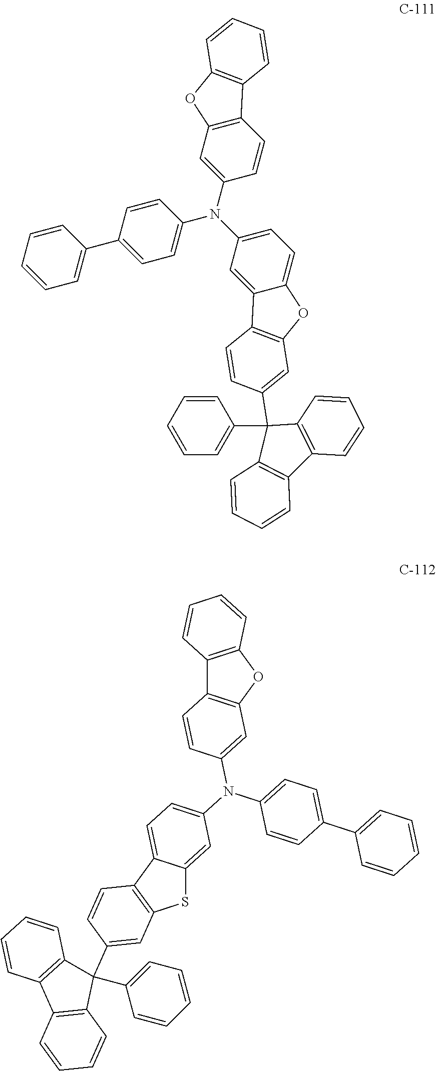 [SMILES: c1ccc(-c2ccc(N(c3ccc4c(c3)oc3ccccc34)c3ccc4oc5cc(C6(c7ccccc7)c7ccccc7-c7ccccc76)ccc5c4c3)cc2)cc1 ; c1ccc(-c2ccc(N(c3ccc4c(c3)oc3ccccc34)c3ccc4c(c3)sc3cc(C5(c6ccccc6)c6ccccc6-c6ccccc65)ccc34)cc2)cc1]

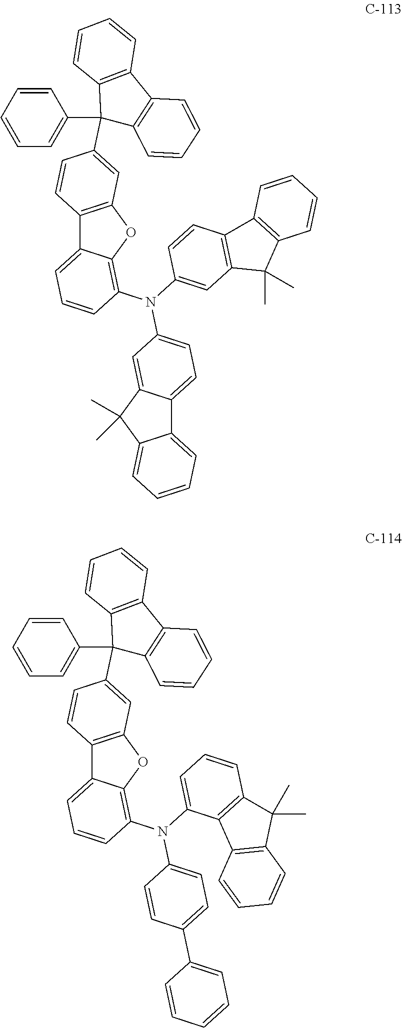 [SMILES: CC1(C)c2ccccc2-c2ccc(N(c3ccc4c(c3)C(C)(C)c3ccccc3-4)c3cccc4c3oc3cc(C5(c6ccccc6)c6ccccc6-c6ccccc65)ccc34)cc21 ; CC1(C)c2ccccc2-c2c(N(c3ccc(-c4ccccc4)cc3)c3cccc4c3oc3cc(C5(c6ccccc6)c6ccccc6-c6ccccc65)ccc34)cccc21]

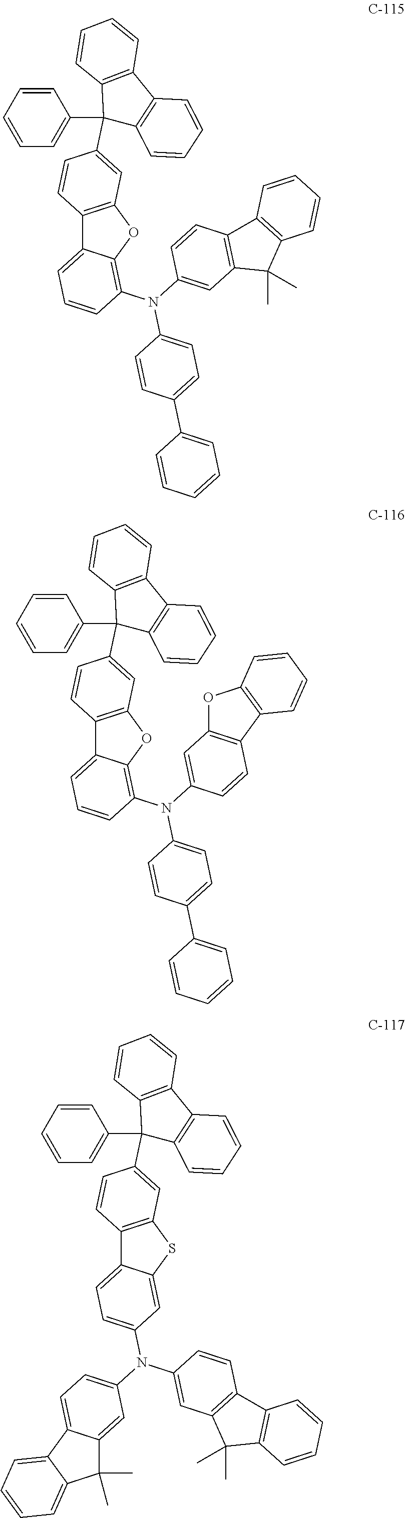 [SMILES: CC1(C)c2ccccc2-c2ccc(N(c3ccc(-c4ccccc4)cc3)c3cccc4c3oc3cc(C5(c6ccccc6)c6ccccc6-c6ccccc65)ccc34)cc21 ; c1ccc(-c2ccc(N(c3ccc4c(c3)oc3ccccc34)c3cccc4c3oc3cc(C5(c6ccccc6)c6ccccc6-c6ccccc65)ccc34)cc2)cc1 ; CC1(C)c2ccccc2-c2ccc(N(c3ccc4c(c3)C(C)(C)c3ccccc3-4)c3ccc4c(c3)sc3cc(C5(c6ccccc6)c6ccccc6-c6ccccc65)ccc34)cc21]

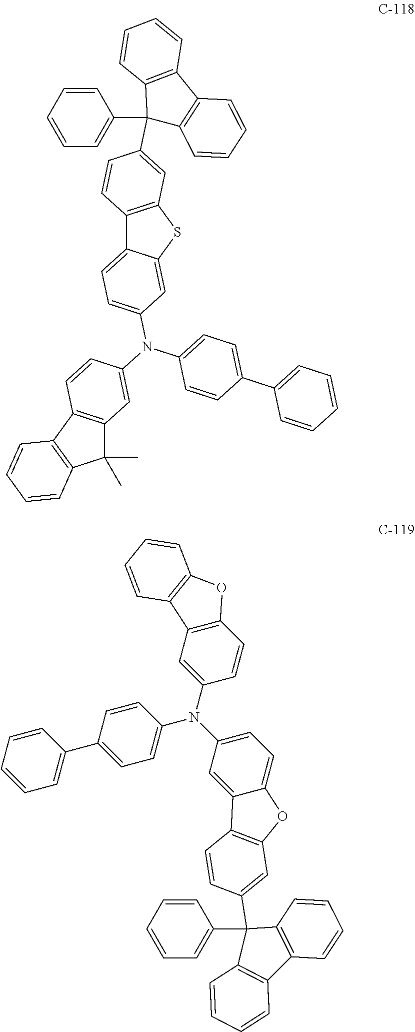 [SMILES: CC1(C)c2ccccc2-c2ccc(N(c3ccc(-c4ccccc4)cc3)c3ccc4c(c3)sc3cc(C5(c6ccccc6)c6ccccc6-c6ccccc65)ccc34)cc21 ; c1ccc(-c2ccc(N(c3ccc4oc5ccccc5c4c3)c3ccc4oc5cc(C6(c7ccccc7)c7ccccc7-c7ccccc76)ccc5c4c3)cc2)cc1]

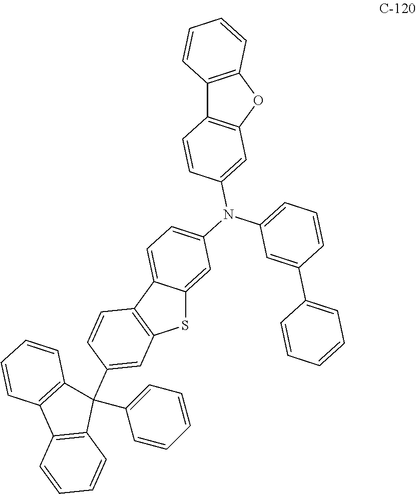 [SMILES: c1ccc(-c2cccc(N(c3ccc4c(c3)oc3ccccc34)c3ccc4c(c3)sc3cc(C5(c6ccccc6)c6ccccc6-c6ccccc65)ccc34)c2)cc1]

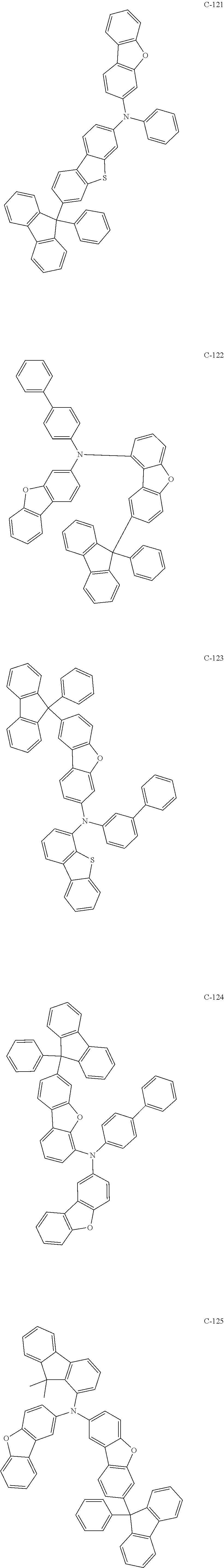 [SMILES: c1ccc(N(c2ccc3c(c2)oc2ccccc23)c2ccc3c(c2)sc2cc(C4(c5ccccc5)c5ccccc5-c5ccccc54)ccc23)cc1 ; c1ccc(-c2ccc(N(c3ccc4c(c3)oc3ccccc34)c3cccc4oc5ccc(C6(c7ccccc7)c7ccccc7-c7ccccc76)cc5c34)cc2)cc1 ; c1ccc(-c2cccc(N(c3ccc4c(c3)oc3ccc(C5(c6ccccc6)c6ccccc6-c6ccccc65)cc34)c3cccc4c3sc3ccccc34)c2)cc1 ; c1ccc(-c2ccc(N(c3ccc4oc5ccccc5c4c3)c3cccc4c3oc3cc(C5(c6ccccc6)c6ccccc6-c6ccccc65)ccc34)cc2)cc1 ; CC1(C)c2ccccc2-c2cccc(N(c3ccc4oc5ccccc5c4c3)c3ccc4oc5cc(C6(c7ccccc7)c7ccccc7-c7ccccc76)ccc5c4c3)c21]

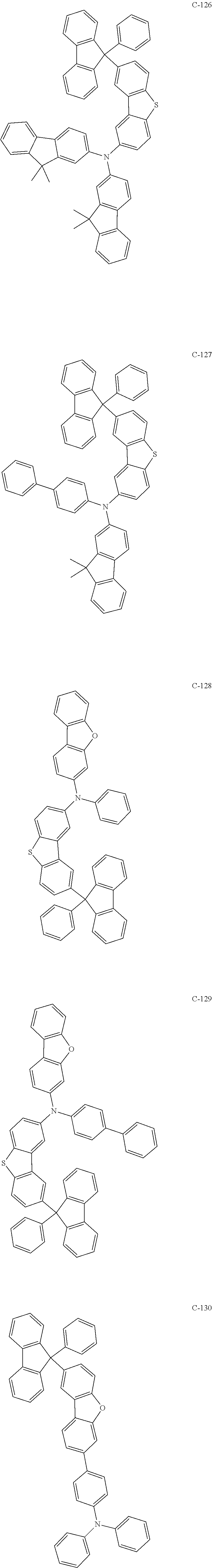 [SMILES: CC1(C)c2ccccc2-c2ccc(N(c3ccc4c(c3)C(C)(C)c3ccccc3-4)c3ccc4sc5ccc(C6(c7ccccc7)c7ccccc7-c7ccccc76)cc5c4c3)cc21 ; CC1(C)c2ccccc2-c2ccc(N(c3ccc(-c4ccccc4)cc3)c3ccc4sc5ccc(C6(c7ccccc7)c7ccccc7-c7ccccc76)cc5c4c3)cc21 ; c1ccc(N(c2ccc3c(c2)oc2ccccc23)c2ccc3sc4ccc(C5(c6ccccc6)c6ccccc6-c6ccccc65)cc4c3c2)cc1 ; c1ccc(-c2ccc(N(c3ccc4c(c3)oc3ccccc34)c3ccc4sc5ccc(C6(c7ccccc7)c7ccccc7-c7ccccc76)cc5c4c3)cc2)cc1 ; c1ccc(N(c2ccccc2)c2ccc(-c3ccc4c(c3)oc3ccc(C5(c6ccccc6)c6ccccc6-c6ccccc65)cc34)cc2)cc1]

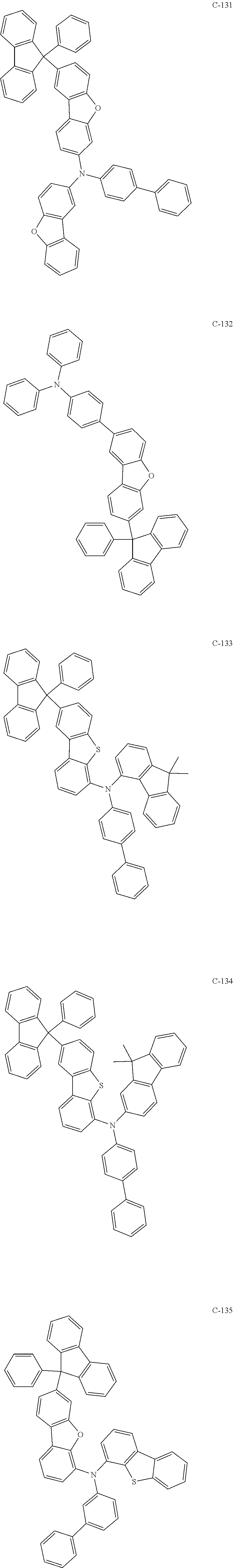 [SMILES: c1ccc(-c2ccc(N(c3ccc4c(c3)oc3ccc(C5(c6ccccc6)c6ccccc6-c6ccccc65)cc34)c3ccc4oc5ccccc5c4c3)cc2)cc1 ; c1ccc(N(c2ccccc2)c2ccc(-c3ccc4oc5cc(C6(c7ccccc7)c7ccccc7-c7ccccc76)ccc5c4c3)cc2)cc1 ; CC1(C)c2ccccc2-c2c(N(c3ccc(-c4ccccc4)cc3)c3cccc4c3sc3ccc(C5(c6ccccc6)c6ccccc6-c6ccccc65)cc34)cccc21 ; CC1(C)c2ccccc2-c2ccc(N(c3ccc(-c4ccccc4)cc3)c3cccc4c3sc3ccc(C5(c6ccccc6)c6ccccc6-c6ccccc65)cc34)cc21 ; c1ccc(-c2cccc(N(c3cccc4c3oc3cc(C5(c6ccccc6)c6ccccc6-c6ccccc65)ccc34)c3cccc4c3sc3ccccc34)c2)cc1]

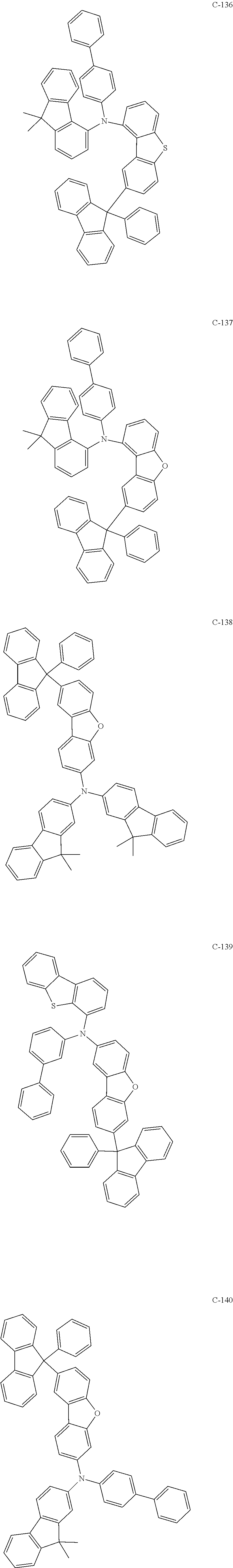

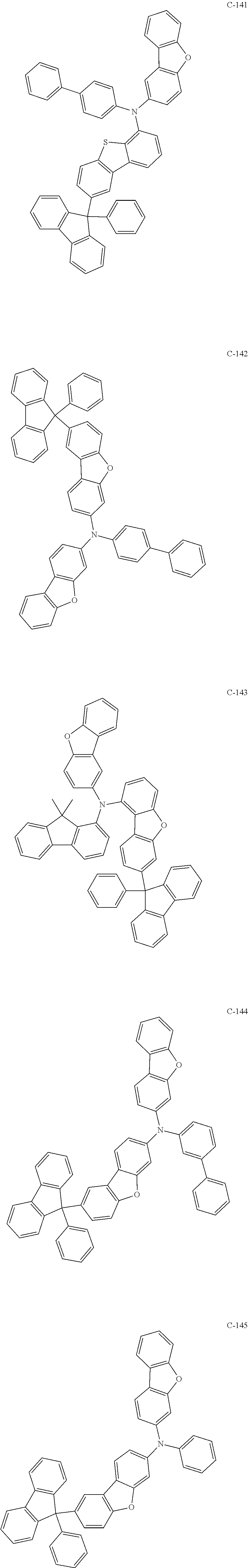 [SMILES: c1ccc(-c2ccc(N(c3ccc4oc5ccccc5c4c3)c3cccc4c3sc3ccc(C5(c6ccccc6)c6ccccc6-c6ccccc65)cc34)cc2)cc1 ; c1ccc(-c2ccc(N(c3ccc4c(c3)oc3ccccc34)c3ccc4c(c3)oc3ccc(C5(c6ccccc6)c6ccccc6-c6ccccc65)cc34)cc2)cc1 ; CC1(C)c2ccccc2-c2cccc(N(c3ccc4oc5ccccc5c4c3)c3cccc4oc5cc(C6(c7ccccc7)c7ccccc7-c7ccccc76)ccc5c34)c21 ; c1ccc(-c2cccc(N(c3ccc4c(c3)oc3ccccc34)c3ccc4c(c3)oc3ccc(C5(c6ccccc6)c6ccccc6-c6ccccc65)cc34)c2)cc1 ; c1ccc(N(c2ccc3c(c2)oc2ccccc23)c2ccc3c(c2)oc2ccc(C4(c5ccccc5)c5ccccc5-c5ccccc54)cc23)cc1]

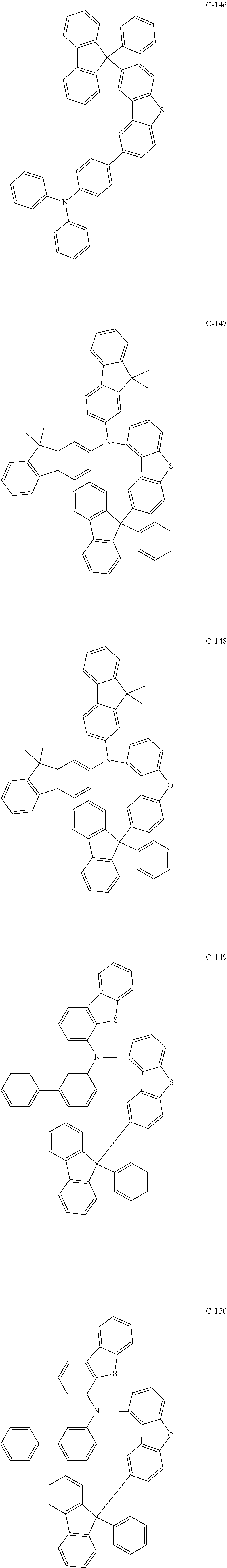 [SMILES: c1ccc(N(c2ccccc2)c2ccc(-c3ccc4sc5ccc(C6(c7ccccc7)c7ccccc7-c7ccccc76)cc5c4c3)cc2)cc1 ; CC1(C)c2ccccc2-c2ccc(N(c3ccc4c(c3)C(C)(C)c3ccccc3-4)c3cccc4sc5ccc(C6(c7ccccc7)c7ccccc7-c7ccccc76)cc5c34)cc21 ; CC1(C)c2ccccc2-c2ccc(N(c3ccc4c(c3)C(C)(C)c3ccccc3-4)c3cccc4oc5ccc(C6(c7ccccc7)c7ccccc7-c7ccccc76)cc5c34)cc21 ; c1ccc(-c2cccc(N(c3cccc4c3sc3ccccc34)c3cccc4sc5ccc(C6(c7ccccc7)c7ccccc7-c7ccccc76)cc5c34)c2)cc1 ; c1ccc(-c2cccc(N(c3cccc4c3sc3ccccc34)c3cccc4oc5ccc(C6(c7ccccc7)c7ccccc7-c7ccccc76)cc5c34)c2)cc1]

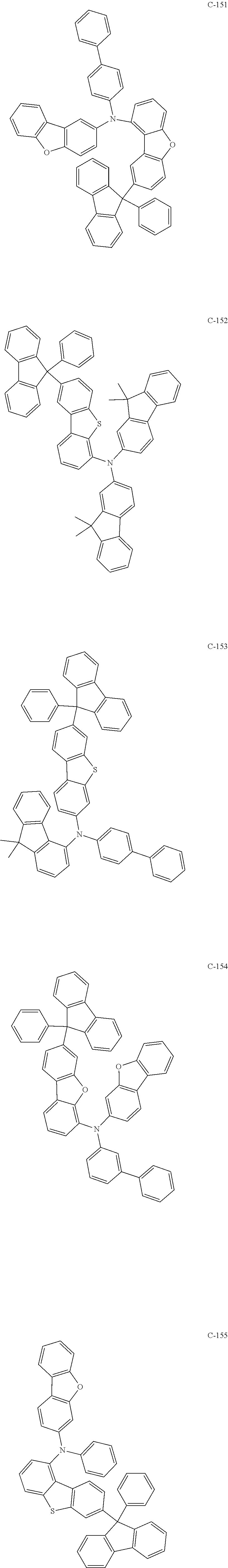 [SMILES: c1ccc(-c2ccc(N(c3ccc4oc5ccccc5c4c3)c3cccc4oc5ccc(C6(c7ccccc7)c7ccccc7-c7ccccc76)cc5c34)cc2)cc1 ; CC1(C)c2ccccc2-c2ccc(N(c3ccc4c(c3)C(C)(C)c3ccccc3-4)c3cccc4c3sc3ccc(C5(c6ccccc6)c6ccccc6-c6ccccc65)cc34)cc21 ; CC1(C)c2ccccc2-c2c(N(c3ccc(-c4ccccc4)cc3)c3ccc4c(c3)sc3cc(C5(c6ccccc6)c6ccccc6-c6ccccc65)ccc34)cccc21 ; c1ccc(-c2cccc(N(c3ccc4c(c3)oc3ccccc34)c3cccc4c3oc3cc(C5(c6ccccc6)c6ccccc6-c6ccccc65)ccc34)c2)cc1 ; c1ccc(N(c2ccc3c(c2)oc2ccccc23)c2cccc3sc4cc(C5(c6ccccc6)c6ccccc6-c6ccccc65)ccc4c23)cc1]

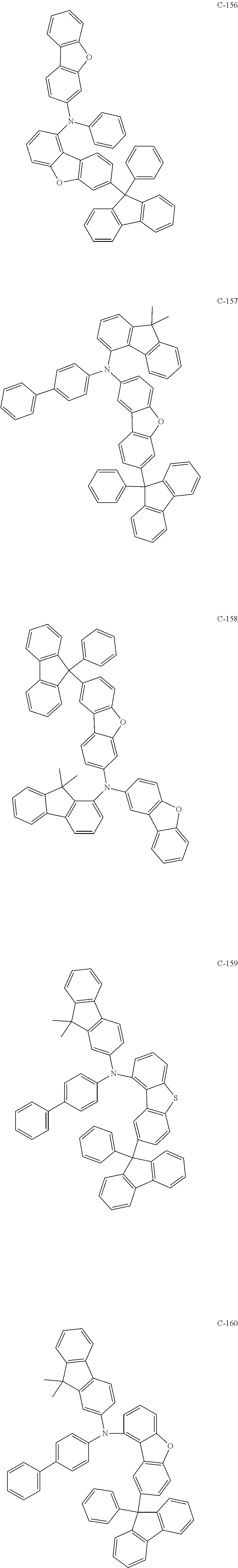 [SMILES: c1ccc(N(c2ccc3c(c2)oc2ccccc23)c2cccc3oc4cc(C5(c6ccccc6)c6ccccc6-c6ccccc65)ccc4c23)cc1 ; CC1(C)c2ccccc2-c2c(N(c3ccc(-c4ccccc4)cc3)c3ccc4oc5cc(C6(c7ccccc7)c7ccccc7-c7ccccc76)ccc5c4c3)cccc21 ; CC1(C)c2ccccc2-c2cccc(N(c3ccc4c(c3)oc3ccc(C5(c6ccccc6)c6ccccc6-c6ccccc65)cc34)c3ccc4oc5ccccc5c4c3)c21 ; CC1(C)c2ccccc2-c2ccc(N(c3ccc(-c4ccccc4)cc3)c3cccc4sc5ccc(C6(c7ccccc7)c7ccccc7-c7ccccc76)cc5c34)cc21 ; CC1(C)c2ccccc2-c2ccc(N(c3ccc(-c4ccccc4)cc3)c3cccc4oc5ccc(C6(c7ccccc7)c7ccccc7-c7ccccc76)cc5c34)cc21]

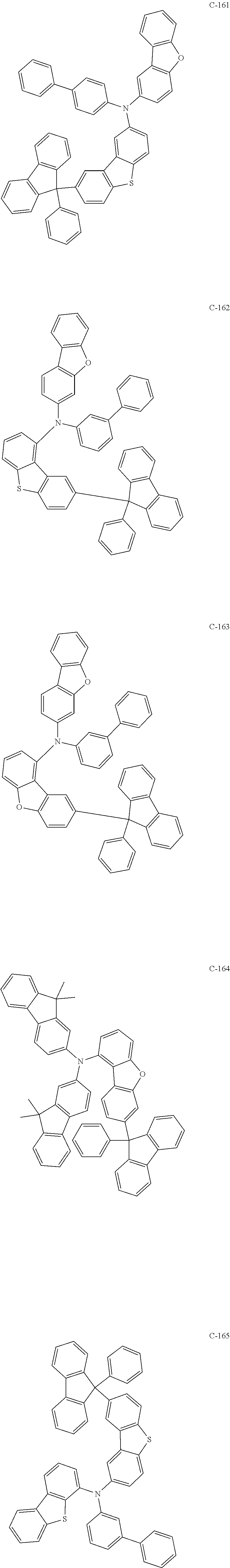 [SMILES: c1ccc(-c2ccc(N(c3ccc4oc5ccccc5c4c3)c3ccc4sc5ccc(C6(c7ccccc7)c7ccccc7-c7ccccc76)cc5c4c3)cc2)cc1 ; c1ccc(-c2cccc(N(c3ccc4c(c3)oc3ccccc34)c3cccc4sc5ccc(C6(c7ccccc7)c7ccccc7-c7ccccc76)cc5c34)c2)cc1 ; c1ccc(-c2cccc(N(c3ccc4c(c3)oc3ccccc34)c3cccc4oc5ccc(C6(c7ccccc7)c7ccccc7-c7ccccc76)cc5c34)c2)cc1 ; CC1(C)c2ccccc2-c2ccc(N(c3ccc4c(c3)C(C)(C)c3ccccc3-4)c3cccc4oc5cc(C6(c7ccccc7)c7ccccc7-c7ccccc76)ccc5c34)cc21 ; c1ccc(-c2cccc(N(c3ccc4sc5ccc(C6(c7ccccc7)c7ccccc7-c7ccccc76)cc5c4c3)c3cccc4c3sc3ccccc34)c2)cc1]

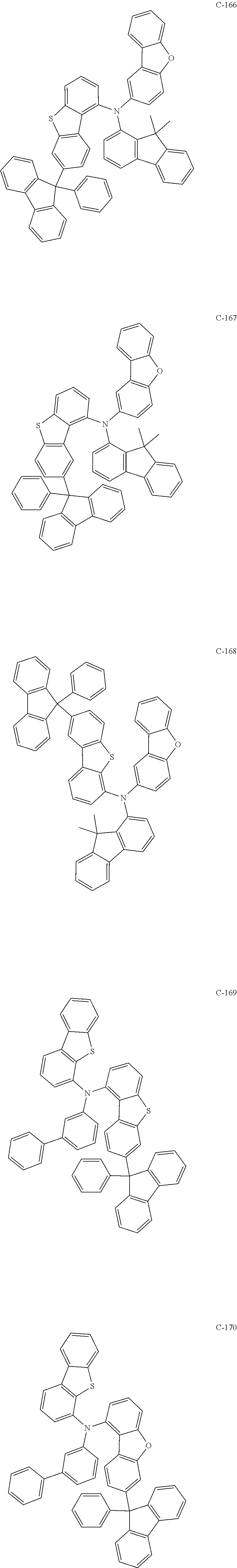 [SMILES: CC1(C)c2ccccc2-c2cccc(N(c3ccc4oc5ccccc5c4c3)c3cccc4sc5cc(C6(c7ccccc7)c7ccccc7-c7ccccc76)ccc5c34)c21 ; CC1(C)c2ccccc2-c2cccc(N(c3ccc4oc5ccccc5c4c3)c3cccc4sc5ccc(C6(c7ccccc7)c7ccccc7-c7ccccc76)cc5c34)c21 ; CC1(C)c2ccccc2-c2cccc(N(c3ccc4oc5ccccc5c4c3)c3cccc4c3sc3ccc(C5(c6ccccc6)c6ccccc6-c6ccccc65)cc34)c21 ; c1ccc(-c2cccc(N(c3cccc4c3sc3ccccc34)c3cccc4sc5cc(C6(c7ccccc7)c7ccccc7-c7ccccc76)ccc5c34)c2)cc1 ; c1ccc(-c2cccc(N(c3cccc4c3sc3ccccc34)c3cccc4oc5cc(C6(c7ccccc7)c7ccccc7-c7ccccc76)ccc5c34)c2)cc1]

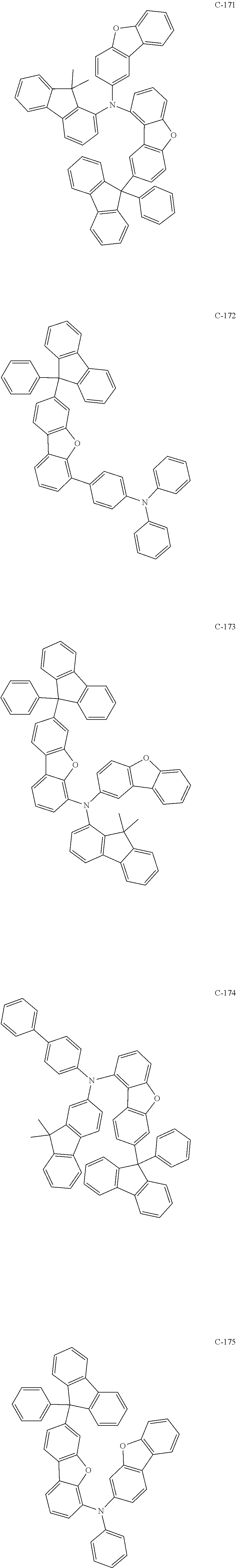 [SMILES: CC1(C)c2ccccc2-c2cccc(N(c3ccc4oc5ccccc5c4c3)c3cccc4oc5ccc(C6(c7ccccc7)c7ccccc7-c7ccccc76)cc5c34)c21 ; c1ccc(N(c2ccccc2)c2ccc(-c3cccc4c3oc3cc(C5(c6ccccc6)c6ccccc6-c6ccccc65)ccc34)cc2)cc1 ; CC1(C)c2ccccc2-c2cccc(N(c3ccc4oc5ccccc5c4c3)c3cccc4c3oc3cc(C5(c6ccccc6)c6ccccc6-c6ccccc65)ccc34)c21 ; CC1(C)c2ccccc2-c2ccc(N(c3ccc(-c4ccccc4)cc3)c3cccc4oc5cc(C6(c7ccccc7)c7ccccc7-c7ccccc76)ccc5c34)cc21 ; c1ccc(N(c2ccc3c(c2)oc2ccccc23)c2cccc3c2oc2cc(C4(c5ccccc5)c5ccccc5-c5ccccc54)ccc23)cc1]

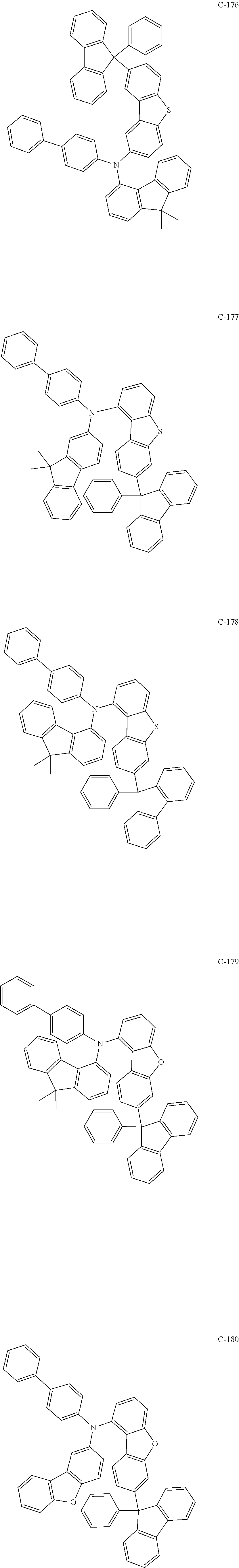

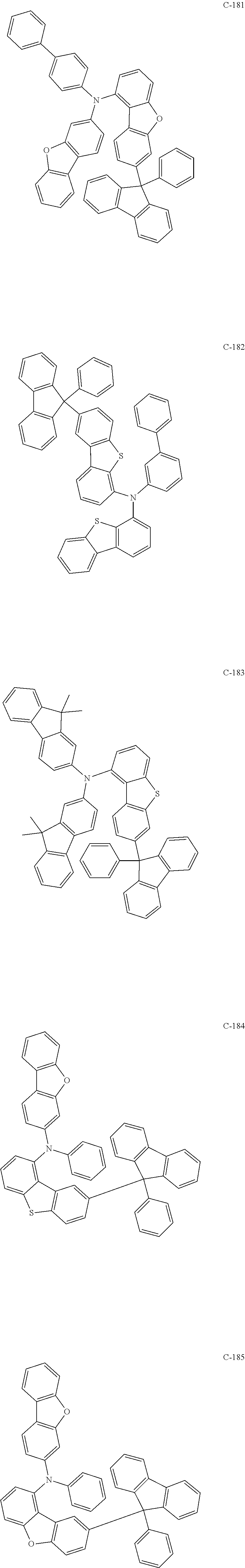 [SMILES: c1ccc(-c2ccc(N(c3ccc4c(c3)oc3ccccc34)c3cccc4oc5cc(C6(c7ccccc7)c7ccccc7-c7ccccc76)ccc5c34)cc2)cc1 ; c1ccc(-c2cccc(N(c3cccc4c3sc3ccccc34)c3cccc4c3sc3ccc(C5(c6ccccc6)c6ccccc6-c6ccccc65)cc34)c2)cc1 ; CC1(C)c2ccccc2-c2ccc(N(c3ccc4c(c3)C(C)(C)c3ccccc3-4)c3cccc4sc5cc(C6(c7ccccc7)c7ccccc7-c7ccccc76)ccc5c34)cc21 ; c1ccc(N(c2ccc3c(c2)oc2ccccc23)c2cccc3sc4ccc(C5(c6ccccc6)c6ccccc6-c6ccccc65)cc4c23)cc1 ; c1ccc(N(c2ccc3c(c2)oc2ccccc23)c2cccc3oc4ccc(C5(c6ccccc6)c6ccccc6-c6ccccc65)cc4c23)cc1]

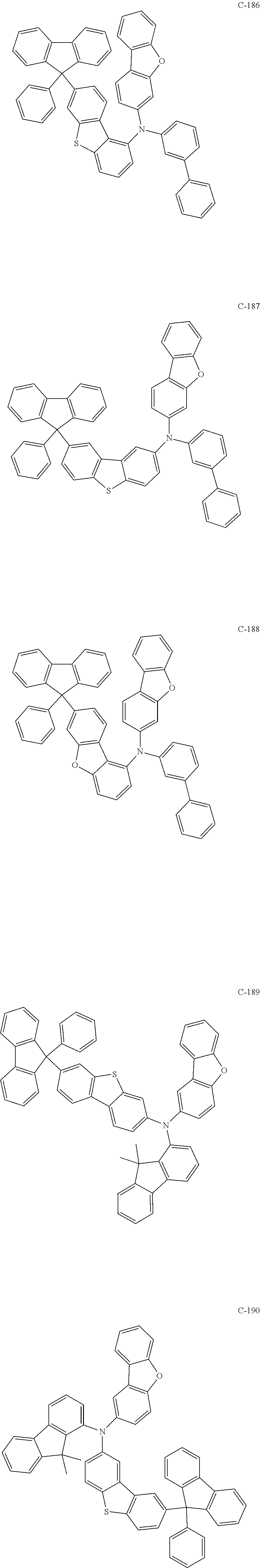 [SMILES: c1ccc(-c2cccc(N(c3ccc4c(c3)oc3ccccc34)c3cccc4sc5cc(C6(c7ccccc7)c7ccccc7-c7ccccc76)ccc5c34)c2)cc1 ; c1ccc(-c2cccc(N(c3ccc4c(c3)oc3ccccc34)c3ccc4sc5ccc(C6(c7ccccc7)c7ccccc7-c7ccccc76)cc5c4c3)c2)cc1 ; c1ccc(-c2cccc(N(c3ccc4c(c3)oc3ccccc34)c3cccc4oc5cc(C6(c7ccccc7)c7ccccc7-c7ccccc76)ccc5c34)c2)cc1 ; CC1(C)c2ccccc2-c2cccc(N(c3ccc4c(c3)sc3cc(C5(c6ccccc6)c6ccccc6-c6ccccc65)ccc34)c3ccc4oc5ccccc5c4c3)c21 ; CC1(C)c2ccccc2-c2cccc(N(c3ccc4oc5ccccc5c4c3)c3ccc4sc5ccc(C6(c7ccccc7)c7ccccc7-c7ccccc76)cc5c4c3)c21]

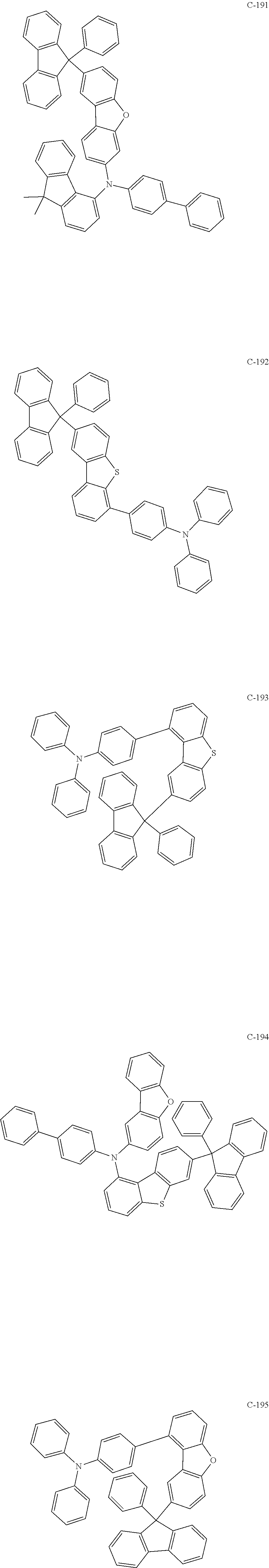 [SMILES: CC1(C)c2ccccc2-c2c(N(c3ccc(-c4ccccc4)cc3)c3ccc4c(c3)oc3ccc(C5(c6ccccc6)c6ccccc6-c6ccccc65)cc34)cccc21 ; c1ccc(N(c2ccccc2)c2ccc(-c3cccc4c3sc3ccc(C5(c6ccccc6)c6ccccc6-c6ccccc65)cc34)cc2)cc1 ; c1ccc(N(c2ccccc2)c2ccc(-c3cccc4sc5ccc(C6(c7ccccc7)c7ccccc7-c7ccccc76)cc5c34)cc2)cc1 ; c1ccc(-c2ccc(N(c3ccc4oc5ccccc5c4c3)c3cccc4sc5cc(C6(c7ccccc7)c7ccccc7-c7ccccc76)ccc5c34)cc2)cc1 ; c1ccc(N(c2ccccc2)c2ccc(-c3cccc4oc5ccc(C6(c7ccccc7)c7ccccc7-c7ccccc76)cc5c34)cc2)cc1]

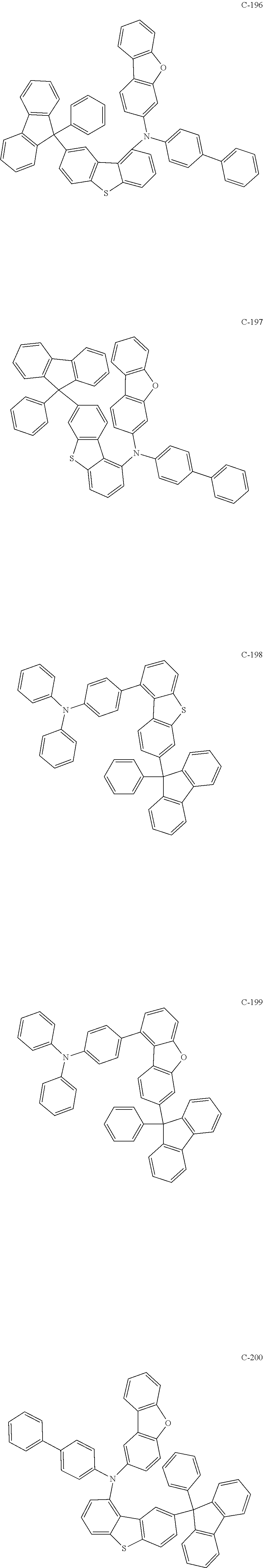

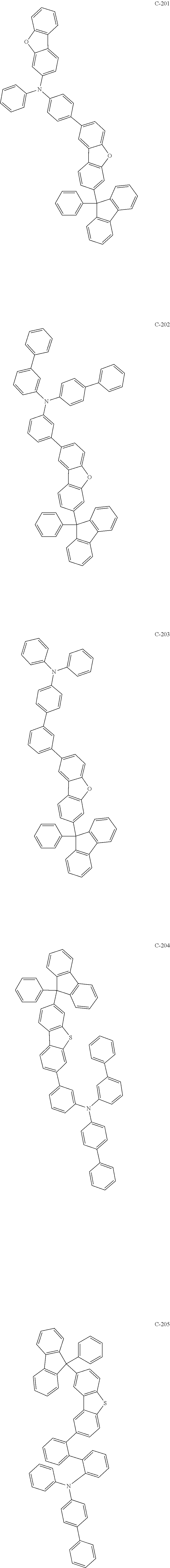

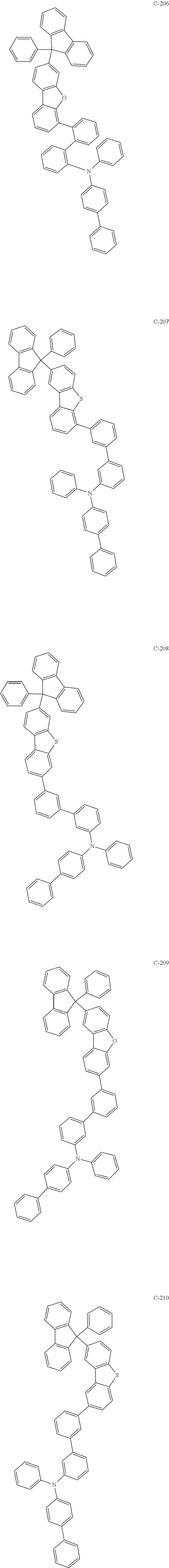

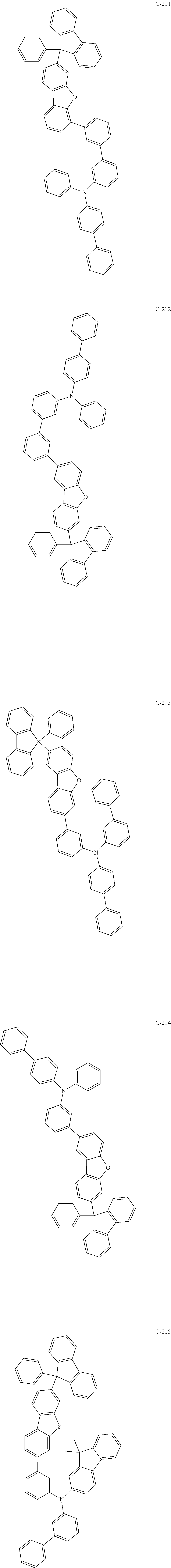

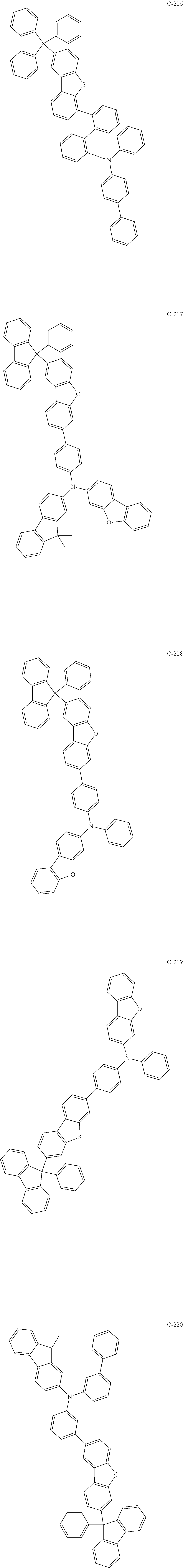

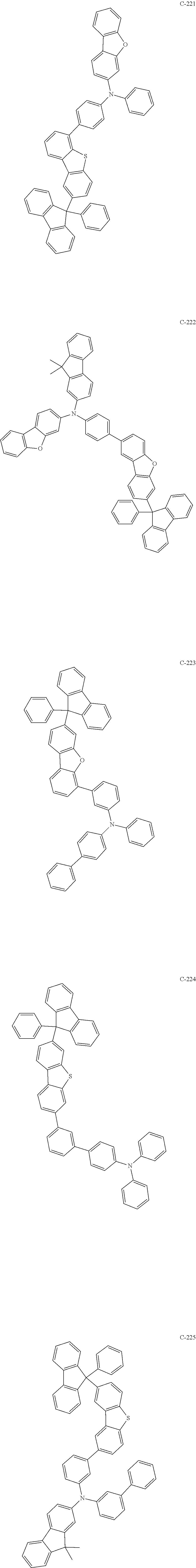

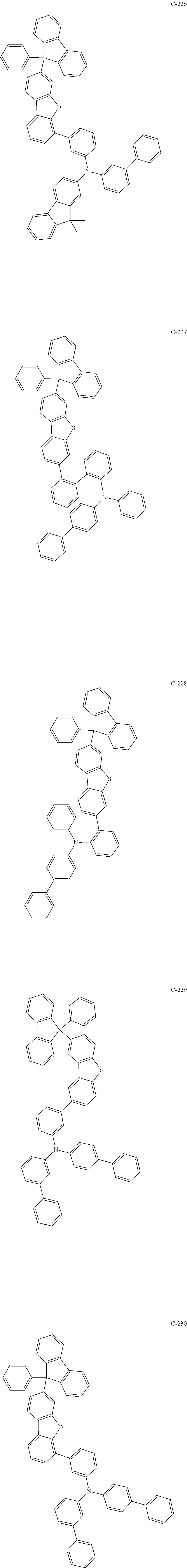

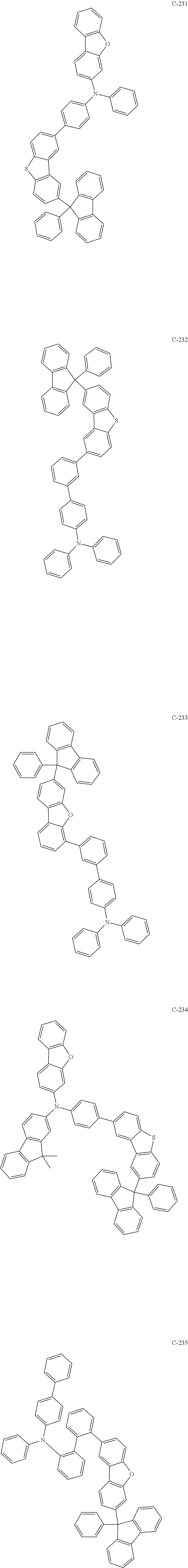

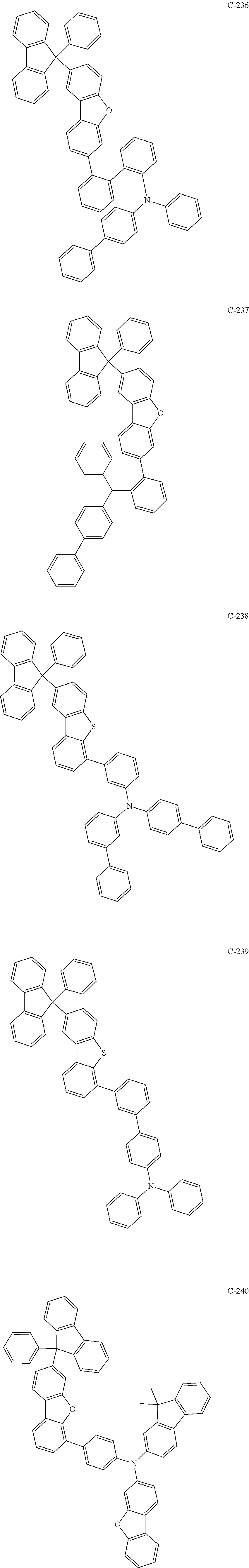

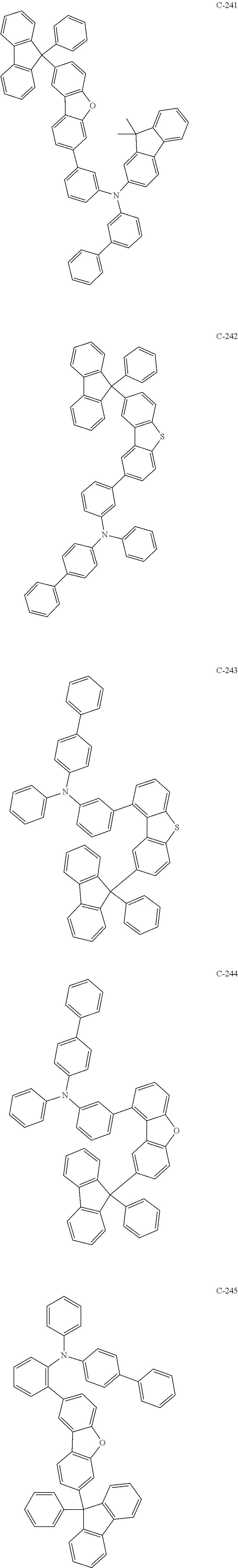 [SMILES: CC1(C)c2ccccc2-c2ccc(N(c3cccc(-c4ccccc4)c3)c3cccc(-c4ccc5c(c4)oc4ccc(C6(c7ccccc7)c7ccccc7-c7ccccc76)cc45)c3)cc21 ; c1ccc(-c2ccc(N(c3ccccc3)c3cccc(-c4ccc5sc6ccc(C7(c8ccccc8)c8ccccc8-c8ccccc87)cc6c5c4)c3)cc2)cc1 ; c1ccc(-c2ccc(N(c3ccccc3)c3cccc(-c4cccc5sc6ccc(C7(c8ccccc8)c8ccccc8-c8ccccc87)cc6c45)c3)cc2)cc1 ; c1ccc(-c2ccc(N(c3ccccc3)c3cccc(-c4cccc5oc6ccc(C7(c8ccccc8)c8ccccc8-c8ccccc87)cc6c45)c3)cc2)cc1 ; c1ccc(-c2ccc(N(c3ccccc3)c3ccccc3-c3ccc4oc5cc(C6(c7ccccc7)c7ccccc7-c7ccccc76)ccc5c4c3)cc2)cc1]

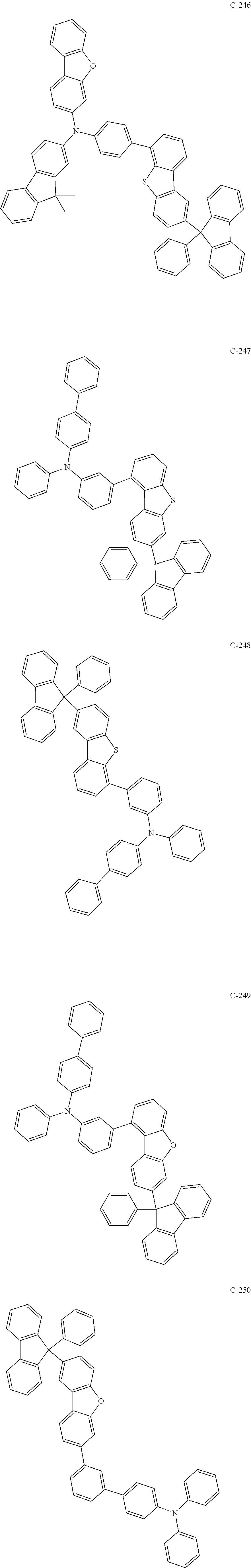

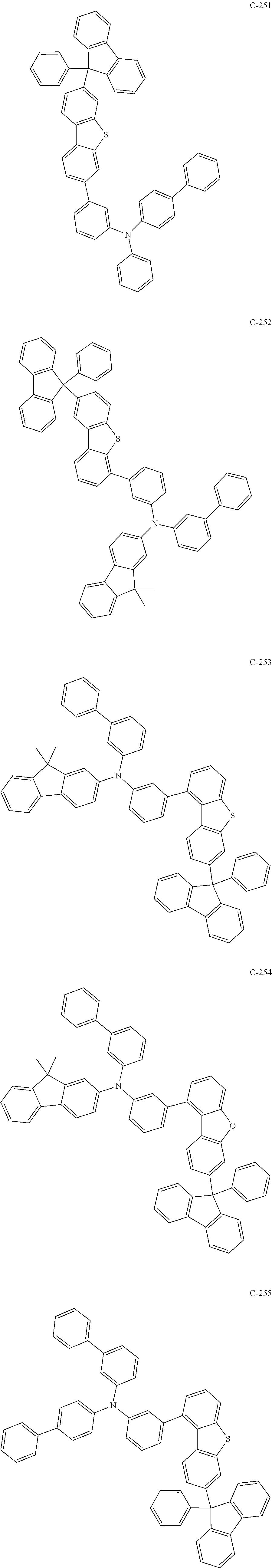

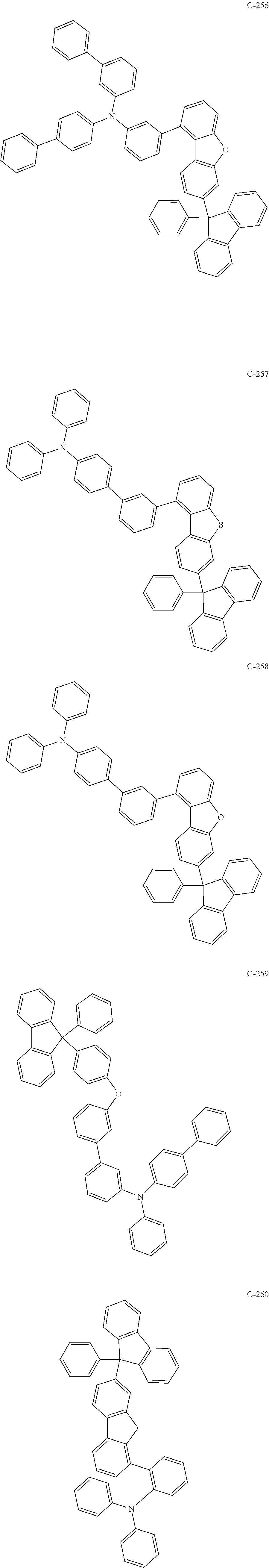 [SMILES: c1ccc(-c2ccc(N(c3cccc(-c4ccccc4)c3)c3cccc(-c4cccc5oc6cc(C7(c8ccccc8)c8ccccc8-c8ccccc87)ccc6c45)c3)cc2)cc1 ; c1ccc(N(c2ccccc2)c2ccc(-c3cccc(-c4cccc5sc6cc(C7(c8ccccc8)c8ccccc8-c8ccccc87)ccc6c45)c3)cc2)cc1 ; c1ccc(N(c2ccccc2)c2ccc(-c3cccc(-c4cccc5oc6cc(C7(c8ccccc8)c8ccccc8-c8ccccc87)ccc6c45)c3)cc2)cc1 ; c1ccc(-c2ccc(N(c3ccccc3)c3cccc(-c4ccc5c(c4)oc4ccc(C6(c7ccccc7)c7ccccc7-c7ccccc76)cc45)c3)cc2)cc1 ; c1ccc(N(c2ccccc2)c2ccccc2-c2cccc3c2Cc2cc(C4(c5ccccc5)c5ccccc5-c5ccccc54)ccc2-3)cc1]

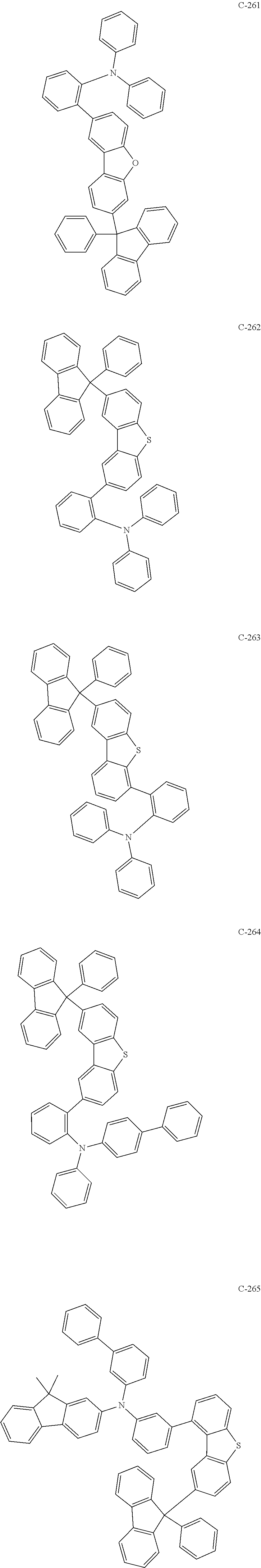 [SMILES: c1ccc(N(c2ccccc2)c2ccccc2-c2ccc3oc4cc(C5(c6ccccc6)c6ccccc6-c6ccccc65)ccc4c3c2)cc1 ; c1ccc(N(c2ccccc2)c2ccccc2-c2ccc3sc4ccc(C5(c6ccccc6)c6ccccc6-c6ccccc65)cc4c3c2)cc1 ; c1ccc(N(c2ccccc2)c2ccccc2-c2cccc3c2sc2ccc(C4(c5ccccc5)c5ccccc5-c5ccccc54)cc23)cc1 ; c1ccc(-c2ccc(N(c3ccccc3)c3ccccc3-c3ccc4sc5ccc(C6(c7ccccc7)c7ccccc7-c7ccccc76)cc5c4c3)cc2)cc1 ; CC1(C)c2ccccc2-c2ccc(N(c3cccc(-c4ccccc4)c3)c3cccc(-c4cccc5sc6ccc(C7(c8ccccc8)c8ccccc8-c8ccccc87)cc6c45)c3)cc21]

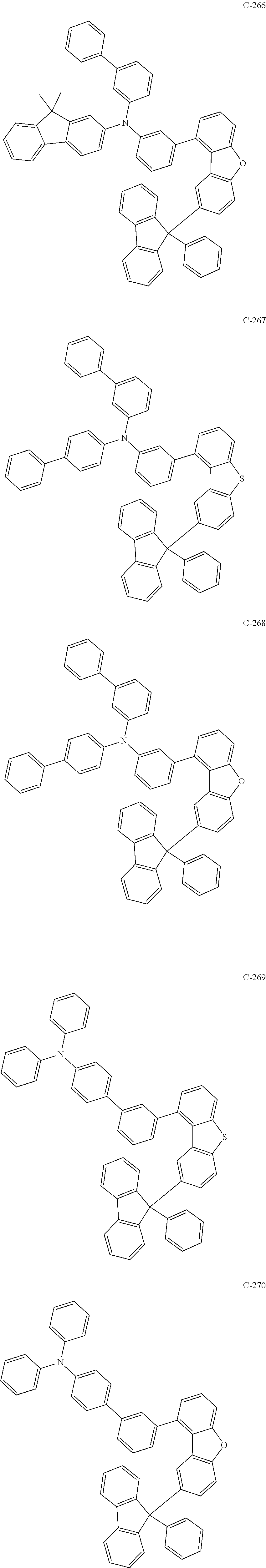

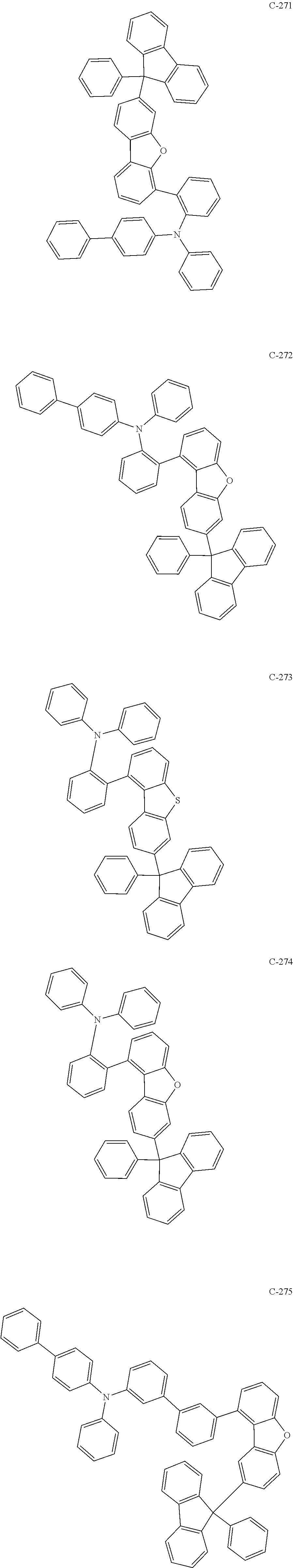 [SMILES: c1ccc(-c2ccc(N(c3ccccc3)c3ccccc3-c3cccc4c3oc3cc(C5(c6ccccc6)c6ccccc6-c6ccccc65)ccc34)cc2)cc1 ; c1ccc(-c2ccc(N(c3ccccc3)c3ccccc3-c3cccc4oc5cc(C6(c7ccccc7)c7ccccc7-c7ccccc76)ccc5c34)cc2)cc1 ; c1ccc(N(c2ccccc2)c2ccccc2-c2cccc3sc4cc(C5(c6ccccc6)c6ccccc6-c6ccccc65)ccc4c23)cc1 ; c1ccc(N(c2ccccc2)c2ccccc2-c2cccc3oc4cc(C5(c6ccccc6)c6ccccc6-c6ccccc65)ccc4c23)cc1 ; c1ccc(-c2ccc(N(c3ccccc3)c3cccc(-c4cccc(-c5cccc6oc7ccc(C8(c9ccccc9)c9ccccc9-c9ccccc98)cc7c56)c4)c3)cc2)cc1]

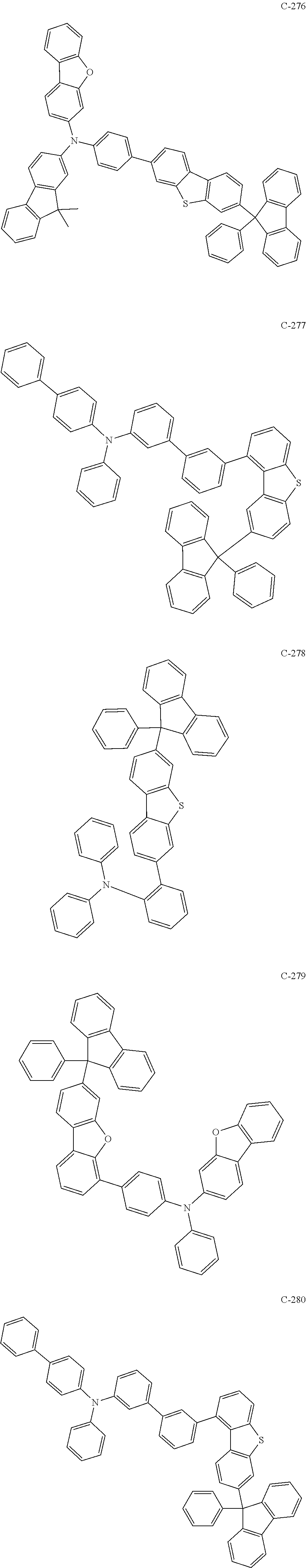

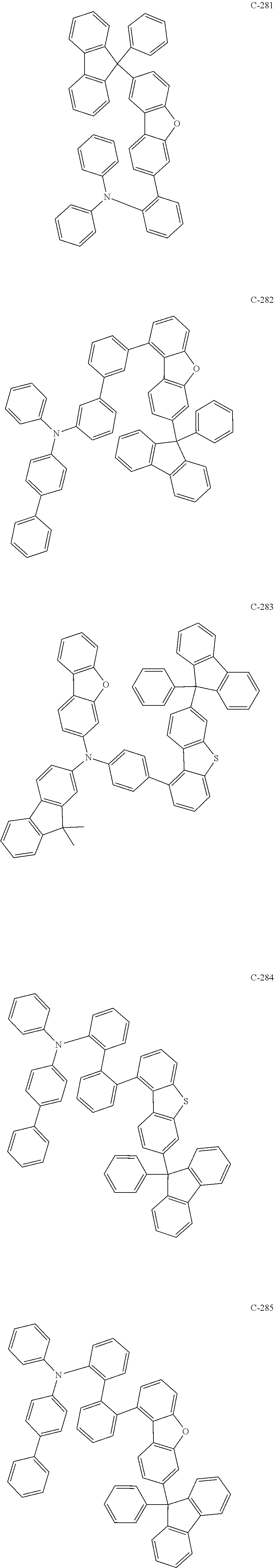

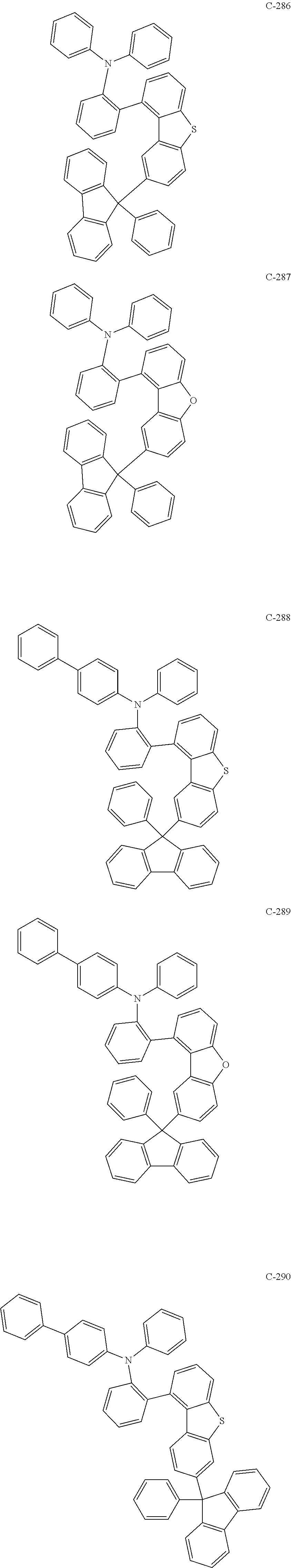

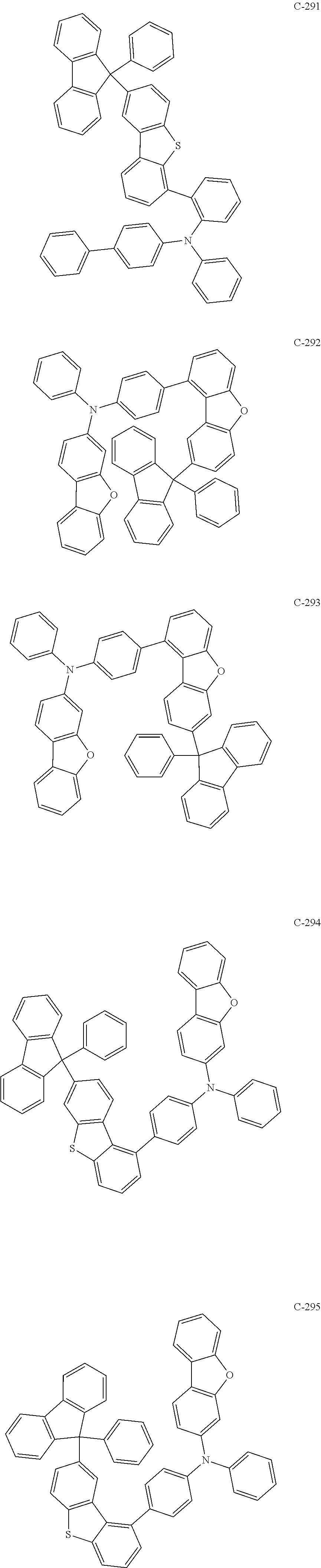 [SMILES: c1ccc(-c2ccc(N(c3ccccc3)c3ccccc3-c3cccc4c3sc3ccc(C5(c6ccccc6)c6ccccc6-c6ccccc65)cc34)cc2)cc1 ; c1ccc(N(c2ccc(-c3cccc4oc5ccc(C6(c7ccccc7)c7ccccc7-c7ccccc76)cc5c34)cc2)c2ccc3c(c2)oc2ccccc23)cc1 ; c1ccc(N(c2ccc(-c3cccc4oc5cc(C6(c7ccccc7)c7ccccc7-c7ccccc76)ccc5c34)cc2)c2ccc3c(c2)oc2ccccc23)cc1 ; c1ccc(N(c2ccc(-c3cccc4sc5cc(C6(c7ccccc7)c7ccccc7-c7ccccc76)ccc5c34)cc2)c2ccc3c(c2)oc2ccccc23)cc1 ; c1ccc(N(c2ccc(-c3cccc4sc5ccc(C6(c7ccccc7)c7ccccc7-c7ccccc76)cc5c34)cc2)c2ccc3c(c2)oc2ccccc23)cc1]

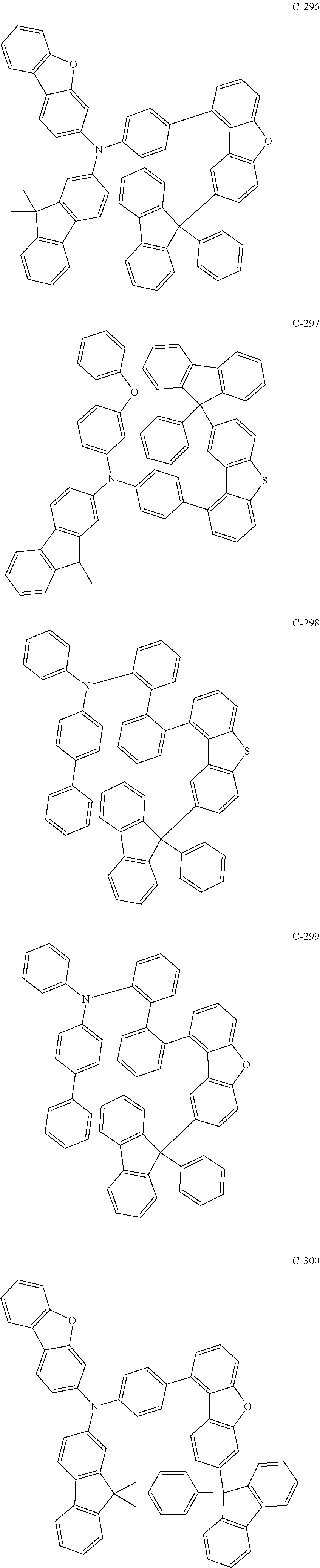

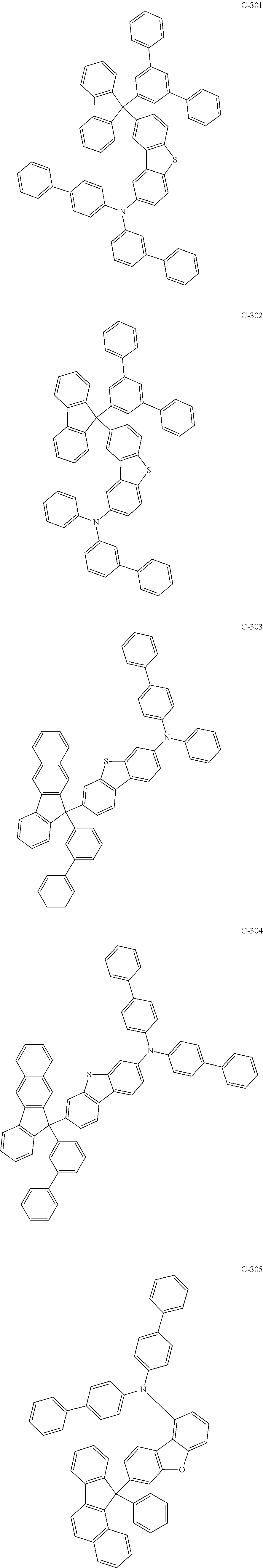

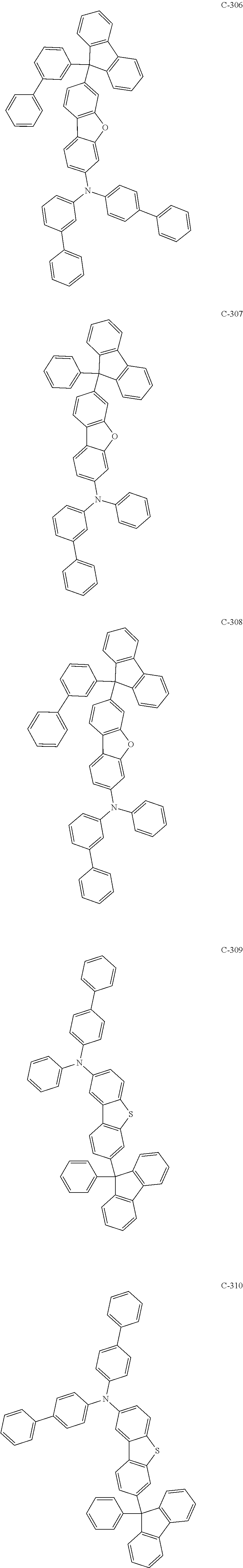 [SMILES: c1ccc(-c2ccc(N(c3cccc(-c4ccccc4)c3)c3ccc4c(c3)oc3cc(C5(c6cccc(-c7ccccc7)c6)c6ccccc6-c6ccccc65)ccc34)cc2)cc1 ; c1ccc(-c2cccc(N(c3ccccc3)c3ccc4c(c3)oc3cc(C5(c6ccccc6)c6ccccc6-c6ccccc65)ccc34)c2)cc1 ; c1ccc(-c2cccc(N(c3ccccc3)c3ccc4c(c3)oc3cc(C5(c6cccc(-c7ccccc7)c6)c6ccccc6-c6ccccc65)ccc34)c2)cc1 ; c1ccc(-c2ccc(N(c3ccccc3)c3ccc4sc5cc(C6(c7ccccc7)c7ccccc7-c7ccccc76)ccc5c4c3)cc2)cc1 ; c1ccc(-c2ccc(N(c3ccc(-c4ccccc4)cc3)c3ccc4sc5cc(C6(c7ccccc7)c7ccccc7-c7ccccc76)ccc5c4c3)cc2)cc1]

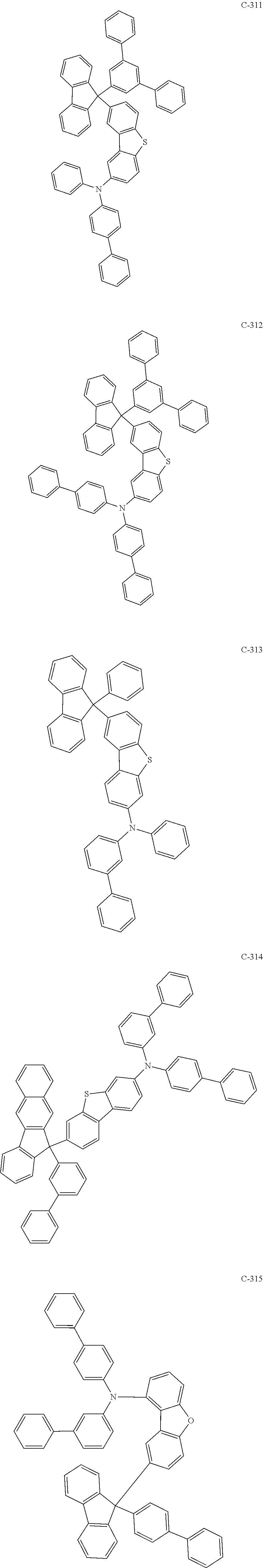

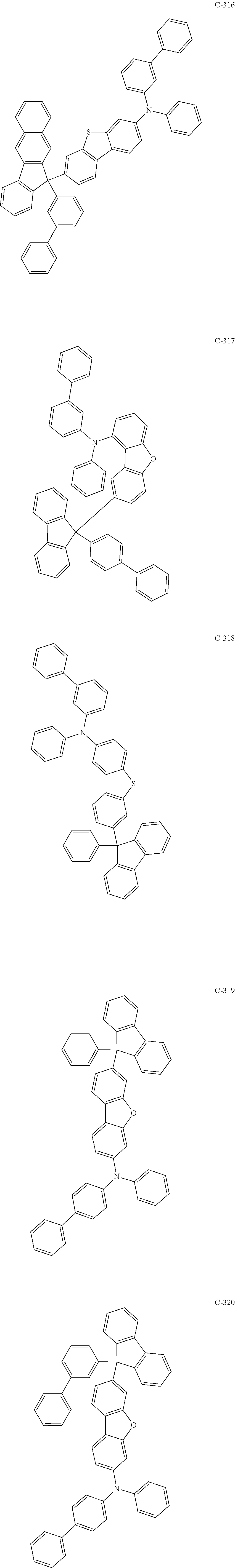 [SMILES: c1ccc(-c2cccc(N(c3ccccc3)c3ccc4c(c3)sc3cc(C5(c6cccc(-c7ccccc7)c6)c6ccccc6-c6cc7ccccc7cc65)ccc34)c2)cc1 ; c1ccc(-c2ccc(C3(c4ccc5oc6cccc(N(c7ccccc7)c7cccc(-c8ccccc8)c7)c6c5c4)c4ccccc4-c4ccccc43)cc2)cc1 ; c1ccc(-c2cccc(N(c3ccccc3)c3ccc4sc5cc(C6(c7ccccc7)c7ccccc7-c7ccccc76)ccc5c4c3)c2)cc1 ; c1ccc(-c2ccc(N(c3ccccc3)c3ccc4c(c3)oc3cc(C5(c6ccccc6)c6ccccc6-c6ccccc65)ccc34)cc2)cc1 ; c1ccc(-c2ccc(N(c3ccccc3)c3ccc4c(c3)oc3cc(C5(c6cccc(-c7ccccc7)c6)c6ccccc6-c6ccccc65)ccc34)cc2)cc1]

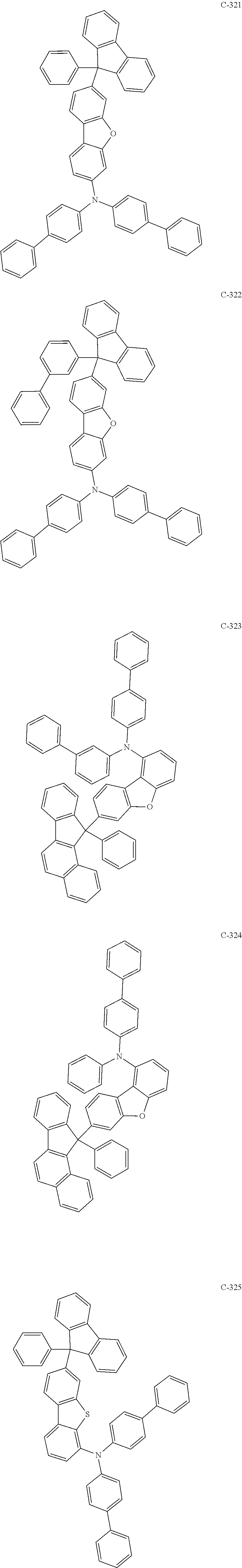 [SMILES: c1ccc(-c2ccc(N(c3ccc(-c4ccccc4)cc3)c3ccc4c(c3)oc3cc(C5(c6ccccc6)c6ccccc6-c6ccccc65)ccc34)cc2)cc1 ; c1ccc(-c2ccc(N(c3ccc(-c4ccccc4)cc3)c3ccc4c(c3)oc3cc(C5(c6cccc(-c7ccccc7)c6)c6ccccc6-c6ccccc65)ccc34)cc2)cc1 ; c1ccc(-c2ccc(N(c3cccc(-c4ccccc4)c3)c3cccc4oc5cc(C6(c7ccccc7)c7ccccc7-c7ccc8ccccc8c76)ccc5c34)cc2)cc1 ; c1ccc(-c2ccc(N(c3ccccc3)c3cccc4oc5cc(C6(c7ccccc7)c7ccccc7-c7ccc8ccccc8c76)ccc5c34)cc2)cc1 ; c1ccc(-c2ccc(N(c3ccc(-c4ccccc4)cc3)c3cccc4c3sc3cc(C5(c6ccccc6)c6ccccc6-c6ccccc65)ccc34)cc2)cc1]

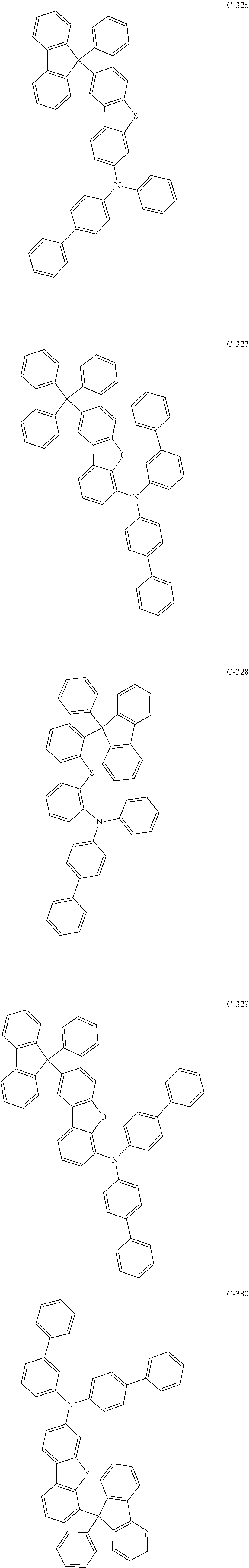 [SMILES: c1ccc(-c2ccc(N(c3ccccc3)c3ccc4c(c3)sc3ccc(C5(c6ccccc6)c6ccccc6-c6ccccc65)cc34)cc2)cc1 ; c1ccc(-c2ccc(N(c3cccc(-c4ccccc4)c3)c3cccc4c3oc3ccc(C5(c6ccccc6)c6ccccc6-c6ccccc65)cc34)cc2)cc1 ; c1ccc(-c2ccc(N(c3ccccc3)c3cccc4c3sc3c(C5(c6ccccc6)c6ccccc6-c6ccccc65)cccc34)cc2)cc1 ; c1ccc(-c2ccc(N(c3ccc(-c4ccccc4)cc3)c3cccc4c3oc3ccc(C5(c6ccccc6)c6ccccc6-c6ccccc65)cc34)cc2)cc1 ; c1ccc(-c2ccc(N(c3cccc(-c4ccccc4)c3)c3ccc4c(c3)sc3c(C5(c6ccccc6)c6ccccc6-c6ccccc65)cccc34)cc2)cc1]

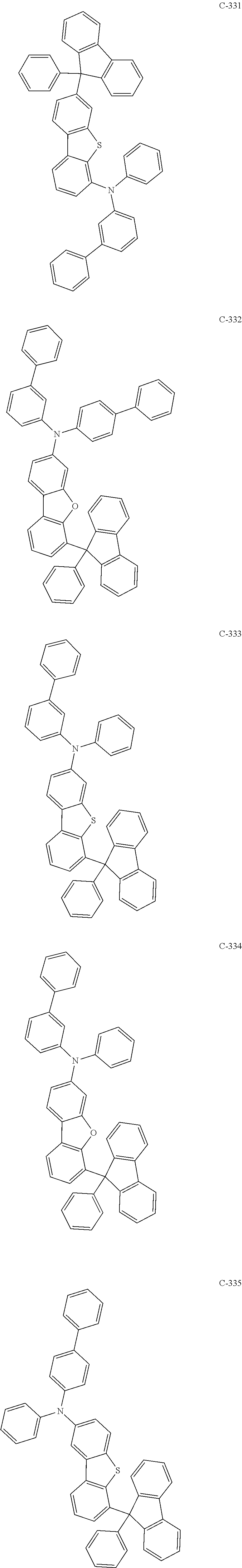 [SMILES: c1ccc(-c2cccc(N(c3ccccc3)c3cccc4c3sc3cc(C5(c6ccccc6)c6ccccc6-c6ccccc65)ccc34)c2)cc1 ; c1ccc(-c2ccc(N(c3cccc(-c4ccccc4)c3)c3ccc4c(c3)oc3c(C5(c6ccccc6)c6ccccc6-c6ccccc65)cccc34)cc2)cc1 ; c1ccc(-c2cccc(N(c3ccccc3)c3ccc4c(c3)sc3c(C5(c6ccccc6)c6ccccc6-c6ccccc65)cccc34)c2)cc1 ; c1ccc(-c2cccc(N(c3ccccc3)c3ccc4c(c3)oc3c(C5(c6ccccc6)c6ccccc6-c6ccccc65)cccc34)c2)cc1 ; c1ccc(-c2ccc(N(c3ccccc3)c3ccc4sc5c(C6(c7ccccc7)c7ccccc7-c7ccccc76)cccc5c4c3)cc2)cc1]

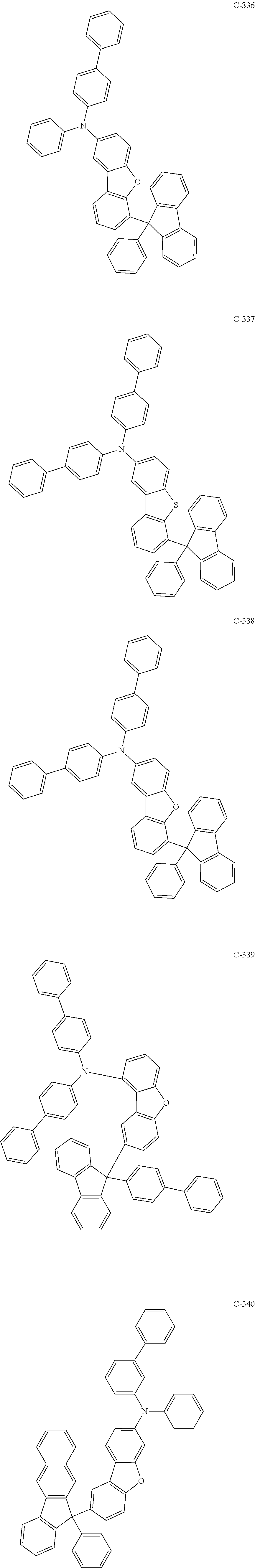 [SMILES: c1ccc(-c2ccc(N(c3ccccc3)c3ccc4oc5c(C6(c7ccccc7)c7ccccc7-c7ccccc76)cccc5c4c3)cc2)cc1 ; c1ccc(-c2ccc(N(c3ccc(-c4ccccc4)cc3)c3ccc4sc5c(C6(c7ccccc7)c7ccccc7-c7ccccc76)cccc5c4c3)cc2)cc1 ; c1ccc(-c2ccc(N(c3ccc(-c4ccccc4)cc3)c3ccc4oc5c(C6(c7ccccc7)c7ccccc7-c7ccccc76)cccc5c4c3)cc2)cc1 ; c1ccc(-c2ccc(N(c3ccc(-c4ccccc4)cc3)c3cccc4oc5ccc(C6(c7ccc(-c8ccccc8)cc7)c7ccccc7-c7ccccc76)cc5c34)cc2)cc1 ; c1ccc(-c2cccc(N(c3ccccc3)c3ccc4c(c3)oc3ccc(C5(c6ccccc6)c6ccccc6-c6cc7ccccc7cc65)cc34)c2)cc1]

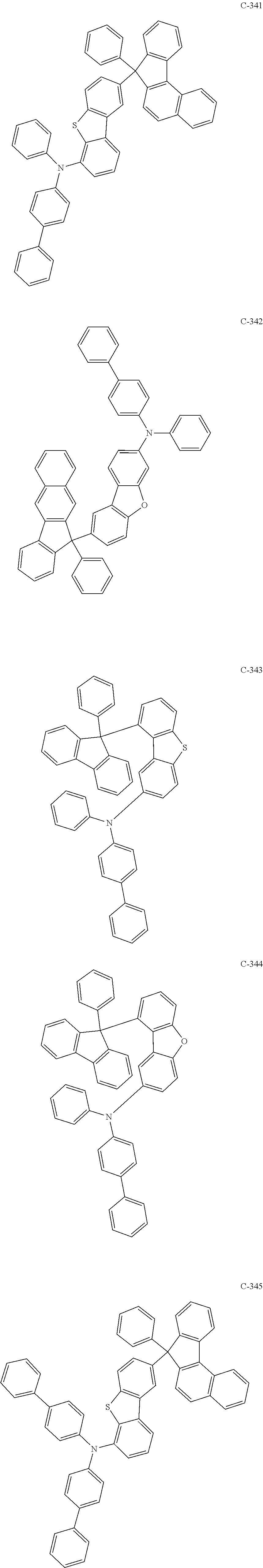 [SMILES: c1ccc(-c2ccc(N(c3ccccc3)c3cccc4c3sc3ccc(C5(c6ccccc6)c6ccccc6-c6c5ccc5ccccc65)cc34)cc2)cc1 ; c1ccc(-c2ccc(N(c3ccccc3)c3ccc4c(c3)oc3ccc(C5(c6ccccc6)c6ccccc6-c6cc7ccccc7cc65)cc34)cc2)cc1 ; c1ccc(-c2ccc(N(c3ccccc3)c3ccc4sc5cccc(C6(c7ccccc7)c7ccccc7-c7ccccc76)c5c4c3)cc2)cc1 ; c1ccc(-c2ccc(N(c3ccccc3)c3ccc4oc5cccc(C6(c7ccccc7)c7ccccc7-c7ccccc76)c5c4c3)cc2)cc1 ; c1ccc(-c2ccc(N(c3ccc(-c4ccccc4)cc3)c3cccc4c3sc3ccc(C5(c6ccccc6)c6ccccc6-c6c5ccc5ccccc65)cc34)cc2)cc1]

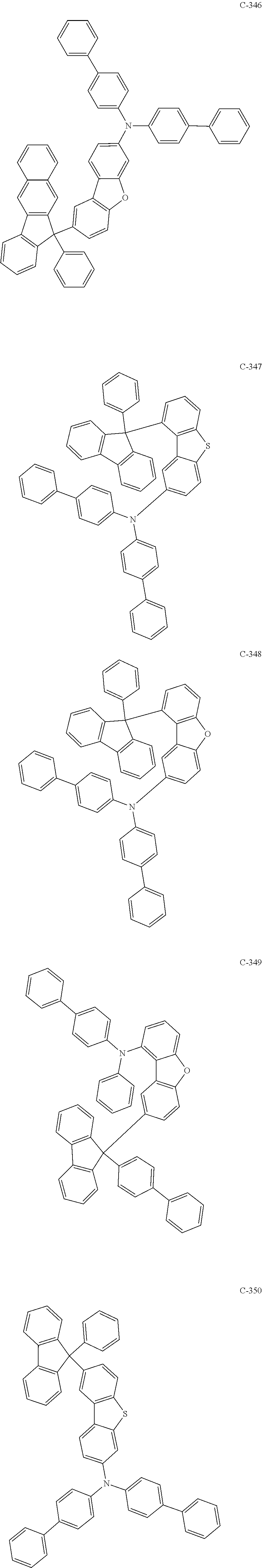 [SMILES: c1ccc(-c2ccc(N(c3ccc(-c4ccccc4)cc3)c3ccc4c(c3)oc3ccc(C5(c6ccccc6)c6ccccc6-c6cc7ccccc7cc65)cc34)cc2)cc1 ; c1ccc(-c2ccc(N(c3ccc(-c4ccccc4)cc3)c3ccc4sc5cccc(C6(c7ccccc7)c7ccccc7-c7ccccc76)c5c4c3)cc2)cc1 ; c1ccc(-c2ccc(N(c3ccc(-c4ccccc4)cc3)c3ccc4oc5cccc(C6(c7ccccc7)c7ccccc7-c7ccccc76)c5c4c3)cc2)cc1 ; c1ccc(-c2ccc(N(c3ccccc3)c3cccc4oc5ccc(C6(c7ccc(-c8ccccc8)cc7)c7ccccc7-c7ccccc76)cc5c34)cc2)cc1 ; c1ccc(-c2ccc(N(c3ccc(-c4ccccc4)cc3)c3ccc4c(c3)sc3ccc(C5(c6ccccc6)c6ccccc6-c6ccccc65)cc34)cc2)cc1]

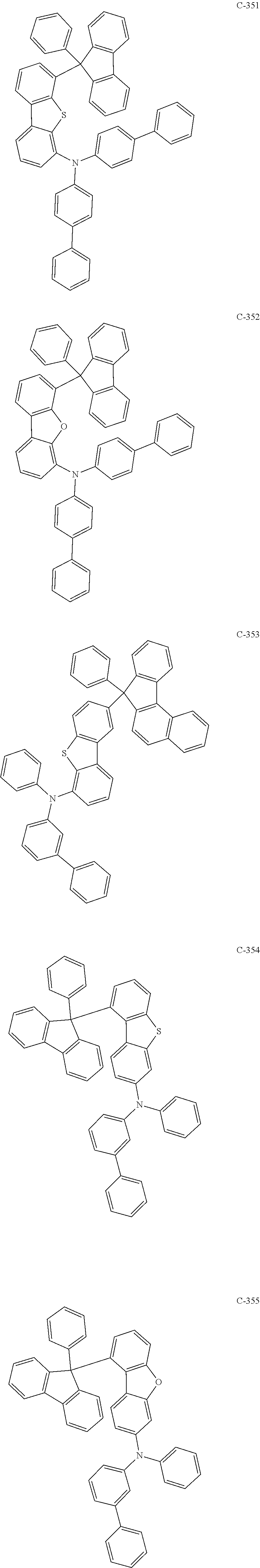 [SMILES: c1ccc(-c2ccc(N(c3ccc(-c4ccccc4)cc3)c3cccc4c3sc3c(C5(c6ccccc6)c6ccccc6-c6ccccc65)cccc34)cc2)cc1 ; c1ccc(-c2ccc(N(c3ccc(-c4ccccc4)cc3)c3cccc4c3oc3c(C5(c6ccccc6)c6ccccc6-c6ccccc65)cccc34)cc2)cc1 ; c1ccc(-c2cccc(N(c3ccccc3)c3cccc4c3sc3ccc(C5(c6ccccc6)c6ccccc6-c6c5ccc5ccccc65)cc34)c2)cc1 ; c1ccc(-c2cccc(N(c3ccccc3)c3ccc4c(c3)sc3cccc(C5(c6ccccc6)c6ccccc6-c6ccccc65)c34)c2)cc1 ; c1ccc(-c2cccc(N(c3ccccc3)c3ccc4c(c3)oc3cccc(C5(c6ccccc6)c6ccccc6-c6ccccc65)c34)c2)cc1]

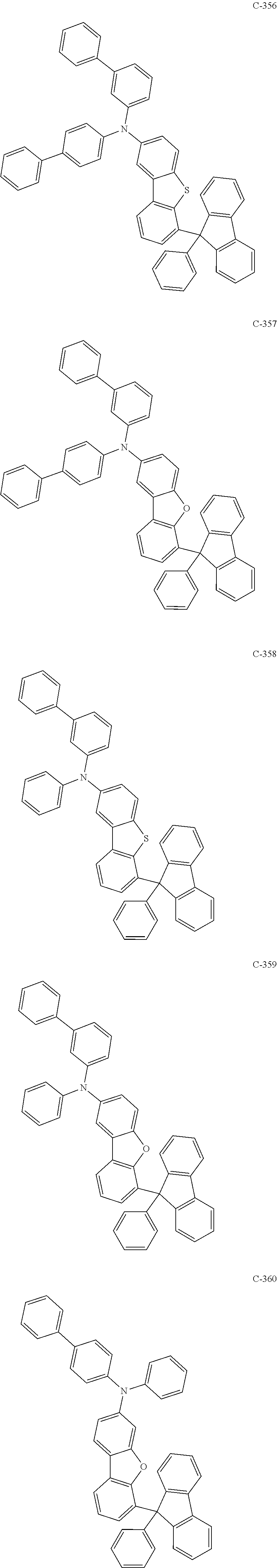 [SMILES: c1ccc(-c2ccc(N(c3cccc(-c4ccccc4)c3)c3ccc4sc5c(C6(c7ccccc7)c7ccccc7-c7ccccc76)cccc5c4c3)cc2)cc1 ; c1ccc(-c2ccc(N(c3cccc(-c4ccccc4)c3)c3ccc4oc5c(C6(c7ccccc7)c7ccccc7-c7ccccc76)cccc5c4c3)cc2)cc1 ; c1ccc(-c2cccc(N(c3ccccc3)c3ccc4sc5c(C6(c7ccccc7)c7ccccc7-c7ccccc76)cccc5c4c3)c2)cc1 ; c1ccc(-c2cccc(N(c3ccccc3)c3ccc4oc5c(C6(c7ccccc7)c7ccccc7-c7ccccc76)cccc5c4c3)c2)cc1 ; c1ccc(-c2ccc(N(c3ccccc3)c3ccc4c(c3)oc3c(C5(c6ccccc6)c6ccccc6-c6ccccc65)cccc34)cc2)cc1]

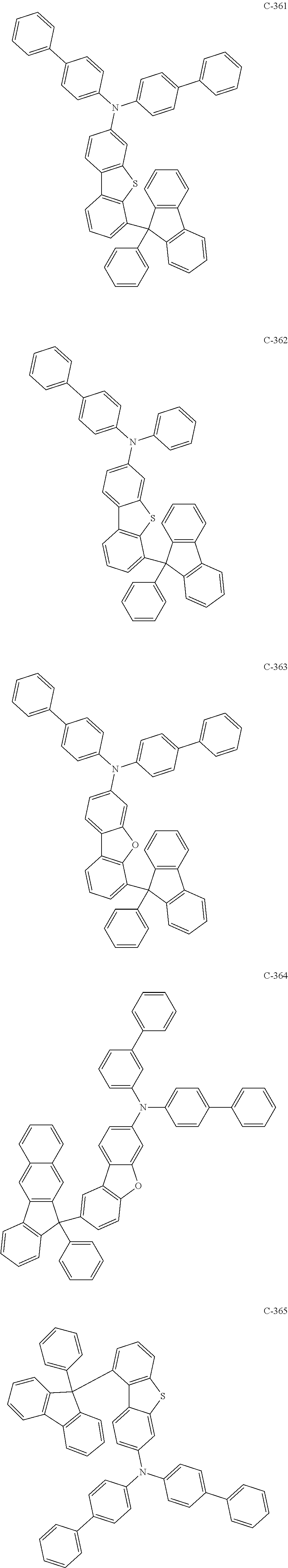 [SMILES: c1ccc(-c2ccc(N(c3ccc(-c4ccccc4)cc3)c3ccc4c(c3)sc3c(C5(c6ccccc6)c6ccccc6-c6ccccc65)cccc34)cc2)cc1 ; c1ccc(-c2ccc(N(c3ccccc3)c3ccc4c(c3)sc3c(C5(c6ccccc6)c6ccccc6-c6ccccc65)cccc34)cc2)cc1 ; c1ccc(-c2ccc(N(c3ccc(-c4ccccc4)cc3)c3ccc4c(c3)oc3c(C5(c6ccccc6)c6ccccc6-c6ccccc65)cccc34)cc2)cc1 ; c1ccc(-c2ccc(N(c3cccc(-c4ccccc4)c3)c3ccc4c(c3)oc3ccc(C5(c6ccccc6)c6ccccc6-c6cc7ccccc7cc65)cc34)cc2)cc1 ; c1ccc(-c2ccc(N(c3ccc(-c4ccccc4)cc3)c3ccc4c(c3)sc3cccc(C5(c6ccccc6)c6ccccc6-c6ccccc65)c34)cc2)cc1]

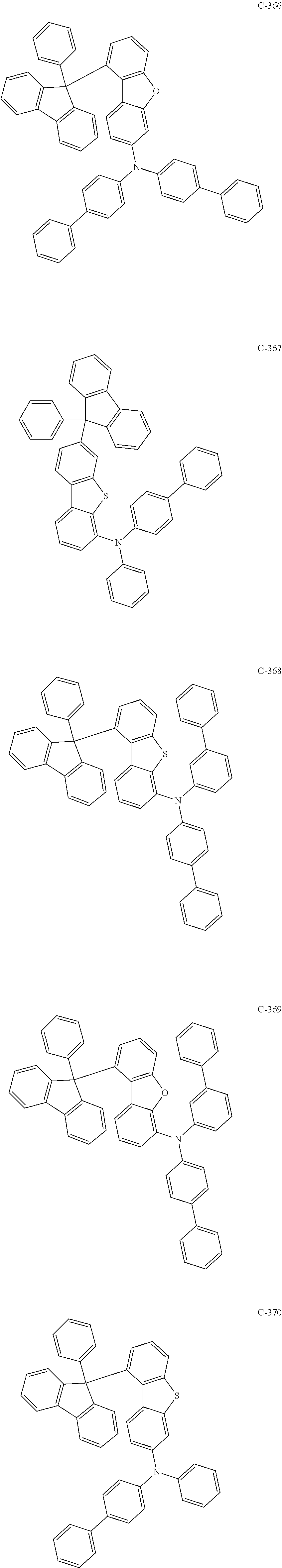 [SMILES: c1ccc(-c2ccc(N(c3ccc(-c4ccccc4)cc3)c3ccc4c(c3)oc3cccc(C5(c6ccccc6)c6ccccc6-c6ccccc65)c34)cc2)cc1 ; c1ccc(-c2ccc(N(c3ccccc3)c3cccc4c3sc3cc(C5(c6ccccc6)c6ccccc6-c6ccccc65)ccc34)cc2)cc1 ; c1ccc(-c2ccc(N(c3cccc(-c4ccccc4)c3)c3cccc4c3sc3cccc(C5(c6ccccc6)c6ccccc6-c6ccccc65)c34)cc2)cc1 ; c1ccc(-c2ccc(N(c3cccc(-c4ccccc4)c3)c3cccc4c3oc3cccc(C5(c6ccccc6)c6ccccc6-c6ccccc65)c34)cc2)cc1 ; c1ccc(-c2ccc(N(c3ccccc3)c3ccc4c(c3)sc3cccc(C5(c6ccccc6)c6ccccc6-c6ccccc65)c34)cc2)cc1]

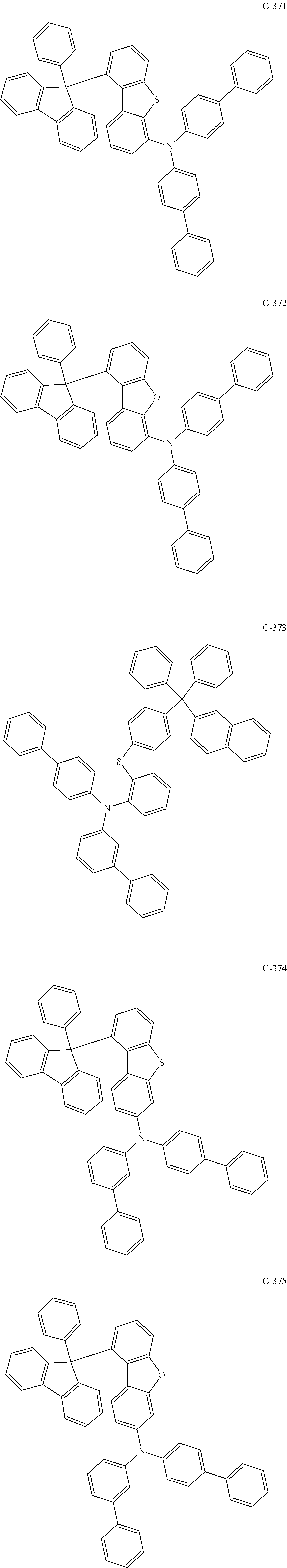 [SMILES: c1ccc(-c2ccc(N(c3ccc(-c4ccccc4)cc3)c3cccc4c3sc3cccc(C5(c6ccccc6)c6ccccc6-c6ccccc65)c34)cc2)cc1 ; c1ccc(-c2ccc(N(c3ccc(-c4ccccc4)cc3)c3cccc4c3oc3cccc(C5(c6ccccc6)c6ccccc6-c6ccccc65)c34)cc2)cc1 ; c1ccc(-c2ccc(N(c3cccc(-c4ccccc4)c3)c3cccc4c3sc3ccc(C5(c6ccccc6)c6ccccc6-c6c5ccc5ccccc65)cc34)cc2)cc1 ; c1ccc(-c2ccc(N(c3cccc(-c4ccccc4)c3)c3ccc4c(c3)sc3cccc(C5(c6ccccc6)c6ccccc6-c6ccccc65)c34)cc2)cc1 ; c1ccc(-c2ccc(N(c3cccc(-c4ccccc4)c3)c3ccc4c(c3)oc3cccc(C5(c6ccccc6)c6ccccc6-c6ccccc65)c34)cc2)cc1]

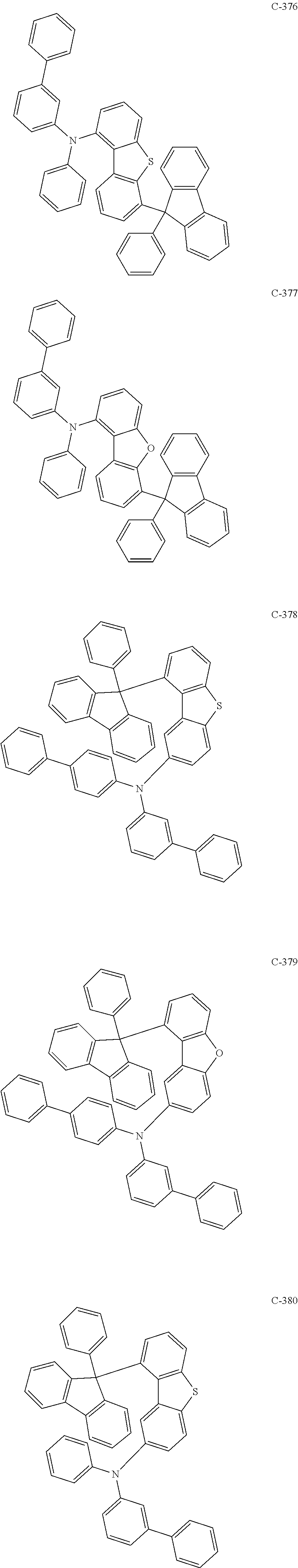 [SMILES: c1ccc(-c2cccc(N(c3ccccc3)c3cccc4sc5c(C6(c7ccccc7)c7ccccc7-c7ccccc76)cccc5c34)c2)cc1 ; c1ccc(-c2cccc(N(c3ccccc3)c3cccc4oc5c(C6(c7ccccc7)c7ccccc7-c7ccccc76)cccc5c34)c2)cc1 ; c1ccc(-c2ccc(N(c3cccc(-c4ccccc4)c3)c3ccc4sc5cccc(C6(c7ccccc7)c7ccccc7-c7ccccc76)c5c4c3)cc2)cc1 ; c1ccc(-c2ccc(N(c3cccc(-c4ccccc4)c3)c3ccc4oc5cccc(C6(c7ccccc7)c7ccccc7-c7ccccc76)c5c4c3)cc2)cc1 ; c1ccc(-c2cccc(N(c3ccccc3)c3ccc4sc5cccc(C6(c7ccccc7)c7ccccc7-c7ccccc76)c5c4c3)c2)cc1]

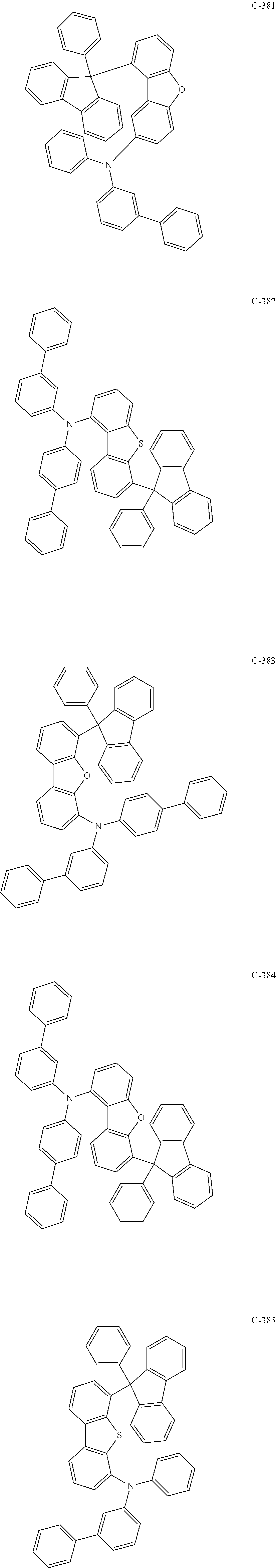 [SMILES: c1ccc(-c2cccc(N(c3ccccc3)c3ccc4oc5cccc(C6(c7ccccc7)c7ccccc7-c7ccccc76)c5c4c3)c2)cc1 ; c1ccc(-c2ccc(N(c3cccc(-c4ccccc4)c3)c3cccc4sc5c(C6(c7ccccc7)c7ccccc7-c7ccccc76)cccc5c34)cc2)cc1 ; c1ccc(-c2ccc(N(c3cccc(-c4ccccc4)c3)c3cccc4c3oc3c(C5(c6ccccc6)c6ccccc6-c6ccccc65)cccc34)cc2)cc1 ; c1ccc(-c2ccc(N(c3cccc(-c4ccccc4)c3)c3cccc4oc5c(C6(c7ccccc7)c7ccccc7-c7ccccc76)cccc5c34)cc2)cc1 ; c1ccc(-c2cccc(N(c3ccccc3)c3cccc4c3sc3c(C5(c6ccccc6)c6ccccc6-c6ccccc65)cccc34)c2)cc1]

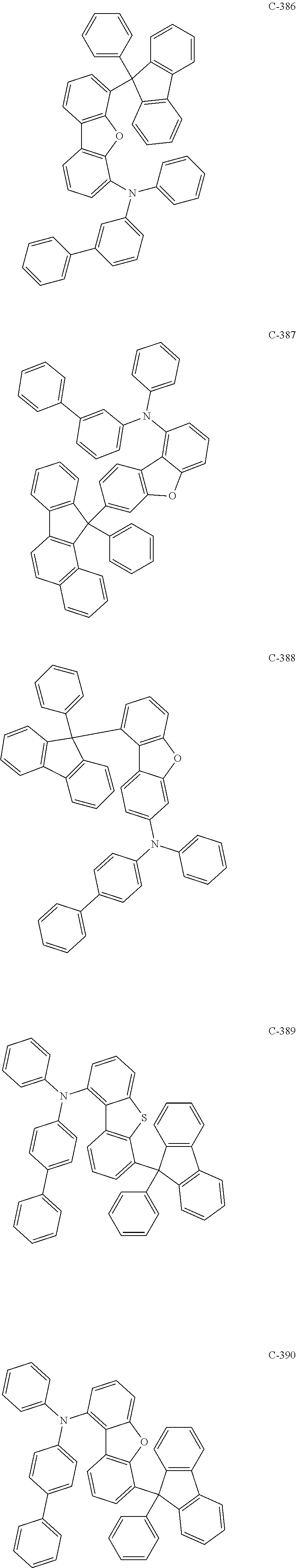 [SMILES: c1ccc(-c2cccc(N(c3ccccc3)c3cccc4c3oc3c(C5(c6ccccc6)c6ccccc6-c6ccccc65)cccc34)c2)cc1 ; c1ccc(-c2cccc(N(c3ccccc3)c3cccc4oc5cc(C6(c7ccccc7)c7ccccc7-c7ccc8ccccc8c76)ccc5c34)c2)cc1 ; c1ccc(-c2ccc(N(c3ccccc3)c3ccc4c(c3)oc3cccc(C5(c6ccccc6)c6ccccc6-c6ccccc65)c34)cc2)cc1 ; c1ccc(-c2ccc(N(c3ccccc3)c3cccc4sc5c(C6(c7ccccc7)c7ccccc7-c7ccccc76)cccc5c34)cc2)cc1 ; c1ccc(-c2ccc(N(c3ccccc3)c3cccc4oc5c(C6(c7ccccc7)c7ccccc7-c7ccccc76)cccc5c34)cc2)cc1]

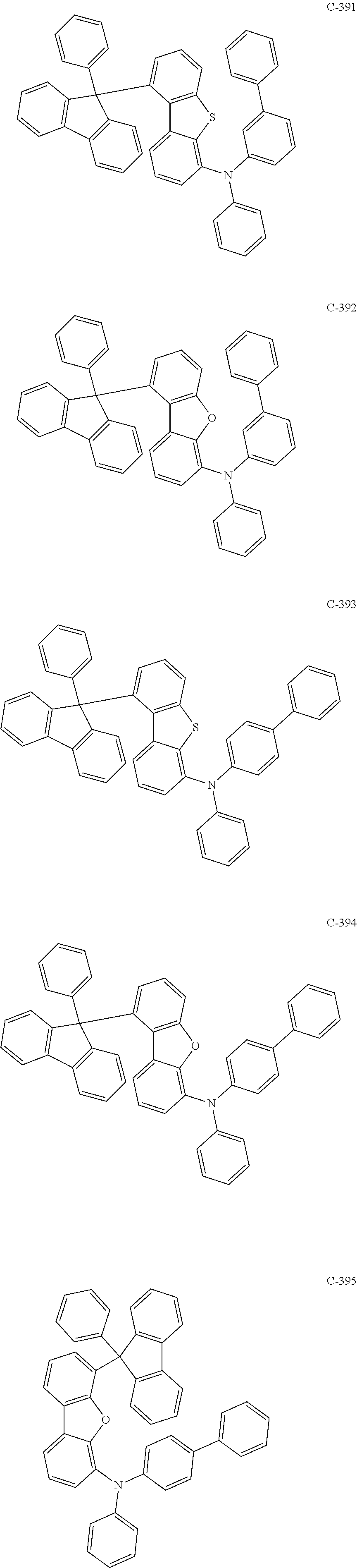 [SMILES: c1ccc(-c2cccc(N(c3ccccc3)c3cccc4c3sc3cccc(C5(c6ccccc6)c6ccccc6-c6ccccc65)c34)c2)cc1 ; c1ccc(-c2cccc(N(c3ccccc3)c3cccc4c3oc3cccc(C5(c6ccccc6)c6ccccc6-c6ccccc65)c34)c2)cc1 ; c1ccc(-c2ccc(N(c3ccccc3)c3cccc4c3sc3cccc(C5(c6ccccc6)c6ccccc6-c6ccccc65)c34)cc2)cc1 ; c1ccc(-c2ccc(N(c3ccccc3)c3cccc4c3oc3cccc(C5(c6ccccc6)c6ccccc6-c6ccccc65)c34)cc2)cc1 ; c1ccc(-c2ccc(N(c3ccccc3)c3cccc4c3oc3c(C5(c6ccccc6)c6ccccc6-c6ccccc65)cccc34)cc2)cc1]

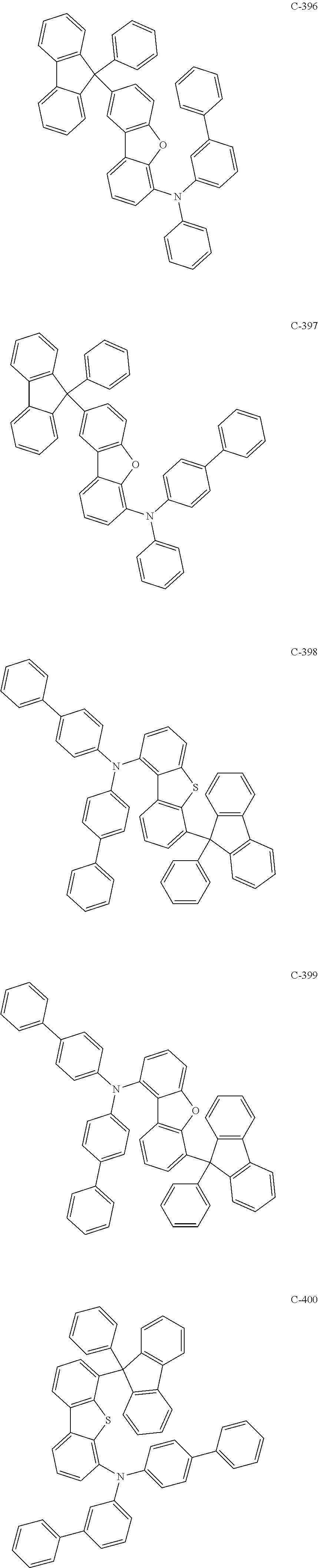 [SMILES: c1ccc(-c2cccc(N(c3ccccc3)c3cccc4c3oc3ccc(C5(c6ccccc6)c6ccccc6-c6ccccc65)cc34)c2)cc1 ; c1ccc(-c2ccc(N(c3ccccc3)c3cccc4c3oc3ccc(C5(c6ccccc6)c6ccccc6-c6ccccc65)cc34)cc2)cc1 ; c1ccc(-c2ccc(N(c3ccc(-c4ccccc4)cc3)c3cccc4sc5c(C6(c7ccccc7)c7ccccc7-c7ccccc76)cccc5c34)cc2)cc1 ; c1ccc(-c2ccc(N(c3ccc(-c4ccccc4)cc3)c3cccc4oc5c(C6(c7ccccc7)c7ccccc7-c7ccccc76)cccc5c34)cc2)cc1 ; c1ccc(-c2ccc(N(c3cccc(-c4ccccc4)c3)c3cccc4c3sc3c(C5(c6ccccc6)c6ccccc6-c6ccccc65)cccc34)cc2)cc1]

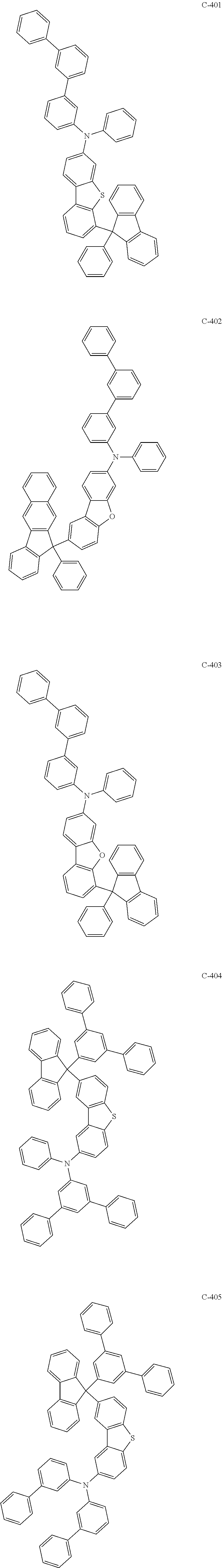

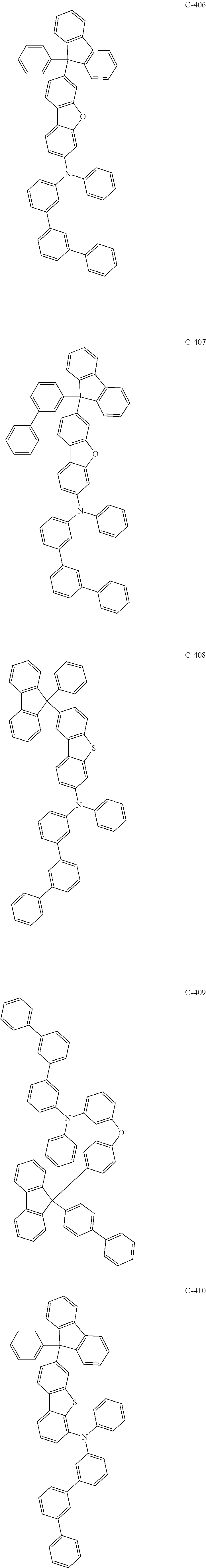

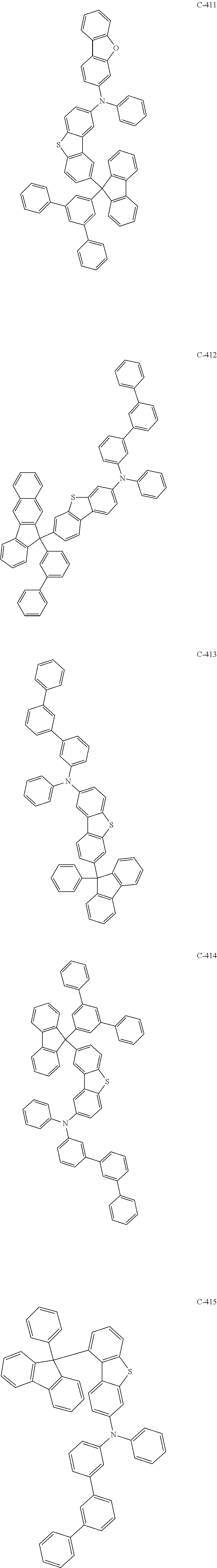

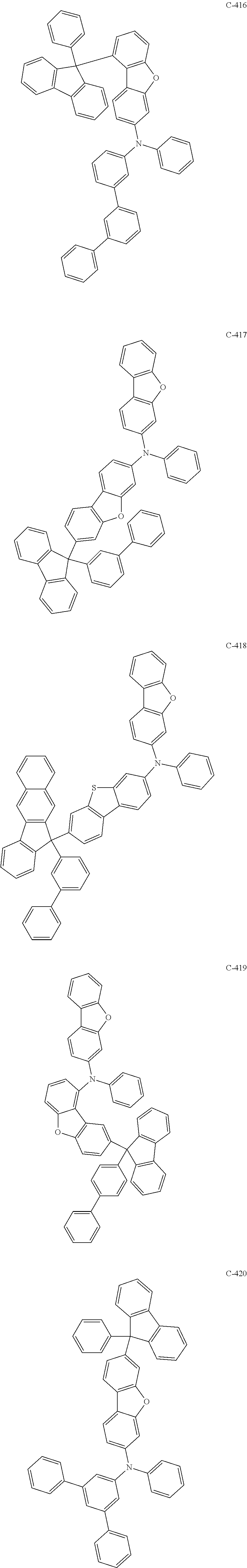

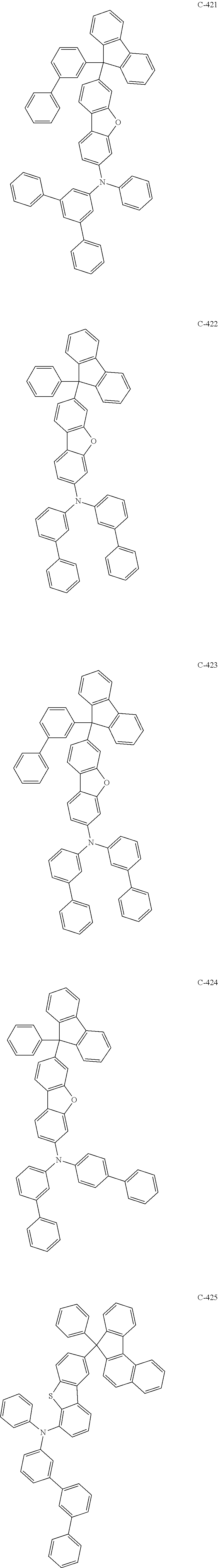

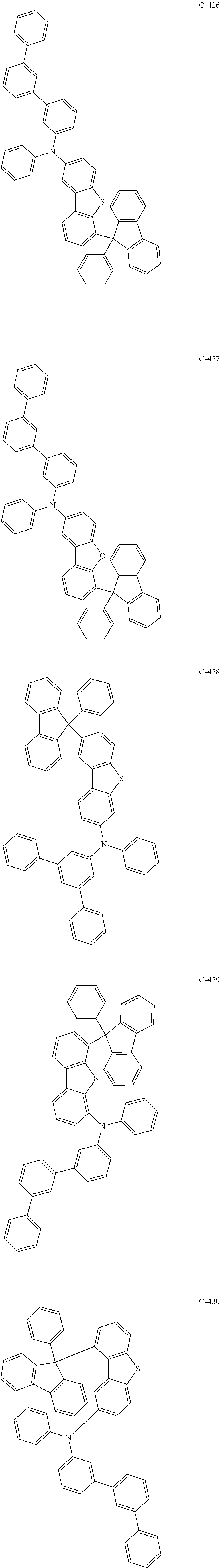 [SMILES: c1ccc(-c2cccc(-c3cccc(N(c4ccccc4)c4ccc5sc6c(C7(c8ccccc8)c8ccccc8-c8ccccc87)cccc6c5c4)c3)c2)cc1 ; c1ccc(-c2cccc(-c3cccc(N(c4ccccc4)c4ccc5oc6c(C7(c8ccccc8)c8ccccc8-c8ccccc87)cccc6c5c4)c3)c2)cc1 ; c1ccc(-c2cc(-c3ccccc3)cc(N(c3ccccc3)c3ccc4c(c3)sc3ccc(C5(c6ccccc6)c6ccccc6-c6ccccc65)cc34)c2)cc1 ; c1ccc(-c2cccc(-c3cccc(N(c4ccccc4)c4cccc5c4sc4c(C6(c7ccccc7)c7ccccc7-c7ccccc76)cccc45)c3)c2)cc1 ; c1ccc(-c2cccc(-c3cccc(N(c4ccccc4)c4ccc5sc6cccc(C7(c8ccccc8)c8ccccc8-c8ccccc87)c6c5c4)c3)c2)cc1]

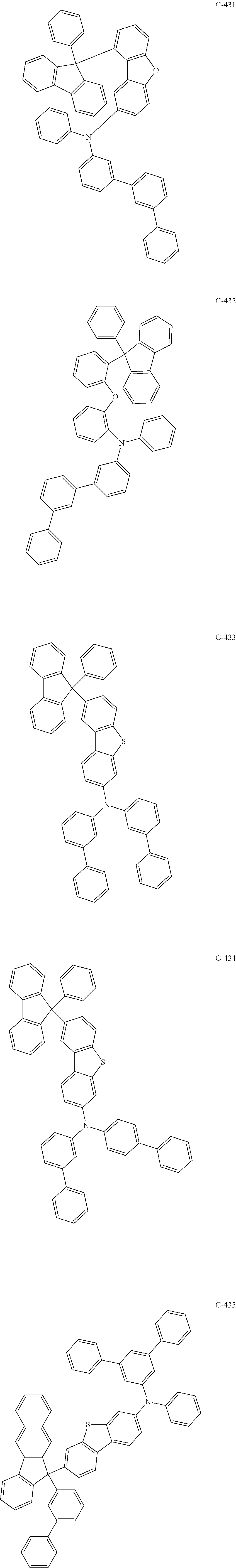

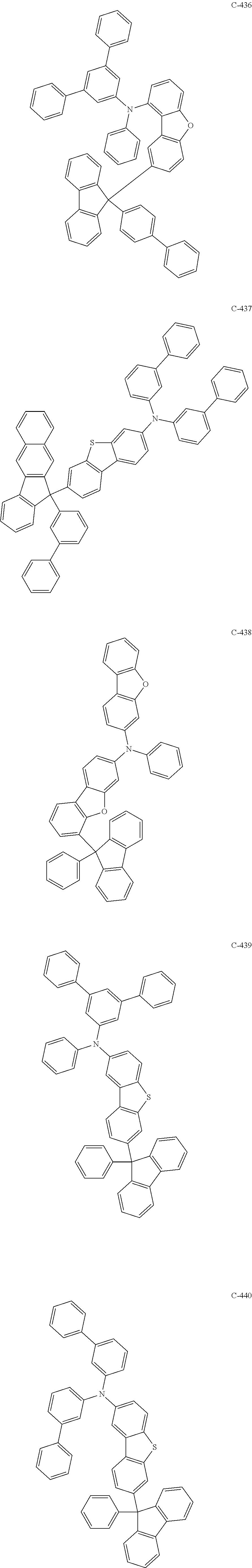

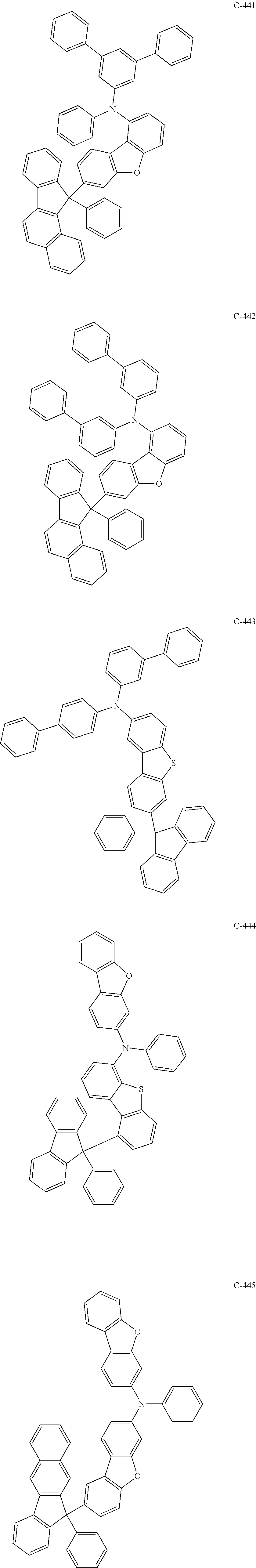 [SMILES: c1ccc(-c2cc(-c3ccccc3)cc(N(c3ccccc3)c3cccc4oc5cc(C6(c7ccccc7)c7ccccc7-c7ccc8ccccc8c76)ccc5c34)c2)cc1 ; c1ccc(-c2cccc(N(c3cccc(-c4ccccc4)c3)c3cccc4oc5cc(C6(c7ccccc7)c7ccccc7-c7ccc8ccccc8c76)ccc5c34)c2)cc1 ; c1ccc(-c2ccc(N(c3cccc(-c4ccccc4)c3)c3ccc4sc5cc(C6(c7ccccc7)c7ccccc7-c7ccccc76)ccc5c4c3)cc2)cc1 ; c1ccc(N(c2ccc3c(c2)oc2ccccc23)c2cccc3c2sc2cccc(C4(c5ccccc5)c5ccccc5-c5ccccc54)c23)cc1 ; c1ccc(N(c2ccc3c(c2)oc2ccccc23)c2ccc3c(c2)oc2ccc(C4(c5ccccc5)c5ccccc5-c5cc6ccccc6cc54)cc23)cc1]

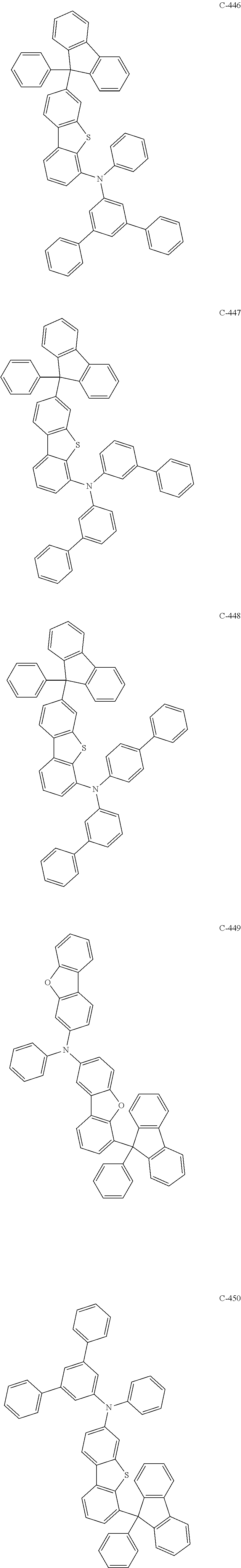 [SMILES: c1ccc(-c2cc(-c3ccccc3)cc(N(c3ccccc3)c3cccc4c3sc3cc(C5(c6ccccc6)c6ccccc6-c6ccccc65)ccc34)c2)cc1 ; c1ccc(-c2cccc(N(c3cccc(-c4ccccc4)c3)c3cccc4c3sc3cc(C5(c6ccccc6)c6ccccc6-c6ccccc65)ccc34)c2)cc1 ; c1ccc(-c2ccc(N(c3cccc(-c4ccccc4)c3)c3cccc4c3sc3cc(C5(c6ccccc6)c6ccccc6-c6ccccc65)ccc34)cc2)cc1 ; c1ccc(N(c2ccc3c(c2)oc2ccccc23)c2ccc3oc4c(C5(c6ccccc6)c6ccccc6-c6ccccc65)cccc4c3c2)cc1 ; c1ccc(-c2cc(-c3ccccc3)cc(N(c3ccccc3)c3ccc4c(c3)sc3c(C5(c6ccccc6)c6ccccc6-c6ccccc65)cccc34)c2)cc1]

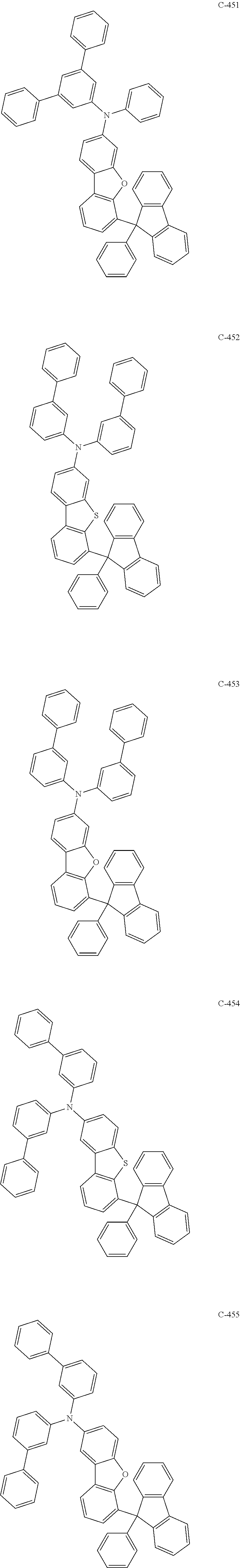 [SMILES: c1ccc(-c2cc(-c3ccccc3)cc(N(c3ccccc3)c3ccc4c(c3)oc3c(C5(c6ccccc6)c6ccccc6-c6ccccc65)cccc34)c2)cc1 ; c1ccc(-c2cccc(N(c3cccc(-c4ccccc4)c3)c3ccc4c(c3)sc3c(C5(c6ccccc6)c6ccccc6-c6ccccc65)cccc34)c2)cc1 ; c1ccc(-c2cccc(N(c3cccc(-c4ccccc4)c3)c3ccc4c(c3)oc3c(C5(c6ccccc6)c6ccccc6-c6ccccc65)cccc34)c2)cc1 ; c1ccc(-c2cccc(N(c3cccc(-c4ccccc4)c3)c3ccc4sc5c(C6(c7ccccc7)c7ccccc7-c7ccccc76)cccc5c4c3)c2)cc1 ; c1ccc(-c2cccc(N(c3cccc(-c4ccccc4)c3)c3ccc4oc5c(C6(c7ccccc7)c7ccccc7-c7ccccc76)cccc5c4c3)c2)cc1]

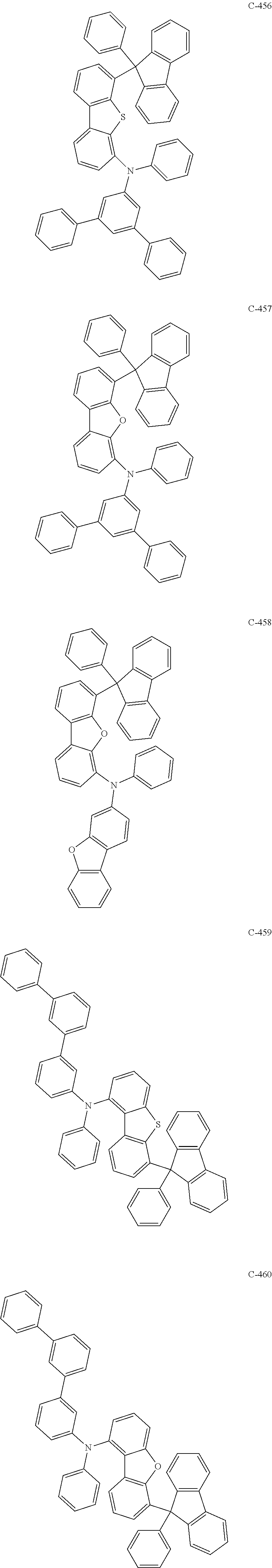 [SMILES: c1ccc(-c2cc(-c3ccccc3)cc(N(c3ccccc3)c3cccc4c3sc3c(C5(c6ccccc6)c6ccccc6-c6ccccc65)cccc34)c2)cc1 ; c1ccc(-c2cc(-c3ccccc3)cc(N(c3ccccc3)c3cccc4c3oc3c(C5(c6ccccc6)c6ccccc6-c6ccccc65)cccc34)c2)cc1 ; c1ccc(N(c2ccc3c(c2)oc2ccccc23)c2cccc3c2oc2c(C4(c5ccccc5)c5ccccc5-c5ccccc54)cccc23)cc1 ; c1ccc(-c2cccc(-c3cccc(N(c4ccccc4)c4cccc5sc6c(C7(c8ccccc8)c8ccccc8-c8ccccc87)cccc6c45)c3)c2)cc1 ; c1ccc(-c2cccc(-c3cccc(N(c4ccccc4)c4cccc5oc6c(C7(c8ccccc8)c8ccccc8-c8ccccc87)cccc6c45)c3)c2)cc1]

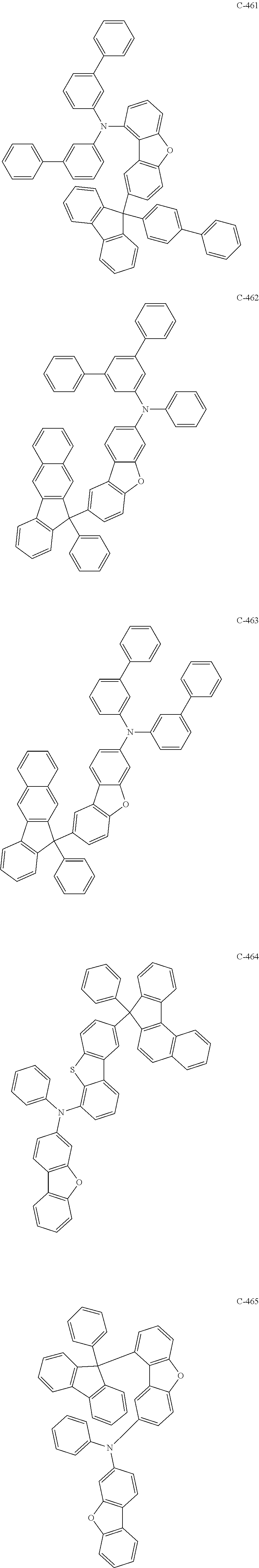 [SMILES: c1ccc(-c2ccc(C3(c4ccc5oc6cccc(N(c7cccc(-c8ccccc8)c7)c7cccc(-c8ccccc8)c7)c6c5c4)c4ccccc4-c4ccccc43)cc2)cc1 ; c1ccc(-c2cc(-c3ccccc3)cc(N(c3ccccc3)c3ccc4c(c3)oc3ccc(C5(c6ccccc6)c6ccccc6-c6cc7ccccc7cc65)cc34)c2)cc1 ; c1ccc(-c2cccc(N(c3cccc(-c4ccccc4)c3)c3ccc4c(c3)oc3ccc(C5(c6ccccc6)c6ccccc6-c6cc7ccccc7cc65)cc34)c2)cc1 ; c1ccc(N(c2ccc3c(c2)oc2ccccc23)c2cccc3c2sc2ccc(C4(c5ccccc5)c5ccccc5-c5c4ccc4ccccc54)cc23)cc1 ; c1ccc(N(c2ccc3c(c2)oc2ccccc23)c2ccc3oc4cccc(C5(c6ccccc6)c6ccccc6-c6ccccc65)c4c3c2)cc1]

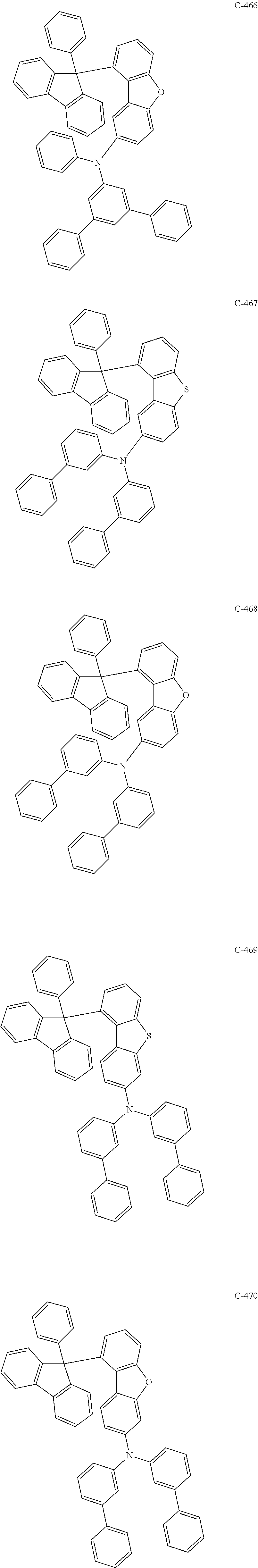 [SMILES: c1ccc(-c2cc(-c3ccccc3)cc(N(c3ccccc3)c3ccc4oc5cccc(C6(c7ccccc7)c7ccccc7-c7ccccc76)c5c4c3)c2)cc1 ; c1ccc(-c2cccc(N(c3cccc(-c4ccccc4)c3)c3ccc4sc5cccc(C6(c7ccccc7)c7ccccc7-c7ccccc76)c5c4c3)c2)cc1 ; c1ccc(-c2cccc(N(c3cccc(-c4ccccc4)c3)c3ccc4oc5cccc(C6(c7ccccc7)c7ccccc7-c7ccccc76)c5c4c3)c2)cc1 ; c1ccc(-c2cccc(N(c3cccc(-c4ccccc4)c3)c3ccc4c(c3)sc3cccc(C5(c6ccccc6)c6ccccc6-c6ccccc65)c34)c2)cc1 ; c1ccc(-c2cccc(N(c3cccc(-c4ccccc4)c3)c3ccc4c(c3)oc3cccc(C5(c6ccccc6)c6ccccc6-c6ccccc65)c34)c2)cc1]

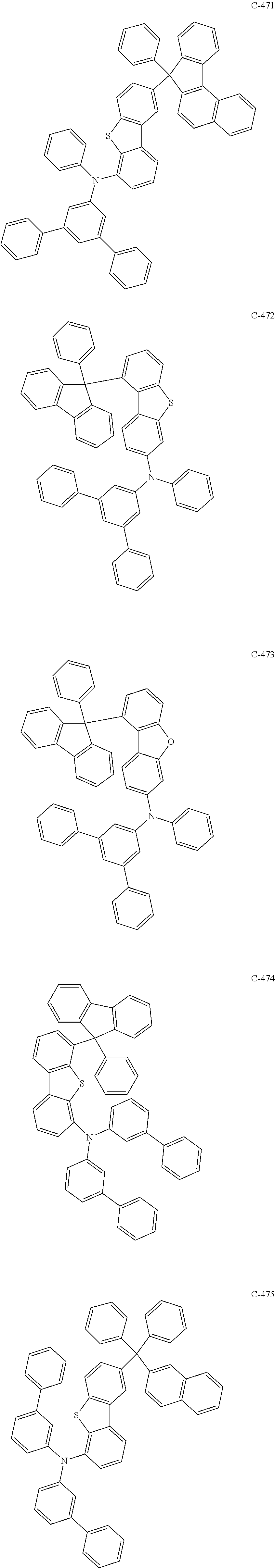 [SMILES: c1ccc(-c2cc(-c3ccccc3)cc(N(c3ccccc3)c3cccc4c3sc3ccc(C5(c6ccccc6)c6ccccc6-c6c5ccc5ccccc65)cc34)c2)cc1 ; c1ccc(-c2cc(-c3ccccc3)cc(N(c3ccccc3)c3ccc4c(c3)sc3cccc(C5(c6ccccc6)c6ccccc6-c6ccccc65)c34)c2)cc1 ; c1ccc(-c2cc(-c3ccccc3)cc(N(c3ccccc3)c3ccc4c(c3)oc3cccc(C5(c6ccccc6)c6ccccc6-c6ccccc65)c34)c2)cc1 ; c1ccc(-c2cccc(N(c3cccc(-c4ccccc4)c3)c3cccc4c3sc3c(C5(c6ccccc6)c6ccccc6-c6ccccc65)cccc34)c2)cc1 ; c1ccc(-c2cccc(N(c3cccc(-c4ccccc4)c3)c3cccc4c3sc3ccc(C5(c6ccccc6)c6ccccc6-c6c5ccc5ccccc65)cc34)c2)cc1]

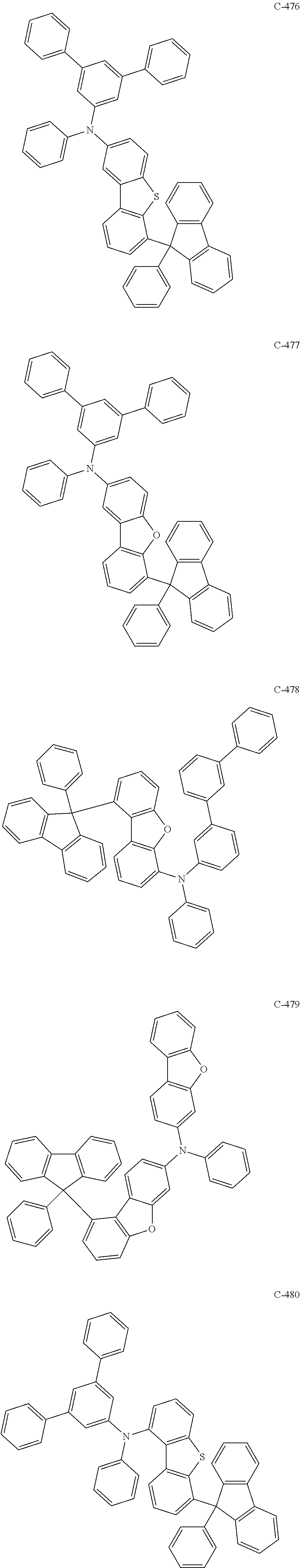 [SMILES: c1ccc(-c2cc(-c3ccccc3)cc(N(c3ccccc3)c3ccc4sc5c(C6(c7ccccc7)c7ccccc7-c7ccccc76)cccc5c4c3)c2)cc1 ; c1ccc(-c2cc(-c3ccccc3)cc(N(c3ccccc3)c3ccc4oc5c(C6(c7ccccc7)c7ccccc7-c7ccccc76)cccc5c4c3)c2)cc1 ; c1ccc(-c2cccc(-c3cccc(N(c4ccccc4)c4cccc5c4oc4cccc(C6(c7ccccc7)c7ccccc7-c7ccccc76)c45)c3)c2)cc1 ; c1ccc(N(c2ccc3c(c2)oc2ccccc23)c2ccc3c(c2)oc2cccc(C4(c5ccccc5)c5ccccc5-c5ccccc54)c23)cc1 ; c1ccc(-c2cc(-c3ccccc3)cc(N(c3ccccc3)c3cccc4sc5c(C6(c7ccccc7)c7ccccc7-c7ccccc76)cccc5c34)c2)cc1]

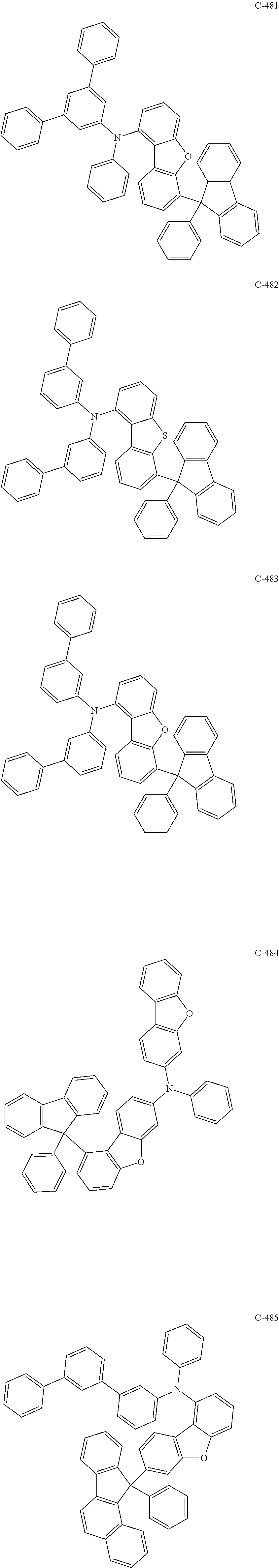 [SMILES: c1ccc(-c2cc(-c3ccccc3)cc(N(c3ccccc3)c3cccc4oc5c(C6(c7ccccc7)c7ccccc7-c7ccccc76)cccc5c34)c2)cc1 ; c1ccc(-c2cccc(N(c3cccc(-c4ccccc4)c3)c3cccc4sc5c(C6(c7ccccc7)c7ccccc7-c7ccccc76)cccc5c34)c2)cc1 ; c1ccc(-c2cccc(N(c3cccc(-c4ccccc4)c3)c3cccc4oc5c(C6(c7ccccc7)c7ccccc7-c7ccccc76)cccc5c34)c2)cc1 ; c1ccc(N(c2ccc3c(c2)oc2ccccc23)c2ccc3c(c2)oc2cccc(C4(c5ccccc5)c5ccccc5-c5ccccc54)c23)cc1 ; c1ccc(-c2cccc(-c3cccc(N(c4ccccc4)c4cccc5oc6cc(C7(c8ccccc8)c8ccccc8-c8ccc9ccccc9c87)ccc6c45)c3)c2)cc1]

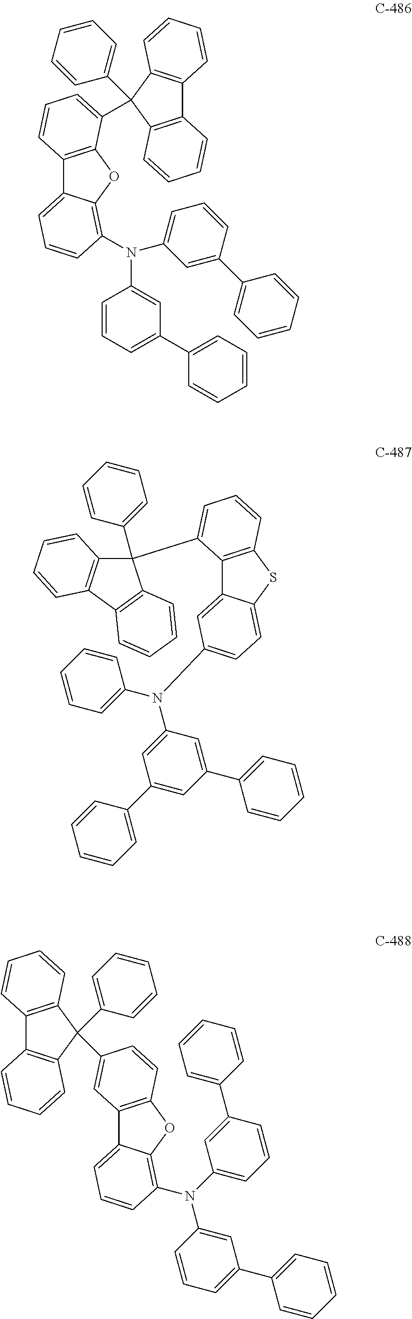 [SMILES: c1ccc(-c2cccc(N(c3cccc(-c4ccccc4)c3)c3cccc4c3oc3c(C5(c6ccccc6)c6ccccc6-c6ccccc65)cccc34)c2)cc1 ; c1ccc(-c2cc(-c3ccccc3)cc(N(c3ccccc3)c3ccc4sc5cccc(C6(c7ccccc7)c7ccccc7-c7ccccc76)c5c4c3)c2)cc1 ; c1ccc(-c2cccc(N(c3cccc(-c4ccccc4)c3)c3cccc4c3oc3ccc(C5(c6ccccc6)c6ccccc6-c6ccccc65)cc34)c2)cc1]

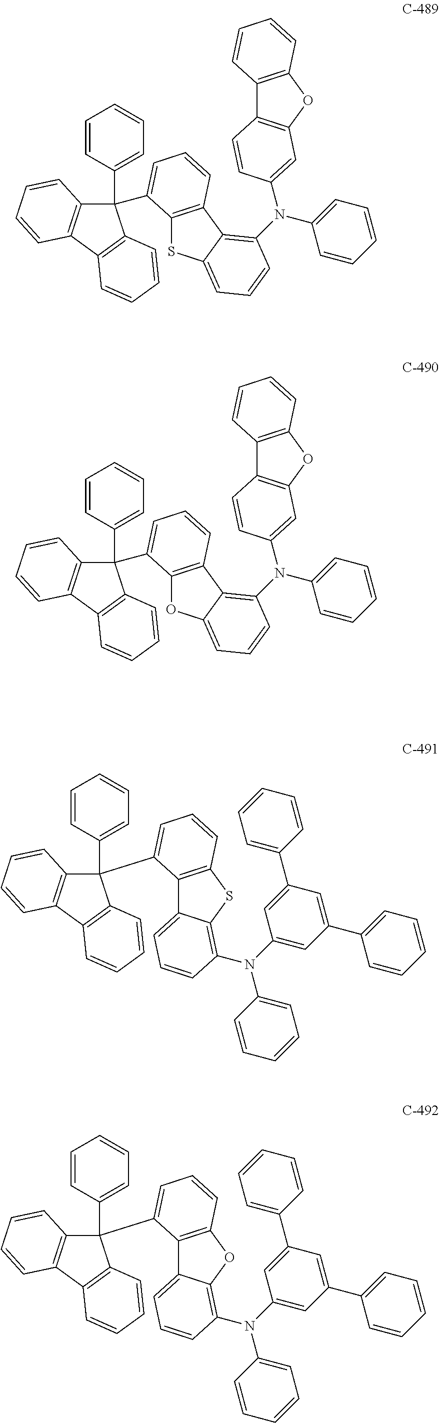 [SMILES: c1ccc(N(c2ccc3c(c2)oc2ccccc23)c2cccc3sc4c(C5(c6ccccc6)c6ccccc6-c6ccccc65)cccc4c23)cc1 ; c1ccc(N(c2ccc3c(c2)oc2ccccc23)c2cccc3oc4c(C5(c6ccccc6)c6ccccc6-c6ccccc65)cccc4c23)cc1 ; c1ccc(-c2cc(-c3ccccc3)cc(N(c3ccccc3)c3cccc4c3sc3cccc(C5(c6ccccc6)c6ccccc6-c6ccccc65)c34)c2)cc1 ; c1ccc(-c2cc(-c3ccccc3)cc(N(c3ccccc3)c3cccc4c3oc3cccc(C5(c6ccccc6)c6ccccc6-c6ccccc65)c34)c2)cc1]

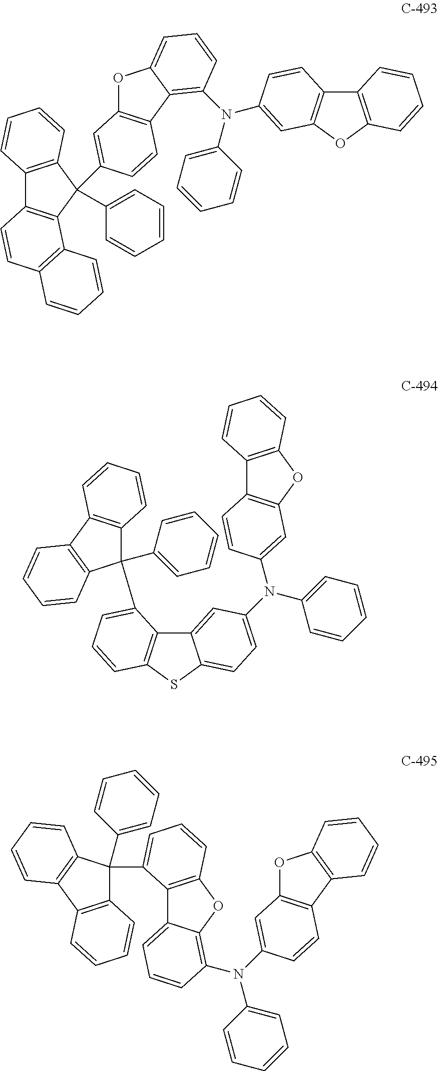 [SMILES: c1ccc(N(c2ccc3c(c2)oc2ccccc23)c2cccc3oc4cc(C5(c6ccccc6)c6ccccc6-c6ccc7ccccc7c65)ccc4c23)cc1 ; c1ccc(N(c2ccc3c(c2)oc2ccccc23)c2ccc3sc4cccc(C5(c6ccccc6)c6ccccc6-c6ccccc65)c4c3c2)cc1 ; c1ccc(N(c2ccc3c(c2)oc2ccccc23)c2cccc3c2oc2cccc(C4(c5ccccc5)c5ccccc5-c5ccccc54)c23)cc1]

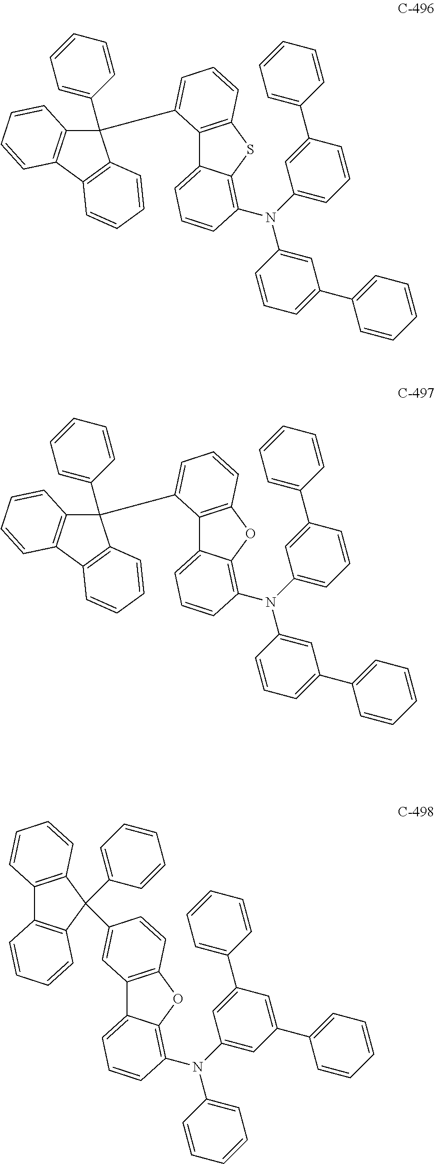 [SMILES: c1ccc(-c2cccc(N(c3cccc(-c4ccccc4)c3)c3cccc4c3sc3cccc(C5(c6ccccc6)c6ccccc6-c6ccccc65)c34)c2)cc1 ; c1ccc(-c2cccc(N(c3cccc(-c4ccccc4)c3)c3cccc4c3oc3cccc(C5(c6ccccc6)c6ccccc6-c6ccccc65)c34)c2)cc1 ; c1ccc(-c2cc(-c3ccccc3)cc(N(c3ccccc3)c3cccc4c3oc3ccc(C5(c6ccccc6)c6ccccc6-c6ccccc65)cc34)c2)cc1]

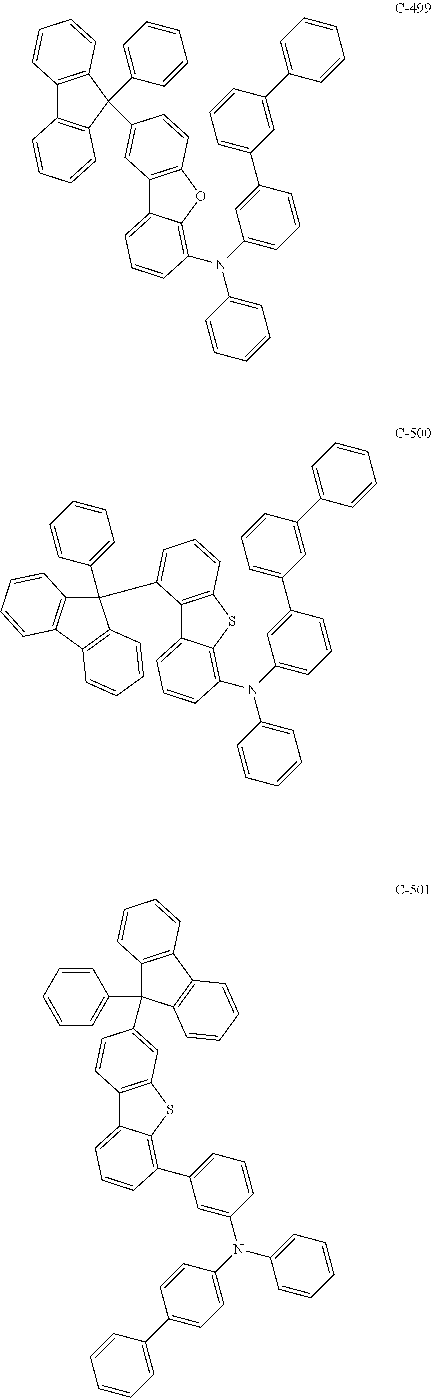 [SMILES: c1ccc(-c2cccc(-c3cccc(N(c4ccccc4)c4cccc5c4oc4ccc(C6(c7ccccc7)c7ccccc7-c7ccccc76)cc45)c3)c2)cc1 ; c1ccc(-c2cccc(-c3cccc(N(c4ccccc4)c4cccc5c4sc4cccc(C6(c7ccccc7)c7ccccc7-c7ccccc76)c45)c3)c2)cc1 ; c1ccc(-c2ccc(N(c3ccccc3)c3cccc(-c4cccc5c4sc4cc(C6(c7ccccc7)c7ccccc7-c7ccccc76)ccc45)c3)cc2)cc1]

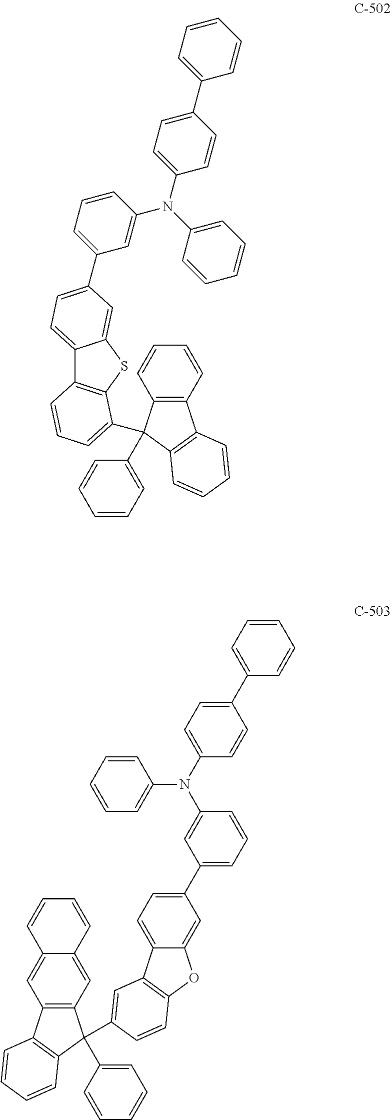 [SMILES: c1ccc(-c2ccc(N(c3ccccc3)c3cccc(-c4ccc5c(c4)sc4c(C6(c7ccccc7)c7ccccc7-c7ccccc76)cccc45)c3)cc2)cc1 ; c1ccc(-c2ccc(N(c3ccccc3)c3cccc(-c4ccc5c(c4)oc4ccc(C6(c7ccccc7)c7ccccc7-c7cc8ccccc8cc76)cc45)c3)cc2)cc1]

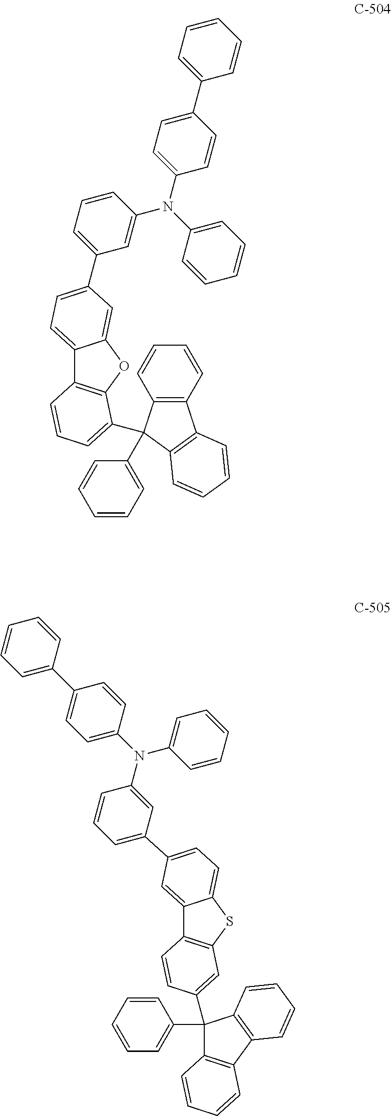 [SMILES: c1ccc(-c2ccc(N(c3ccccc3)c3cccc(-c4ccc5c(c4)oc4c(C6(c7ccccc7)c7ccccc7-c7ccccc76)cccc45)c3)cc2)cc1 ; c1ccc(-c2ccc(N(c3ccccc3)c3cccc(-c4ccc5sc6cc(C7(c8ccccc8)c8ccccc8-c8ccccc87)ccc6c5c4)c3)cc2)cc1]

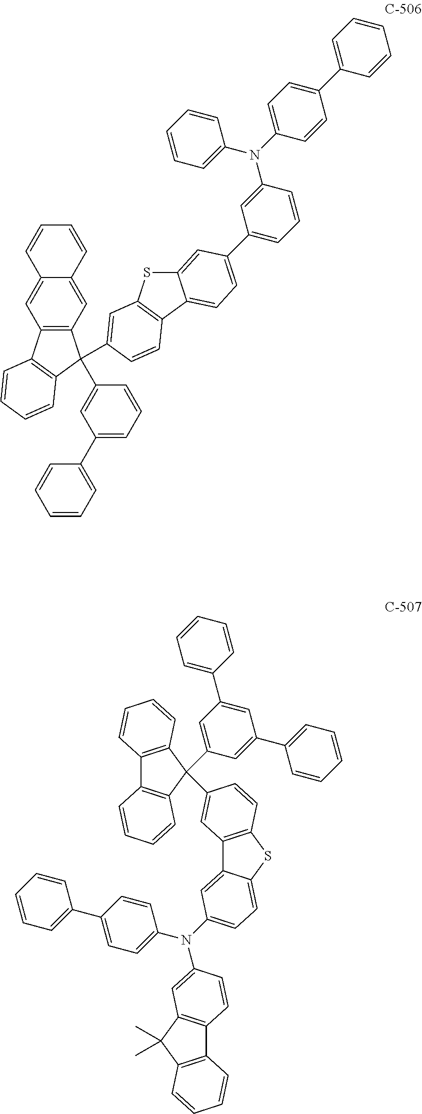 [SMILES: c1ccc(-c2ccc(N(c3ccccc3)c3cccc(-c4ccc5c(c4)sc4cc(C6(c7cccc(-c8ccccc8)c7)c7ccccc7-c7cc8ccccc8cc76)ccc45)c3)cc2)cc1 ; CC1(C)c2ccccc2-c2ccc(N(c3ccc(-c4ccccc4)cc3)c3ccc4sc5ccc(C6(c7cc(-c8ccccc8)cc(-c8ccccc8)c7)c7ccccc7-c7ccccc76)cc5c4c3)cc21]

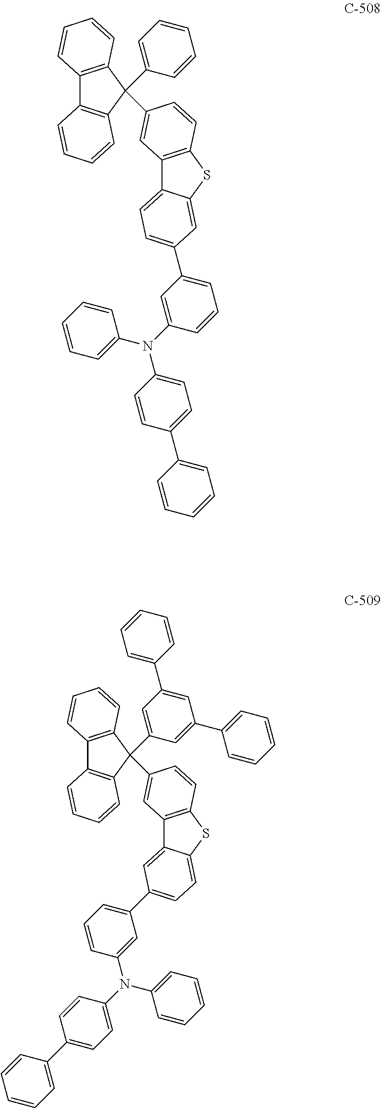 [SMILES: c1ccc(-c2ccc(N(c3ccccc3)c3cccc(-c4ccc5c(c4)sc4ccc(C6(c7ccccc7)c7ccccc7-c7ccccc76)cc45)c3)cc2)cc1 ; c1ccc(-c2ccc(N(c3ccccc3)c3cccc(-c4ccc5sc6ccc(C7(c8cc(-c9ccccc9)cc(-c9ccccc9)c8)c8ccccc8-c8ccccc87)cc6c5c4)c3)cc2)cc1]

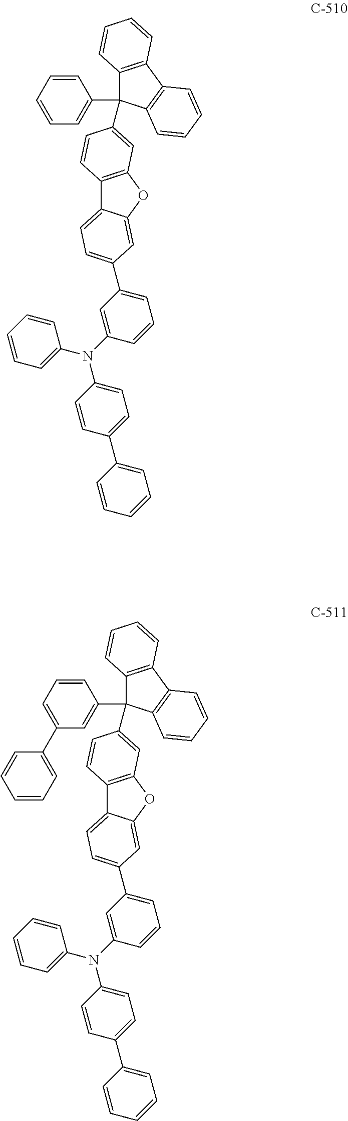 [SMILES: c1ccc(-c2ccc(N(c3ccccc3)c3cccc(-c4ccc5c(c4)oc4cc(C6(c7ccccc7)c7ccccc7-c7ccccc76)ccc45)c3)cc2)cc1 ; c1ccc(-c2ccc(N(c3ccccc3)c3cccc(-c4ccc5c(c4)oc4cc(C6(c7cccc(-c8ccccc8)c7)c7ccccc7-c7ccccc76)ccc45)c3)cc2)cc1]

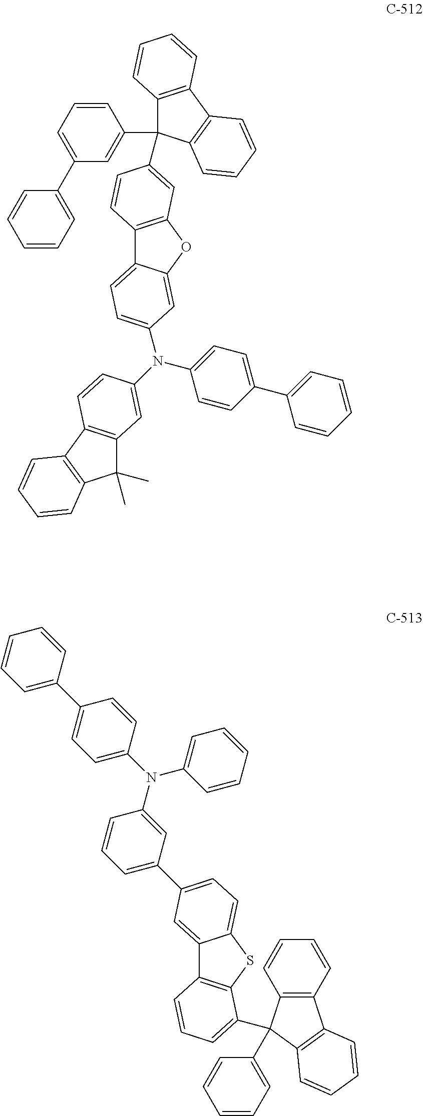 [SMILES: CC1(C)c2ccccc2-c2ccc(N(c3ccc(-c4ccccc4)cc3)c3ccc4c(c3)oc3cc(C5(c6cccc(-c7ccccc7)c6)c6ccccc6-c6ccccc65)ccc34)cc21 ; c1ccc(-c2ccc(N(c3ccccc3)c3cccc(-c4ccc5sc6c(C7(c8ccccc8)c8ccccc8-c8ccccc87)cccc6c5c4)c3)cc2)cc1]

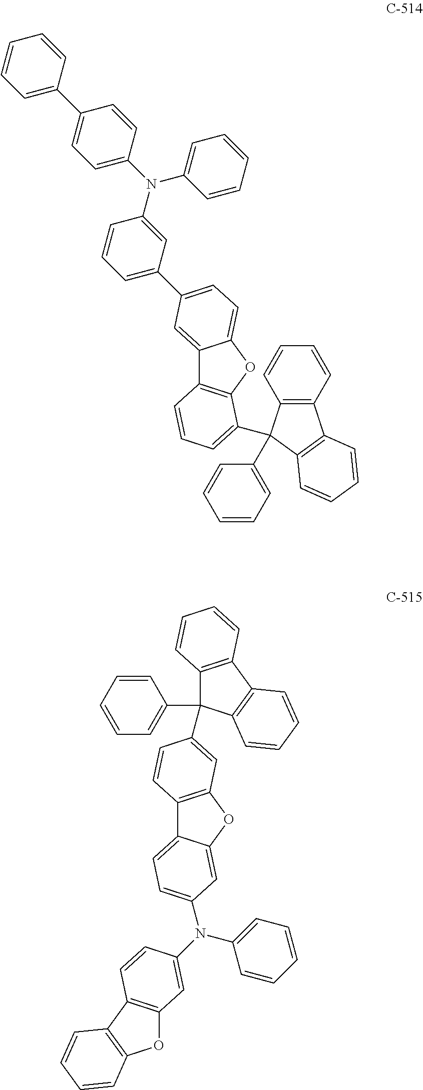 [SMILES: c1ccc(-c2ccc(N(c3ccccc3)c3cccc(-c4ccc5oc6c(C7(c8ccccc8)c8ccccc8-c8ccccc87)cccc6c5c4)c3)cc2)cc1 ; c1ccc(N(c2ccc3c(c2)oc2ccccc23)c2ccc3c(c2)oc2cc(C4(c5ccccc5)c5ccccc5-c5ccccc54)ccc23)cc1]

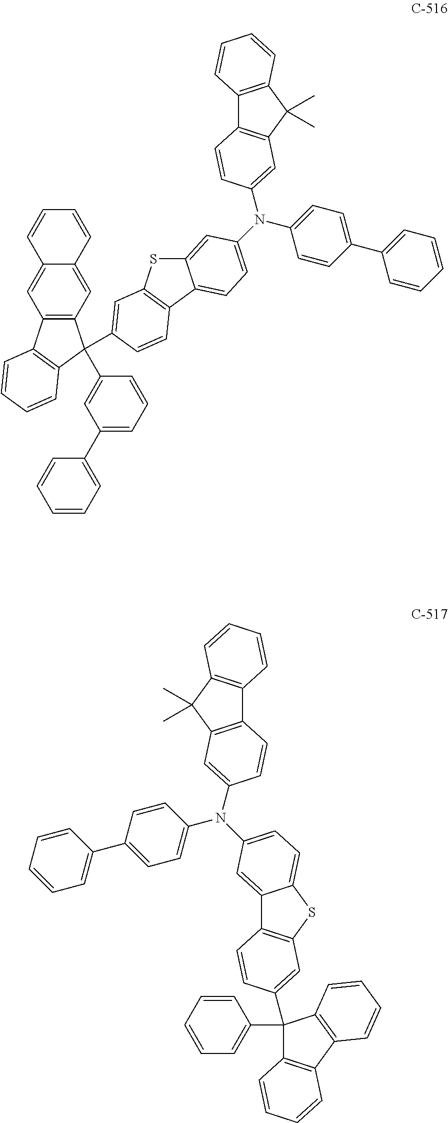 [SMILES: CC1(C)c2ccccc2-c2ccc(N(c3ccc(-c4ccccc4)cc3)c3ccc4c(c3)sc3cc(C5(c6cccc(-c7ccccc7)c6)c6ccccc6-c6cc7ccccc7cc65)ccc34)cc21 ; CC1(C)c2ccccc2-c2ccc(N(c3ccc(-c4ccccc4)cc3)c3ccc4sc5cc(C6(c7ccccc7)c7ccccc7-c7ccccc76)ccc5c4c3)cc21]

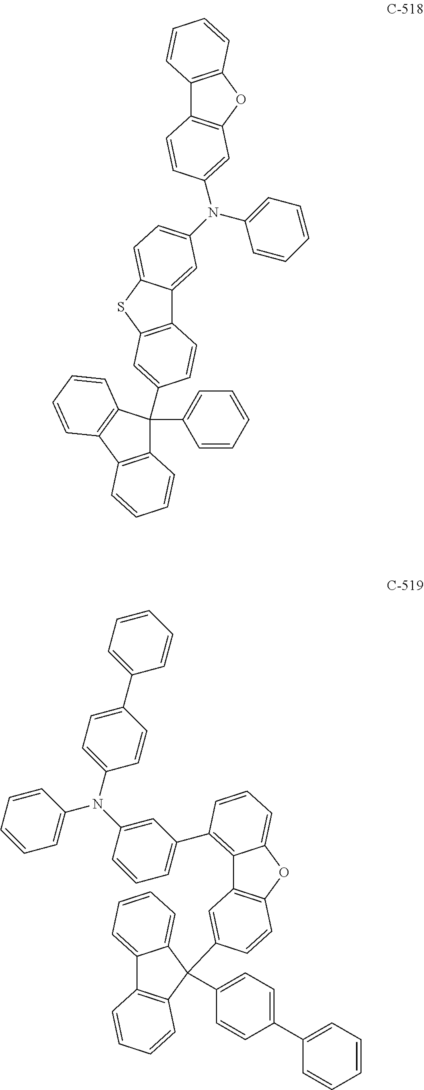 [SMILES: c1ccc(N(c2ccc3c(c2)oc2ccccc23)c2ccc3sc4cc(C5(c6ccccc6)c6ccccc6-c6ccccc65)ccc4c3c2)cc1 ; c1ccc(-c2ccc(N(c3ccccc3)c3cccc(-c4cccc5oc6ccc(C7(c8ccc(-c9ccccc9)cc8)c8ccccc8-c8ccccc87)cc6c45)c3)cc2)cc1]

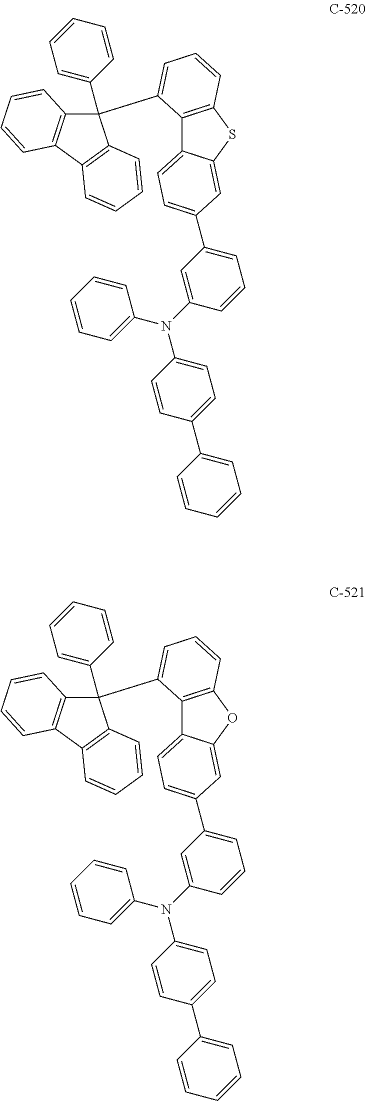 [SMILES: c1ccc(-c2ccc(N(c3ccccc3)c3cccc(-c4ccc5c(c4)sc4cccc(C6(c7ccccc7)c7ccccc7-c7ccccc76)c45)c3)cc2)cc1 ; c1ccc(-c2ccc(N(c3ccccc3)c3cccc(-c4ccc5c(c4)oc4cccc(C6(c7ccccc7)c7ccccc7-c7ccccc76)c45)c3)cc2)cc1]

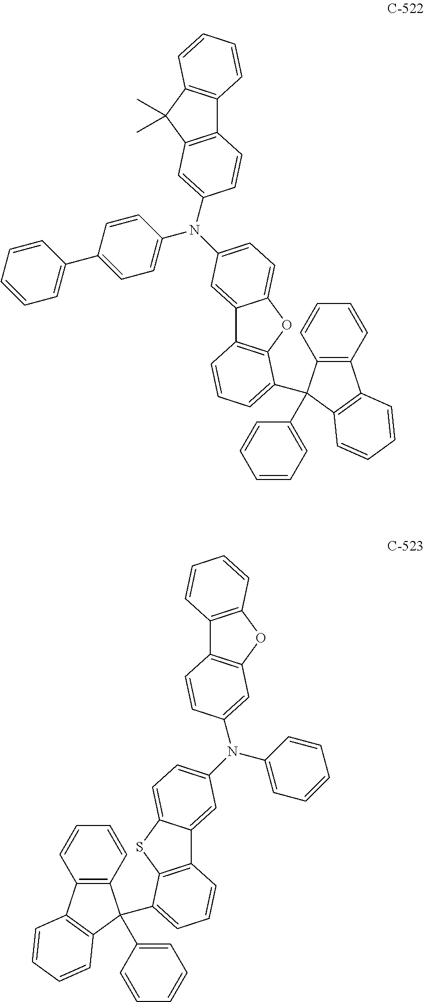 [SMILES: CC1(C)c2ccccc2-c2ccc(N(c3ccc(-c4ccccc4)cc3)c3ccc4oc5c(C6(c7ccccc7)c7ccccc7-c7ccccc76)cccc5c4c3)cc21 ; c1ccc(N(c2ccc3c(c2)oc2ccccc23)c2ccc3sc4c(C5(c6ccccc6)c6ccccc6-c6ccccc65)cccc4c3c2)cc1]

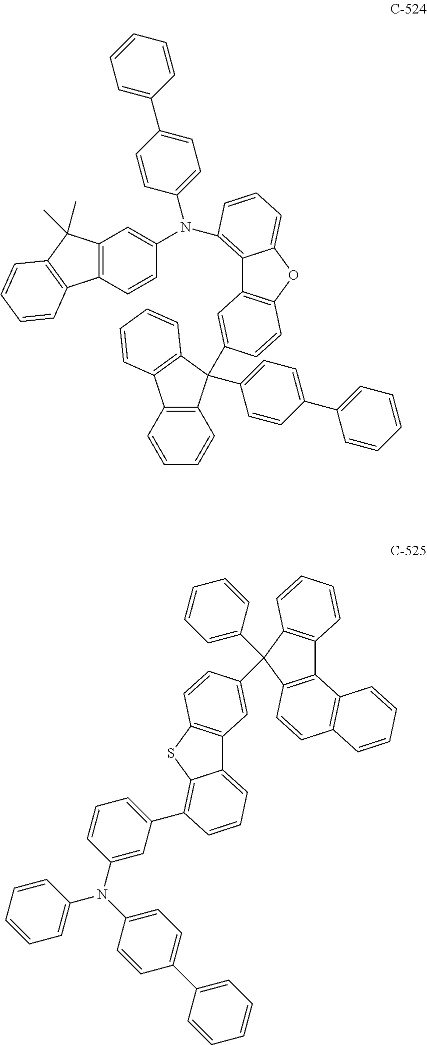 [SMILES: CC1(C)c2ccccc2-c2ccc(N(c3ccc(-c4ccccc4)cc3)c3cccc4oc5ccc(C6(c7ccc(-c8ccccc8)cc7)c7ccccc7-c7ccccc76)cc5c34)cc21 ; c1ccc(-c2ccc(N(c3ccccc3)c3cccc(-c4cccc5c4sc4ccc(C6(c7ccccc7)c7ccccc7-c7c6ccc6ccccc76)cc45)c3)cc2)cc1]

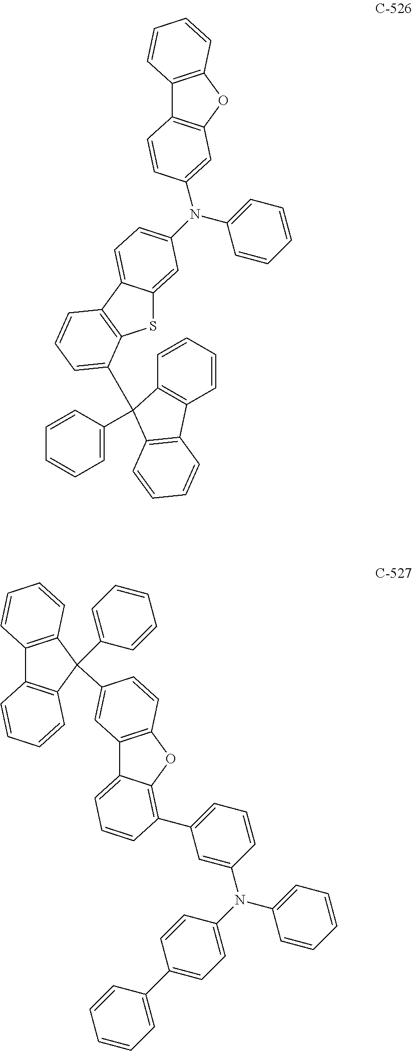 [SMILES: c1ccc(N(c2ccc3c(c2)oc2ccccc23)c2ccc3c(c2)sc2c(C4(c5ccccc5)c5ccccc5-c5ccccc54)cccc23)cc1 ; c1ccc(-c2ccc(N(c3ccccc3)c3cccc(-c4cccc5c4oc4ccc(C6(c7ccccc7)c7ccccc7-c7ccccc76)cc45)c3)cc2)cc1]

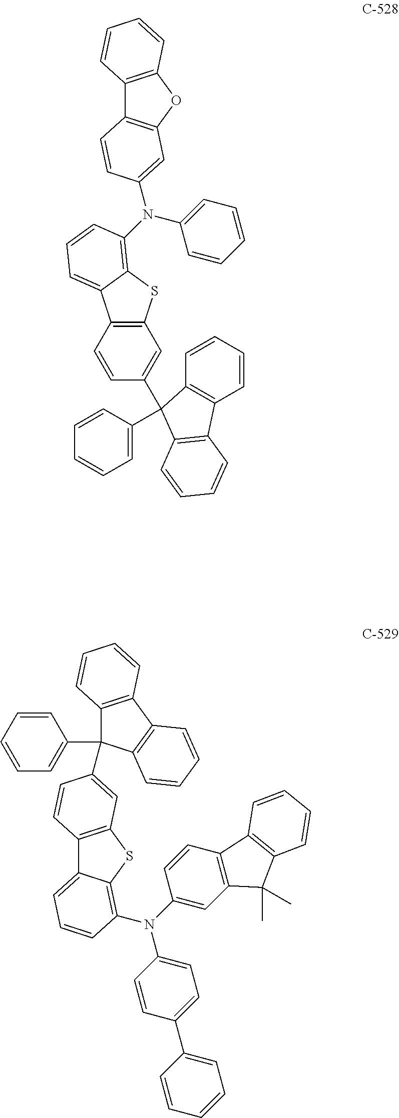 [SMILES: c1ccc(N(c2ccc3c(c2)oc2ccccc23)c2cccc3c2sc2cc(C4(c5ccccc5)c5ccccc5-c5ccccc54)ccc23)cc1 ; CC1(C)c2ccccc2-c2ccc(N(c3ccc(-c4ccccc4)cc3)c3cccc4c3sc3cc(C5(c6ccccc6)c6ccccc6-c6ccccc65)ccc34)cc21]

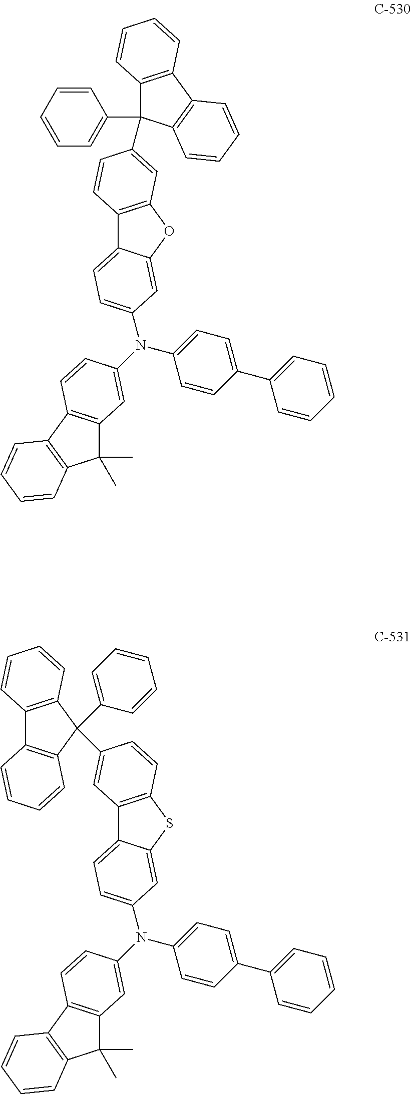 [SMILES: CC1(C)c2ccccc2-c2ccc(N(c3ccc(-c4ccccc4)cc3)c3ccc4c(c3)oc3cc(C5(c6ccccc6)c6ccccc6-c6ccccc65)ccc34)cc21 ; CC1(C)c2ccccc2-c2ccc(N(c3ccc(-c4ccccc4)cc3)c3ccc4c(c3)sc3ccc(C5(c6ccccc6)c6ccccc6-c6ccccc65)cc34)cc21]

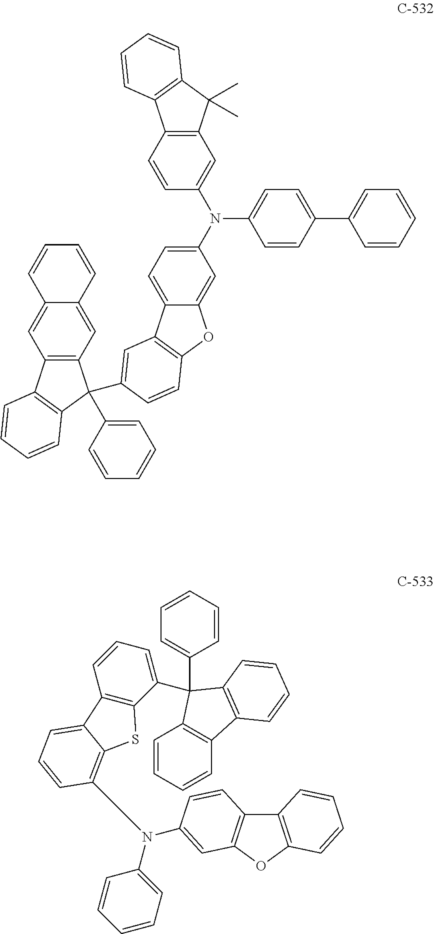 [SMILES: CC1(C)c2ccccc2-c2ccc(N(c3ccc(-c4ccccc4)cc3)c3ccc4c(c3)oc3ccc(C5(c6ccccc6)c6ccccc6-c6cc7ccccc7cc65)cc34)cc21 ; c1ccc(N(c2ccc3c(c2)oc2ccccc23)c2cccc3c2sc2c(C4(c5ccccc5)c5ccccc5-c5ccccc54)cccc23)cc1]

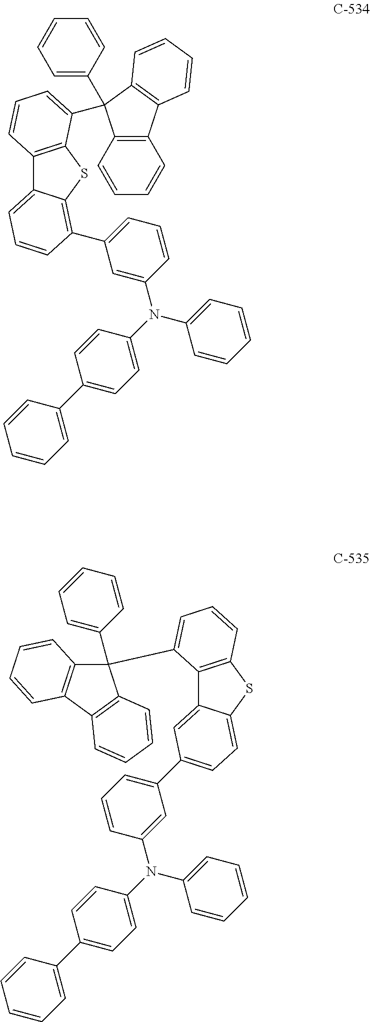 [SMILES: c1ccc(-c2ccc(N(c3ccccc3)c3cccc(-c4cccc5c4sc4c(C6(c7ccccc7)c7ccccc7-c7ccccc76)cccc45)c3)cc2)cc1 ; c1ccc(-c2ccc(N(c3ccccc3)c3cccc(-c4ccc5sc6cccc(C7(c8ccccc8)c8ccccc8-c8ccccc87)c6c5c4)c3)cc2)cc1]

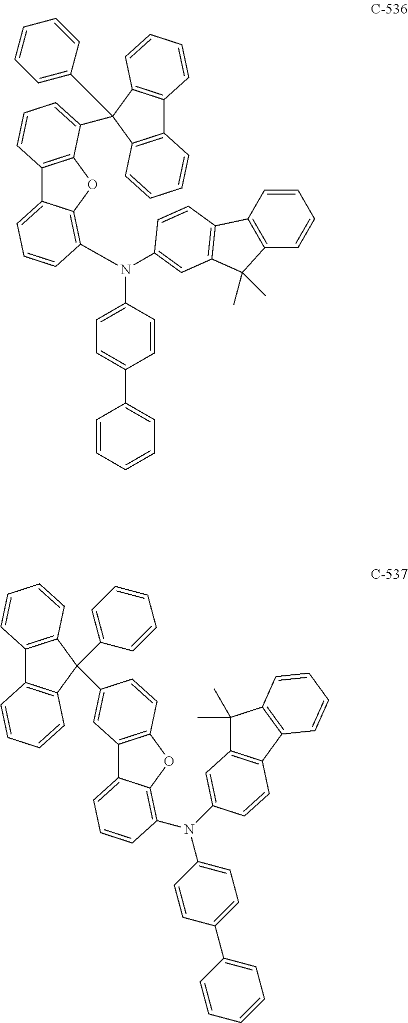 [SMILES: CC1(C)c2ccccc2-c2ccc(N(c3ccc(-c4ccccc4)cc3)c3cccc4c3oc3c(C5(c6ccccc6)c6ccccc6-c6ccccc65)cccc34)cc21 ; CC1(C)c2ccccc2-c2ccc(N(c3ccc(-c4ccccc4)cc3)c3cccc4c3oc3ccc(C5(c6ccccc6)c6ccccc6-c6ccccc65)cc34)cc21]

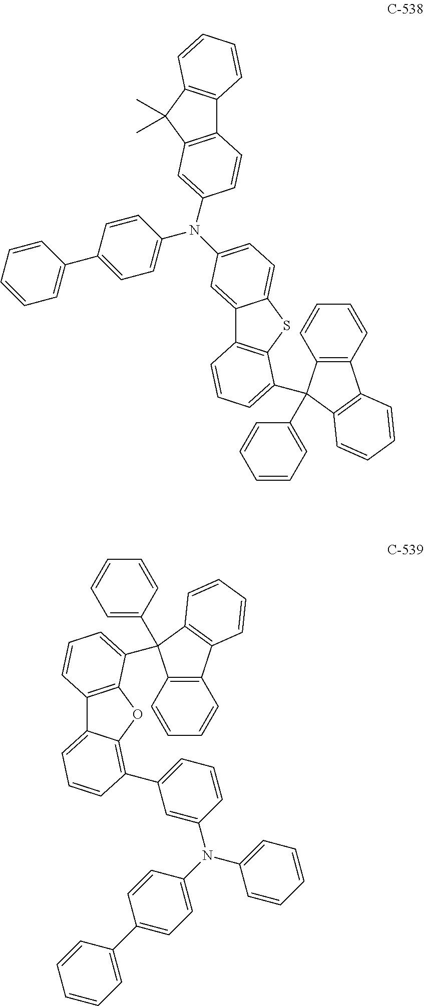 [SMILES: CC1(C)c2ccccc2-c2ccc(N(c3ccc(-c4ccccc4)cc3)c3ccc4sc5c(C6(c7ccccc7)c7ccccc7-c7ccccc76)cccc5c4c3)cc21 ; c1ccc(-c2ccc(N(c3ccccc3)c3cccc(-c4cccc5c4oc4c(C6(c7ccccc7)c7ccccc7-c7ccccc76)cccc45)c3)cc2)cc1]

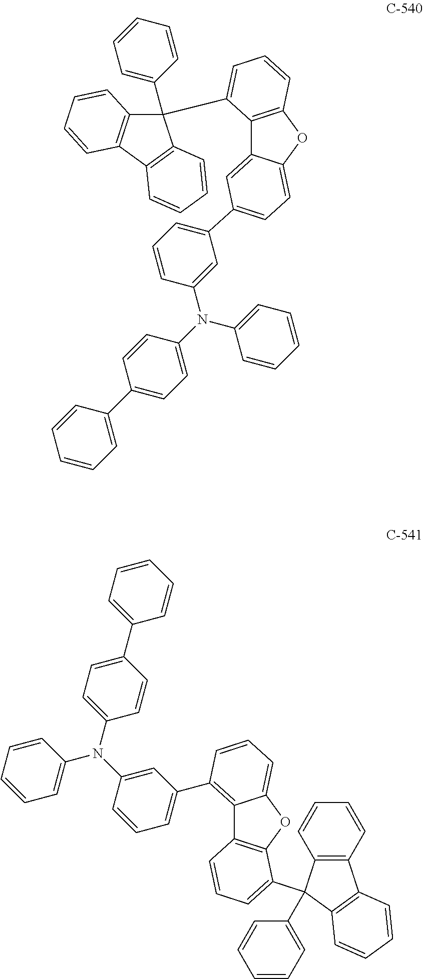 [SMILES: c1ccc(-c2ccc(N(c3ccccc3)c3cccc(-c4ccc5oc6cccc(C7(c8ccccc8)c8ccccc8-c8ccccc87)c6c5c4)c3)cc2)cc1 ; c1ccc(-c2ccc(N(c3ccccc3)c3cccc(-c4cccc5oc6c(C7(c8ccccc8)c8ccccc8-c8ccccc87)cccc6c45)c3)cc2)cc1]

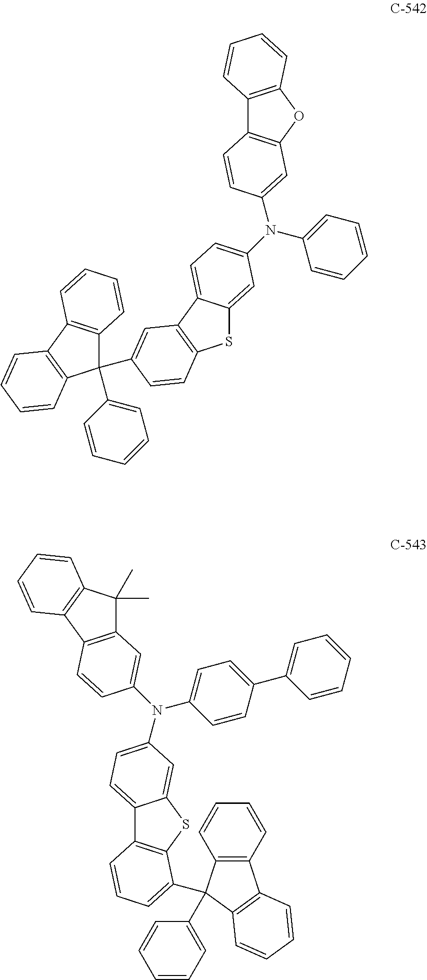 [SMILES: c1ccc(N(c2ccc3c(c2)oc2ccccc23)c2ccc3c(c2)sc2ccc(C4(c5ccccc5)c5ccccc5-c5ccccc54)cc23)cc1 ; CC1(C)c2ccccc2-c2ccc(N(c3ccc(-c4ccccc4)cc3)c3ccc4c(c3)sc3c(C5(c6ccccc6)c6ccccc6-c6ccccc65)cccc34)cc21]

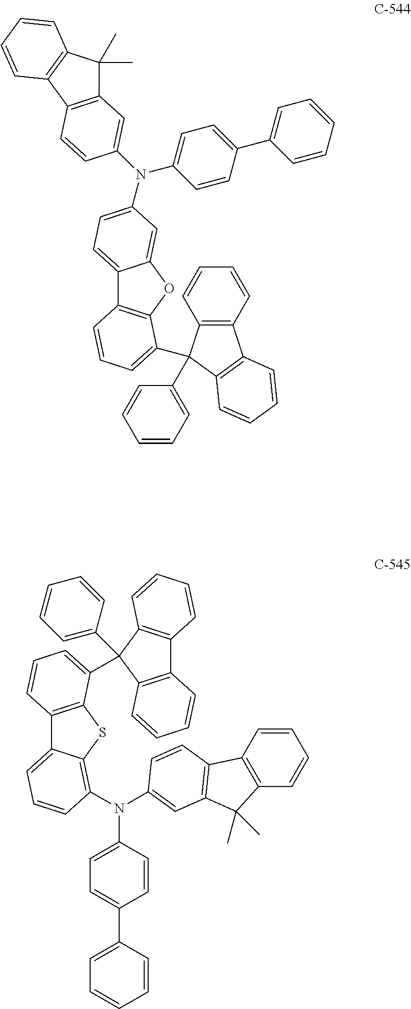 [SMILES: CC1(C)c2ccccc2-c2ccc(N(c3ccc(-c4ccccc4)cc3)c3ccc4c(c3)oc3c(C5(c6ccccc6)c6ccccc6-c6ccccc65)cccc34)cc21 ; CC1(C)c2ccccc2-c2ccc(N(c3ccc(-c4ccccc4)cc3)c3cccc4c3sc3c(C5(c6ccccc6)c6ccccc6-c6ccccc65)cccc34)cc21]

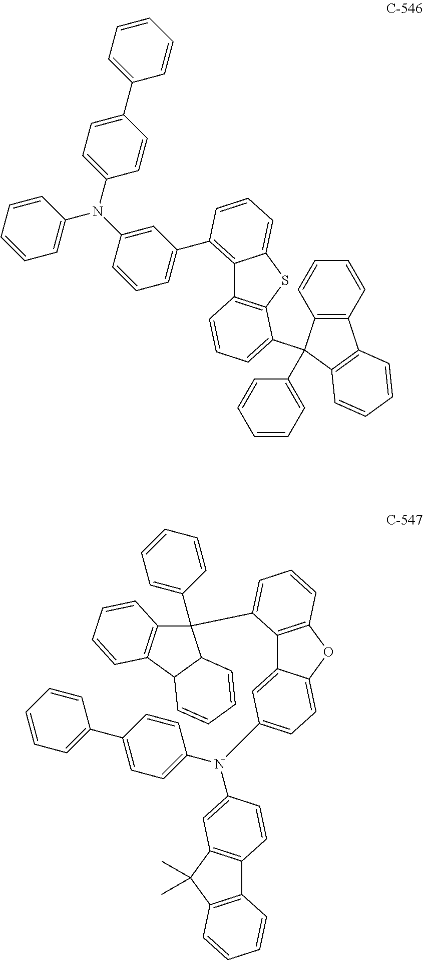 [SMILES: c1ccc(-c2ccc(N(c3ccccc3)c3cccc(-c4cccc5sc6c(C7(c8ccccc8)c8ccccc8-c8ccccc87)cccc6c45)c3)cc2)cc1 ; CC1(C)c2ccccc2-c2ccc(N(c3ccc(-c4ccccc4)cc3)c3ccc4oc5cccc(C6(c7ccccc7)c7ccccc7C7C=CC=CC76)c5c4c3)cc21]

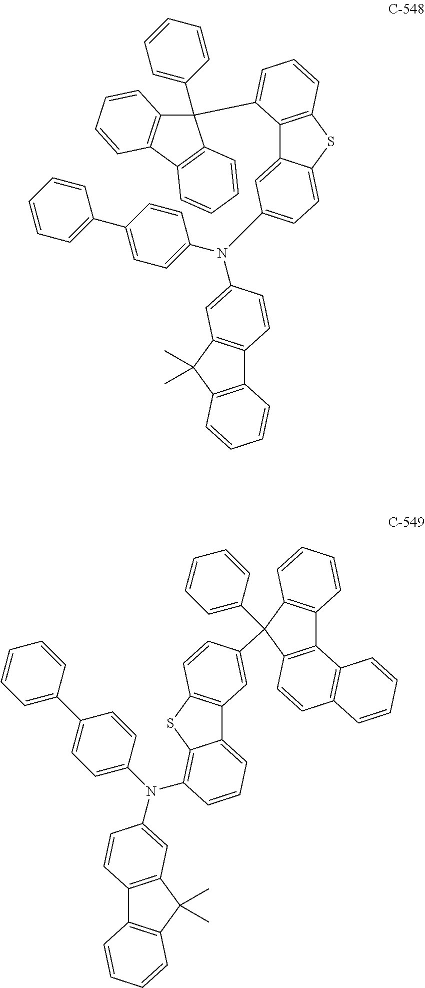 [SMILES: CC1(C)c2ccccc2-c2ccc(N(c3ccc(-c4ccccc4)cc3)c3ccc4sc5cccc(C6(c7ccccc7)c7ccccc7-c7ccccc76)c5c4c3)cc21 ; CC1(C)c2ccccc2-c2ccc(N(c3ccc(-c4ccccc4)cc3)c3cccc4c3sc3ccc(C5(c6ccccc6)c6ccccc6-c6c5ccc5ccccc65)cc34)cc21]

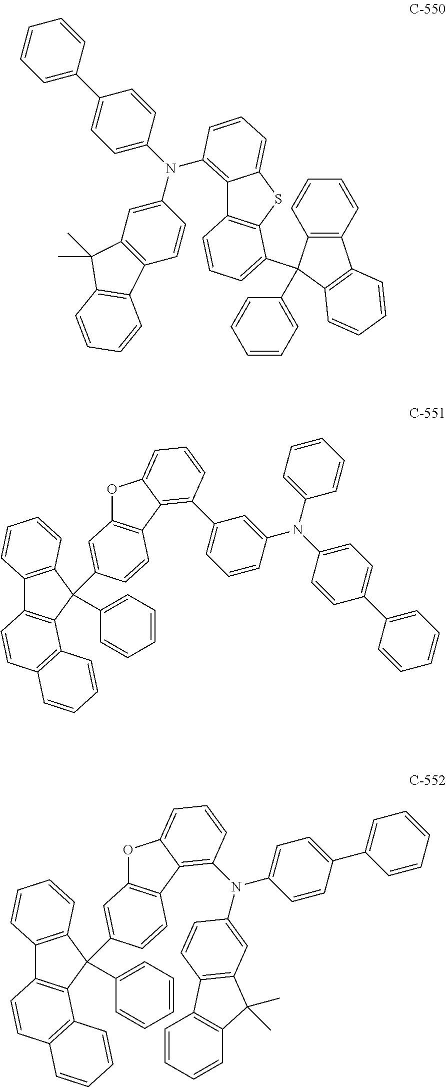 [SMILES: CC1(C)c2ccccc2-c2ccc(N(c3ccc(-c4ccccc4)cc3)c3cccc4sc5c(C6(c7ccccc7)c7ccccc7-c7ccccc76)cccc5c34)cc21 ; c1ccc(-c2ccc(N(c3ccccc3)c3cccc(-c4cccc5oc6cc(C7(c8ccccc8)c8ccccc8-c8ccc9ccccc9c87)ccc6c45)c3)cc2)cc1 ; CC1(C)c2ccccc2-c2ccc(N(c3ccc(-c4ccccc4)cc3)c3cccc4oc5cc(C6(c7ccccc7)c7ccccc7-c7ccc8ccccc8c76)ccc5c34)cc21]

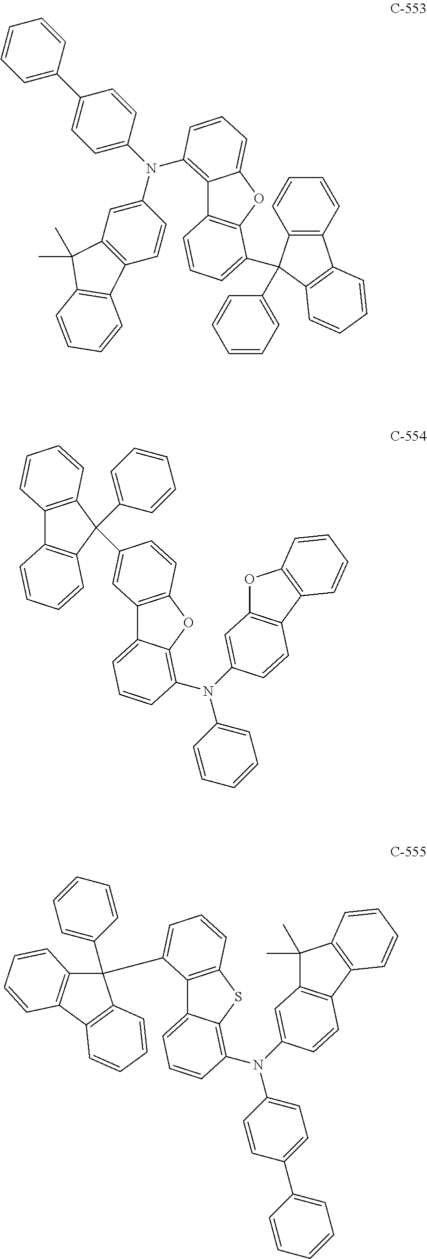 [SMILES: CC1(C)c2ccccc2-c2ccc(N(c3ccc(-c4ccccc4)cc3)c3cccc4oc5c(C6(c7ccccc7)c7ccccc7-c7ccccc76)cccc5c34)cc21 ; c1ccc(N(c2ccc3c(c2)oc2ccccc23)c2cccc3c2oc2ccc(C4(c5ccccc5)c5ccccc5-c5ccccc54)cc23)cc1 ; CC1(C)c2ccccc2-c2ccc(N(c3ccc(-c4ccccc4)cc3)c3cccc4c3sc3cccc(C5(c6ccccc6)c6ccccc6-c6ccccc65)c34)cc21]

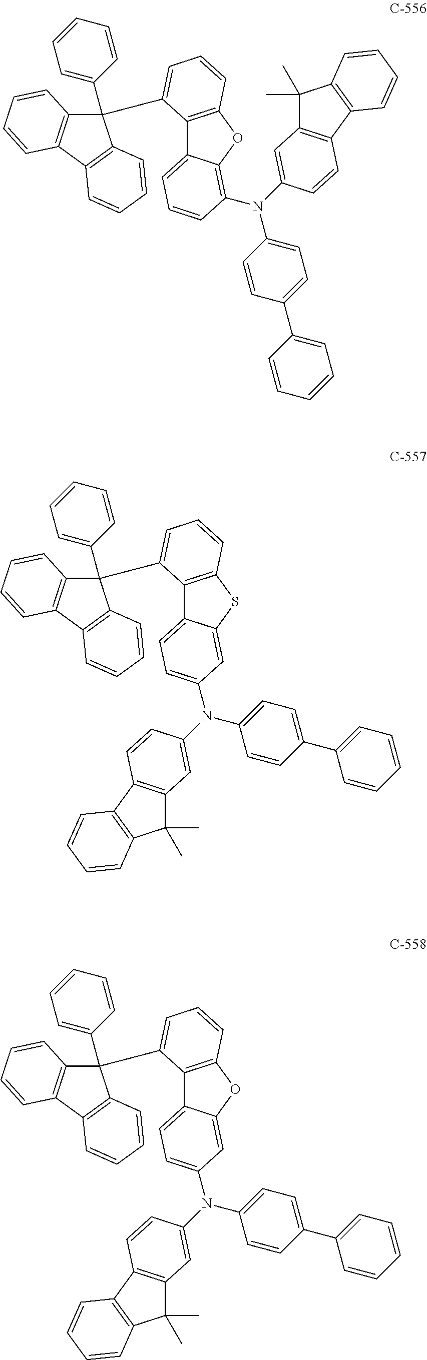 [SMILES: CC1(C)c2ccccc2-c2ccc(N(c3ccc(-c4ccccc4)cc3)c3cccc4c3oc3cccc(C5(c6ccccc6)c6ccccc6-c6ccccc65)c34)cc21 ; CC1(C)c2ccccc2-c2ccc(N(c3ccc(-c4ccccc4)cc3)c3ccc4c(c3)sc3cccc(C5(c6ccccc6)c6ccccc6-c6ccccc65)c34)cc21 ; CC1(C)c2ccccc2-c2ccc(N(c3ccc(-c4ccccc4)cc3)c3ccc4c(c3)oc3cccc(C5(c6ccccc6)c6ccccc6-c6ccccc65)c34)cc21]

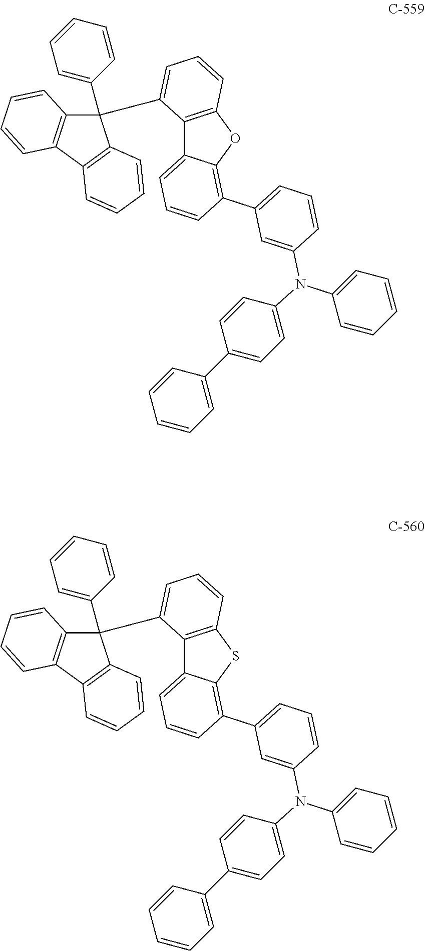 [SMILES: c1ccc(-c2ccc(N(c3ccccc3)c3cccc(-c4cccc5c4oc4cccc(C6(c7ccccc7)c7ccccc7-c7ccccc76)c45)c3)cc2)cc1 ; c1ccc(-c2ccc(N(c3ccccc3)c3cccc(-c4cccc5c4sc4cccc(C6(c7ccccc7)c7ccccc7-c7ccccc76)c45)c3)cc2)cc1]

The compound represented by formula 1 according to the present disclosure may be prepared by a synthetic method known to one skilled in the art, for example, according to the following reaction schemes 1 to 4, but is not limited thereto.

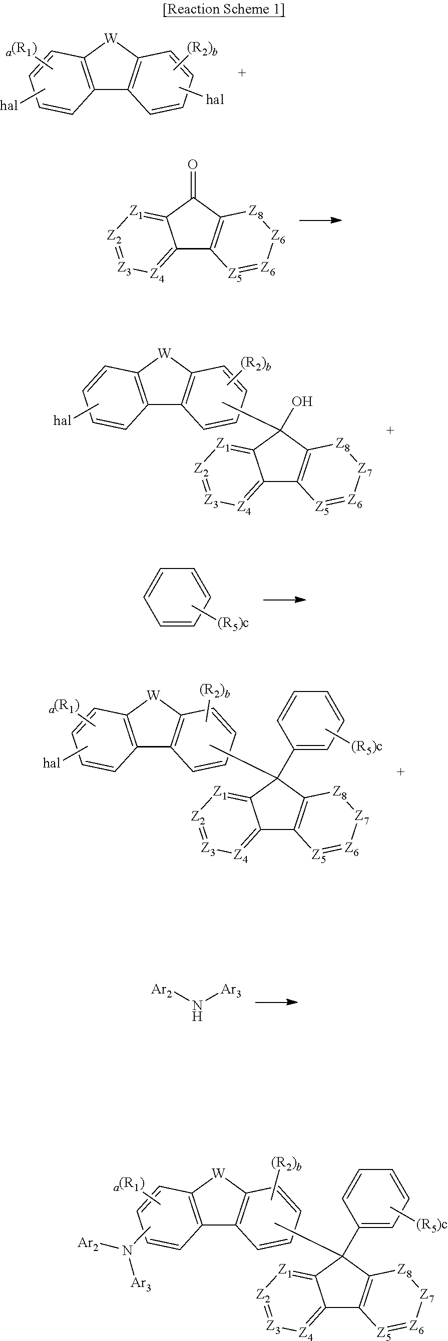

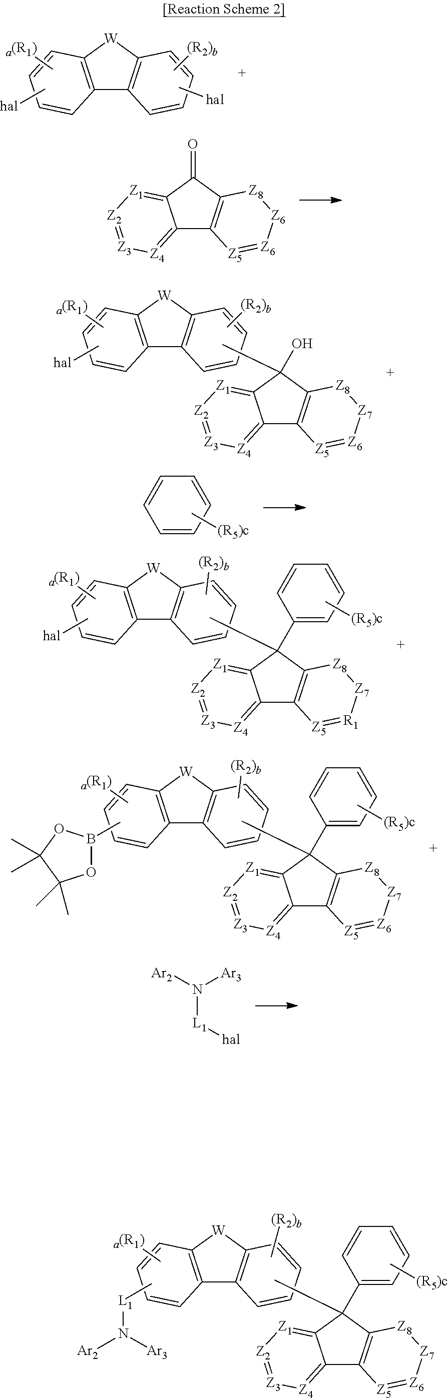

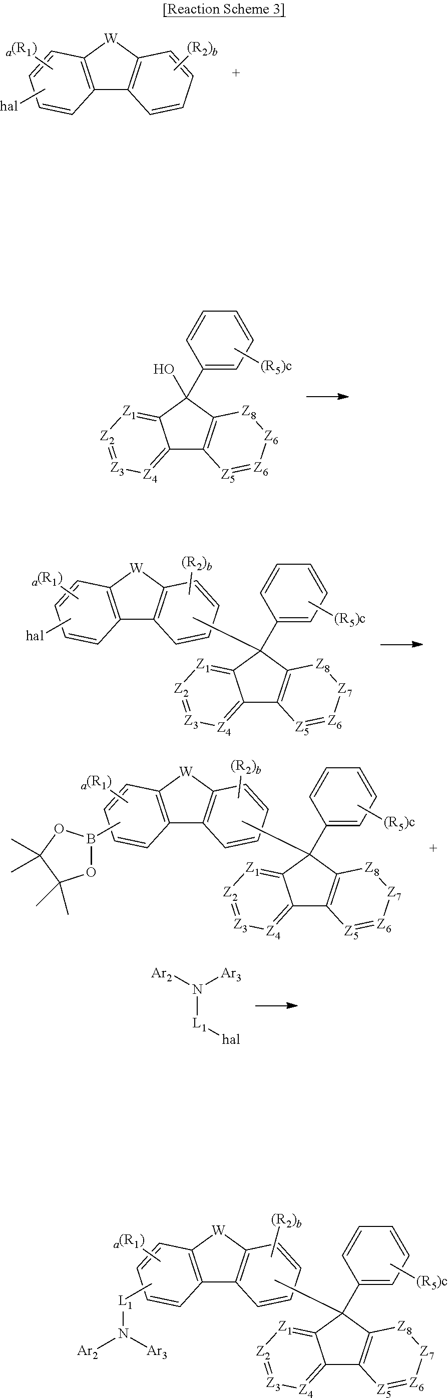

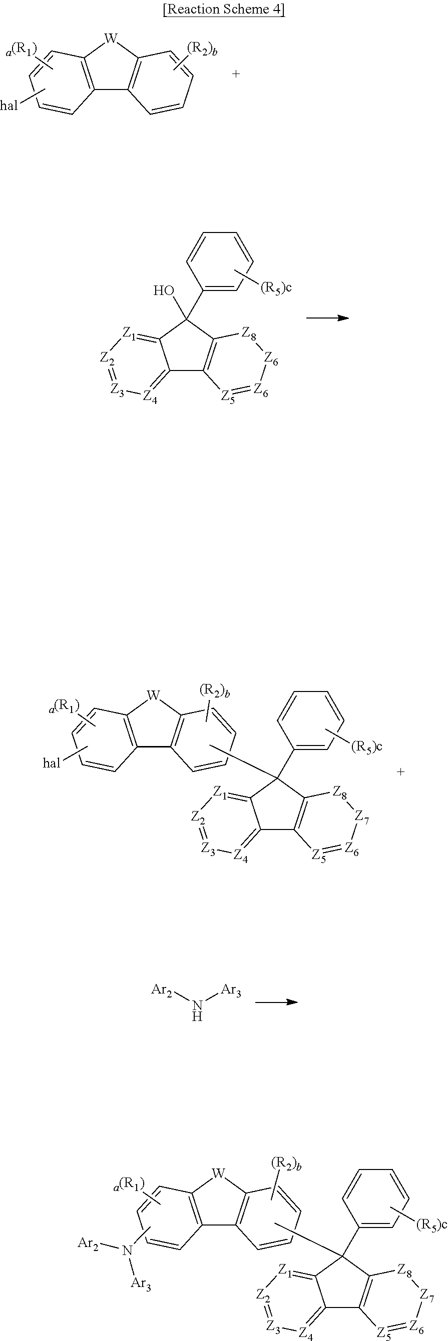

In reaction schemes 1 to 4, the definition of each substituent is as defined in formulas 1 and 2, and Hal represents a halogen atom.

Although illustrative synthesis examples of the compounds represented by formula 1 or 2 of the present disclosure are described above, one skilled in the art will be able to readily understand that all of them are based on a Buchwald-Hartwig cross-coupling reaction, an N-arylation reaction, a H-mont-mediated etherification reaction, a Miyaura borylation reaction, a Suzuki cross-coupling reaction, an intramolecular acid-induced cyclization reaction, a Pd(II)-catalyzed oxidative cyclization reaction, a Grignard reaction, a Heck reaction, a Cyclic Dehydration reaction, an SN₁ substitution reaction, an SN₂ substitution reaction, a Phosphine-mediated reductive cyclization reaction, etc., and the reactions above proceed even when substituents, which are defined in formula 1 or 2 but are not specified in the specific synthesis examples, are bonded.

The present disclosure provides an organic electroluminescent material comprising the organic electroluminescence compound represented by formula 1, and an organic electroluminescent device comprising the material. The material may consist solely of the organic electroluminescent compound of the present disclosure, and may further comprise conventional materials included in the organic electroluminescent material.

The organic electroluminescent compound represented by formula 1 of the present disclosure may be included in at least one layer of a light-emitting layer, a hole injection layer, a hole transport layer, a hole auxiliary layer, a light-emitting auxiliary layer, an electron transport layer, an electron buffer layer, an electron injection layer, an interlayer, a hole blocking layer, and an electron blocking layer. Preferably, it may be included in a hole transport zone, which may be a second hole transport layer, and which may comprise at least one of the hole auxiliary layer and the light-emitting auxiliary layer.

The organic electroluminescent device according to the present disclosure may comprise a first electrode, a second electrode, and at least one organic layer between the first electrode and the second electrode. One of the first and second electrodes may be an anode and the other may be a cathode. The organic layer comprise an light-emitting layer, and may further comprise at least one layer selected from a hole injection layer, a hole transport layer, a hole auxiliary layer, a light-emitting auxiliary layer, an electron transport layer, an electron buffer layer, an electron injection layer, an interlayer, a hole blocking layer, and an electron blocking layer. Each of the layers may be a multi-layer.

The present disclosure comprises a hole transport zone between an anode and a light-emitting layer, wherein the hole transport zone may comprise at least one of a hole injection layer, a hole transport layer, a hole auxiliary layer, a light-emitting auxiliary layer and an electron blocker layer. The hole injection layer, the hole transport layer, the hole auxiliary layer, the light-emitting auxiliary layer and the electron blocker layer may be a single layer or a multi-layer of which two or more layers are stacked. The hole injection layer may be a multi-layer for the purpose of lowering a hole injection barrier (or hole injection voltage) from the anode to the hole transport layer or the electron blocking layer, and each layer may use two compounds simultaneously. The electron blocking layer is positioned between the hole transport layer (or the hole injection layer) and the light-emitting layer, and blocks overflow of electrons from the light-emitting layer to confine excitons in the light emission layer, thereby preventing light-emitting leakage. According to one embodiment of the present disclosure, at least one layer of the electron transport zone may comprise the compound represented by formula 1.

Furthermore, the hole transport zone may comprise at least one of a p-doped hole injection layer, a hole transport layer and a light-emitting auxiliary layer. Here, the p-doped hole injection layer means a hole injection layer doped with a p-dopant. A p-dopant is a material that allows a layer to have a p semiconductor property. The p-semiconductor property refers to the property of injecting or transporting holes to the HOMO (highest occupied molecular orbital) energy level, i.e., a property of a material having a high conductivity of holes. The p-dopant may preferably be substantially uniformly distributed in a p-doped layer, which can be achieved by co-deposition of the hole injection material with the p-dopant. Further, the p-dopant may be included in an amount of about 0.01 to about 50 wt %, preferably about 0.1 to about 20 wt %, more preferably about 1 to about 10 wt % based on the hole injection material.

A p-dopant according to one embodiment of the present disclosure may comprise at least one compound represented by any one of the following formulas P-1 to P-7.

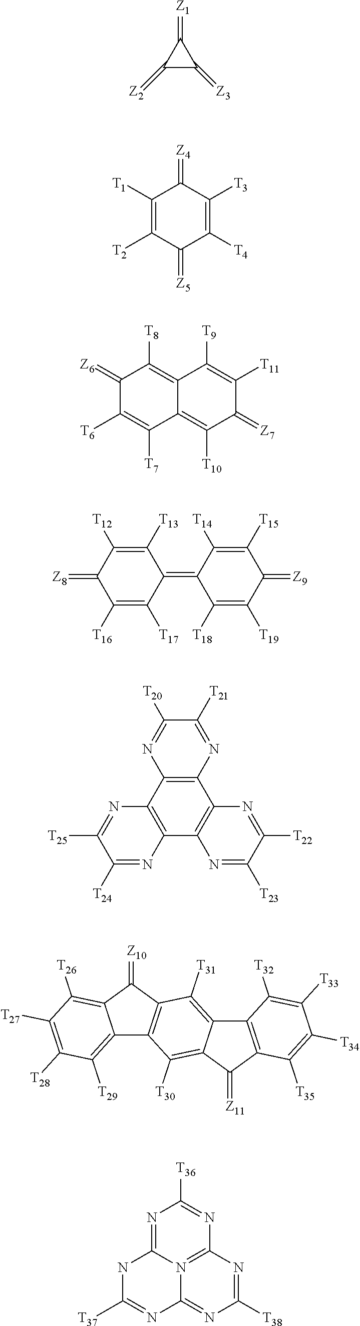

In formulas P-1 to P-7, T₁ to T₄ and T₆ to T₃₈ each independently represent hydrogen, deuterium, a halogen, a substituted or unsubstituted (C1-C50)fluorinated alkyl, a cyano, a substituted or unsubstituted (C1-C50)alkoxy, a substituted or unsubstituted (C1-C50)alkyl, a substituent or unsubstituted (C6-C50) aryl, or a substituted or unsubstituted (3- to 30-membered)heteroaryl. In formulas P-1 to P-7, ═Z₁ to ═Z₁₁ may be each independently represented by any one of the following formulas:

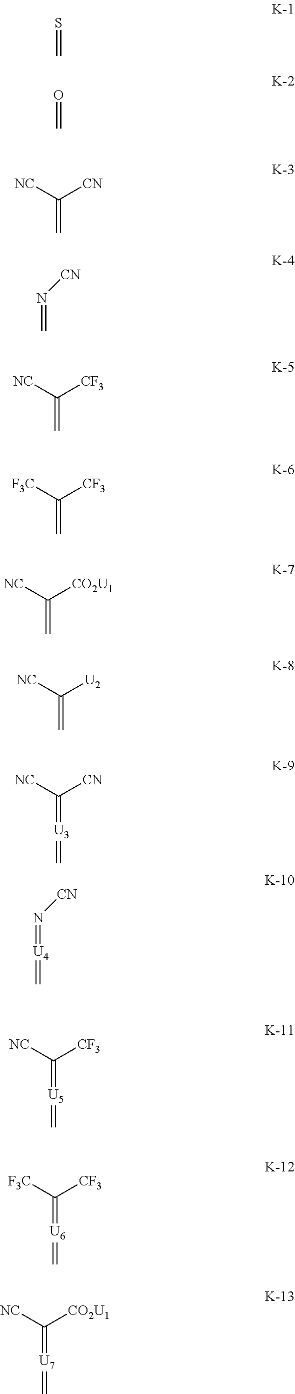

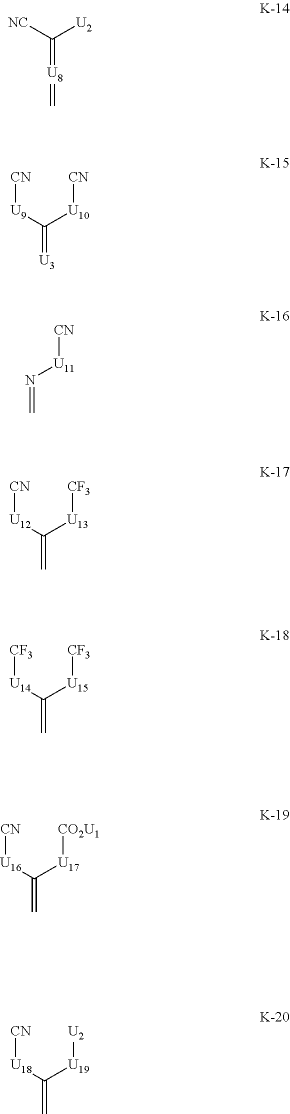

In the aforementioned formulas, U₁ and U₂ each independently represent hydrogen, deuterium, a halogen, a substituted or unsubstituted (C1-C50)fluorinated alkyl, a cyano, a substituted or unsubstituted (C1-C50)alkoxy, a substituted and unsubstituted (C1-C50)alkyl, a substituted and unsubstituted (C6-C50)aryl, or a substituted or unsubstituted (3- to 30-membered)heteroaryl. U₃ to U₈ each independently represent a substituted or unsubstituted (C1-C50)fluorinated alkanediylidene, a substituted or unsubstituted (C1-C50)alkanediylidene, a substituted or unsubstituted (C6-C50)aryldiylidene, or a substituted or unsubstituted (3- to 30-membered)heteroaryldiylidene. U₉ to U₁₉ each independently represent a substituted or unsubstituted (C1-C50)fluorinated alkylene, a substituted or unsubstituted (C1-C50)alkylene, a substituted or unsubstituted (C6-C50)arylene, or a substituted or unsubstituted (3- to 30-membered)heteroarylene.

The p-dopant in the present disclosure may be specifically exemplified by the following compounds, but is not limited thereto.

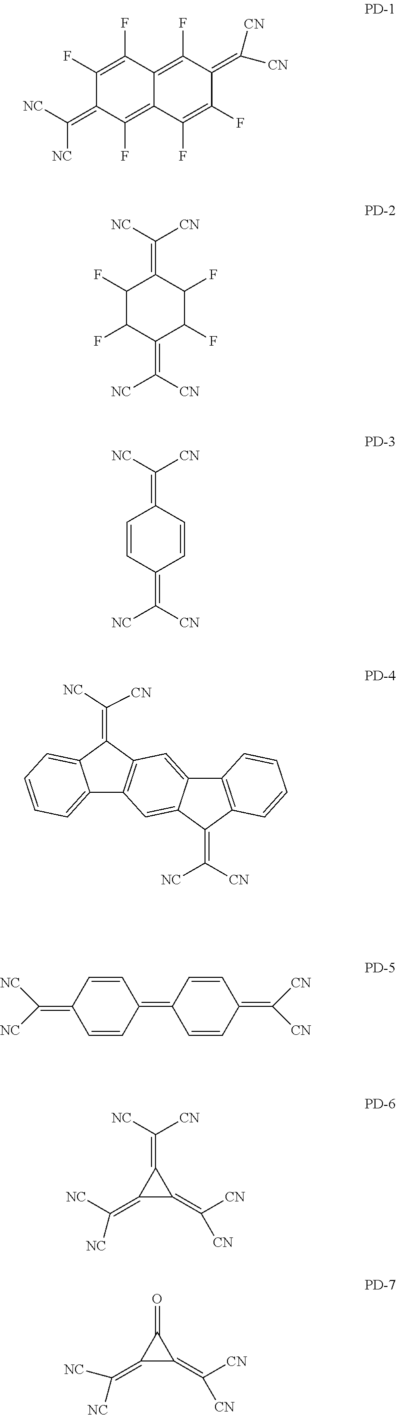

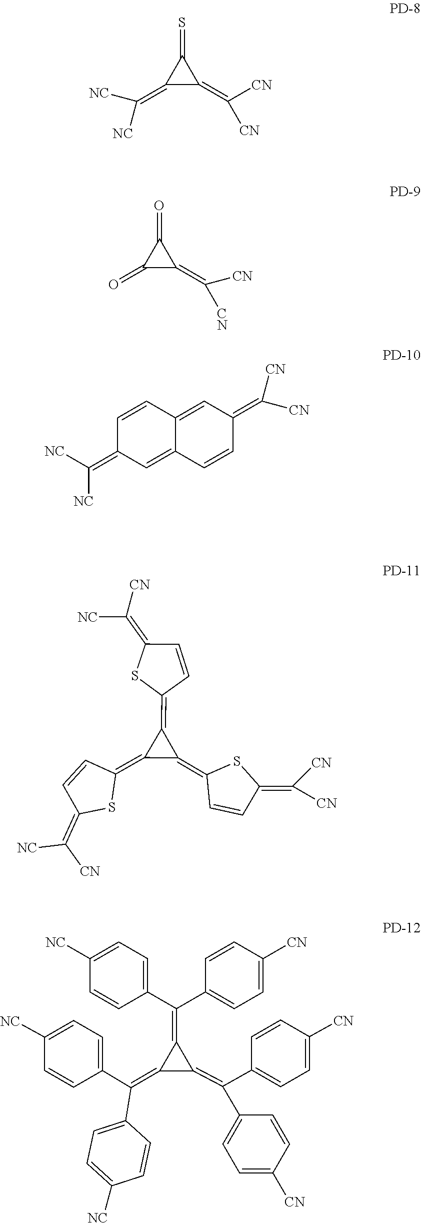 [SMILES: N#CC(C#N)=c1c(=S)c1=C(C#N)C#N ; N#CC(C#N)=C1C(=O)C1=O ; N#CC(C#N)=c1ccc2cc(=C(C#N)C#N)ccc2c1 ; N#CC(C#N)=c1ccc(=C2C(=c3ccc(=C(C#N)C#N)s3)C2=c2ccc(=C(C#N)C#N)s2)s1 ; N#Cc1ccc(C(=C2C(=C(c3ccc(C#N)cc3)c3ccc(C#N)cc3)C2=C(c2ccc(C#N)cc2)c2ccc(C#N)cc2)c2ccc(C#N)cc2)cc1]

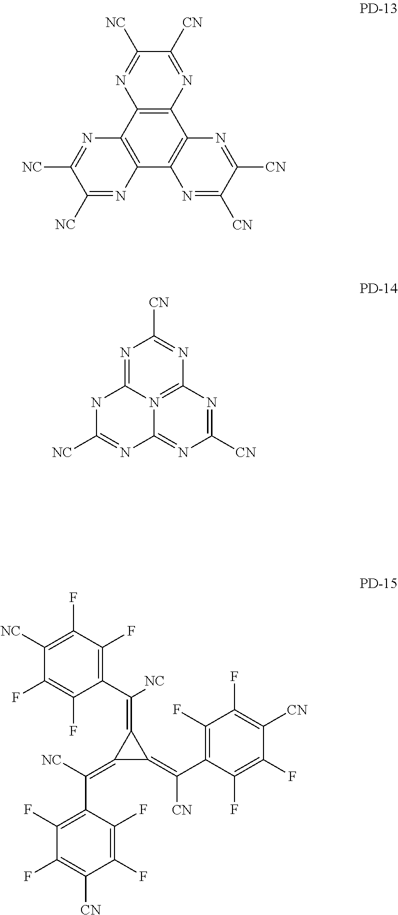

The present disclosure comprises an electron transport zone between a light-emitting layer and a cathode, wherein the electron transport zone may comprise at least one of a hole blocking layer, an electron transport layer, an electron buffer layer, and an electron injection layer. The hole blocking layer, the electron transport layer, the electron buffer layer, and the electron injection layer may each be a single layer or a multi-layer in which two or more layers are stacked. The electron injection layer may be further doped with an n-dopant. The electron buffer layer may a multi-layer for the purpose of controlling the electron injection and improving interfacial properties between the light-emitting layer and the electron injection layer, and each layer may comprise two compounds simultaneously. The hole blocking layer or the electron transport layer may also be a multi-layer, and each layer may comprise a plurality of compounds simultaneously.

The light-emitting auxiliary layer may be a layer positioned between an anode and a light-emitting layer, or between a cathode and a light-emitting layer. When the light-emitting auxiliary layer is positioned between the anode and the light-emitting layer, it may be used for facilitating the injection and/or transportation of holes or blocking the overflow of electrons. When the light-emitting auxiliary layer is positioned between the cathode and the light-emitting layer, it may also be used to facilitate the injection and/or transportation of electrons or blocking an overflow of holes. In addition, the hole auxiliary layer is positioned between the hole transport layer (or the hole injection layer) and the light-emitting layer, and can exhibit an effect of facilitating or blocking the transport rate (or injection rate) of holes, thereby controlling charge balance. When an organic electroluminescent device includes two or more hole transport layers, the hole transport layer further included may be used as a hole auxiliary layer or an electron blocking layer. The light-emitting auxiliary layer, the hole auxiliary layer, or the electron blocking layer may have an effect of improving efficiency and/or lifespan of an organic electroluminescent device.

The first electrode and the second electrode may each be formed of a transparent conductive material, or a transflective or reflective conductive material. Depending on the kind of the material forming the first electrode and the second electrode, the organic electroluminescent device may be of a front emission type, a back emission type or a both-sided emission type.

In the organic electroluminescent device of the present disclosure, the organic material layer may further comprise at least one metal selected from the group consisting of Group 1, Group 2, Group 4 transition metals, Group 5 transition metal, lanthanide metal, and organometallics of d-transition elements, or at least one complex compounds comprising such a metal.

In addition, the organic electroluminescent device of the present disclosure can emit white light by further including at least one light-emitting layer including a blue, red or green light-emitting compound known in the art in addition to the compound of the present disclosure. If necessary, a yellow or orange light-emitting layer may be further included.

In the organic electroluminescent device of the present disclosure, preferably, at least one layer (hereinafter, “a surface layer”) selected from a chalcogenide layer, a halogenated metal layer, and a metal oxide layer may be placed on an inner surface(s) of one or both electrode(s). Specifically, a chalcogenide (including oxides) layer of silicon and aluminum is preferably placed on an anode surface of an electroluminescent medium layer, and a halogenated metal layer or a metal oxide layer is preferably placed on a cathode surface of an electroluminescent medium layer. The operation stability for the organic electroluminescent device may be obtained by the surface layer. Preferably, the chalcogenide includes SiO_{X} (1≤X≤2), AlO_{X} (1≤X≤1.5), SiON, SiAlON, etc.; the halogenated metal includes LiF, MgF₂, CaF₂, a rare earth metal fluoride, etc.; and the metal oxide includes Cs₂O, Li₂O, MgO, SrO, BaO, CaO, etc.

Further, in the organic electroluminescent device of the present disclosure, preferably, a mixed region of an electron transport compound and a reductive dopant, or a mixed region of a hole transport compound and an oxidative dopant may be placed on at least one surface of a pair of electrodes. In this case, the electron transport compound is reduced to an anion, and thus it becomes easier to inject and transport electrons from the mixed region to an electroluminescent medium. Furthermore, the hole transport compound is oxidized to a cation, and thus it becomes easier to inject and transport holes from the mixed region to the electroluminescent medium. Preferably, the oxidative dopant includes various Lewis acids and acceptor compounds, and the reductive dopant includes alkali metals, alkali metal compounds, alkaline earth metals, rare-earth metals, and mixtures thereof. A reductive dopant layer may be employed as a charge generating layer to prepare an organic electroluminescent device having two or more light-emitting layers and emitting white light.

An organic electroluminescent material according to one embodiment of the present disclosure may be used as a light-emitting material for a white organic light-emitting device. The white organic light-emitting device has been suggested to have various structures such as a side-by-side method or a stacking method, etc., according to the arrangement of R(Red), G (Green) or YG (yellowish green), and B (blue) light-emitting units, or CCM (color conversion material) method. In addition, an organic electroluminescent material according to one embodiment of the present disclosure may also be applied to the organic electroluminescent device comprising a QD (quantum dot).

Each layer of the organic electroluminescent device of the present disclosure can be formed by either dry film-forming methods such as vacuum evaporation, sputtering, plasma, ion plating, etc., or wet film-forming methods such as ink jet printing, nozzle printing, slot coating, spin coating, dip coating, flow coating, etc. When using a wet film-forming method, a thin film can be formed by dissolving or diffusing the materials forming each layer into any suitable solvent such as ethanol, chloroform, tetrahydrofuran, dioxane, etc. The solvent is not particularly limited as long as the material forming each layer is soluble or dispersible in the solvents, which do not cause any problems in forming a film.

In addition, it is possible to produce a display device, for example, smart phones, tablets, notebooks, PCs, TVs, or cars; or a lighting system, for example, an outdoor or indoor lighting system by using the organic electroluminescent device according to the present disclosure.

Hereinafter, the preparation method of the compound of the present disclosure, and the properties thereof will be explained in detail with reference to the representative compounds of the present disclosure. However, the present disclosure is not limited to the following examples.

### Example 1: Preparation of Compound C-31

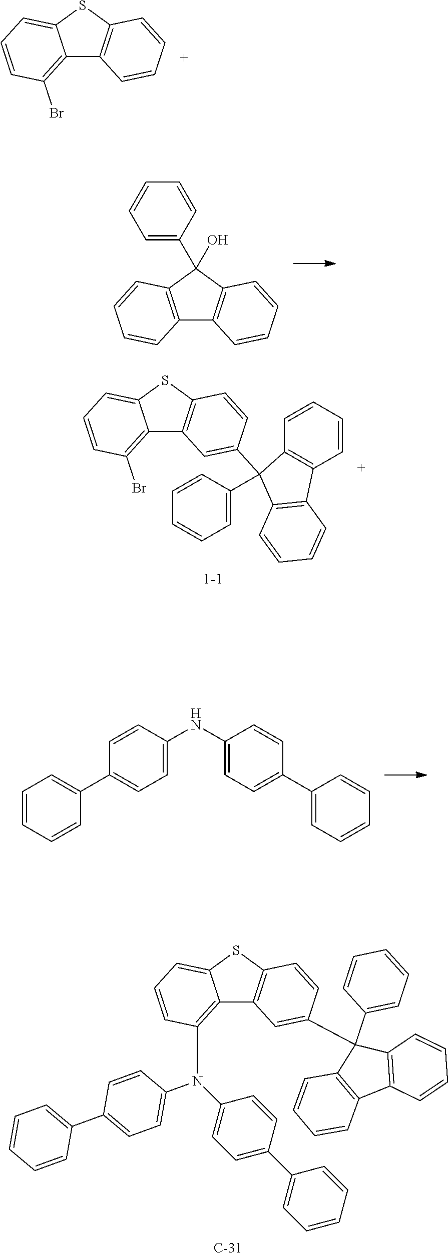

### 1) Synthesis of Compound 1-1

1-bromo-dibenzothiophene (100 g, 423 mmol), 9-phenyl-9-fluorenol (109 g, 423 mmol), and 2000 mL of methylene chloride were added to a reaction vessel, and then phosphorus pentoxide (15 g, 105 mmol) was slowly added dropwise and reacted for 2 hours. After completion of the reaction, the reaction was terminated with water, and then the organic layer was extracted with ethyl acetate. After the residual moisture was removed with magnesium sulfate, the organic layer was dried, and separated by column chromatography to obtain compound 1-1 (20 g, yield: 10%).

### 2) Synthesis of Compound C-31

Compound 1-1 (5 g, 9.93 mmol), bis-biphenylamine (3.3 g, 10.4 mmol), tris(dibenzylideneacetone)dipalladium(0) (0.45 g, 0.49 mmol), tri-tert-butylphosphine (0.5 mL, 0.99 mmol), sodium tert-butoxide (2.4 g, 24.8 mmol), and 50 mL of toluene were added to a reaction vessel, and then the mixture was stirred under reflux for 2 hours. After completion of the reaction, the mixture was washed with distilled water, and then the organic layer was extracted with ethyl acetate. After the residual moisture was removed with magnesium sulfate, the organic layer was dried, and separated by column chromatography to obtain compound C-31 (6.8 g, yield: 92%).

| Compound | MW | M.P. |
|---|---|---|
| C-31 | 743.97 | 281 °C |

### Example 2: Preparation of Compound C-72

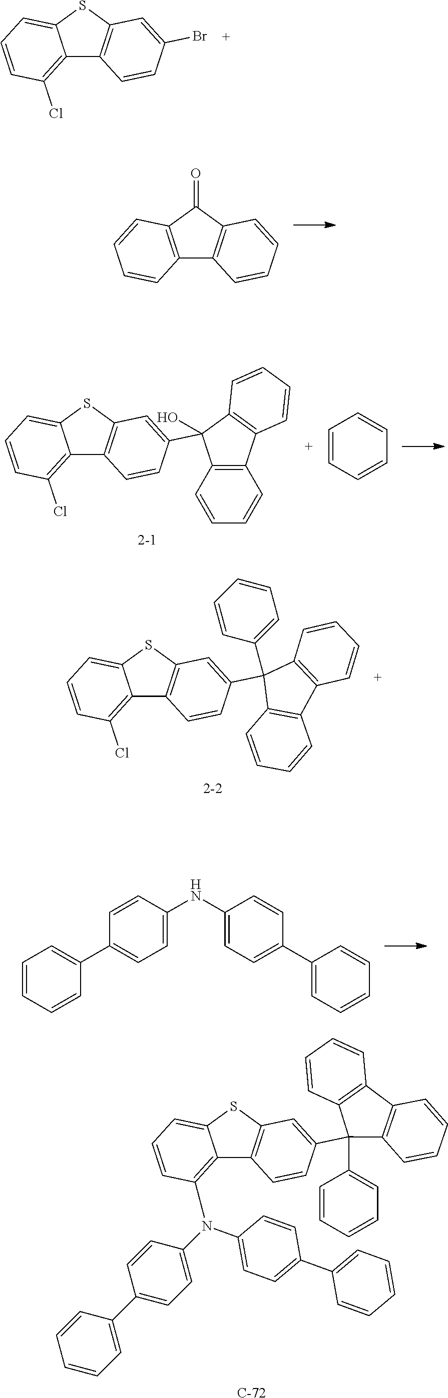

### 1) Synthesis of Compound 2-1

7-bromo-1-chloro-dibenzothiophene (62 g, 208 mmol) and 1000 mL of tetrahydrofuran (THF) were added to a reaction vessel in a nitrogen atmosphere, and then cooled to −78 °C. Then, n-butyllithium (n-BuLi) (100 mL, 250 mmol) was slowly added dropwise. Fluorenone (37 g, 208 mmol) was then added dropwise, and reacted for 12 hours. After completion of the reaction, the reaction was terminated with water, and the organic layer was extracted with ethyl acetate. After the residual moisture was removed with magnesium sulfate, the organic layer was dried, and separated by column chromatography to obtain compound 2-1 (58 g, yield: 70%).

### 2) Synthesis of Compound 2-2

Compound 2-1 (39 g, 98.4 mmol), 8 mL of sulfuric acid, and 330 mL of benzene were added to a reaction vessel, and then the mixture was stirred under reflux for 2 hours. After completion of the reaction, the mixture was washed with distilled water, and then the organic layer was extracted with ethyl acetate. After the residual moisture was removed with magnesium sulfate, the organic layer was dried, and separated by column chromatography to obtain compound 2-2 (18 g, yield: 41%).

3) Synthesis of compound H1-72

Compound 2-2 (5 g, 10.9 mmol), bis-biphenylamine (3.5 g, 10.9 mmol), tris(dibenzylideneacetone)dipalladium(0) (0.5 g, 0.54 mmol), tri-tert-butylphosphine (0.5 mL, 1.09 mmol), sodium tert-butoxide (2.6 g, 27.2 mmol), and 55 mL of toluene were added to a reaction vessel, and then the mixture was stirred under reflux for 2 hours. After completion of the reaction, the mixture was washed with distilled water, and then the organic layer was extracted with ethyl acetate. After the residual moisture was removed with magnesium sulfate, the organic layer was dried, and separated by column chromatography to obtain compound C-72 (6.4 g, yield: 79%).

| Compound | MW | M.P. |
|---|---|---|
| C-72 | 743.97 | 240 °C |

Hereinafter, the luminous efficiency and lifespan properties of an organic electroluminescent device (OLED) comprising the organic electroluminescent compound according to the present disclosure will be explained in detail. However, the following examples merely illustrate the properties of an OLED according to the present disclosure, but the present disclosure is not limited to the following examples.

### Device Example 1: Producing an OLED Comprising the Organic Electroluminescent Compound According to the Present Disclosure

An OLED according to the present disclosure was produced. A transparent electrode indium tin oxide (ITO) thin film (10 Ω/sq) on a glass substrate for an OLED (GEOMATEC CO., LTD., Japan) was subjected to an ultrasonic washing with acetone and isopropyl alcohol, sequentially, and then was stored in isopropanol. The ITO substrate was then mounted on a substrate holder of a vacuum vapor deposition apparatus. Compound PD-15 was introduced into a cell of the vacuum vapor deposition apparatus, and compound HT-1 was introduced into another cell. The two materials were evaporated at different rates, and compound PD-15 was deposited in an amount of 7 wt % based to the total amount of compound PD-15 and compound HT-1 to form a hole injection layer with a thickness of 10 nm. Subsequently, compound HT-1 was deposited as a first hole transport layer with a thickness of 75 nm on the hole injection layer. Next, compound C-31 was deposited as a second hole transport layer with a thickness of 5 nm on the first hole transport layer. After forming the hole injection layer and the hole transport layers, a light-emitting layer was deposited thereon as follows. Compound BH was introduced into a cell of the vacuum vapor deposition apparatus as a host, and compound BD was introduced into another cell as a dopant. The two materials were evaporated at different rates, and the dopant was deposited in a doping amount of 2 wt % based on the total amount of the host and the dopant to form a light-emitting layer having a thickness of 20 nm on the second hole transport layer. Subsequently, compounds HBL was deposited as an electron buffer material having a thickness of 5 nm on the light-emitting layer. Compounds ETL-1:EIL-1 were deposited at a weight ratio of 5:5 as an electron transport layer having a thickness of 30 nm. After depositing compound EIL-1 as an electron injection layer having a thickness of 2 nm on the electron transport layer, an Al cathode having a thickness of 80 nm was deposited on the electron injection layer by another vacuum vapor deposition apparatus. Thus, an OLED was produced.

### Device Example 2: Producing an OLED Comprising the Organic Electroluminescent Compound According to the Present Disclosure

An OLED was produced in the same manner as in Device Example 1, except that compound C-72 was used as the second hole transport layer.

### Comparative Example 1: Producing an OLED not Comprising the Organic Electroluminescent Compound According to the Present Disclosure

An OLED was produced in the same manner as in Device Example 1, except that the first hole transport layer was deposited to a thickness of 80 nm without the second hole transport layer.

The driving voltage, the luminous efficiency, and CIE 1931 color coordinate at a luminance of 1,000 nit, and the time taken for luminance to decrease from 100% to 95% at a luminance of 1,700 nit (lifespan: T95) of the OLEDs produced in the Device Examples and the Comparative Example are provided in Table 1 below.

TABLE 1

| | First | Second | | | CIE | |
|---|---|---|---|---|---|---|
| | Hole | Hole | Driving | Luminous | Color | Lifespan |
| | Transport | Transport | Voltage | Efficiency | Coordinate | (T95) |
| | Layer | Layer | (V) | (cd/A) | (x, y) | [hr] |
| Device | HT-1 | C-31 | 3.8 | 6.0 | (0.133, 0.076) | 126 |
| Example 1 | | | | | | |
| Device | HT-1 | C-72 | 3.8 | 6.0 | (0.133, 0.076) | 107 |
| Example 2 | | | | | | |
| Comparative | HT-1 | — | 3.7 | 5.7 | (0.132, 0.078) | 66 |
| Example 1 | | | | | | |

From Table 1 above, it can be seen that the OLEDs comprising the compound according to present disclosure in the second hole transport layer have significantly improved luminous efficiency and/or lifespan properties compared to the OLED not comprising it.

Generally, it is difficult to improve the lifespan characteristics of a blue light-emitting OLED. This is because the band gap between a blue dopant and a host is relatively high compared to the band gap between a green or red dopant and a host, which results in poor material stability. The present inventors have found that the HOMO level can be controlled, thereby improving the hole injection from the first hole transport layer into the light-emitting layer, and helping to stabilize the device at the interface by including a compound according to the present disclosure in a second hole transport layer between a first hole transport layer and a light-emitting layer. Due to such an effect, it is understood that the compound represented by formula 1 according to the present disclosure can improve not only the luminous efficiency but also the lifespan property of the OLED.

The compounds used in the Devices Examples and the Comparative Example are shown in Table 2 below.

TABLE 2

| | |
|---|---|
| Hole Injection Layer/ Hole Transport Layer | 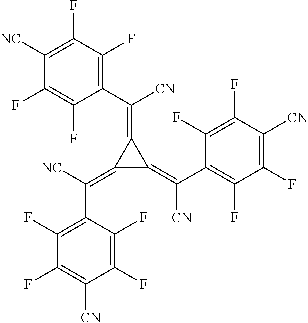 [SMILES: N#CC(=C1C(=C(C#N)c2c(F)c(F)c(C#N)c(F)c2F)C1=C(C#N)c1c(F)c(F)c(C#N)c(F)c1F)c1c(F)c(F)c(C#N)c(F)c1F] |
| | PD-15 |
| | 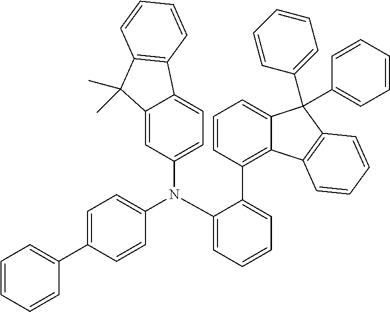 [SMILES: CC1(C)c2ccccc2-c2ccc(N(c3ccc(-c4ccccc4)cc3)c3ccccc3-c3cccc4c3-c3ccccc3C4(c3ccccc3)c3ccccc3)cc21] |
| | HT-1 |
| | 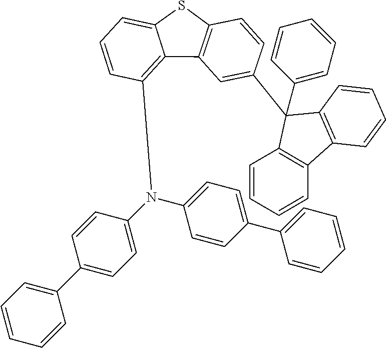 [SMILES: c1ccc(-c2ccc(N(c3ccc(-c4ccccc4)cc3)c3cccc4sc5ccc(C6(c7ccccc7)c7ccccc7-c7ccccc76)cc5c34)cc2)cc1] |
| | C-31 |
| | 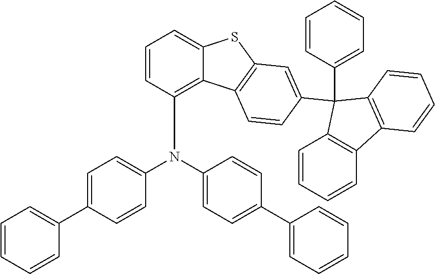 [SMILES: c1ccc(-c2ccc(N(c3ccc(-c4ccccc4)cc3)c3cccc4sc5cc(C6(c7ccccc7)c7ccccc7-c7ccccc76)ccc5c34)cc2)cc1] |
| | C-72 |
| Light- Emitting Layer | 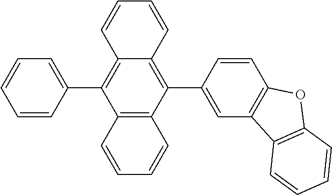 [SMILES: c1ccc(-c2c3ccccc3c(-c3ccc4oc5ccccc5c4c3)c3ccccc23)cc1] |
| | BH |
| | 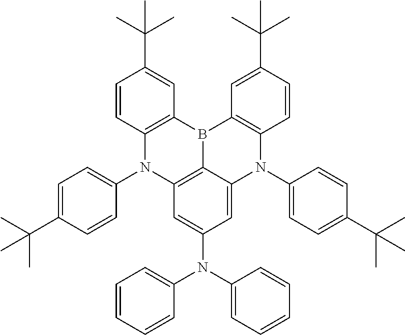 [SMILES: CC(C)(C)c1ccc(N2c3ccc(C(C)(C)C)cc3B3c4cc(C(C)(C)C)ccc4N(c4ccc(C(C)(C)C)cc4)c4cc(N(c5ccccc5)c5ccccc5)cc2c43)cc1] |
| | BD |
| Electron Buffer Layer | 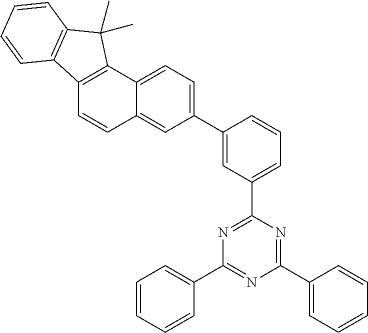 [SMILES: CC1(C)c2ccccc2-c2ccc3cc(-c4cccc(-c5nc(-c6ccccc6)nc(-c6ccccc6)n5)c4)ccc3c21] |
| | HBL |
| Electron Transport Layer/ Electron Injection Layer | 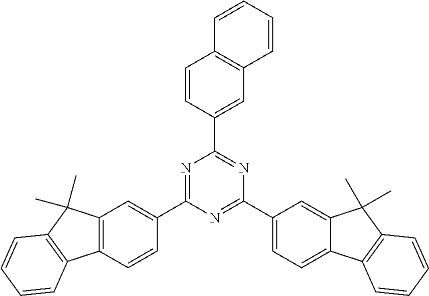 [SMILES: CC1(C)c2ccccc2-c2ccc(-c3nc(-c4ccc5c(c4)C(C)(C)c4ccccc4-5)nc(-c4ccc5ccccc5c4)n3)cc21] |
| | ETL-1 |
| | 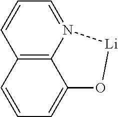 |
| | EIL-1 |

1. An organic electroluminescent compound represented by any one of the following formulas 1-1, 1-3, and 1-4: 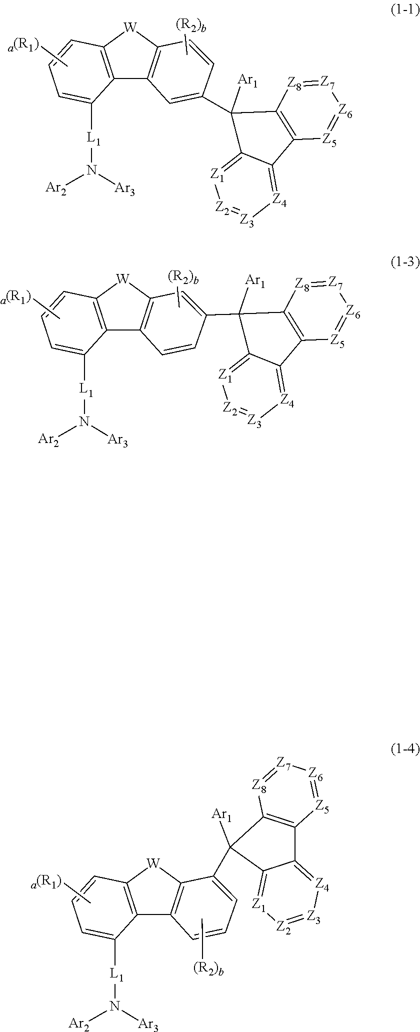
in formulas 1-1, 1-3, and 1-4,
W represents O, S or —N-L₂-R₃;
Z₁ to Z₃ each independently represent CR₄ or N;
L₁ and L₂ each independently represent a single bond, a substituted or unsubstituted (C6-C30)arylene, or a substituted or unsubstituted (3- to 30-membered)heteroarylene;
Ar₁ to Ar₃ each independently represent a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted fused ring group of a (C3-C30) aliphatic ring(s) and a (C6-C30) aromatic ring(s), a substituted or unsubstituted mono- or di-(C1-C30)alkylamino, a substituted or unsubstituted mono- or di-(C2-C30)alkenylamino, a substituted or unsubstituted mono- or di-(C6-C30)arylamino, a substituted or unsubstituted mono- or di-(3- to 30-membered)heteroarylamino, a substituted or unsubstituted (C1-C30)alkyl(C2-C30)alkenylamino, a substituted or unsubstituted (C1-C30)alkyl(C6-C30)arylamino, a substituted or unsubstituted (C1-C30)alkyl(3- to 30-membered)heteroarylamino, a substituted or unsubstituted (C2-C30)alkenyl(C6-C30)arylamino, a substituted or unsubstituted (C2-C30)alkenyl(3- to 30-membered)heteroarylamino, or a substituted or unsubstituted (C6-C30)aryl(3- to 30-membered)heteroarylamino;
R₁ to R₄ each independently represent hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted fused ring group of a (C3-C30) aliphatic ring(s) and a (C6-C30) aromatic ring(s), a substituted or unsubstituted mono- or di-(C1-C30)alkylamino, a substituted or unsubstituted mono- or di-(C2-C30)alkenylamino, a substituted or unsubstituted mono- or di-(C6-C30)arylamino, a substituted or unsubstituted mono- or di-(3- to 30-membered)heteroarylamino, a substituted or unsubstituted (C1-C30)alkyl(C2-C30)alkenylamino, a substituted or unsubstituted (C1-C30)alkyl(C6-C30)arylamino, a substituted or unsubstituted (C1-C30)alkyl(3- to 30-membered)heteroarylamino, a substituted or unsubstituted (C2-C30)alkenyl(C6-C30)arylamino, a substituted or unsubstituted (C2-C30)alkenyl(3- to 30-membered)heteroarylamino, or a substituted or unsubstituted (C6-C30)aryl(3- to 30-membered)heteroarylamino; or two or more of the adjacent R₁ to R₄ may be linked to each other to form a ring(s);
a and b are each independently an integer of 1 to 3; and
if a plurality of each of R₁, R₂ and R₄ are present, each of R₁, each of R₂, and each of R₄ may be the same or different.

2. The organic electroluminescent compound according to claim 1, wherein the substituents of the substituted alkyl, the substituted aryl(ene), the substituted heteroaryl(ene), the substituted cycloalkyl, the substituted alkoxy, the substituted trialkylsilyl, the substituted dialkylarylsilyl, the substituted alkyldiarylsilyl, the substituted triarylsilyl, the substituted fused ring group of an aliphatic ring(s) and an aromatic ring(s), the substituted mono- or di-alkylamino, the substituted mono- or di-alkenylamino, the substituted mono- or di-arylamino, the substituted mono- or di-heteroarylamino, the substituted alkylalkenylamino, the substituted alkylarylamino, the substituted alkylheteroarylamino, the substituted alkenylarylamino, the substituted alkenylheteroarylamino, and the substituted arylheteroarylamino, each independently, are at least one selected from the group consisting of deuterium; a halogen; a cyano; a carboxyl; a nitro; a hydroxyl; a phosphine oxide; a (C1-C30)alkyl; a halo(C1-C30)alkyl; a (C2-C30)alkenyl; a (C2-C30)alkynyl; a (C1-C30)alkoxy; a (C1-C30)alkylthio; a (C3-C30)cycloalkyl; a (C3-C30)cycloalkenyl; a (3- to 7-membered)heterocycloalkyl; a (C6-C30)aryloxy; a (C6-C30)arylthio; a (3- to 30-membered)heteroaryl unsubstituted or substituted with a (C6-C30)aryl(s); a (C6-C30)aryl unsubstituted or substituted with a (3- to 30-membered)heteroaryl(s); a tri(C1-C30)alkylsilyl; a tri(C6-C30)arylsilyl; a di(C1-C30)alkyl(C6-C30)arylsilyl; a (C1-C30)alkyldi(C6-C30)arylsilyl; a fused ring group of a (C3-C30) aliphatic ring(s) and a (C6-C30) aromatic ring(s); an amino; a mono- or di-(C1-C30)alkylamino; a mono- or di-(C2-C30)alkenylamino; a mono- or di-(C6-C30)arylamino unsubstituted or substituted with a (C1-C30)alkyl(s); a mono- or di-(3- to 30-membered)heteroarylamino; a (C1-C30)alkyl(C2-C30)alkenylamino; a (C1-C30)alkyl(C6-C30)arylamino; a (C1-C30)alkyl(3- to 30-membered)heteroarylamino; a (C2-C30)alkenyl(C6-C30)arylamino; a (C2-C30)alkenyl(3- to 30-membered)heteroarylamino; a (C6-C30)aryl(3- to 30-membered)heteroarylamino; a (C1-C30)alkylcarbonyl; a (C1-C30)alkoxycarbonyl; a (C6-C30)arylcarbonyl; a (C6-C30)arylphosphine; a di(C6-C30)arylboronyl; a di(C1-C30)alkylboronyl; a (C1-C30)alkyl(C6-C30)arylboronyl; a (C6-C30)aryl(C1-C30)alkyl; and a (C1-C30)alkyl(C6-C30)aryl.

3. The organic electroluminescent compound according to claim 1, wherein the compound represented by any one of formula 1-1, 1-3, and 1-4 is selected from the following compounds:

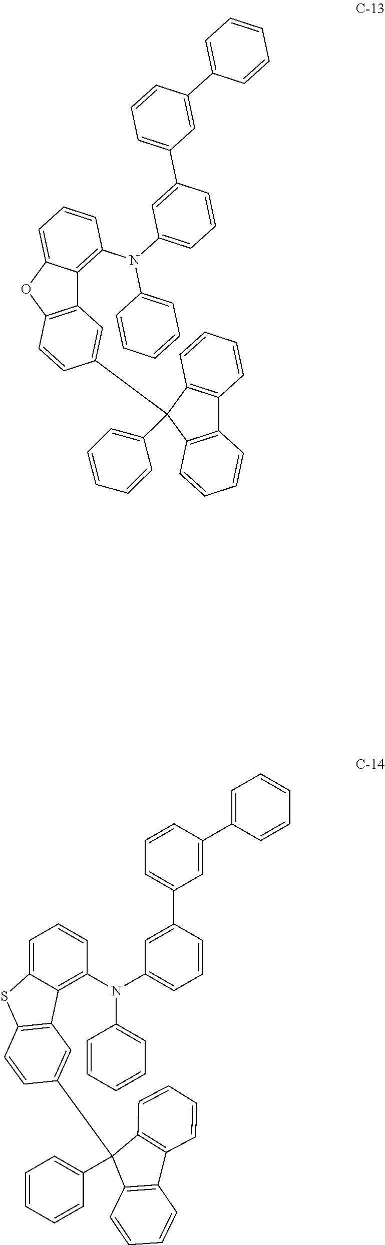 [SMILES: c1ccc(-c2cccc(-c3cccc(N(c4ccccc4)c4cccc5oc6ccc(C7(c8ccccc8)c8ccccc8-c8ccccc87)cc6c45)c3)c2)cc1 ; c1ccc(-c2cccc(-c3cccc(N(c4ccccc4)c4cccc5sc6ccc(C7(c8ccccc8)c8ccccc8-c8ccccc87)cc6c45)c3)c2)cc1]

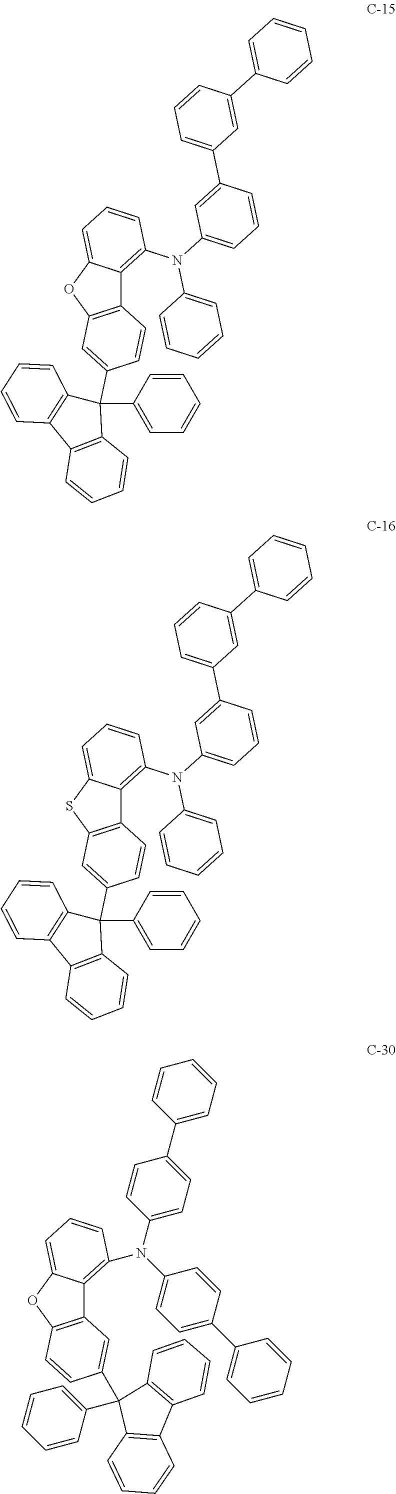 [SMILES: c1ccc(-c2cccc(-c3cccc(N(c4ccccc4)c4cccc5oc6cc(C7(c8ccccc8)c8ccccc8-c8ccccc87)ccc6c45)c3)c2)cc1 ; c1ccc(-c2cccc(-c3cccc(N(c4ccccc4)c4cccc5sc6cc(C7(c8ccccc8)c8ccccc8-c8ccccc87)ccc6c45)c3)c2)cc1 ; c1ccc(-c2ccc(N(c3ccc(-c4ccccc4)cc3)c3cccc4oc5ccc(C6(c7ccccc7)c7ccccc7-c7ccccc76)cc5c34)cc2)cc1]

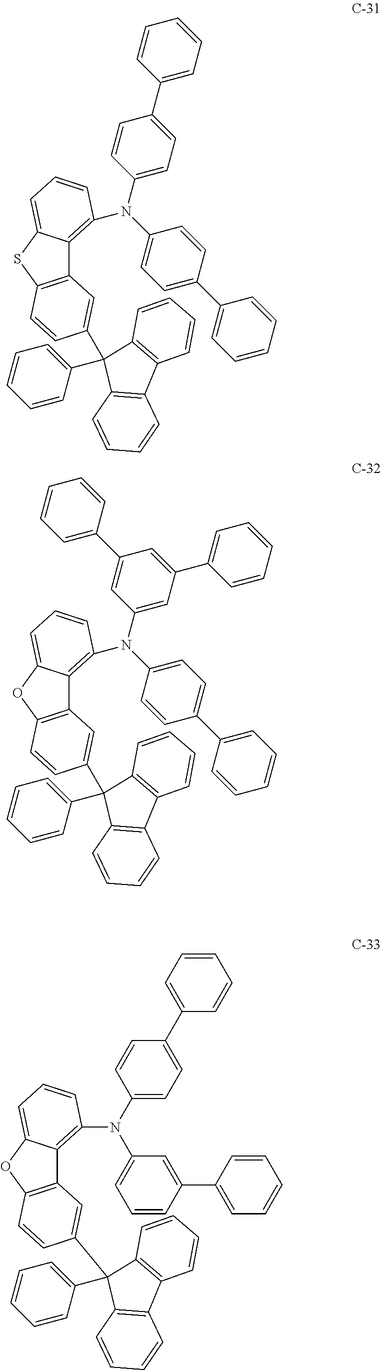

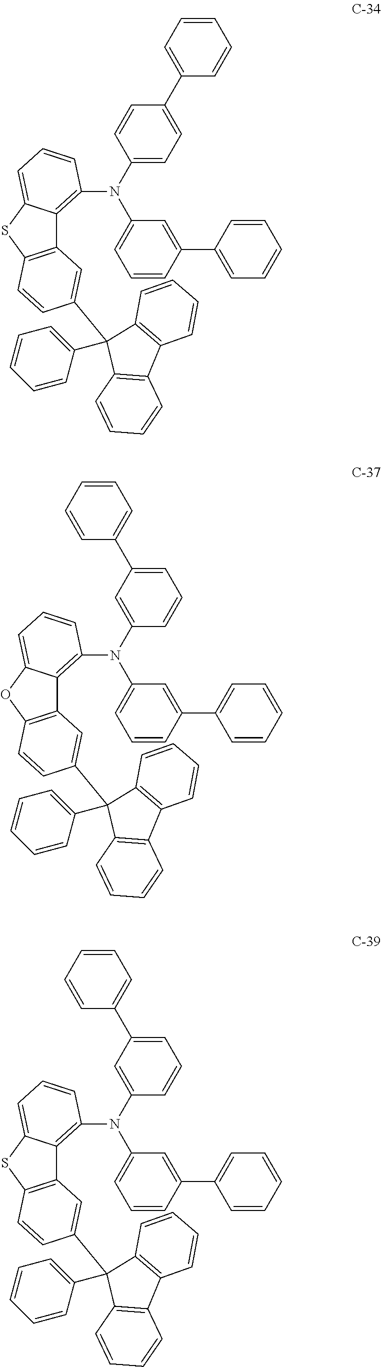 [SMILES: c1ccc(-c2ccc(N(c3cccc(-c4ccccc4)c3)c3cccc4sc5ccc(C6(c7ccccc7)c7ccccc7-c7ccccc76)cc5c34)cc2)cc1 ; c1ccc(-c2cccc(N(c3cccc(-c4ccccc4)c3)c3cccc4oc5ccc(C6(c7ccccc7)c7ccccc7-c7ccccc76)cc5c34)c2)cc1 ; c1ccc(-c2cccc(N(c3cccc(-c4ccccc4)c3)c3cccc4sc5ccc(C6(c7ccccc7)c7ccccc7-c7ccccc76)cc5c34)c2)cc1]

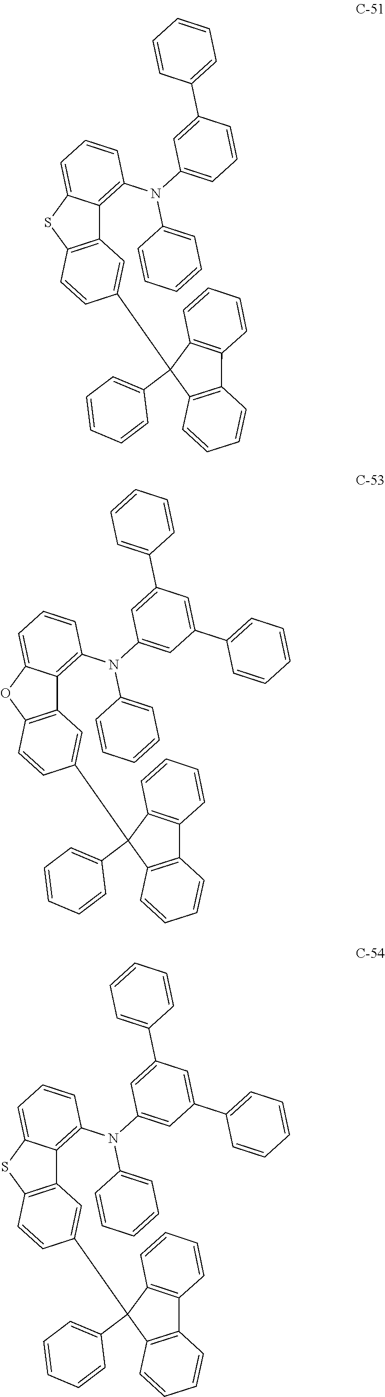 [SMILES: c1ccc(-c2cccc(N(c3ccccc3)c3cccc4sc5ccc(C6(c7ccccc7)c7ccccc7-c7ccccc76)cc5c34)c2)cc1 ; c1ccc(-c2cc(-c3ccccc3)cc(N(c3ccccc3)c3cccc4oc5ccc(C6(c7ccccc7)c7ccccc7-c7ccccc76)cc5c34)c2)cc1 ; c1ccc(-c2cc(-c3ccccc3)cc(N(c3ccccc3)c3cccc4sc5ccc(C6(c7ccccc7)c7ccccc7-c7ccccc76)cc5c34)c2)cc1]

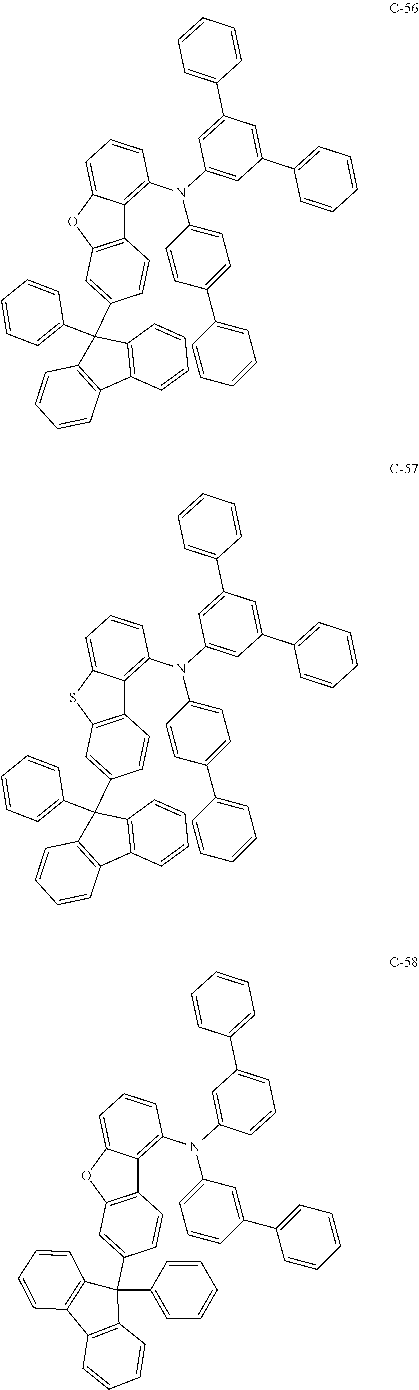 [SMILES: c1ccc(-c2ccc(N(c3cc(-c4ccccc4)cc(-c4ccccc4)c3)c3cccc4oc5cc(C6(c7ccccc7)c7ccccc7-c7ccccc76)ccc5c34)cc2)cc1 ; c1ccc(-c2ccc(N(c3cc(-c4ccccc4)cc(-c4ccccc4)c3)c3cccc4sc5cc(C6(c7ccccc7)c7ccccc7-c7ccccc76)ccc5c34)cc2)cc1 ; c1ccc(-c2cccc(N(c3cccc(-c4ccccc4)c3)c3cccc4oc5cc(C6(c7ccccc7)c7ccccc7-c7ccccc76)ccc5c34)c2)cc1]

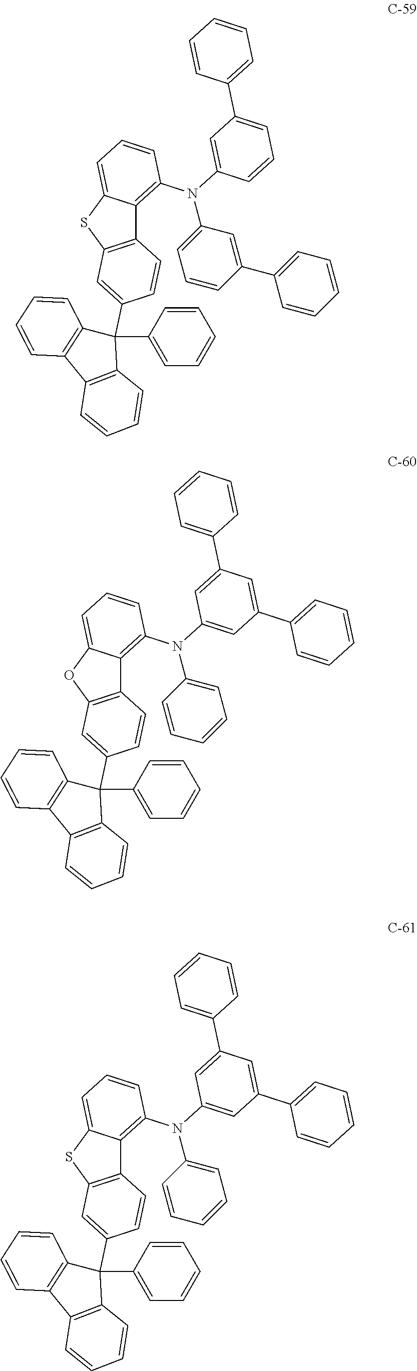 [SMILES: c1ccc(-c2cccc(N(c3cccc(-c4ccccc4)c3)c3cccc4sc5cc(C6(c7ccccc7)c7ccccc7-c7ccccc76)ccc5c34)c2)cc1 ; c1ccc(-c2cc(-c3ccccc3)cc(N(c3ccccc3)c3cccc4oc5cc(C6(c7ccccc7)c7ccccc7-c7ccccc76)ccc5c34)c2)cc1 ; c1ccc(-c2cc(-c3ccccc3)cc(N(c3ccccc3)c3cccc4sc5cc(C6(c7ccccc7)c7ccccc7-c7ccccc76)ccc5c34)c2)cc1]

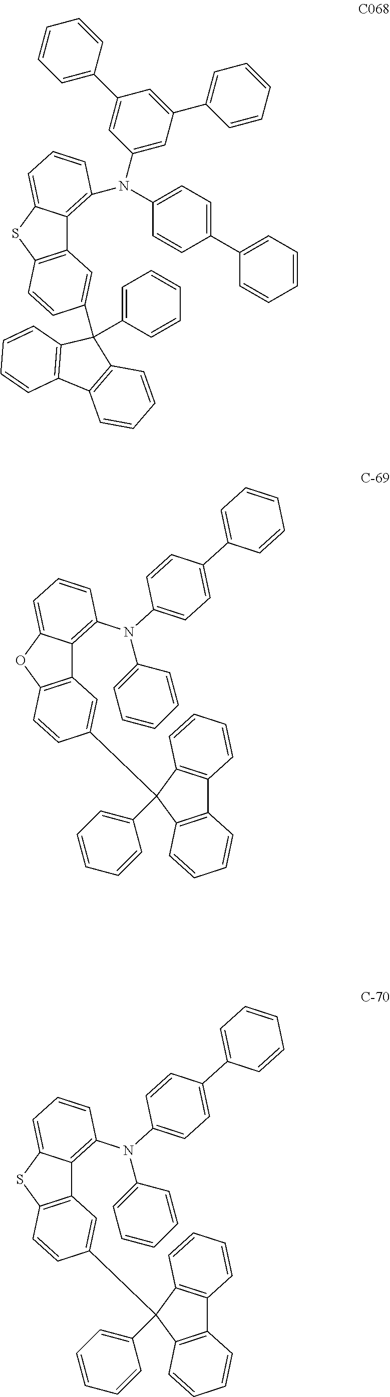 [SMILES: c1ccc(-c2ccc(N(c3cc(-c4ccccc4)cc(-c4ccccc4)c3)c3cccc4sc5ccc(C6(c7ccccc7)c7ccccc7-c7ccccc76)cc5c34)cc2)cc1 ; c1ccc(-c2ccc(N(c3ccccc3)c3cccc4oc5ccc(C6(c7ccccc7)c7ccccc7-c7ccccc76)cc5c34)cc2)cc1 ; c1ccc(-c2ccc(N(c3ccccc3)c3cccc4sc5ccc(C6(c7ccccc7)c7ccccc7-c7ccccc76)cc5c34)cc2)cc1]

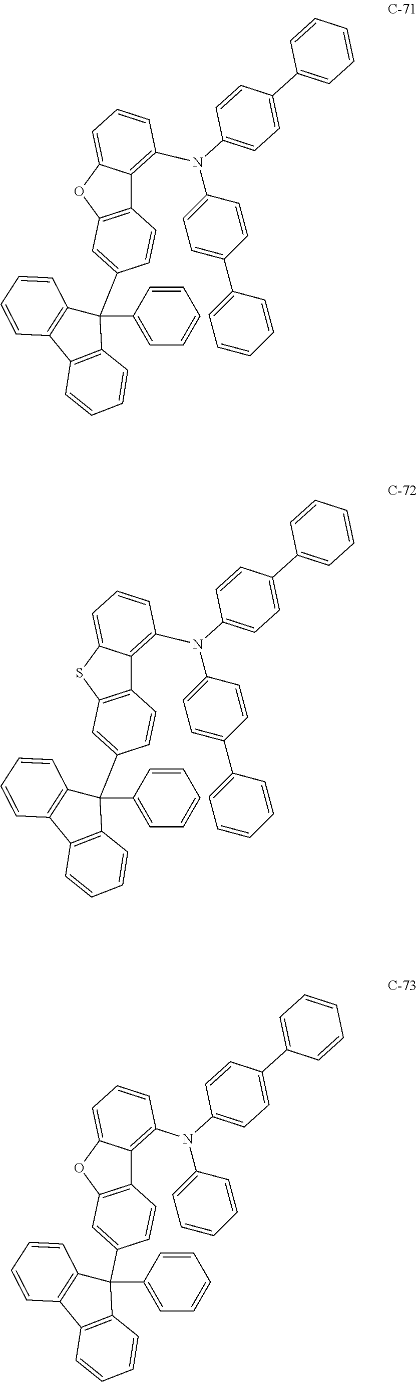 [SMILES: c1ccc(-c2ccc(N(c3ccc(-c4ccccc4)cc3)c3cccc4oc5cc(C6(c7ccccc7)c7ccccc7-c7ccccc76)ccc5c34)cc2)cc1 ; c1ccc(-c2ccc(N(c3ccc(-c4ccccc4)cc3)c3cccc4sc5cc(C6(c7ccccc7)c7ccccc7-c7ccccc76)ccc5c34)cc2)cc1 ; c1ccc(-c2ccc(N(c3ccccc3)c3cccc4oc5cc(C6(c7ccccc7)c7ccccc7-c7ccccc76)ccc5c34)cc2)cc1]

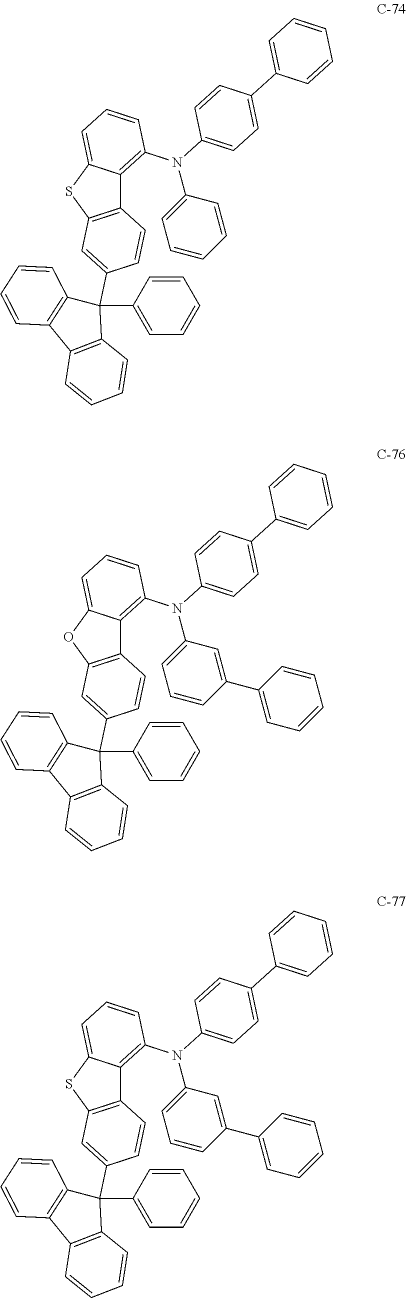 [SMILES: c1ccc(-c2ccc(N(c3ccccc3)c3cccc4sc5cc(C6(c7ccccc7)c7ccccc7-c7ccccc76)ccc5c34)cc2)cc1 ; c1ccc(-c2ccc(N(c3cccc(-c4ccccc4)c3)c3cccc4oc5cc(C6(c7ccccc7)c7ccccc7-c7ccccc76)ccc5c34)cc2)cc1 ; c1ccc(-c2ccc(N(c3cccc(-c4ccccc4)c3)c3cccc4sc5cc(C6(c7ccccc7)c7ccccc7-c7ccccc76)ccc5c34)cc2)cc1]

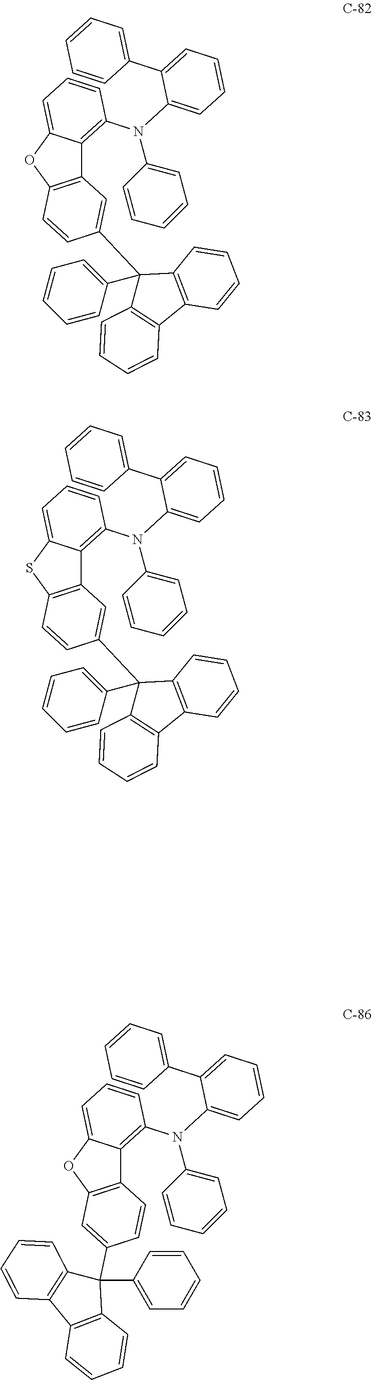 [SMILES: c1ccc(-c2ccccc2N(c2ccccc2)c2cccc3oc4ccc(C5(c6ccccc6)c6ccccc6-c6ccccc65)cc4c23)cc1 ; c1ccc(-c2ccccc2N(c2ccccc2)c2cccc3sc4ccc(C5(c6ccccc6)c6ccccc6-c6ccccc65)cc4c23)cc1 ; c1ccc(-c2ccccc2N(c2ccccc2)c2cccc3oc4cc(C5(c6ccccc6)c6ccccc6-c6ccccc65)ccc4c23)cc1]

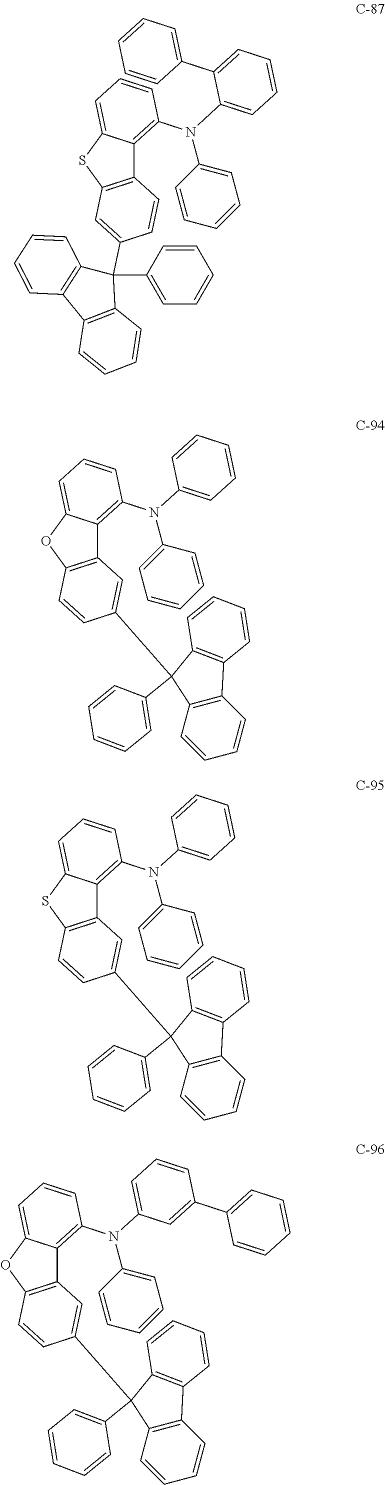

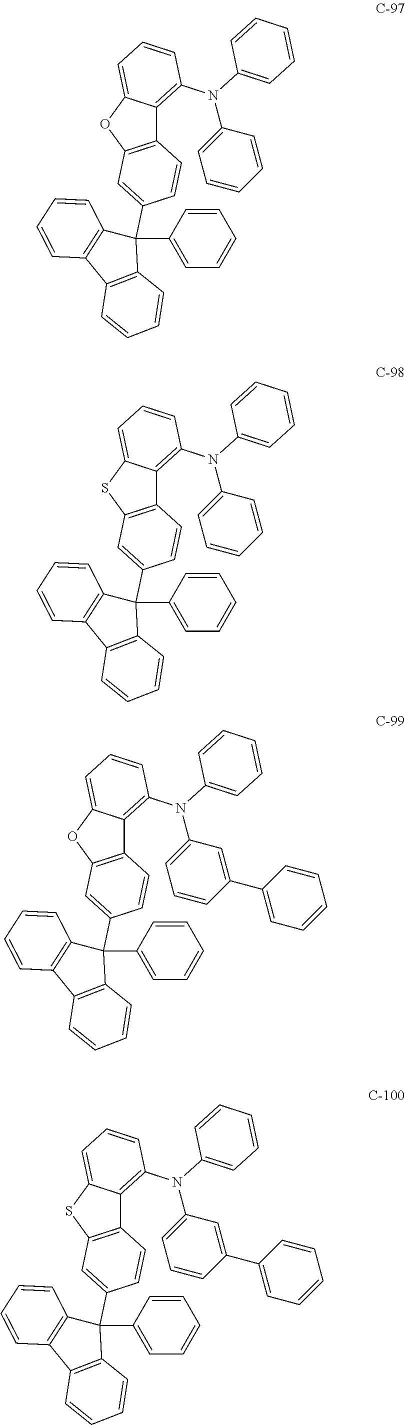 [SMILES: c1ccc(N(c2ccccc2)c2cccc3oc4cc(C5(c6ccccc6)c6ccccc6-c6ccccc65)ccc4c23)cc1 ; c1ccc(N(c2ccccc2)c2cccc3sc4cc(C5(c6ccccc6)c6ccccc6-c6ccccc65)ccc4c23)cc1 ; c1ccc(-c2cccc(N(c3ccccc3)c3cccc4oc5cc(C6(c7ccccc7)c7ccccc7-c7ccccc76)ccc5c34)c2)cc1 ; c1ccc(-c2cccc(N(c3ccccc3)c3cccc4sc5cc(C6(c7ccccc7)c7ccccc7-c7ccccc76)ccc5c34)c2)cc1]

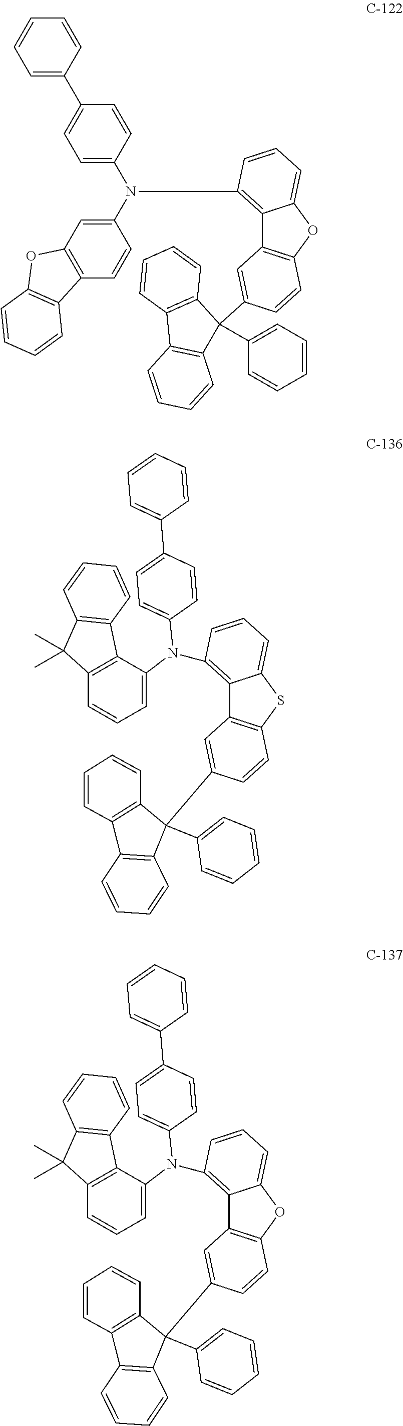 [SMILES: c1ccc(-c2ccc(N(c3ccc4c(c3)oc3ccccc34)c3cccc4oc5ccc(C6(c7ccccc7)c7ccccc7-c7ccccc76)cc5c34)cc2)cc1 ; CC1(C)c2ccccc2-c2c(N(c3ccc(-c4ccccc4)cc3)c3cccc4sc5ccc(C6(c7ccccc7)c7ccccc7-c7ccccc76)cc5c34)cccc21 ; CC1(C)c2ccccc2-c2c(N(c3ccc(-c4ccccc4)cc3)c3cccc4oc5ccc(C6(c7ccccc7)c7ccccc7-c7ccccc76)cc5c34)cccc21]

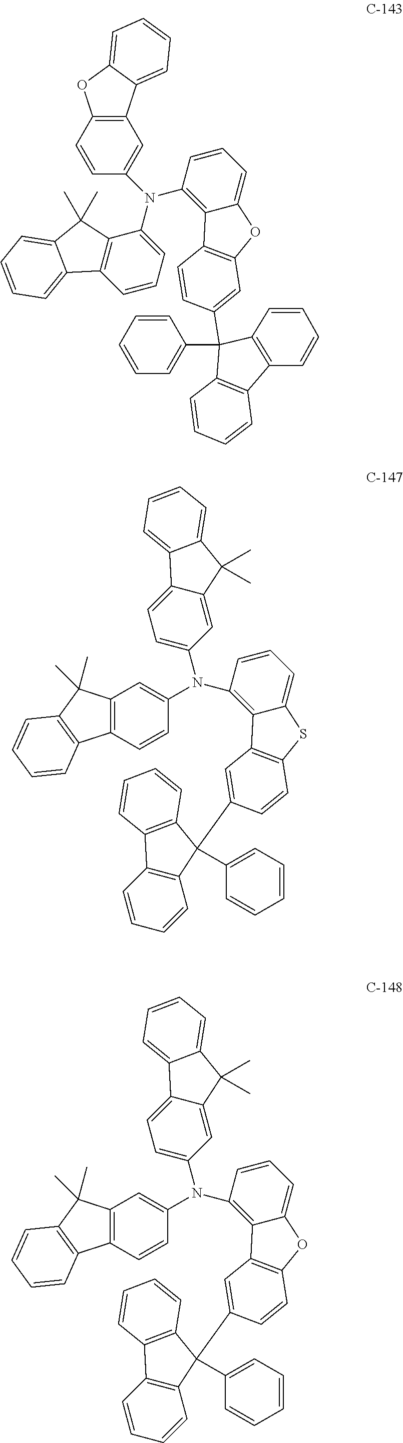 [SMILES: CC1(C)c2ccccc2-c2cccc(N(c3ccc4oc5ccccc5c4c3)c3cccc4oc5cc(C6(c7ccccc7)c7ccccc7-c7ccccc76)ccc5c34)c21 ; CC1(C)c2ccccc2-c2ccc(N(c3ccc4c(c3)C(C)(C)c3ccccc3-4)c3cccc4sc5ccc(C6(c7ccccc7)c7ccccc7-c7ccccc76)cc5c34)cc21 ; CC1(C)c2ccccc2-c2ccc(N(c3ccc4c(c3)C(C)(C)c3ccccc3-4)c3cccc4oc5ccc(C6(c7ccccc7)c7ccccc7-c7ccccc76)cc5c34)cc21]

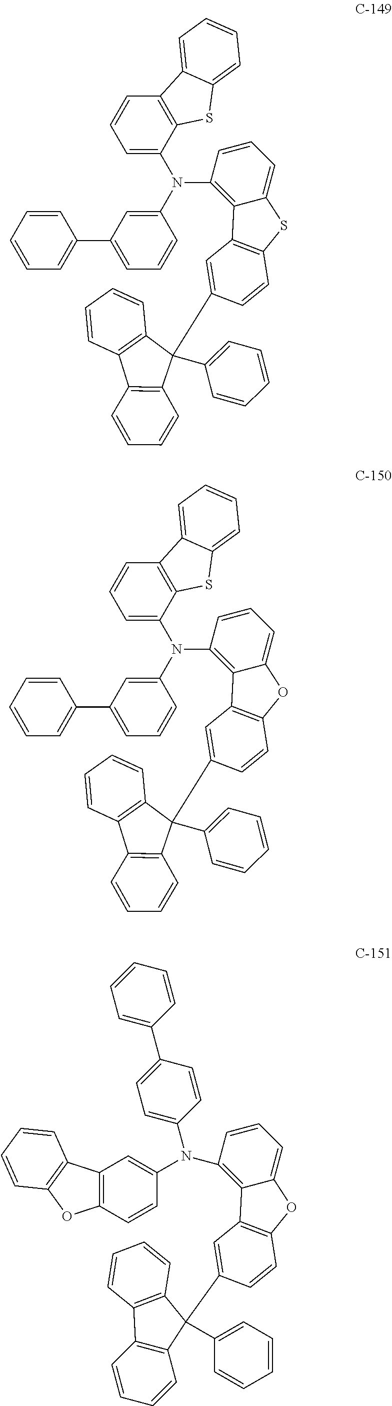 [SMILES: c1ccc(-c2cccc(N(c3cccc4c3sc3ccccc34)c3cccc4sc5ccc(C6(c7ccccc7)c7ccccc7-c7ccccc76)cc5c34)c2)cc1 ; c1ccc(-c2cccc(N(c3cccc4c3sc3ccccc34)c3cccc4oc5ccc(C6(c7ccccc7)c7ccccc7-c7ccccc76)cc5c34)c2)cc1 ; c1ccc(-c2ccc(N(c3ccc4oc5ccccc5c4c3)c3cccc4oc5ccc(C6(c7ccccc7)c7ccccc7-c7ccccc76)cc5c34)cc2)cc1]

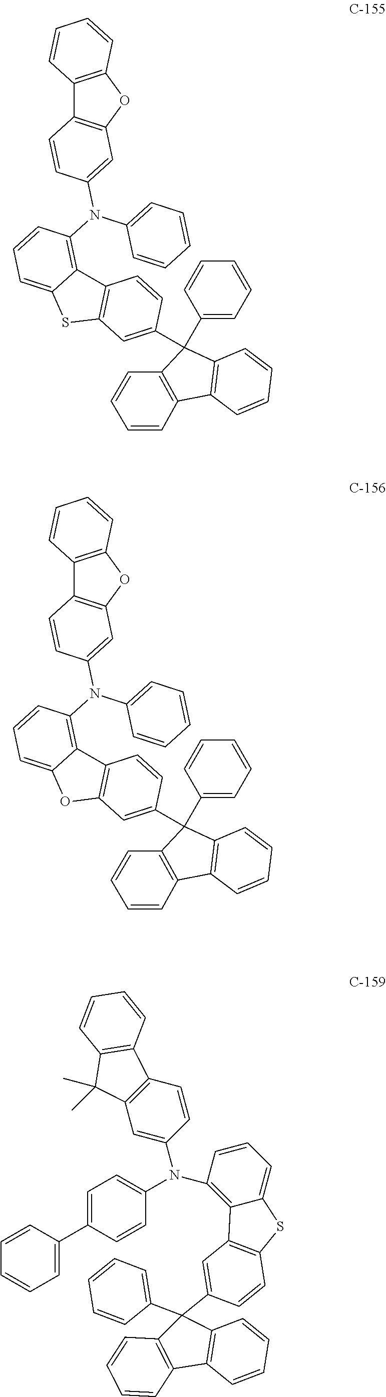 [SMILES: c1ccc(N(c2ccc3c(c2)oc2ccccc23)c2cccc3sc4cc(C5(c6ccccc6)c6ccccc6-c6ccccc65)ccc4c23)cc1 ; c1ccc(N(c2ccc3c(c2)oc2ccccc23)c2cccc3oc4cc(C5(c6ccccc6)c6ccccc6-c6ccccc65)ccc4c23)cc1 ; CC1(C)c2ccccc2-c2ccc(N(c3ccc(-c4ccccc4)cc3)c3cccc4sc5ccc(C6(c7ccccc7)c7ccccc7-c7ccccc76)cc5c34)cc21]

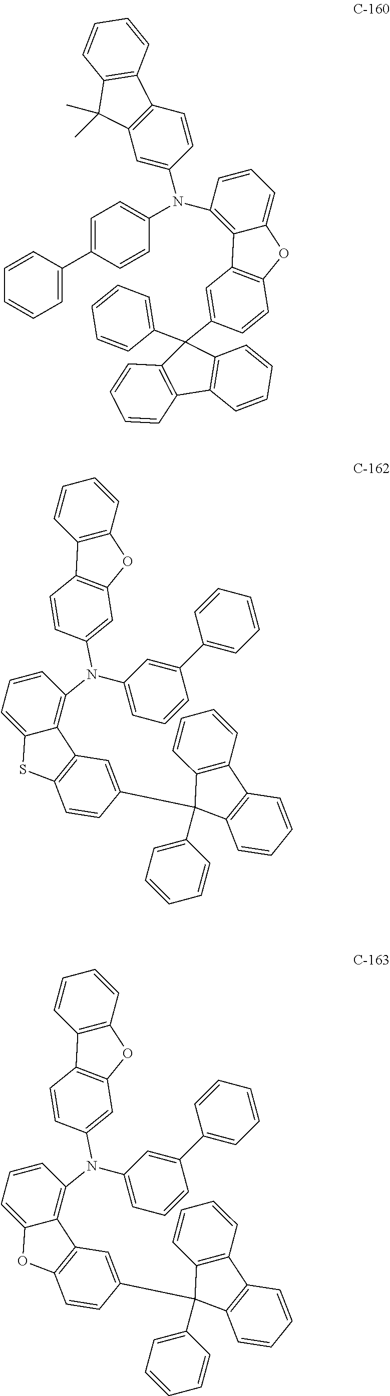 [SMILES: CC1(C)c2ccccc2-c2ccc(N(c3ccc(-c4ccccc4)cc3)c3cccc4oc5ccc(C6(c7ccccc7)c7ccccc7-c7ccccc76)cc5c34)cc21 ; c1ccc(-c2cccc(N(c3ccc4c(c3)oc3ccccc34)c3cccc4sc5ccc(C6(c7ccccc7)c7ccccc7-c7ccccc76)cc5c34)c2)cc1 ; c1ccc(-c2cccc(N(c3ccc4c(c3)oc3ccccc34)c3cccc4oc5ccc(C6(c7ccccc7)c7ccccc7-c7ccccc76)cc5c34)c2)cc1]

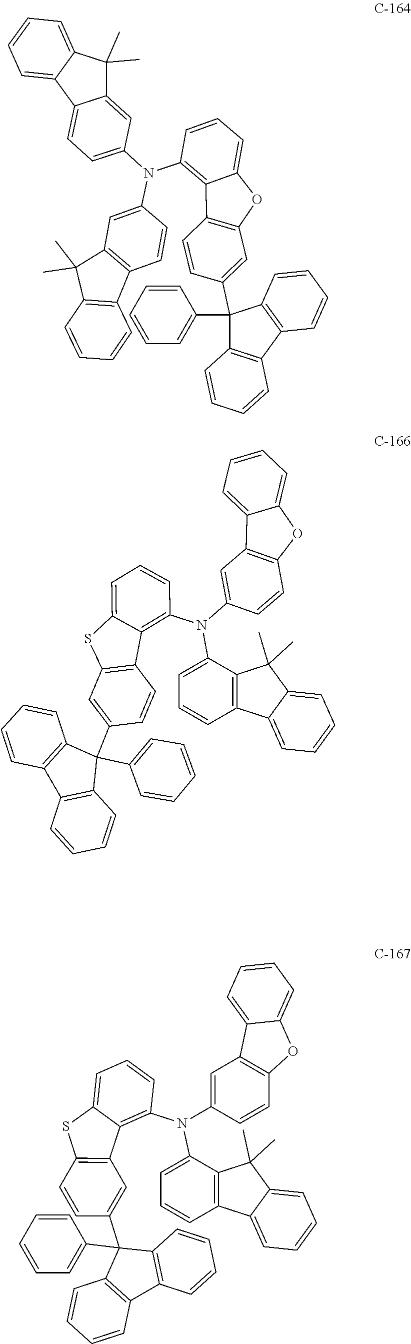

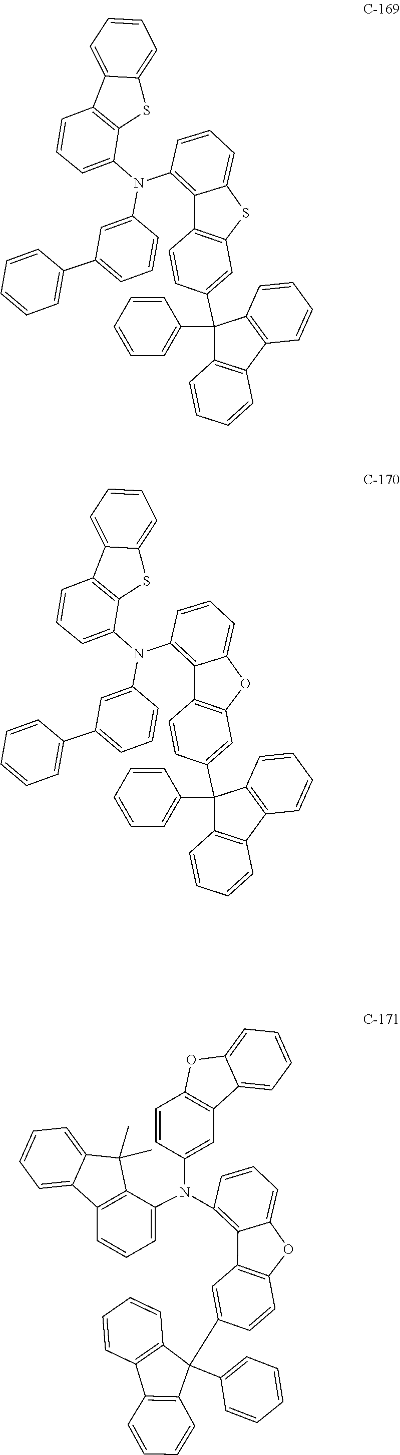 [SMILES: c1ccc(-c2cccc(N(c3cccc4c3sc3ccccc34)c3cccc4sc5cc(C6(c7ccccc7)c7ccccc7-c7ccccc76)ccc5c34)c2)cc1 ; c1ccc(-c2cccc(N(c3cccc4c3sc3ccccc34)c3cccc4oc5cc(C6(c7ccccc7)c7ccccc7-c7ccccc76)ccc5c34)c2)cc1 ; CC1(C)c2ccccc2-c2cccc(N(c3ccc4oc5ccccc5c4c3)c3cccc4oc5ccc(C6(c7ccccc7)c7ccccc7-c7ccccc76)cc5c34)c21]

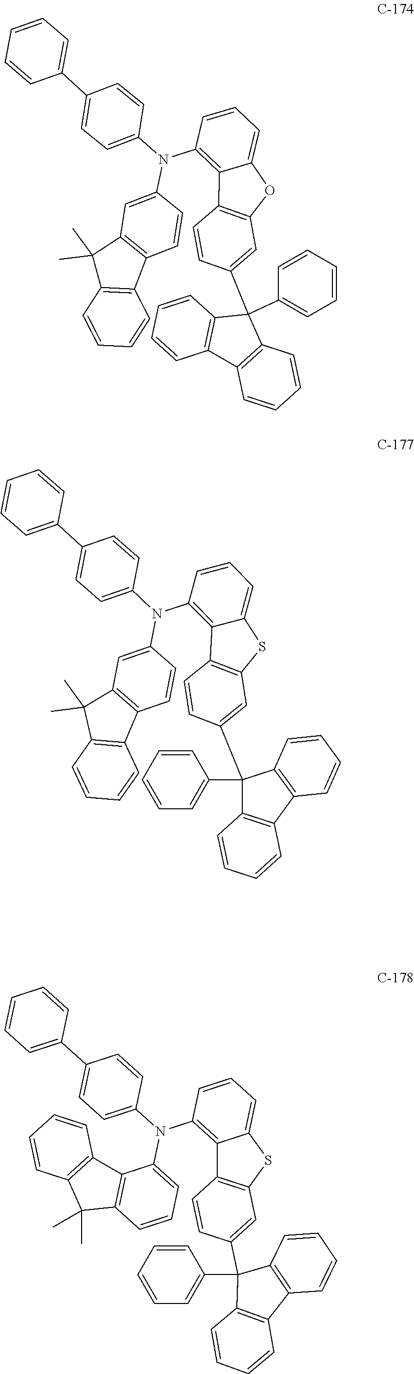 [SMILES: CC1(C)c2ccccc2-c2ccc(N(c3ccc(-c4ccccc4)cc3)c3cccc4oc5cc(C6(c7ccccc7)c7ccccc7-c7ccccc76)ccc5c34)cc21 ; CC1(C)c2ccccc2-c2ccc(N(c3ccc(-c4ccccc4)cc3)c3cccc4sc5cc(C6(c7ccccc7)c7ccccc7-c7ccccc76)ccc5c34)cc21 ; CC1(C)c2ccccc2-c2c(N(c3ccc(-c4ccccc4)cc3)c3cccc4sc5cc(C6(c7ccccc7)c7ccccc7-c7ccccc76)ccc5c34)cccc21]

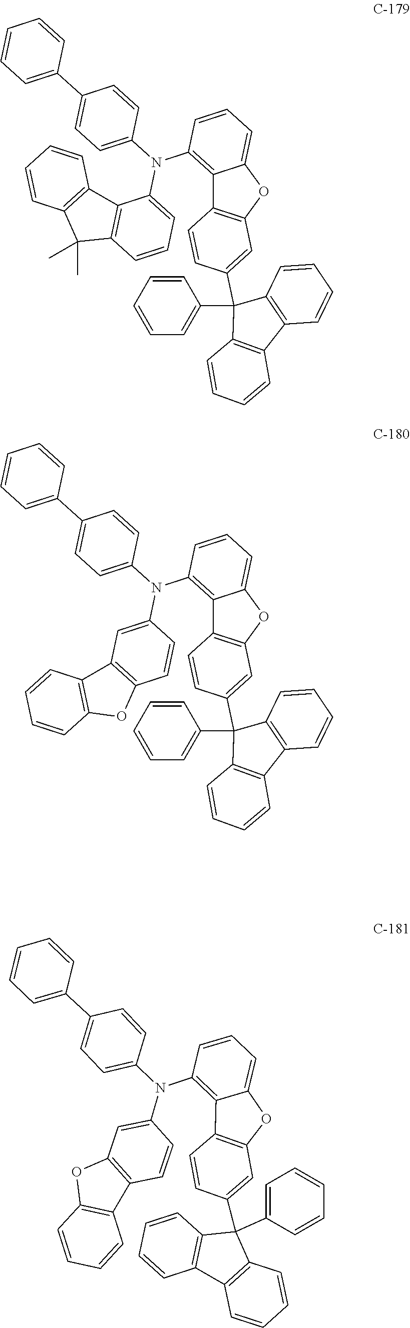 [SMILES: CC1(C)c2ccccc2-c2c(N(c3ccc(-c4ccccc4)cc3)c3cccc4oc5cc(C6(c7ccccc7)c7ccccc7-c7ccccc76)ccc5c34)cccc21 ; c1ccc(-c2ccc(N(c3ccc4oc5ccccc5c4c3)c3cccc4oc5cc(C6(c7ccccc7)c7ccccc7-c7ccccc76)ccc5c34)cc2)cc1 ; c1ccc(-c2ccc(N(c3ccc4c(c3)oc3ccccc34)c3cccc4oc5cc(C6(c7ccccc7)c7ccccc7-c7ccccc76)ccc5c34)cc2)cc1]

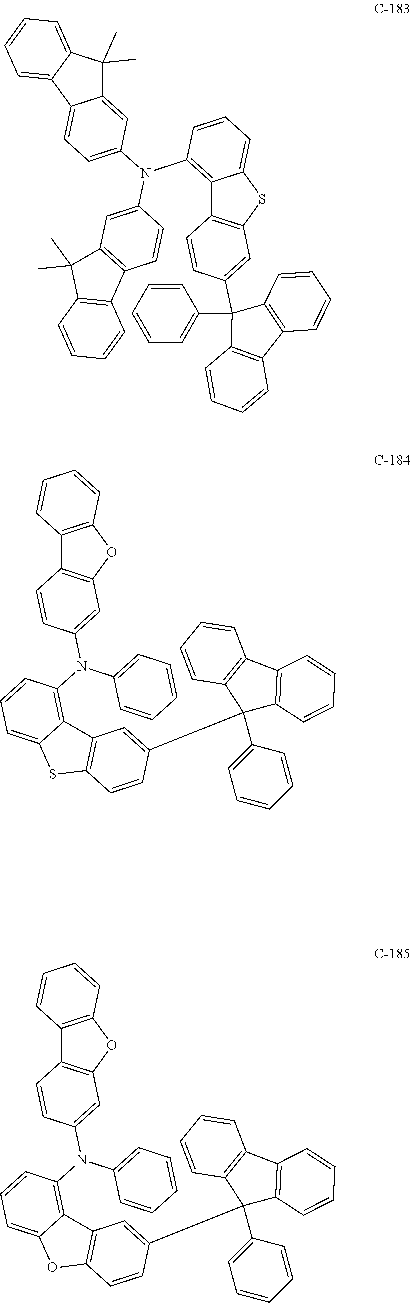 [SMILES: CC1(C)c2ccccc2-c2ccc(N(c3ccc4c(c3)C(C)(C)c3ccccc3-4)c3cccc4sc5cc(C6(c7ccccc7)c7ccccc7-c7ccccc76)ccc5c34)cc21 ; c1ccc(N(c2ccc3c(c2)oc2ccccc23)c2cccc3sc4ccc(C5(c6ccccc6)c6ccccc6-c6ccccc65)cc4c23)cc1 ; c1ccc(N(c2ccc3c(c2)oc2ccccc23)c2cccc3oc4ccc(C5(c6ccccc6)c6ccccc6-c6ccccc65)cc4c23)cc1]

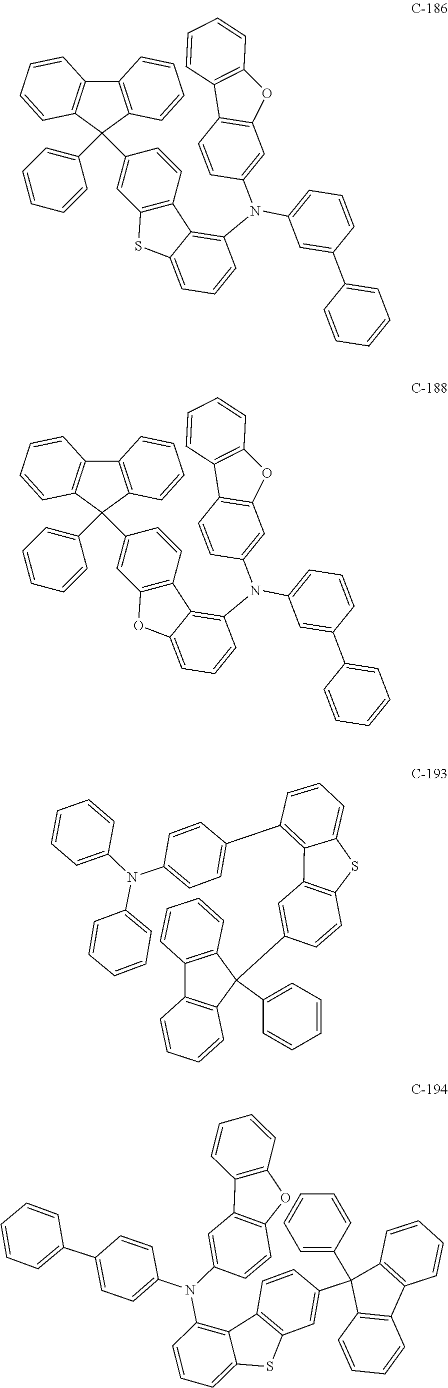 [SMILES: c1ccc(-c2cccc(N(c3ccc4c(c3)oc3ccccc34)c3cccc4sc5cc(C6(c7ccccc7)c7ccccc7-c7ccccc76)ccc5c34)c2)cc1 ; c1ccc(-c2cccc(N(c3ccc4c(c3)oc3ccccc34)c3cccc4oc5cc(C6(c7ccccc7)c7ccccc7-c7ccccc76)ccc5c34)c2)cc1 ; c1ccc(N(c2ccccc2)c2ccc(-c3cccc4sc5ccc(C6(c7ccccc7)c7ccccc7-c7ccccc76)cc5c34)cc2)cc1 ; c1ccc(-c2ccc(N(c3ccc4oc5ccccc5c4c3)c3cccc4sc5cc(C6(c7ccccc7)c7ccccc7-c7ccccc76)ccc5c34)cc2)cc1]

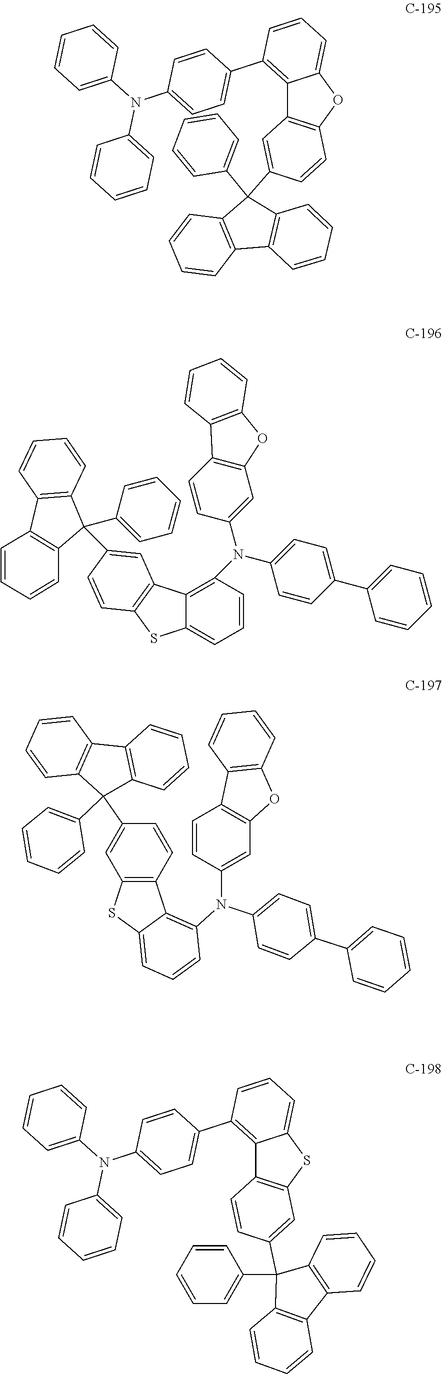 [SMILES: c1ccc(N(c2ccccc2)c2ccc(-c3cccc4oc5ccc(C6(c7ccccc7)c7ccccc7-c7ccccc76)cc5c34)cc2)cc1 ; c1ccc(-c2ccc(N(c3ccc4c(c3)oc3ccccc34)c3cccc4sc5ccc(C6(c7ccccc7)c7ccccc7-c7ccccc76)cc5c34)cc2)cc1 ; c1ccc(-c2ccc(N(c3ccc4c(c3)oc3ccccc34)c3cccc4sc5cc(C6(c7ccccc7)c7ccccc7-c7ccccc76)ccc5c34)cc2)cc1 ; c1ccc(N(c2ccccc2)c2ccc(-c3cccc4sc5cc(C6(c7ccccc7)c7ccccc7-c7ccccc76)ccc5c34)cc2)cc1]

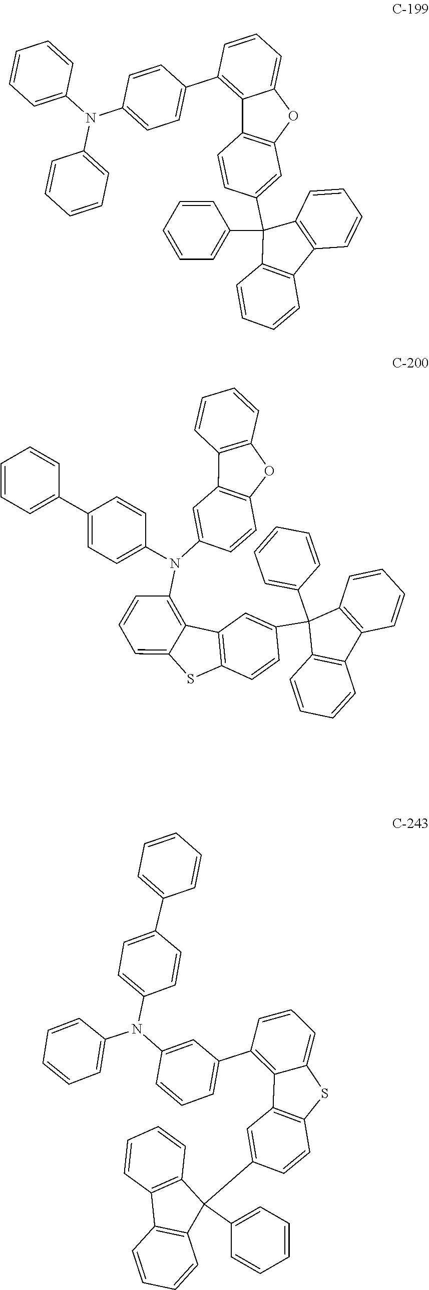 [SMILES: c1ccc(N(c2ccccc2)c2ccc(-c3cccc4oc5cc(C6(c7ccccc7)c7ccccc7-c7ccccc76)ccc5c34)cc2)cc1 ; c1ccc(-c2ccc(N(c3ccc4oc5ccccc5c4c3)c3cccc4sc5ccc(C6(c7ccccc7)c7ccccc7-c7ccccc76)cc5c34)cc2)cc1 ; c1ccc(-c2ccc(N(c3ccccc3)c3cccc(-c4cccc5sc6ccc(C7(c8ccccc8)c8ccccc8-c8ccccc87)cc6c45)c3)cc2)cc1]

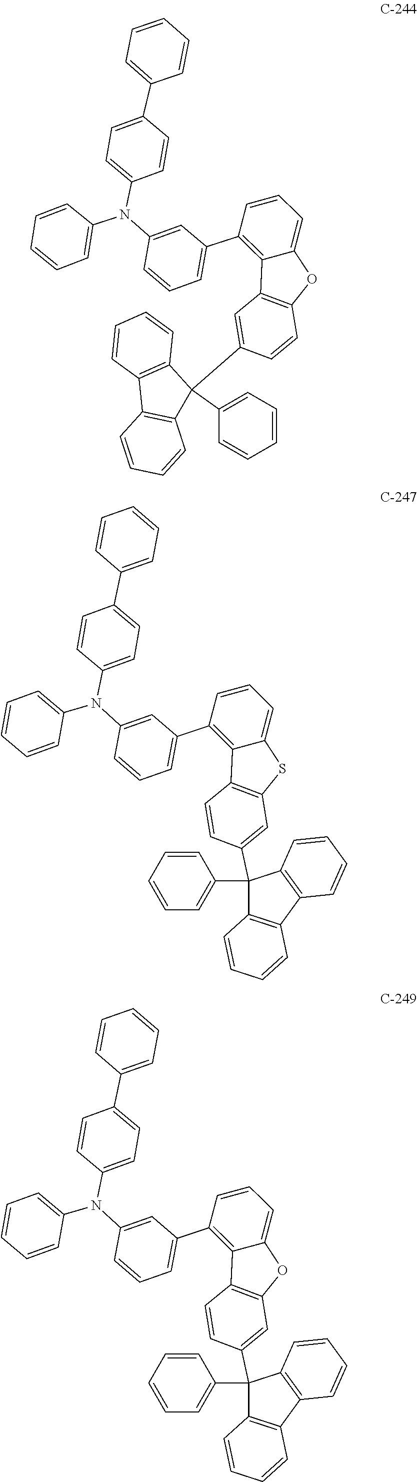

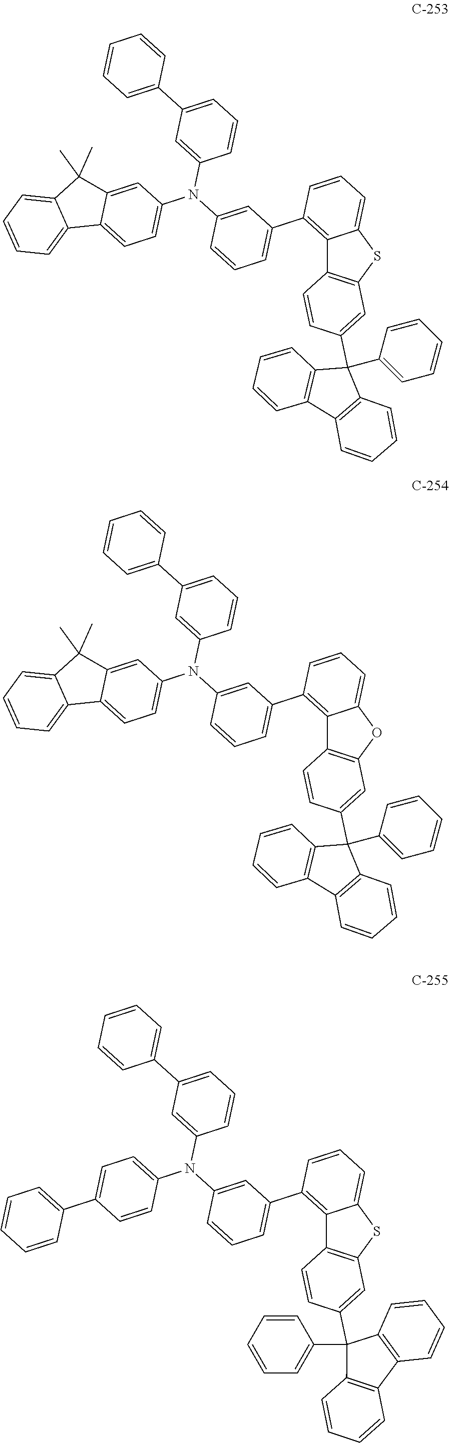 [SMILES: CC1(C)c2ccccc2-c2ccc(N(c3cccc(-c4ccccc4)c3)c3cccc(-c4cccc5sc6cc(C7(c8ccccc8)c8ccccc8-c8ccccc87)ccc6c45)c3)cc21 ; CC1(C)c2ccccc2-c2ccc(N(c3cccc(-c4ccccc4)c3)c3cccc(-c4cccc5oc6cc(C7(c8ccccc8)c8ccccc8-c8ccccc87)ccc6c45)c3)cc21 ; c1ccc(-c2ccc(N(c3cccc(-c4ccccc4)c3)c3cccc(-c4cccc5sc6cc(C7(c8ccccc8)c8ccccc8-c8ccccc87)ccc6c45)c3)cc2)cc1]

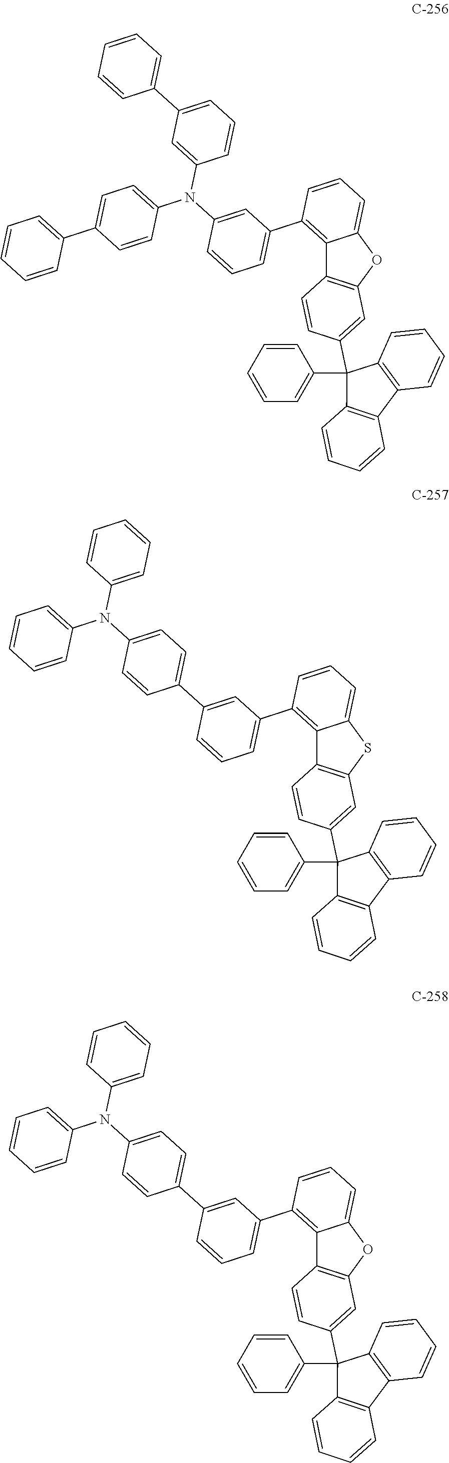 [SMILES: c1ccc(-c2ccc(N(c3cccc(-c4ccccc4)c3)c3cccc(-c4cccc5oc6cc(C7(c8ccccc8)c8ccccc8-c8ccccc87)ccc6c45)c3)cc2)cc1 ; c1ccc(N(c2ccccc2)c2ccc(-c3cccc(-c4cccc5sc6cc(C7(c8ccccc8)c8ccccc8-c8ccccc87)ccc6c45)c3)cc2)cc1 ; c1ccc(N(c2ccccc2)c2ccc(-c3cccc(-c4cccc5oc6cc(C7(c8ccccc8)c8ccccc8-c8ccccc87)ccc6c45)c3)cc2)cc1]

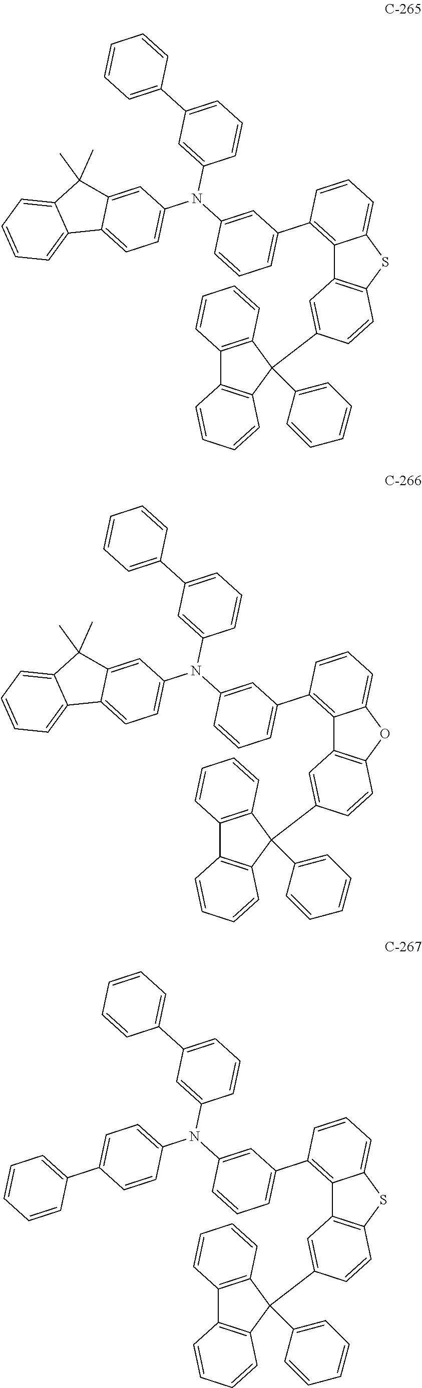 [SMILES: CC1(C)c2ccccc2-c2ccc(N(c3cccc(-c4ccccc4)c3)c3cccc(-c4cccc5sc6ccc(C7(c8ccccc8)c8ccccc8-c8ccccc87)cc6c45)c3)cc21 ; CC1(C)c2ccccc2-c2ccc(N(c3cccc(-c4ccccc4)c3)c3cccc(-c4cccc5oc6ccc(C7(c8ccccc8)c8ccccc8-c8ccccc87)cc6c45)c3)cc21 ; c1ccc(-c2ccc(N(c3cccc(-c4ccccc4)c3)c3cccc(-c4cccc5sc6ccc(C7(c8ccccc8)c8ccccc8-c8ccccc87)cc6c45)c3)cc2)cc1]

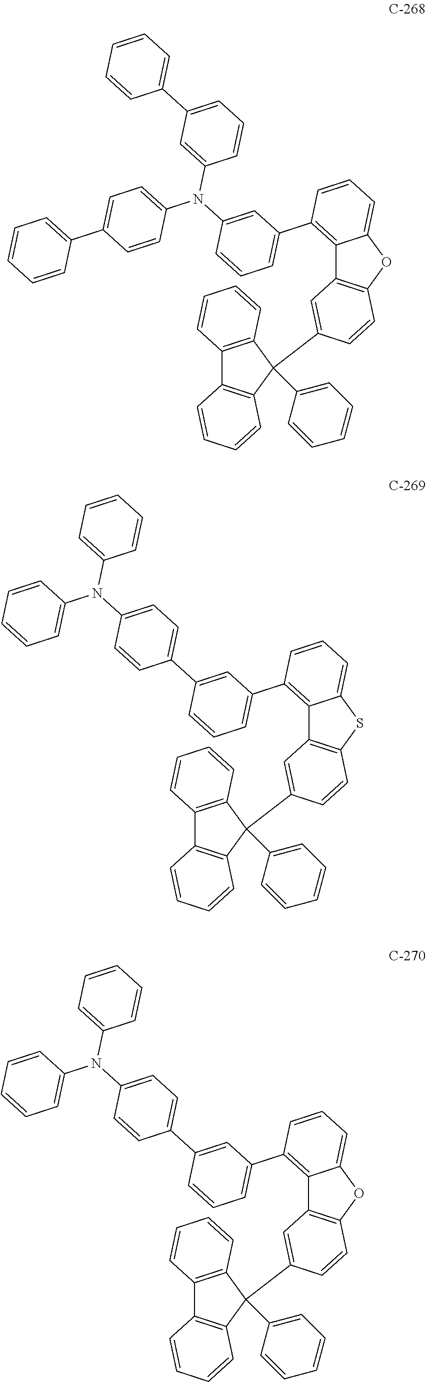 [SMILES: c1ccc(-c2ccc(N(c3cccc(-c4ccccc4)c3)c3cccc(-c4cccc5oc6ccc(C7(c8ccccc8)c8ccccc8-c8ccccc87)cc6c45)c3)cc2)cc1 ; c1ccc(N(c2ccccc2)c2ccc(-c3cccc(-c4cccc5sc6ccc(C7(c8ccccc8)c8ccccc8-c8ccccc87)cc6c45)c3)cc2)cc1 ; c1ccc(N(c2ccccc2)c2ccc(-c3cccc(-c4cccc5oc6ccc(C7(c8ccccc8)c8ccccc8-c8ccccc87)cc6c45)c3)cc2)cc1]

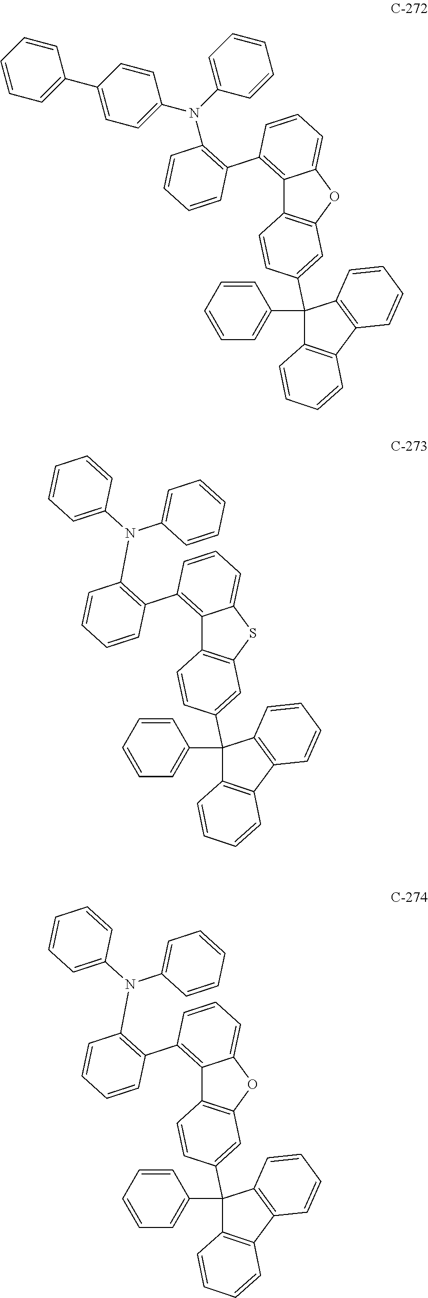 [SMILES: c1ccc(-c2ccc(N(c3ccccc3)c3ccccc3-c3cccc4oc5cc(C6(c7ccccc7)c7ccccc7-c7ccccc76)ccc5c34)cc2)cc1 ; c1ccc(N(c2ccccc2)c2ccccc2-c2cccc3sc4cc(C5(c6ccccc6)c6ccccc6-c6ccccc65)ccc4c23)cc1 ; c1ccc(N(c2ccccc2)c2ccccc2-c2cccc3oc4cc(C5(c6ccccc6)c6ccccc6-c6ccccc65)ccc4c23)cc1]

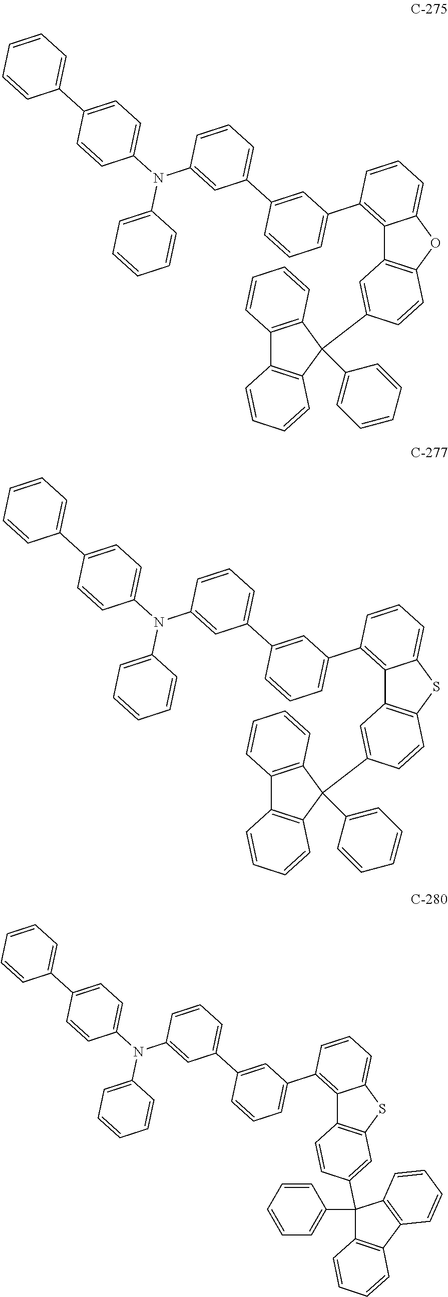 [SMILES: c1ccc(-c2ccc(N(c3ccccc3)c3cccc(-c4cccc(-c5cccc6oc7ccc(C8(c9ccccc9)c9ccccc9-c9ccccc98)cc7c56)c4)c3)cc2)cc1 ; c1ccc(-c2ccc(N(c3ccccc3)c3cccc(-c4cccc(-c5cccc6sc7ccc(C8(c9ccccc9)c9ccccc9-c9ccccc98)cc7c56)c4)c3)cc2)cc1 ; c1ccc(-c2ccc(N(c3ccccc3)c3cccc(-c4cccc(-c5cccc6sc7cc(C8(c9ccccc9)c9ccccc9-c9ccccc98)ccc7c56)c4)c3)cc2)cc1]

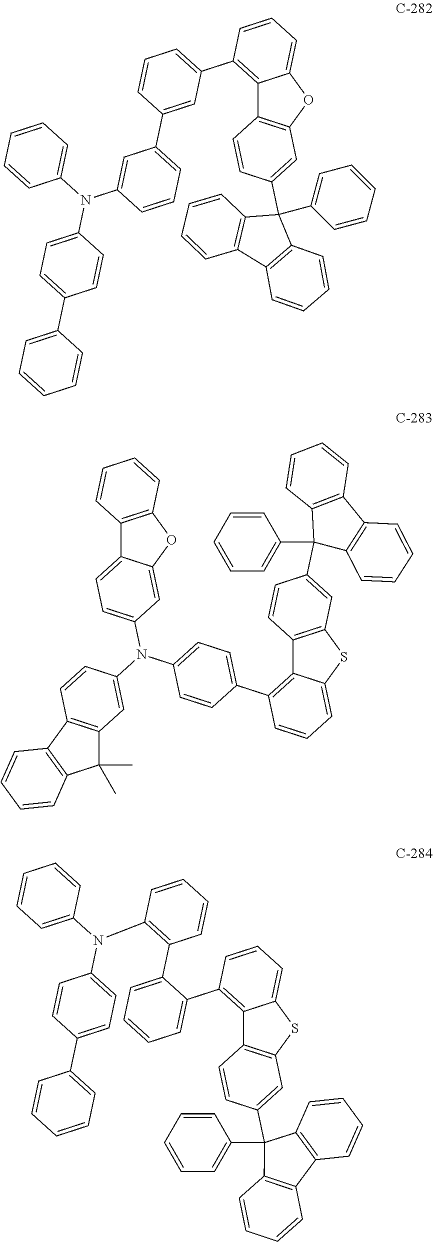 [SMILES: c1ccc(-c2ccc(N(c3ccccc3)c3cccc(-c4cccc(-c5cccc6oc7cc(C8(c9ccccc9)c9ccccc9-c9ccccc98)ccc7c56)c4)c3)cc2)cc1 ; CC1(C)c2ccccc2-c2ccc(N(c3ccc(-c4cccc5sc6cc(C7(c8ccccc8)c8ccccc8-c8ccccc87)ccc6c45)cc3)c3ccc4c(c3)oc3ccccc34)cc21 ; c1ccc(-c2ccc(N(c3ccccc3)c3ccccc3-c3ccccc3-c3cccc4sc5cc(C6(c7ccccc7)c7ccccc7-c7ccccc76)ccc5c34)cc2)cc1]

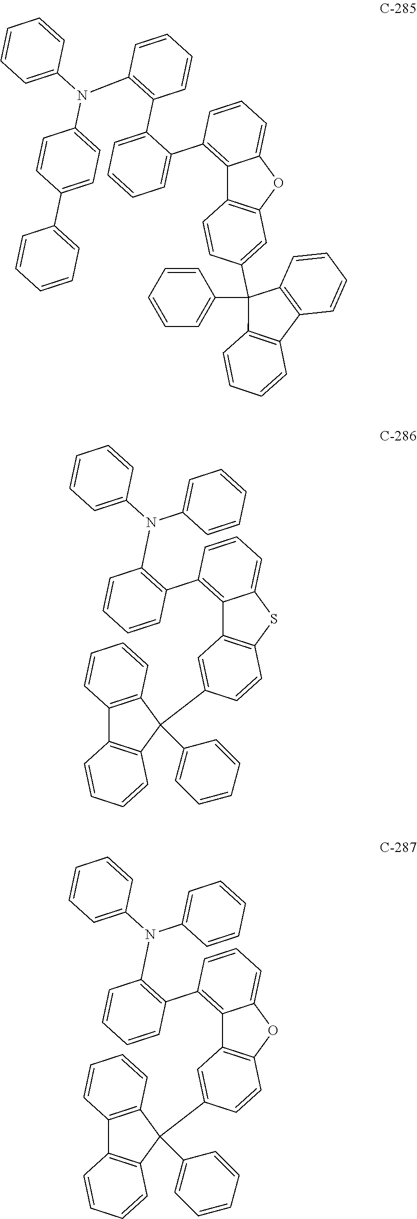 [SMILES: c1ccc(-c2ccc(N(c3ccccc3)c3ccccc3-c3ccccc3-c3cccc4oc5cc(C6(c7ccccc7)c7ccccc7-c7ccccc76)ccc5c34)cc2)cc1 ; c1ccc(N(c2ccccc2)c2ccccc2-c2cccc3sc4ccc(C5(c6ccccc6)c6ccccc6-c6ccccc65)cc4c23)cc1 ; c1ccc(N(c2ccccc2)c2ccccc2-c2cccc3oc4ccc(C5(c6ccccc6)c6ccccc6-c6ccccc65)cc4c23)cc1]

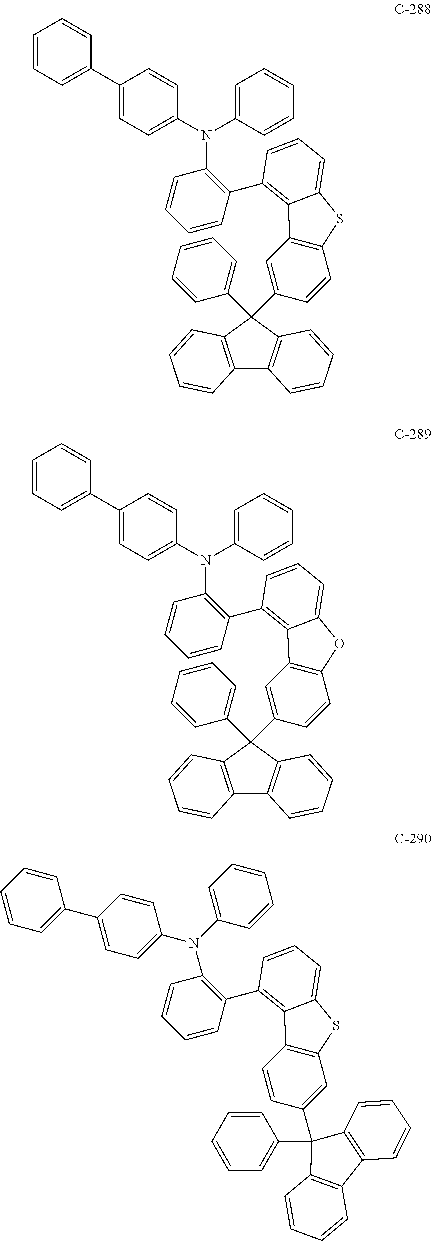 [SMILES: c1ccc(-c2ccc(N(c3ccccc3)c3ccccc3-c3cccc4sc5ccc(C6(c7ccccc7)c7ccccc7-c7ccccc76)cc5c34)cc2)cc1 ; c1ccc(-c2ccc(N(c3ccccc3)c3ccccc3-c3cccc4oc5ccc(C6(c7ccccc7)c7ccccc7-c7ccccc76)cc5c34)cc2)cc1 ; c1ccc(-c2ccc(N(c3ccccc3)c3ccccc3-c3cccc4sc5cc(C6(c7ccccc7)c7ccccc7-c7ccccc76)ccc5c34)cc2)cc1]

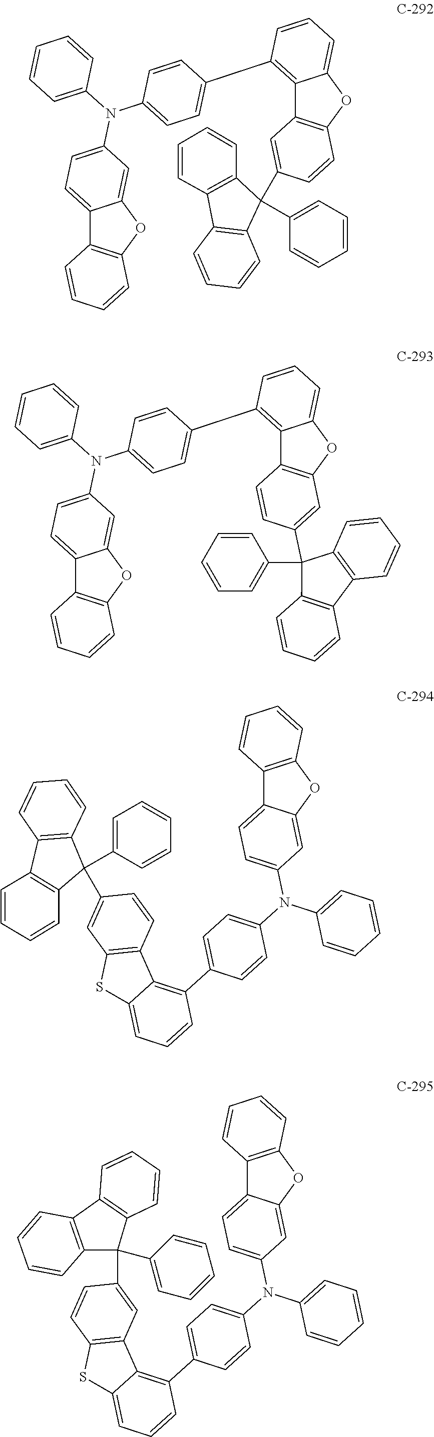 [SMILES: c1ccc(N(c2ccc(-c3cccc4oc5ccc(C6(c7ccccc7)c7ccccc7-c7ccccc76)cc5c34)cc2)c2ccc3c(c2)oc2ccccc23)cc1 ; c1ccc(N(c2ccc(-c3cccc4oc5cc(C6(c7ccccc7)c7ccccc7-c7ccccc76)ccc5c34)cc2)c2ccc3c(c2)oc2ccccc23)cc1 ; c1ccc(N(c2ccc(-c3cccc4sc5cc(C6(c7ccccc7)c7ccccc7-c7ccccc76)ccc5c34)cc2)c2ccc3c(c2)oc2ccccc23)cc1 ; c1ccc(N(c2ccc(-c3cccc4sc5ccc(C6(c7ccccc7)c7ccccc7-c7ccccc76)cc5c34)cc2)c2ccc3c(c2)oc2ccccc23)cc1]

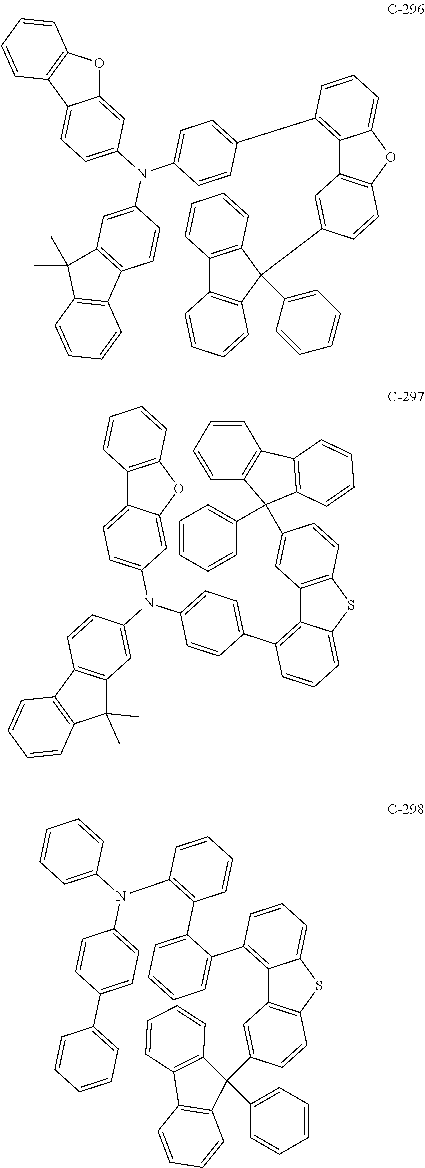 [SMILES: CC1(C)c2ccccc2-c2ccc(N(c3ccc(-c4cccc5oc6ccc(C7(c8ccccc8)c8ccccc8-c8ccccc87)cc6c45)cc3)c3ccc4c(c3)oc3ccccc34)cc21 ; CC1(C)c2ccccc2-c2ccc(N(c3ccc(-c4cccc5sc6ccc(C7(c8ccccc8)c8ccccc8-c8ccccc87)cc6c45)cc3)c3ccc4c(c3)oc3ccccc34)cc21 ; c1ccc(-c2ccc(N(c3ccccc3)c3ccccc3-c3ccccc3-c3cccc4sc5ccc(C6(c7ccccc7)c7ccccc7-c7ccccc76)cc5c34)cc2)cc1]

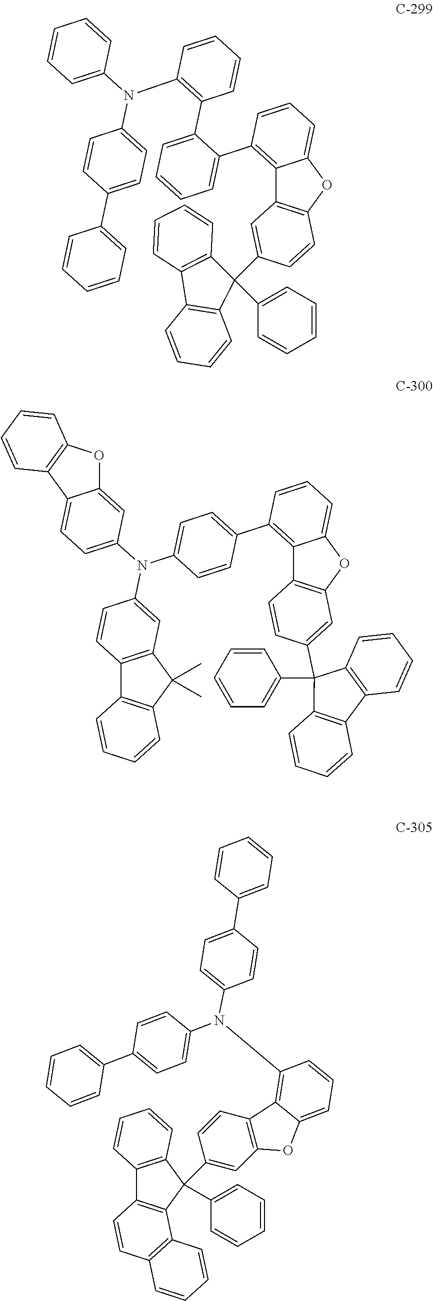 [SMILES: c1ccc(-c2ccc(N(c3ccccc3)c3ccccc3-c3ccccc3-c3cccc4oc5ccc(C6(c7ccccc7)c7ccccc7-c7ccccc76)cc5c34)cc2)cc1 ; CC1(C)c2ccccc2-c2ccc(N(c3ccc(-c4cccc5oc6cc(C7(c8ccccc8)c8ccccc8-c8ccccc87)ccc6c45)cc3)c3ccc4c(c3)oc3ccccc34)cc21 ; c1ccc(-c2ccc(N(c3ccc(-c4ccccc4)cc3)c3cccc4oc5cc(C6(c7ccccc7)c7ccccc7-c7ccc8ccccc8c76)ccc5c34)cc2)cc1]

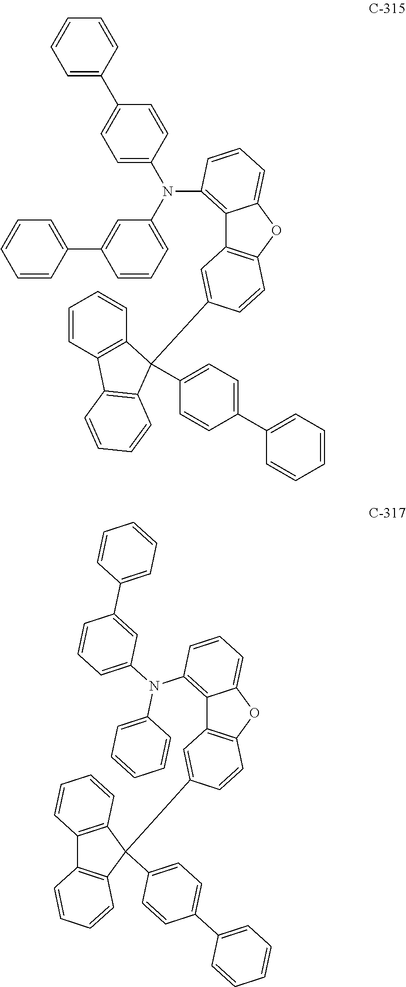 [SMILES: c1ccc(-c2ccc(N(c3cccc(-c4ccccc4)c3)c3cccc4oc5ccc(C6(c7ccc(-c8ccccc8)cc7)c7ccccc7-c7ccccc76)cc5c34)cc2)cc1 ; c1ccc(-c2ccc(C3(c4ccc5oc6cccc(N(c7ccccc7)c7cccc(-c8ccccc8)c7)c6c5c4)c4ccccc4-c4ccccc43)cc2)cc1]

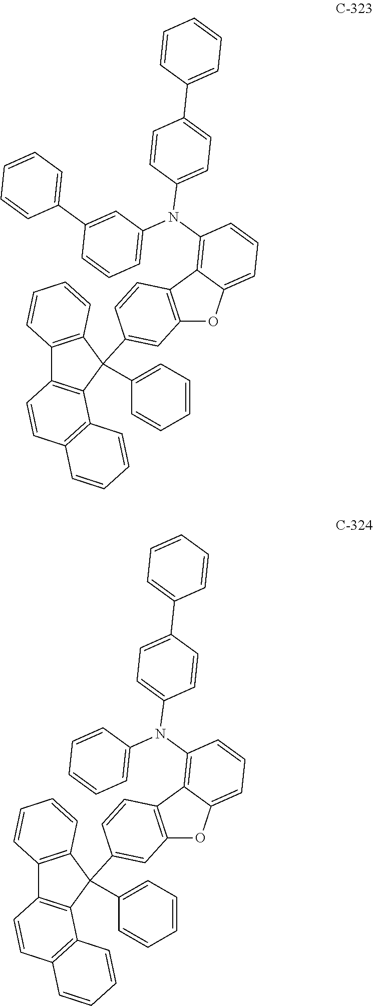 [SMILES: c1ccc(-c2ccc(N(c3cccc(-c4ccccc4)c3)c3cccc4oc5cc(C6(c7ccccc7)c7ccccc7-c7ccc8ccccc8c76)ccc5c34)cc2)cc1 ; c1ccc(-c2ccc(N(c3ccccc3)c3cccc4oc5cc(C6(c7ccccc7)c7ccccc7-c7ccc8ccccc8c76)ccc5c34)cc2)cc1]

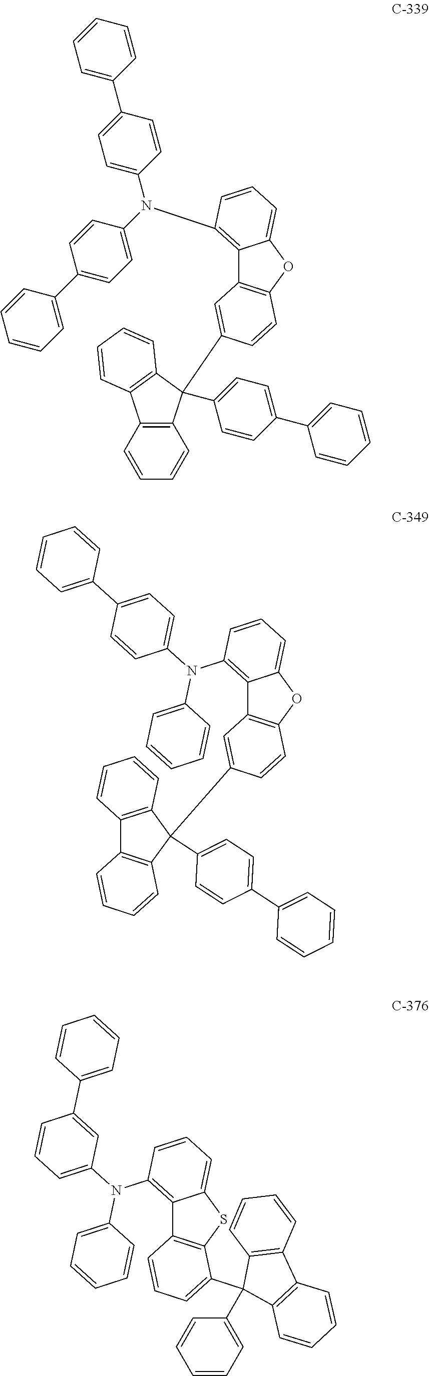 [SMILES: c1ccc(-c2ccc(N(c3ccc(-c4ccccc4)cc3)c3cccc4oc5ccc(C6(c7ccc(-c8ccccc8)cc7)c7ccccc7-c7ccccc76)cc5c34)cc2)cc1 ; c1ccc(-c2ccc(N(c3ccccc3)c3cccc4oc5ccc(C6(c7ccc(-c8ccccc8)cc7)c7ccccc7-c7ccccc76)cc5c34)cc2)cc1 ; c1ccc(-c2cccc(N(c3ccccc3)c3cccc4sc5c(C6(c7ccccc7)c7ccccc7-c7ccccc76)cccc5c34)c2)cc1]

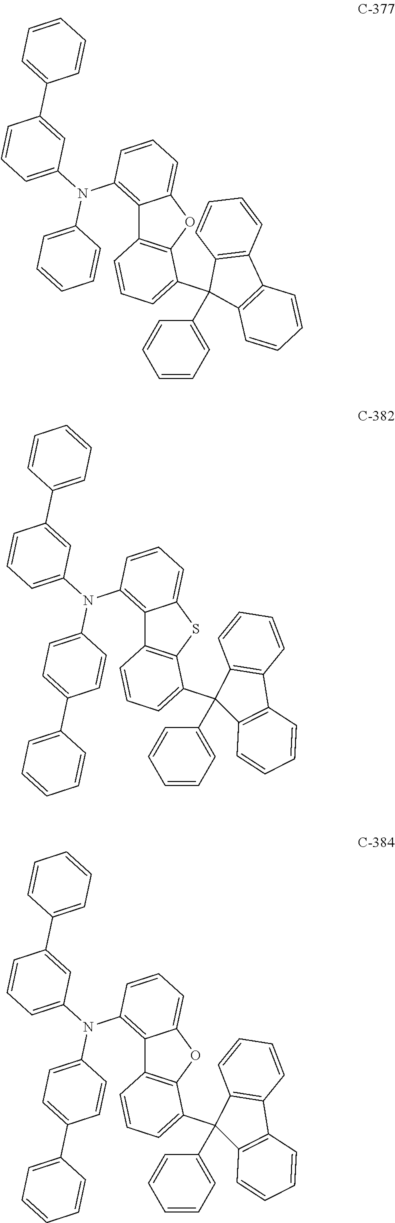 [SMILES: c1ccc(-c2cccc(N(c3ccccc3)c3cccc4oc5c(C6(c7ccccc7)c7ccccc7-c7ccccc76)cccc5c34)c2)cc1 ; c1ccc(-c2ccc(N(c3cccc(-c4ccccc4)c3)c3cccc4sc5c(C6(c7ccccc7)c7ccccc7-c7ccccc76)cccc5c34)cc2)cc1 ; c1ccc(-c2ccc(N(c3cccc(-c4ccccc4)c3)c3cccc4oc5c(C6(c7ccccc7)c7ccccc7-c7ccccc76)cccc5c34)cc2)cc1]

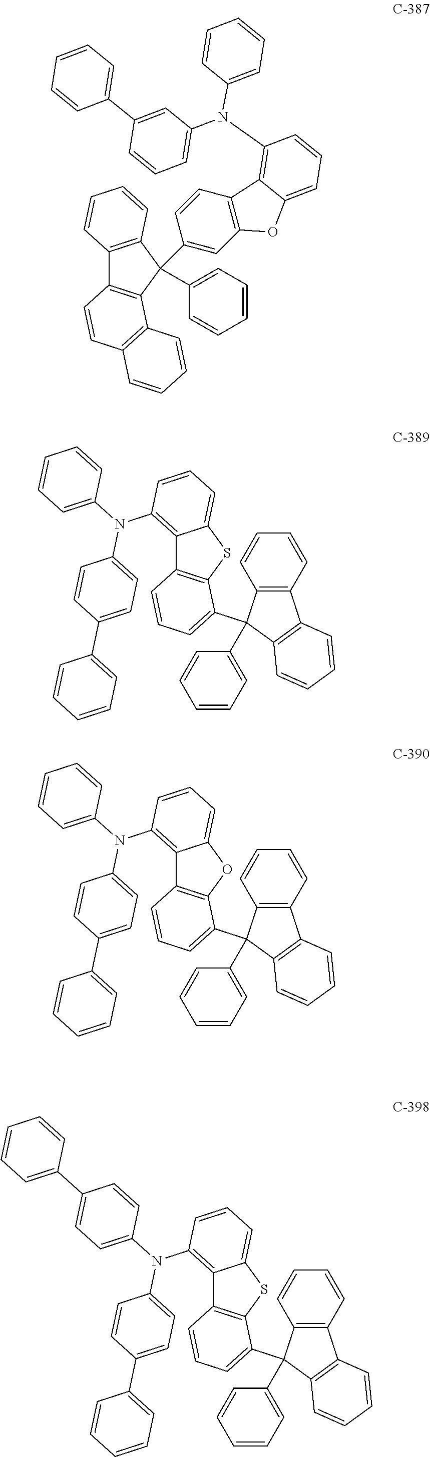

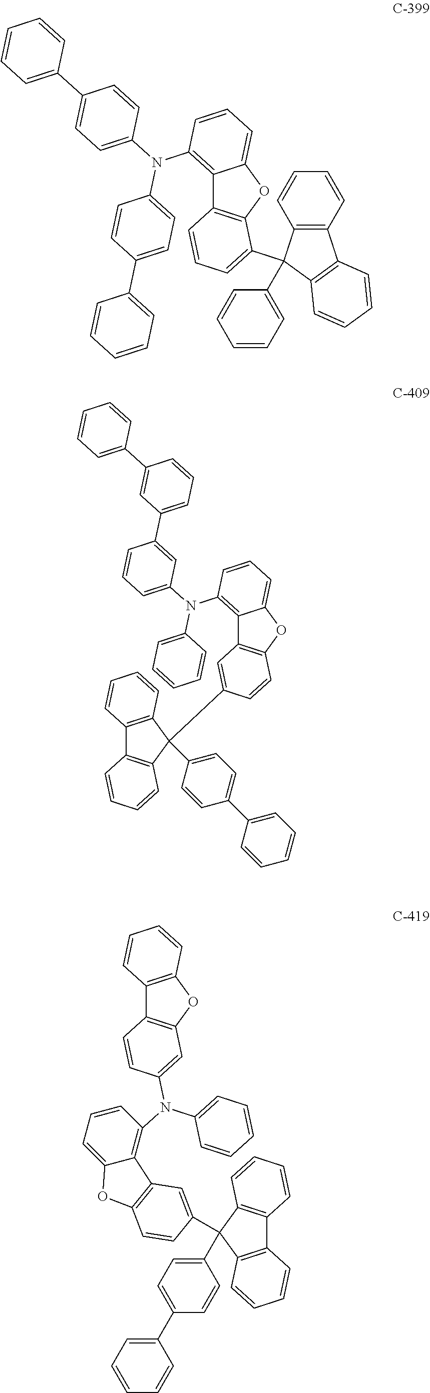 [SMILES: c1ccc(-c2ccc(N(c3ccc(-c4ccccc4)cc3)c3cccc4oc5c(C6(c7ccccc7)c7ccccc7-c7ccccc76)cccc5c34)cc2)cc1 ; c1ccc(-c2ccc(C3(c4ccc5oc6cccc(N(c7ccccc7)c7cccc(-c8cccc(-c9ccccc9)c8)c7)c6c5c4)c4ccccc4-c4ccccc43)cc2)cc1 ; c1ccc(-c2ccc(C3(c4ccc5oc6cccc(N(c7ccccc7)c7ccc8c(c7)oc7ccccc78)c6c5c4)c4ccccc4-c4ccccc43)cc2)cc1]

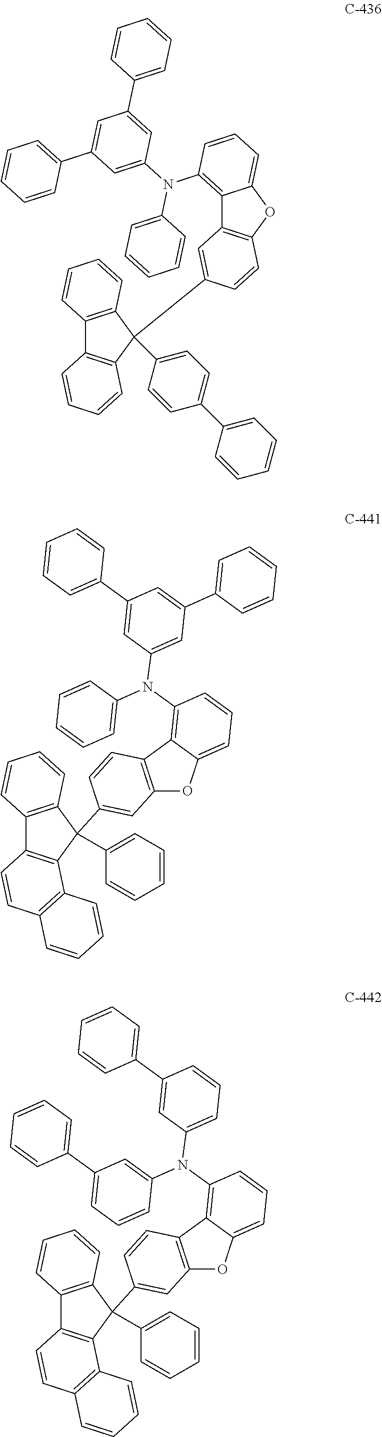 [SMILES: c1ccc(-c2ccc(C3(c4ccc5oc6cccc(N(c7ccccc7)c7cc(-c8ccccc8)cc(-c8ccccc8)c7)c6c5c4)c4ccccc4-c4ccccc43)cc2)cc1 ; c1ccc(-c2cc(-c3ccccc3)cc(N(c3ccccc3)c3cccc4oc5cc(C6(c7ccccc7)c7ccccc7-c7ccc8ccccc8c76)ccc5c34)c2)cc1 ; c1ccc(-c2cccc(N(c3cccc(-c4ccccc4)c3)c3cccc4oc5cc(C6(c7ccccc7)c7ccccc7-c7ccc8ccccc8c76)ccc5c34)c2)cc1]

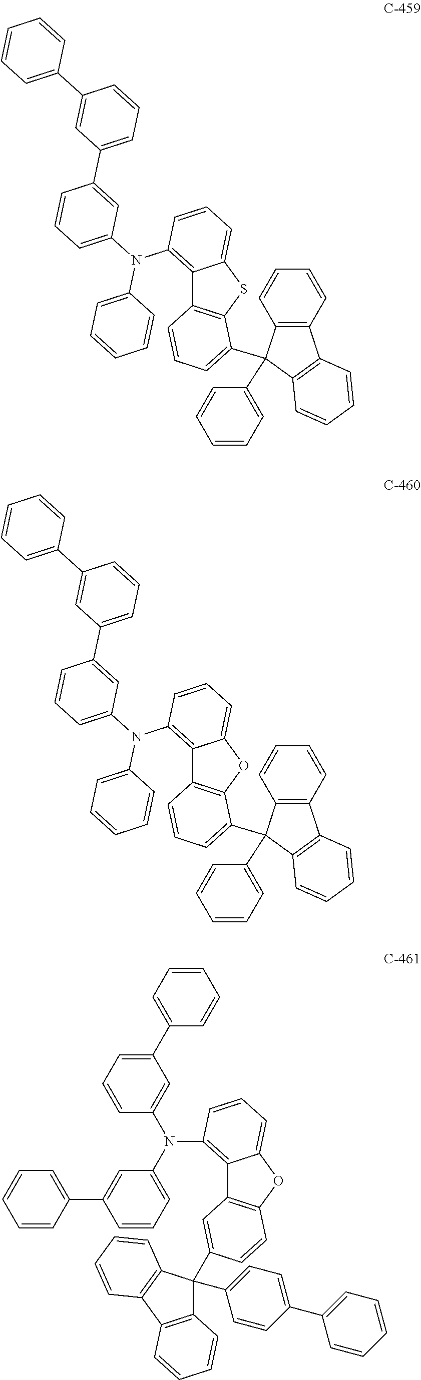 [SMILES: c1ccc(-c2cccc(-c3cccc(N(c4ccccc4)c4cccc5sc6c(C7(c8ccccc8)c8ccccc8-c8ccccc87)cccc6c45)c3)c2)cc1 ; c1ccc(-c2cccc(-c3cccc(N(c4ccccc4)c4cccc5oc6c(C7(c8ccccc8)c8ccccc8-c8ccccc87)cccc6c45)c3)c2)cc1 ; c1ccc(-c2ccc(C3(c4ccc5oc6cccc(N(c7cccc(-c8ccccc8)c7)c7cccc(-c8ccccc8)c7)c6c5c4)c4ccccc4-c4ccccc43)cc2)cc1]

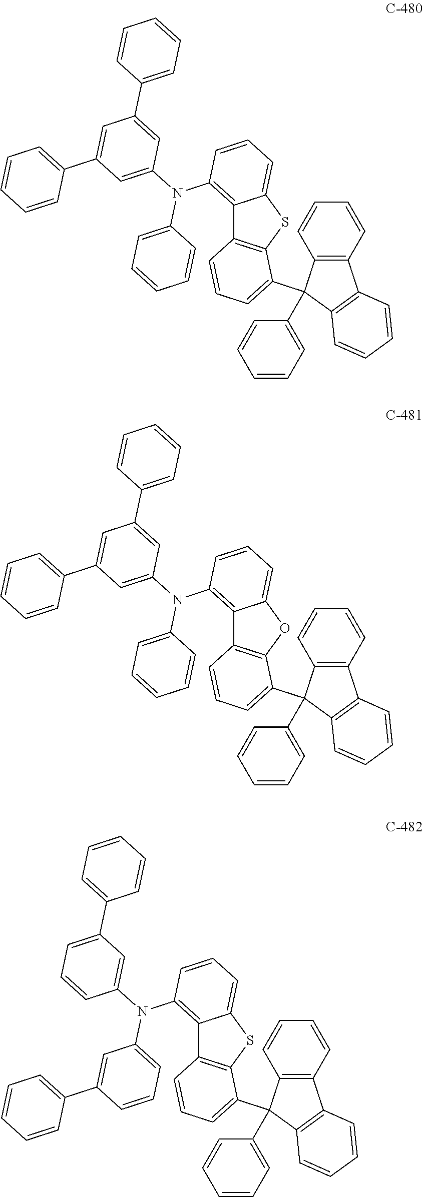 [SMILES: c1ccc(-c2cc(-c3ccccc3)cc(N(c3ccccc3)c3cccc4sc5c(C6(c7ccccc7)c7ccccc7-c7ccccc76)cccc5c34)c2)cc1 ; c1ccc(-c2cc(-c3ccccc3)cc(N(c3ccccc3)c3cccc4oc5c(C6(c7ccccc7)c7ccccc7-c7ccccc76)cccc5c34)c2)cc1 ; c1ccc(-c2cccc(N(c3cccc(-c4ccccc4)c3)c3cccc4sc5c(C6(c7ccccc7)c7ccccc7-c7ccccc76)cccc5c34)c2)cc1]

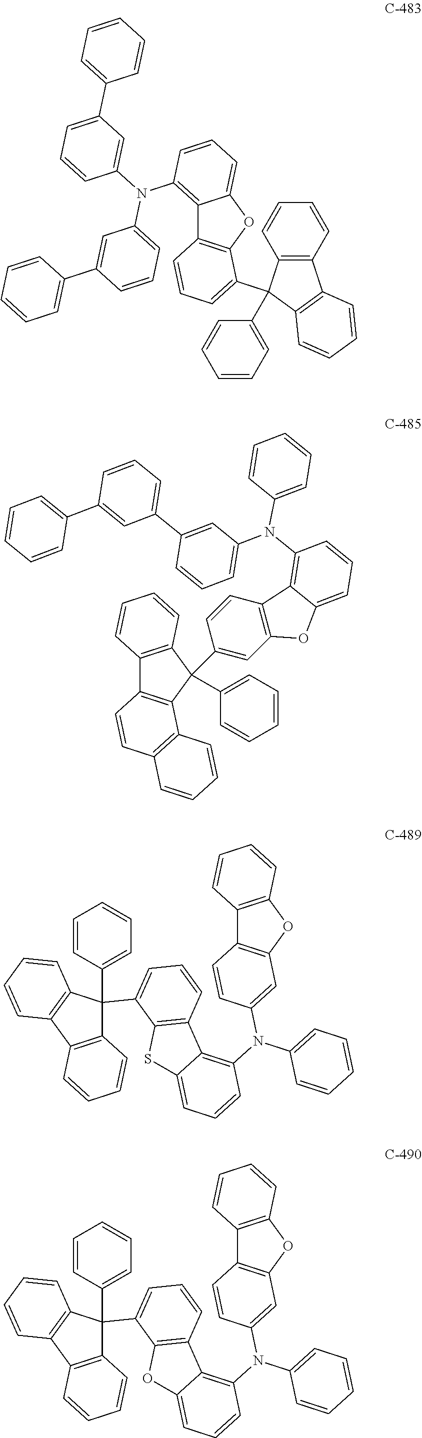 [SMILES: c1ccc(-c2cccc(N(c3cccc(-c4ccccc4)c3)c3cccc4oc5c(C6(c7ccccc7)c7ccccc7-c7ccccc76)cccc5c34)c2)cc1 ; c1ccc(-c2cccc(-c3cccc(N(c4ccccc4)c4cccc5oc6cc(C7(c8ccccc8)c8ccccc8-c8ccc9ccccc9c87)ccc6c45)c3)c2)cc1 ; c1ccc(N(c2ccc3c(c2)oc2ccccc23)c2cccc3sc4c(C5(c6ccccc6)c6ccccc6-c6ccccc65)cccc4c23)cc1 ; c1ccc(N(c2ccc3c(c2)oc2ccccc23)c2cccc3oc4c(C5(c6ccccc6)c6ccccc6-c6ccccc65)cccc4c23)cc1]

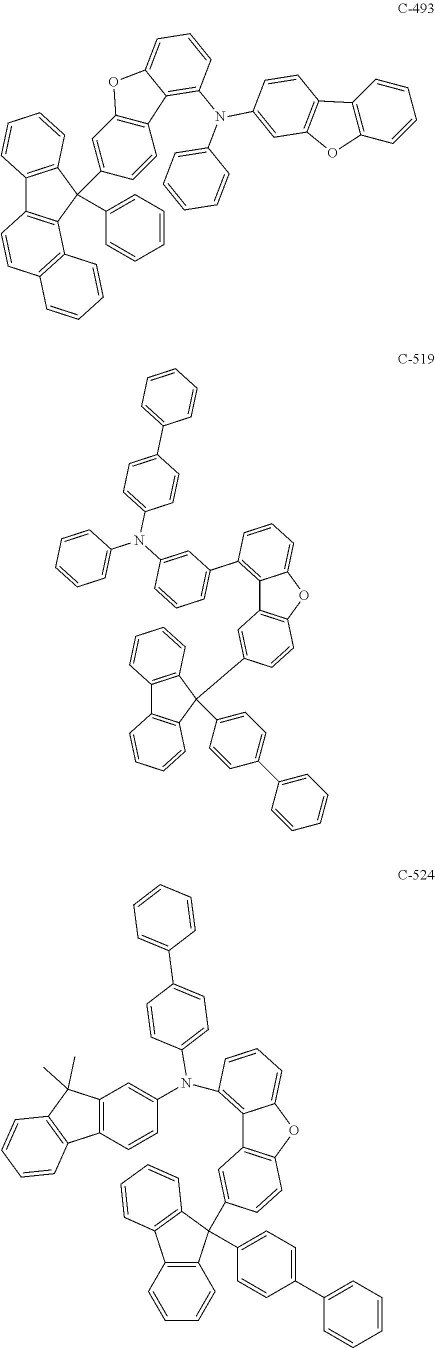 [SMILES: c1ccc(N(c2ccc3c(c2)oc2ccccc23)c2cccc3oc4cc(C5(c6ccccc6)c6ccccc6-c6ccc7ccccc7c65)ccc4c23)cc1 ; c1ccc(-c2ccc(N(c3ccccc3)c3cccc(-c4cccc5oc6ccc(C7(c8ccc(-c9ccccc9)cc8)c8ccccc8-c8ccccc87)cc6c45)c3)cc2)cc1 ; CC1(C)c2ccccc2-c2ccc(N(c3ccc(-c4ccccc4)cc3)c3cccc4oc5ccc(C6(c7ccc(-c8ccccc8)cc7)c7ccccc7-c7ccccc76)cc5c34)cc21]

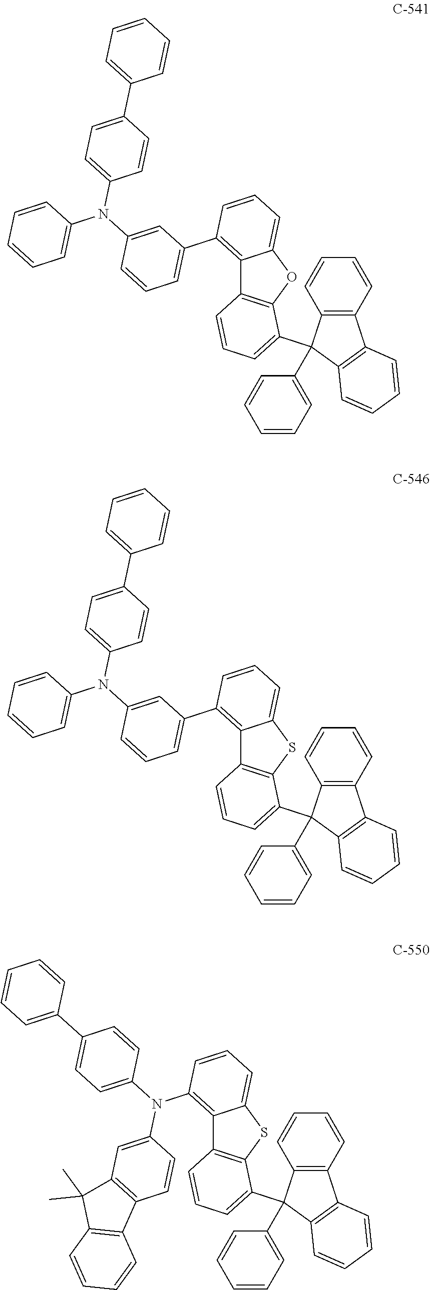 [SMILES: c1ccc(-c2ccc(N(c3ccccc3)c3cccc(-c4cccc5oc6c(C7(c8ccccc8)c8ccccc8-c8ccccc87)cccc6c45)c3)cc2)cc1 ; c1ccc(-c2ccc(N(c3ccccc3)c3cccc(-c4cccc5sc6c(C7(c8ccccc8)c8ccccc8-c8ccccc87)cccc6c45)c3)cc2)cc1 ; CC1(C)c2ccccc2-c2ccc(N(c3ccc(-c4ccccc4)cc3)c3cccc4sc5c(C6(c7ccccc7)c7ccccc7-c7ccccc76)cccc5c34)cc21]

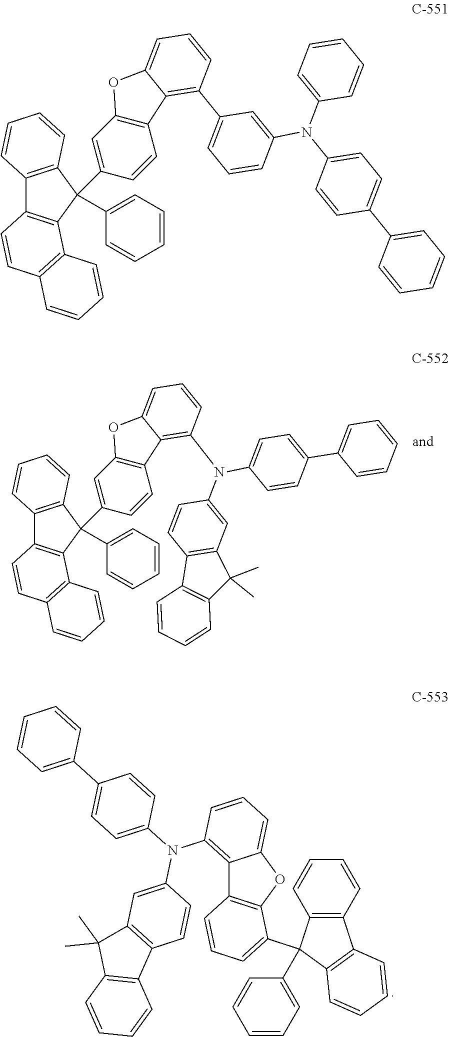 [SMILES: c1ccc(-c2ccc(N(c3ccccc3)c3cccc(-c4cccc5oc6cc(C7(c8ccccc8)c8ccccc8-c8ccc9ccccc9c87)ccc6c45)c3)cc2)cc1 ; CC1(C)c2ccccc2-c2ccc(N(c3ccc(-c4ccccc4)cc3)c3cccc4oc5cc(C6(c7ccccc7)c7ccccc7-c7ccc8ccccc8c76)ccc5c34)cc21 ; CC1(C)c2ccccc2-c2ccc(N(c3ccc(-c4ccccc4)cc3)c3cccc4oc5c(C6(c7ccccc7)c7ccccc7-c7ccccc76)cccc5c34)cc21]

4. An organic electroluminescent material comprising the organic electroluminescent compound according to claim 1.

5. An organic electroluminescent device comprising the organic electroluminescent compound according to claim 1.

6. The organic electroluminescent device according to claim 5, wherein the organic electroluminescent compound is comprised in a hole transport zone.

7. The organic electroluminescent device according to claim 6, wherein the hole transport zone comprises at least one of a p-doped hole injection layer, a hole transport layer, and a light-emitting auxiliary layer.
